(19) United States
(12) Patent Application Publication
(10) Pub. No.: US 2024/0285786 A1
(43) Pub. Date: Aug. 29, 2024

(54) PROTEIN-DRUG CONJUGATES FOR ANTIVIRAL THERAPY

(71) Applicant: Cidara Therapeutics, Inc., San Diego, CA (US)

(72) Inventor: Leslie W. TARI, San Diego, CA (US)

(21) Appl. No.: 18/281,205

(22) PCT Filed: Mar. 11, 2022
(86) PCT No.: PCT/US2022/019974

§ 371 Date: Sep. 8, 2023

Related U.S. Application Data
(60) Provisional application No. 63/159,781, filed on Mar. 11, 2021.

(51) Int. Cl. A61K 47/68

(52) U.S. Cl. CPC A61K 47/6803

(57) ABSTRACT

Compositions and methods for the treatment of viral infections include conjugates containing inhibitors of viral neuraminidase (e.g., zanamivir or an analog thereof) linked to an Fc domain monomer. In particular, conjugates can be used in the treatment of influenza infections.

### SEQUENCE STATEMENT

The instant application contains a Sequence Listing which has been submitted electronically in ASCII format and is hereby incorporated by reference in its entirety. Said ASCII copy, created on Mar. 10, 2022, is named 50945-073WO2_Sequence_Listing_3_10_22_ST25 and is 71,257 bytes in size.

### BACKGROUND

The need for novel antiviral treatments for influenza is significant and especially critical in the medical field. Influenza virus, the causative agent of influenza, or the flu, is responsible for three to five million cases of severe illness annually, and approximately 500,000 deaths worldwide. While most people recover completely from influenza in about one to two weeks, others develop life-threatening complications, such as pneumonia. Thus, influenza can be deadly, especially for the young, old, or chronically ill. People with weak or compromised immune systems, such as people with advanced HIV infection or transplant patients, whose immune systems are medically suppressed to prevent transplant organ rejection, are at greater risk for complications relating to influenza. Pregnant women and young children are also at a high risk for complications.

The development of antiviral treatments for influenza has been a continuing challenge. Several influenza antiviral agents have been approved for use in the clinic, and these agents play important roles in modulating disease severity and controlling pandemics while vaccines are prepared. However, drug-resistant strains have emerged to the most commonly used inhibitors.

Influenza antiviral agents largely target proteins presented on the surface of the influenza virus particle. The envelope of the influenza virus contains two immunodominant glycoproteins, hemagglutinin and neuraminidase, that play key roles in viral infection and spread. Hemagglutinin effects attachment of the virus to the host cell through its interaction with surface sialic acids, thereby initiating entry. Neuraminidase is an exo-glycosidase enzyme that cleaves sialic acids (terminal neuraminic acid residues) from glycan structures on the surface of infected host cells, releasing progeny viruses and allowing the spread of the virus from the host cell to uninfected surrounding cells. Inhibition of neuraminidase therefore serves as a pharmacological target for antiviral drugs. Viral neuraminidase inhibitors used to reduce viral spread have been identified, including oseltamivir (Tamiflu™), zanamivir (Relenza™), and peramivir (Rapivab™).

However, influenza in transplant recipients remains characterized by prolonged viral shedding, increasing the likelihood of developing drug resistant strains. New, more effective therapies for treating influenza are needed.

### SUMMARY

The disclosure relates to conjugates, compositions, methods for inhibiting viral growth, methods for the treatment of viral infections, and methods of synthesizing conjugates. In particular, such conjugates contain dimers of a moiety that inhibits influenza virus neuraminidase (e.g., zanamivir or an analog thereof) conjugated to an Fc monomer or Fc domain. The neuraminidase inhibitor (e.g., zanamivir or an analog thereof) in the conjugates targets neuraminidase on the surface of the viral particle. The Fc monomers or Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of viral particles by immune cells and further enhancing the antiviral activity of the conjugates.

Fc domain monomers in conjugates of the present disclosure may include one or more mutations that contribute to increased half-life and/or efficacy. The one or more mutations may promote or maintain interaction of the Fc domain monomer with an Fc receptor, e.g., the neonatal Fc receptor (FcRn). For example, when an Fc domain monomer is conjugated to one or more therapeutic molecules, the conjugation may interfere with the interaction of the Fc domain monomer with the FcRn. FcRn binding is desirable as it is associated with increased half-life. Fc domain monomer variants described promote small molecule conjugation to amino acid sites of the Fc, where the conjugation of a small molecule to the Fc minimizes the disruption to FcRn binding. In some embodiments, the mutation masks a conjugation site on the Fc domain monomer such that conjugation of a small molecule does not occur at a site that would interfere with interaction with an Fc receptor

An Fc domain monomer described herein may be conjugated to a antiviral agent such as zanamivir or an analog thereof. Zanamivir or an analog thereof may be conjugated to one or more lysine residues of the Fc domain monomer. Mutation of a lysine residue (e.g., K246) to an amino acid residue other than lysine can prevent conjugation of zanamivir or an analog thereof to that position. Where a lysine is in proximity to one or more amino acid residues of the Fc domain monomer that mediate a function (e.g., binding to an Fc receptor), then it may be desirable to prevent conjugation of zanamivir or an analog thereof to the lysine residue in order to prevent disruption of the function of the Fc domain monomer.

Such compositions are useful in methods for the inhibition of viral growth and in methods for the treatment of viral infections, such as those caused by an influenza virus A, influenza virus B and influenza virus C.

In a first aspect, the disclosure features a conjugate described by formula (D-I):

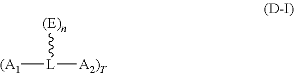

wherein A₁ and A₂ are each, independently, zanamivir or an analog thereof; L is a linker; E is an Fc domain monomer including an amino acid substitution at position 246, wherein the amino acid at position 246 is not a lysine, and wherein numbering is according to the EU index as in Kabat; n is 1 or 2; T is an integer from 1 to 20; and the squiggly line indicates that L is covalently attached to E, or a pharmaceutically acceptable salt thereof.

In some embodiments, each A₁ and each A₂ is independently selected from any one of formulas (A-I)-(A-VII):

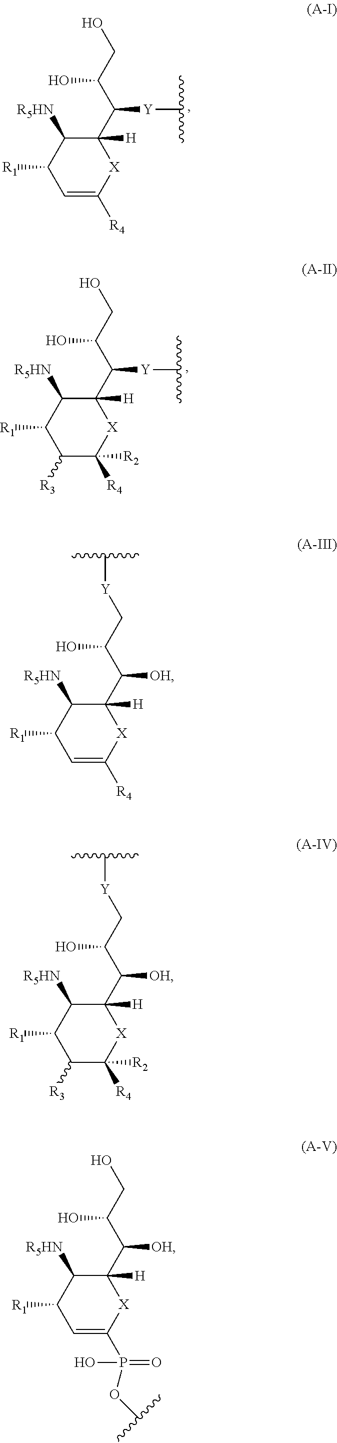

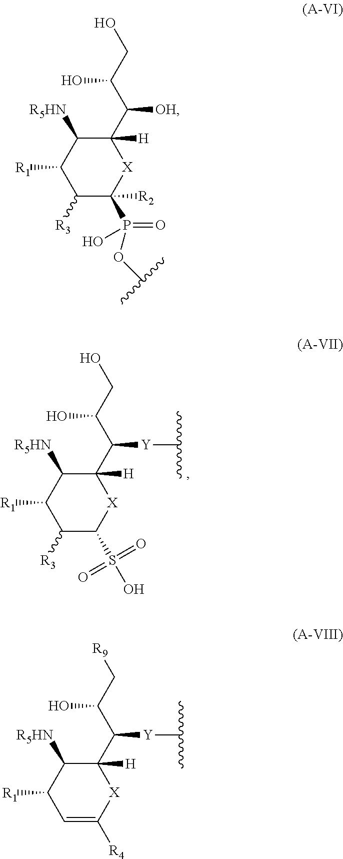

wherein R₁ is selected from —OH, —NH₂, —NHC(═NH)NH₂, and —NHC(═NH)NHR₆; R₂ and R₃ are each independently selected from —H, —OH, —F, —Cl, and —Br; R₄ is selected from —CO₂H, —P(═O)(OH)₂, —SO₃H; R₅ is selected from —COCH₃, —COCF₃, —SO₂CH₃; X is selected from —O— and —S—; Y is selected from

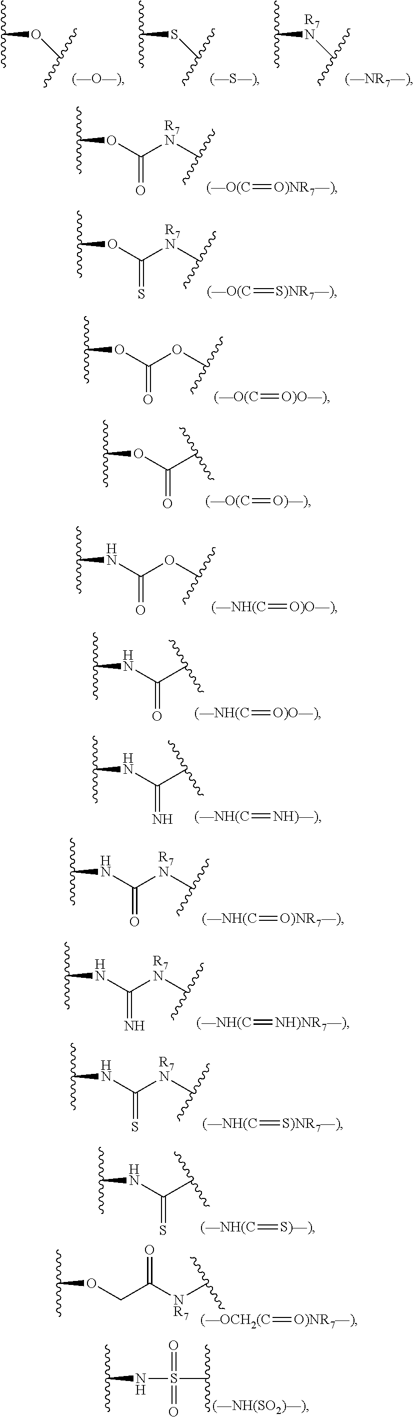

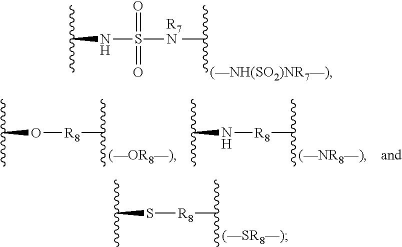

R₆ is selected from

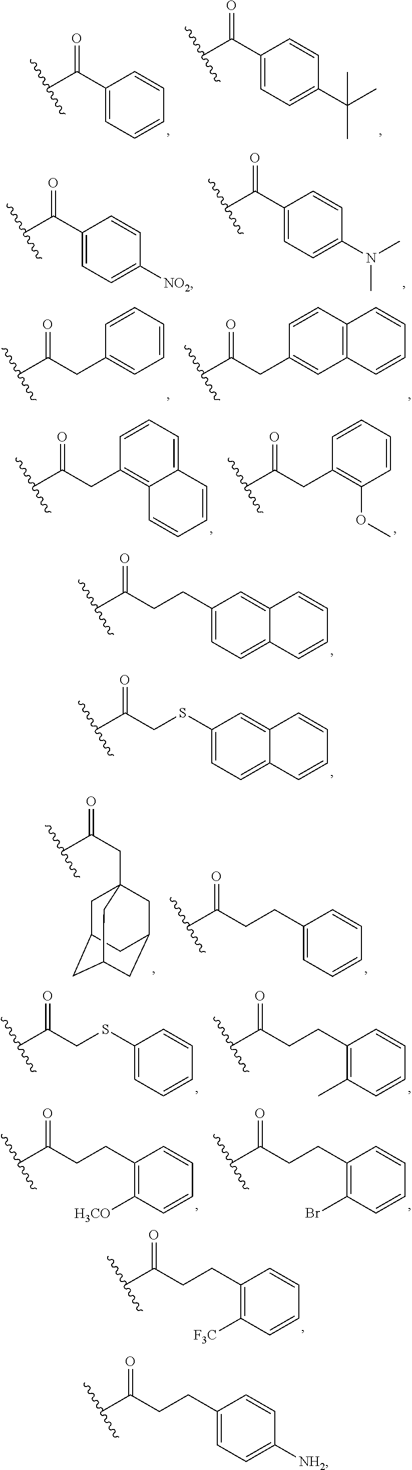

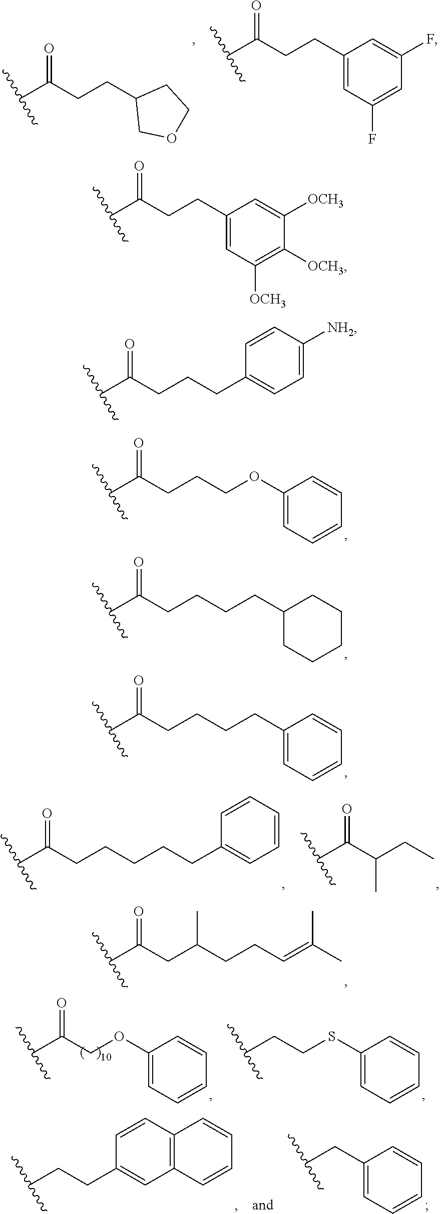

R₇ is selected from H, C1-C20 alkyl, C3-C20 cycloalkyl, C3-C20 heterocycloalkyl; C5-C15 aryl, and C2-C15 heteroaryl;
R₈ is selected from C3-C20 heterocycloalkyl, C5-C15 aryl, and C2-C15 heteroaryl;
R₉ is selected from —H, a halogen (e.g., Cl or F), —OR₁₀, —NHC(═O)R₇, optionally substituted C1-C20 alkyl, C3-C20 cycloalkyl, C3-C20 heterocycloalkyl; C5-C15 aryl, and C2-C15 heteroaryl; and
R₁₀ is selected from C1-C20 alkyl, C3-C20 cycloalkyl, C3-C20 heterocycloalkyl; C5-C15 aryl, and C2-C15 heteroaryl.

In some embodiments, the conjugate is described by formula (D-II):

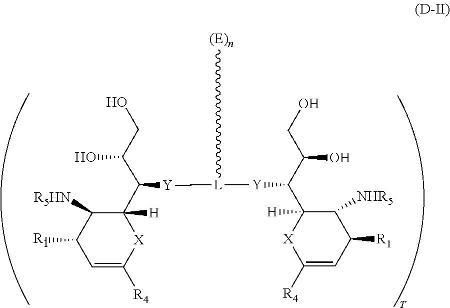

or a pharmaceutically acceptable salt thereof.

In some embodiments, the conjugate is described by formula (D-II-1):

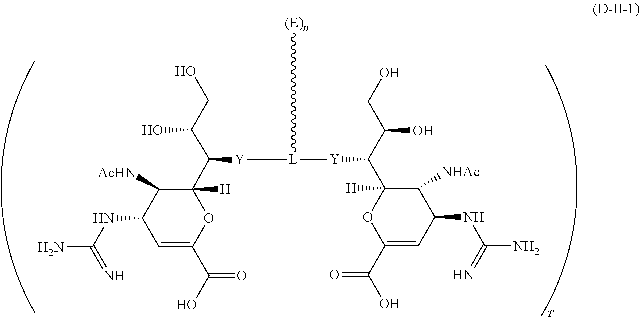

or a pharmaceutically acceptable salt thereof.

In some embodiments, the conjugate is described by formula (D-II-2):

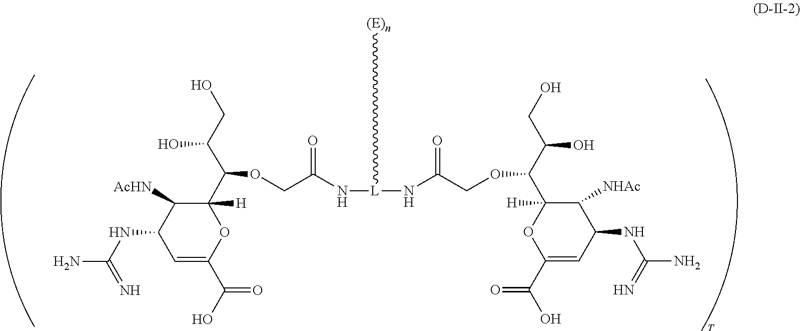

or a pharmaceutically acceptable salt thereof.

In some embodiments, the conjugate is described by formula (D-II-3):

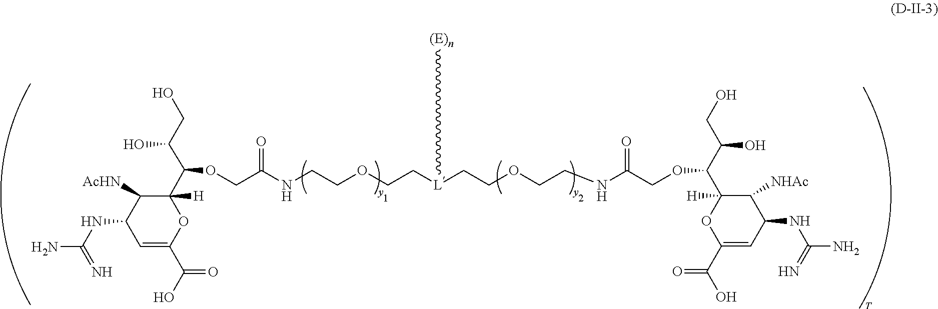

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure selected from:

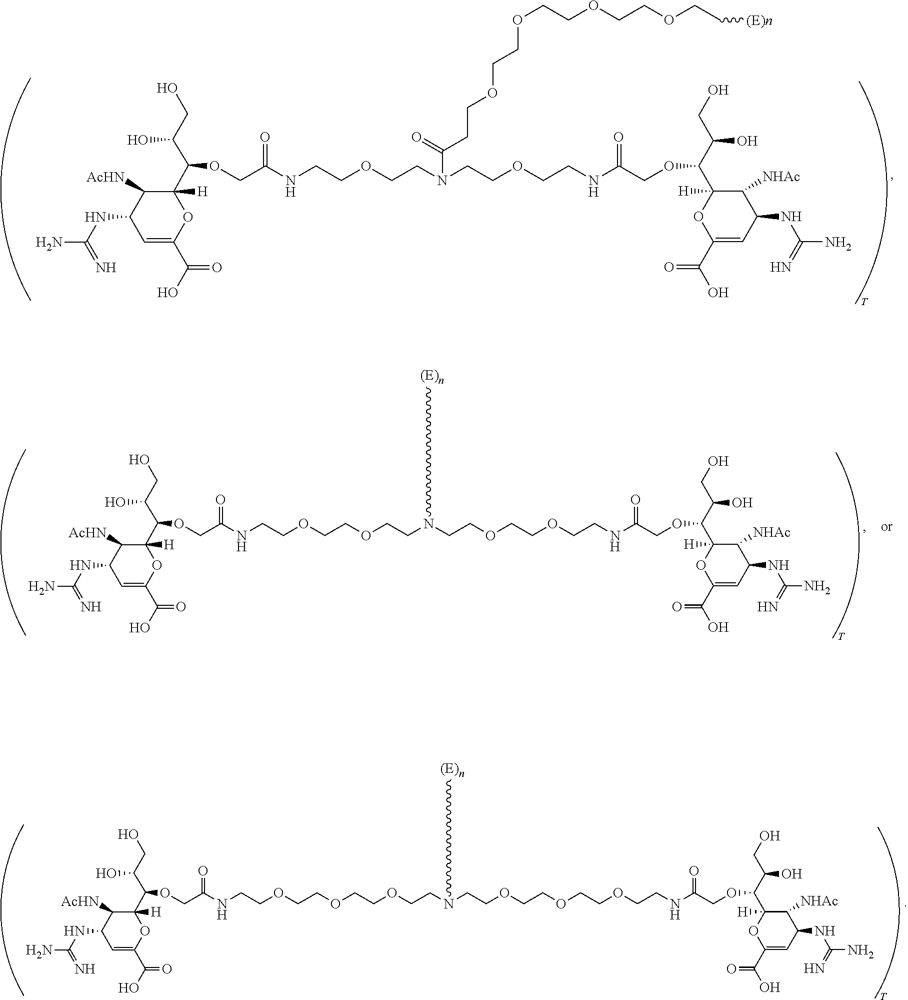

In some embodiments, the conjugate is described by formula (D-II-4):

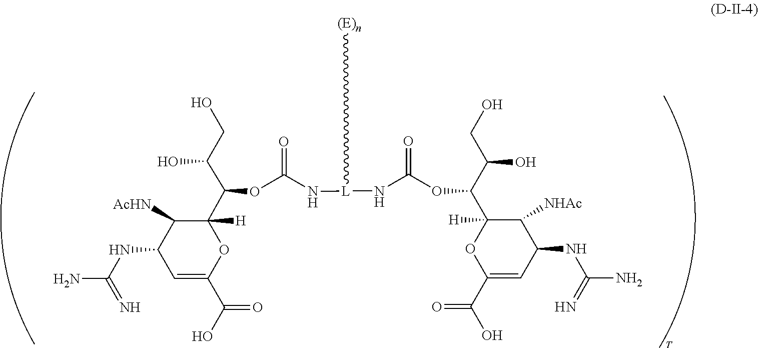

or a pharmaceutically acceptable salt thereof.

In some embodiments, the conjugate is described by formula (D-II-5):

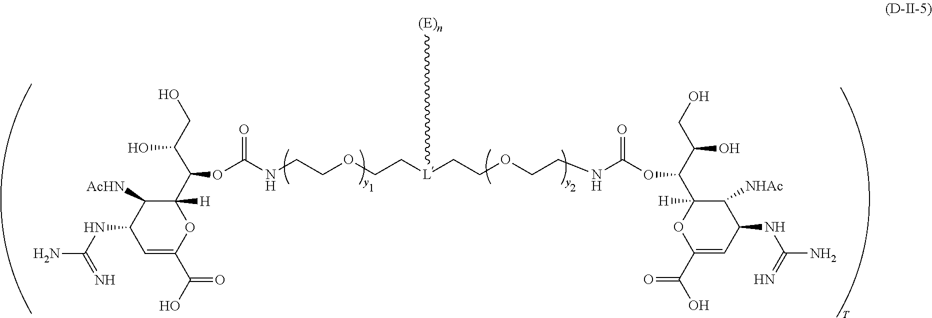

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure selected from:

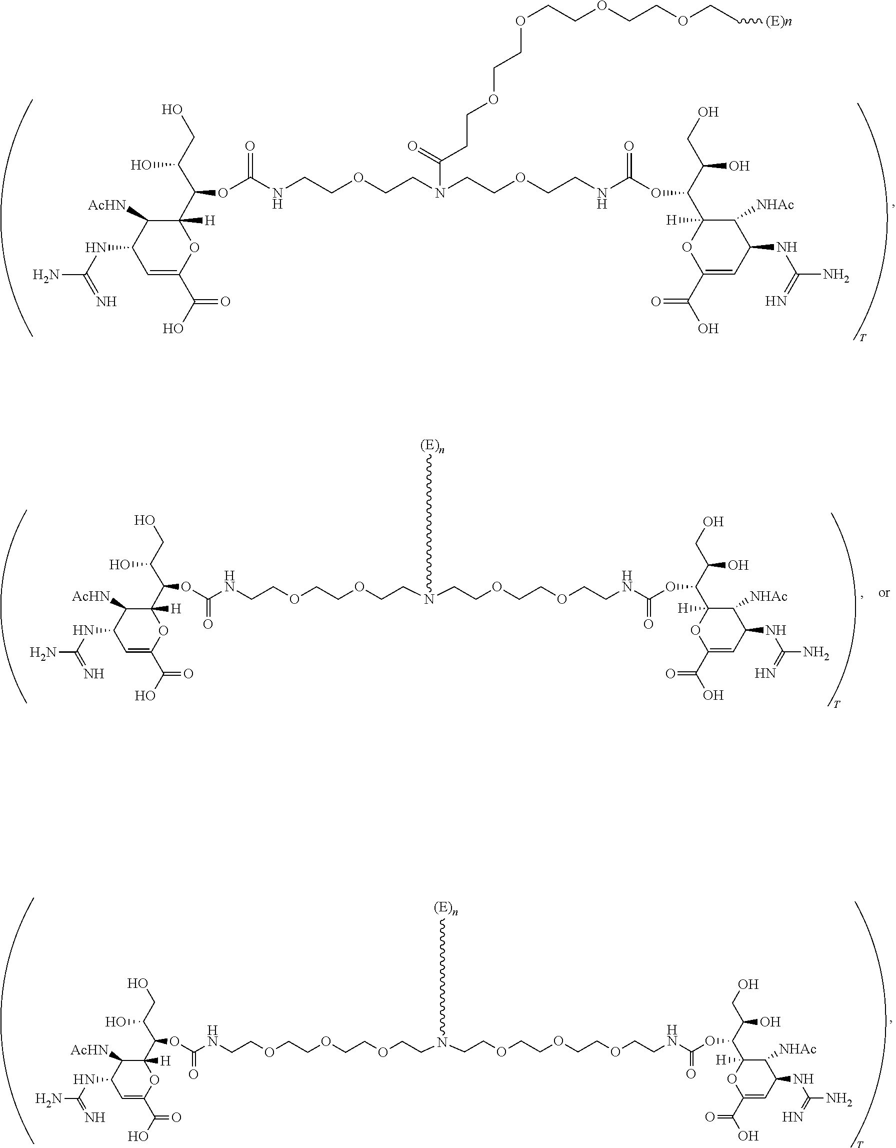

or a pharmaceutically acceptable salt thereof.

In some embodiments, the conjugate has the structure selected from:

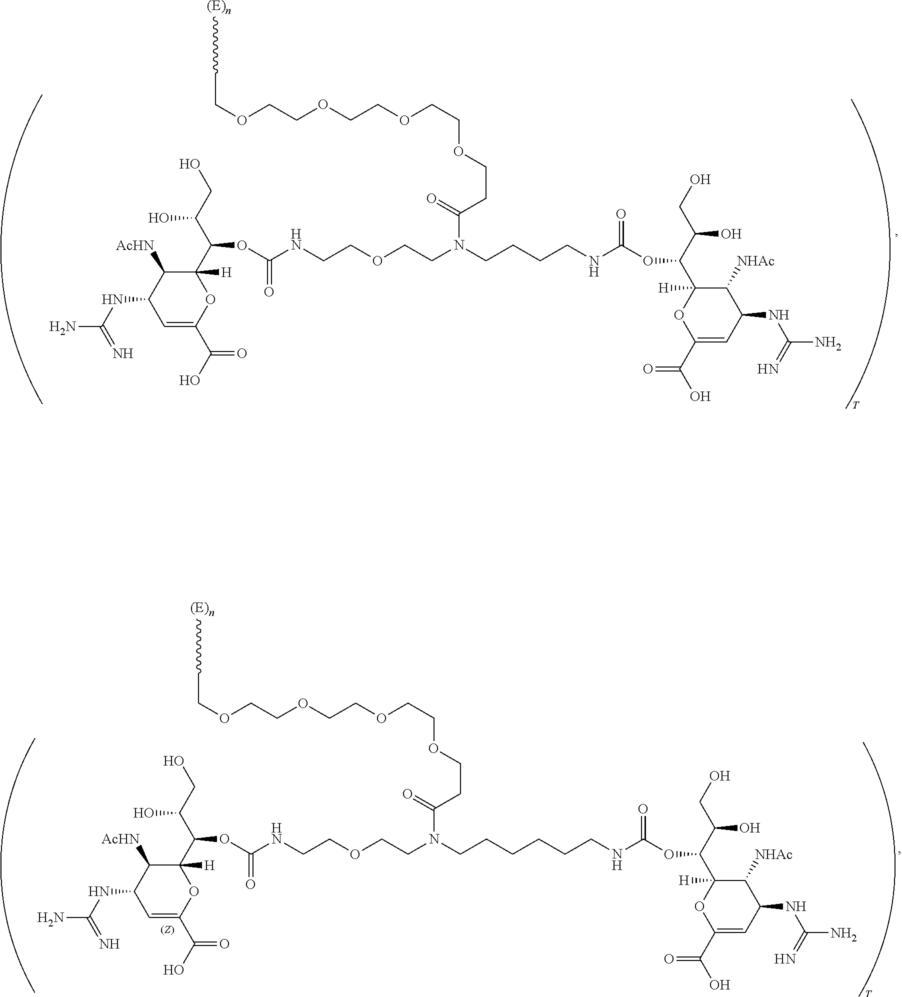

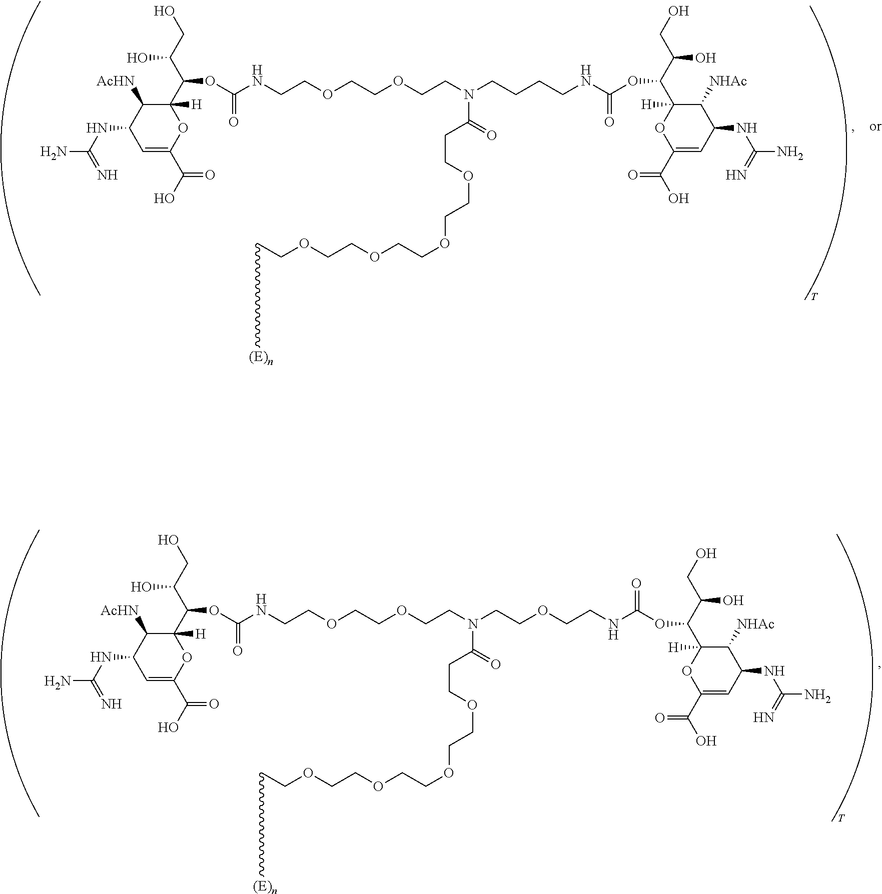

or a pharmaceutically acceptable salt thereof.

In some embodiments, the conjugate is described by formula (D-II-6):

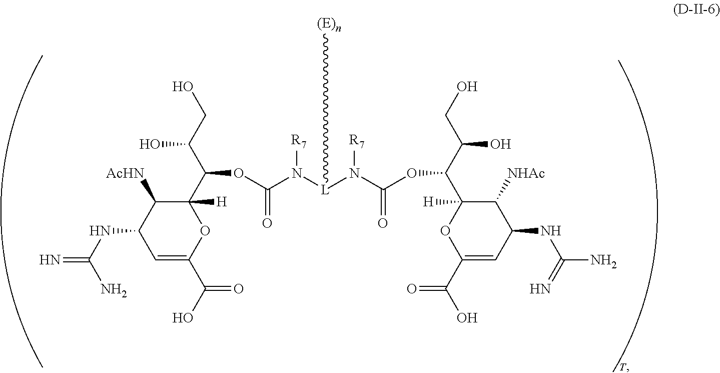

wherein R₇ is selected from H, C1-C20 alkyl, C3-C20 cycloalkyl, C3-C20 heterocycloalkyl; C5-C15 aryl, and C2-C15 heteroaryl; or a pharmaceutically acceptable salt thereof. In some embodiments, R₇ is selected from C1-C20 alkyl (e.g., methyl, ethyl, propyl, or butyl).

In some embodiments, the conjugate is described by formula (D-II-7):

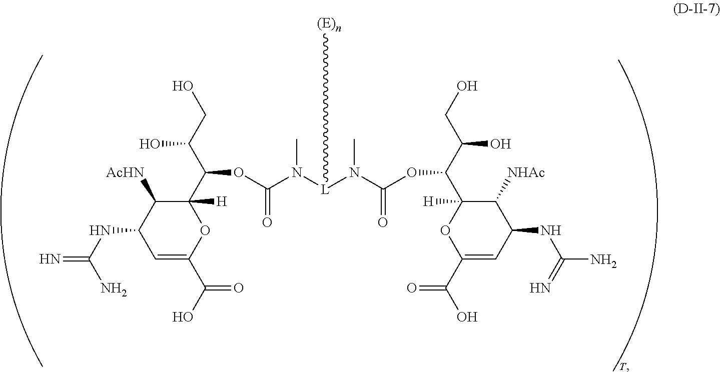

or a pharmaceutically acceptable salt thereof.

In some embodiments, the conjugate is described by formula (D-II-8):

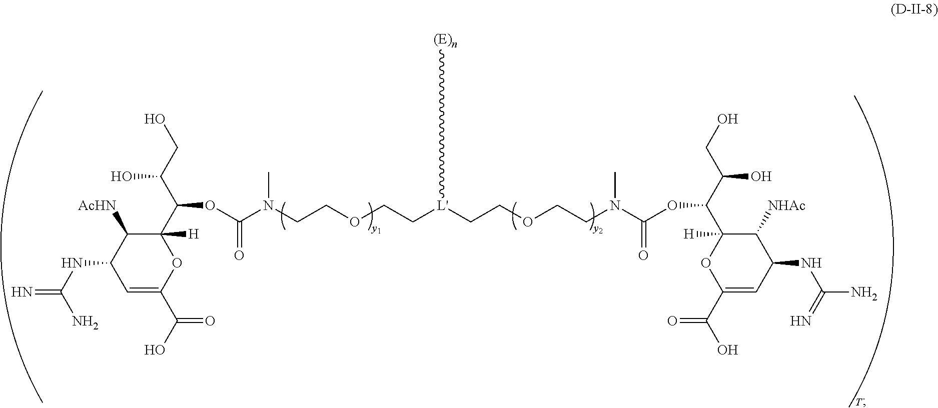

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure

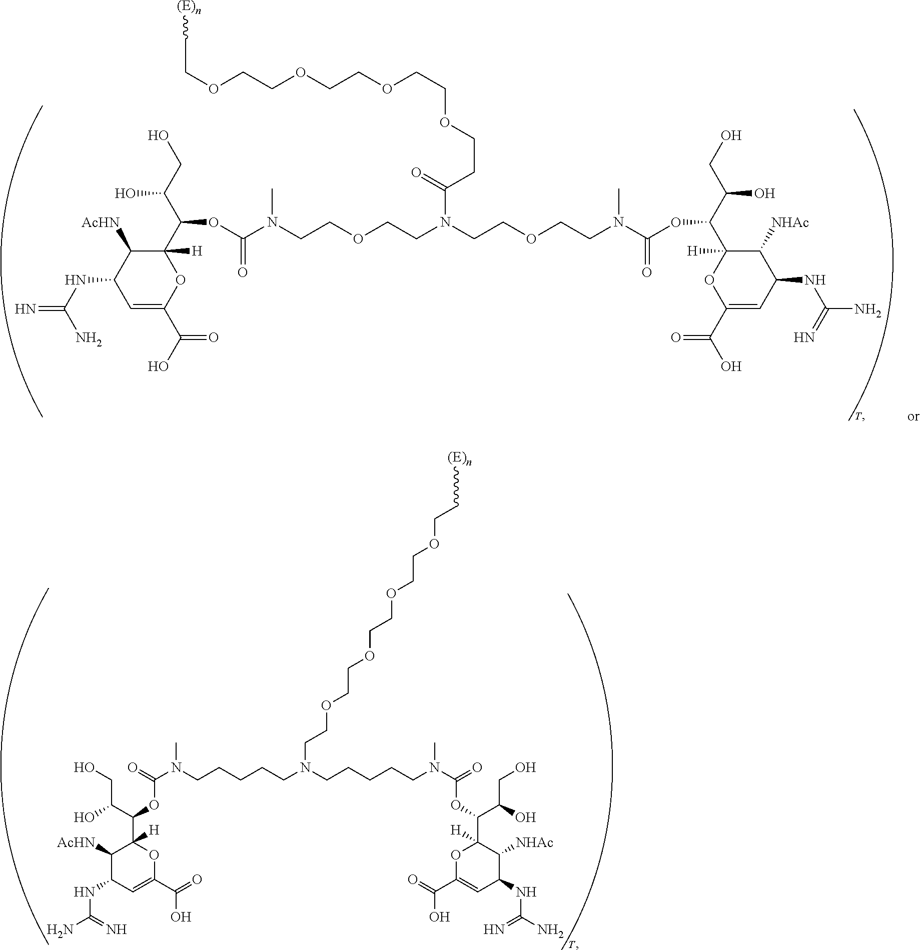

or a pharmaceutically acceptable salt thereof.

In some embodiments, the conjugate has the structure

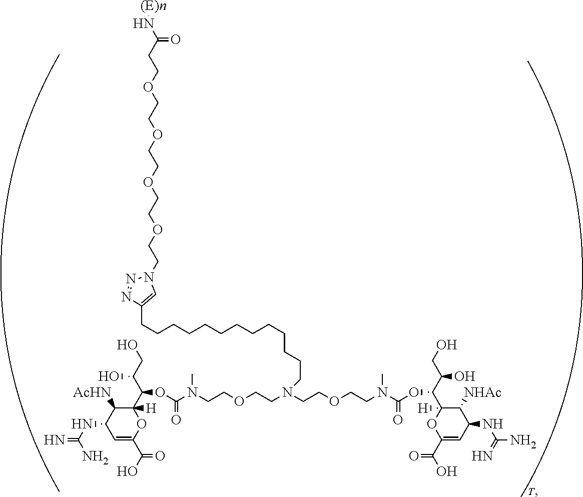

or a pharmaceutically acceptable salt thereof.

In some embodiments, the conjugate has the structure

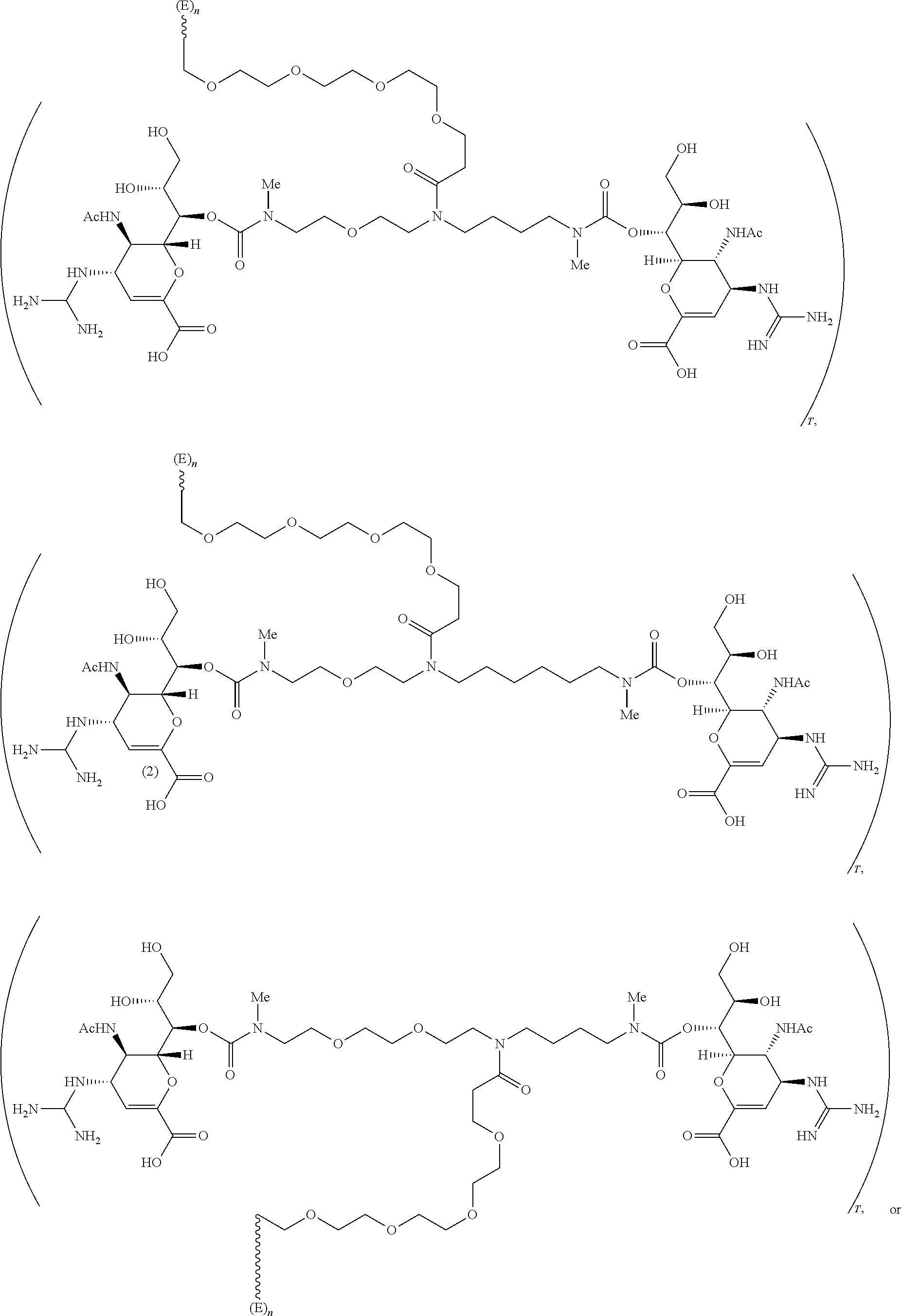

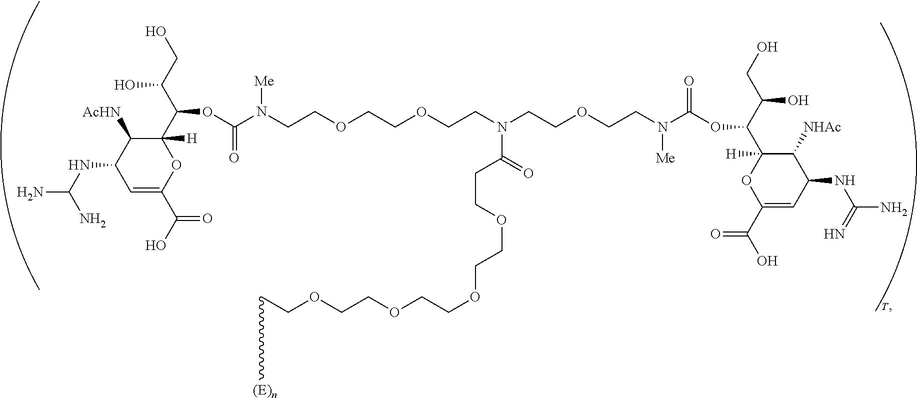

or a pharmaceutically acceptable salt thereof.

In some embodiments, the conjugate has the structure

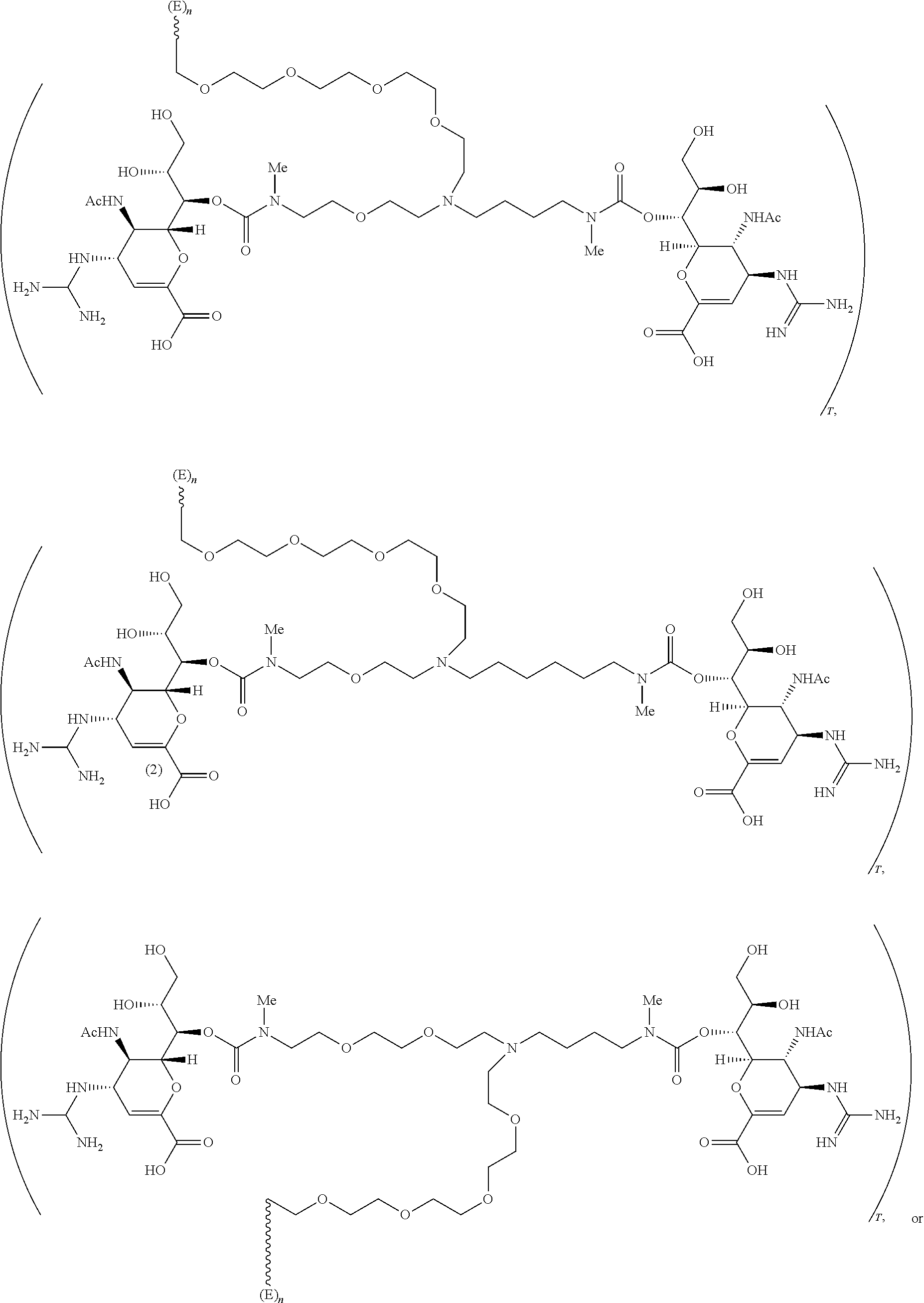

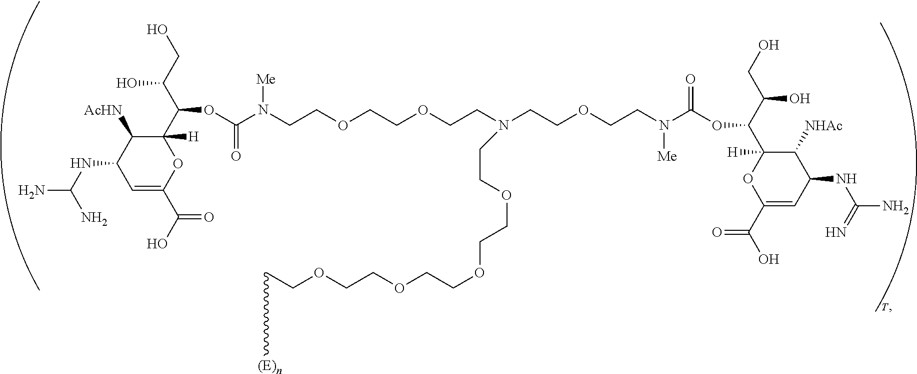

or a pharmaceutically acceptable salt thereof.

In some embodiments, the conjugate is described by formula (D-II-9):

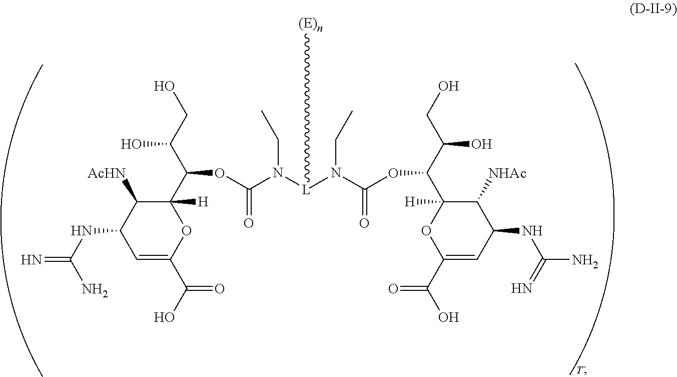

or a pharmaceutically acceptable salt thereof.

In some embodiments, the conjugate is described by formula (D-II-10):

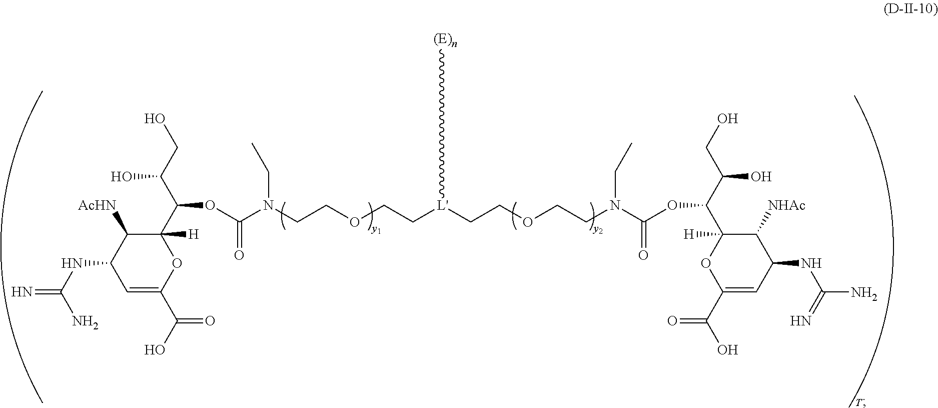

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure

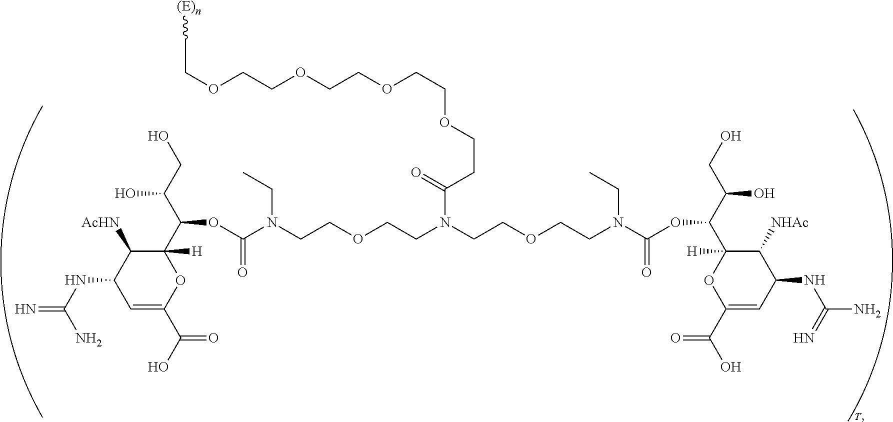

or a pharmaceutically acceptable salt thereof.

In some embodiments, the conjugate has the structure of

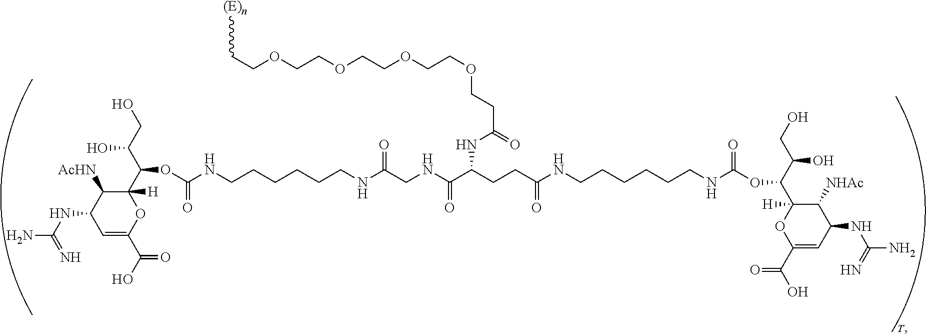

or a pharmaceutically acceptable salt thereof.

In some embodiments, the conjugate is described by formula (D-III):

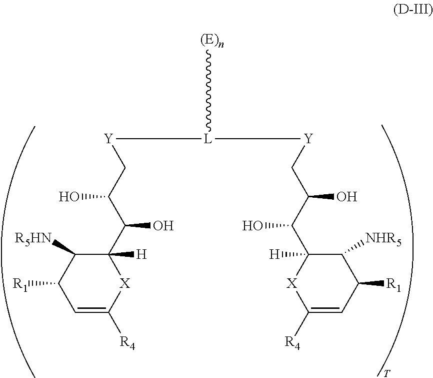

or a pharmaceutically acceptable salt thereof.

In some embodiments, the conjugate is described by formula (D-III-1):

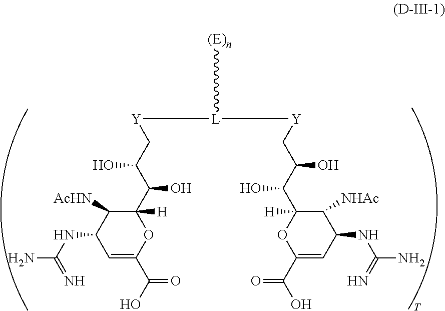

or a pharmaceutically acceptable salt thereof.

In some embodiments, the conjugate is described by formula (D-III-2):

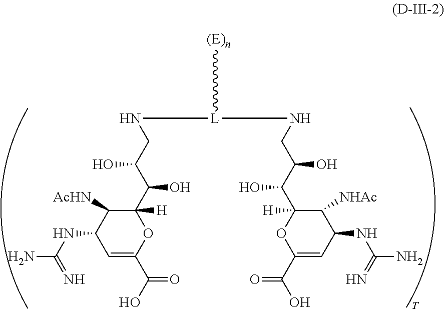

or a pharmaceutically acceptable salt thereof.

In some embodiments, the conjugate is described by formula (D-III-3):

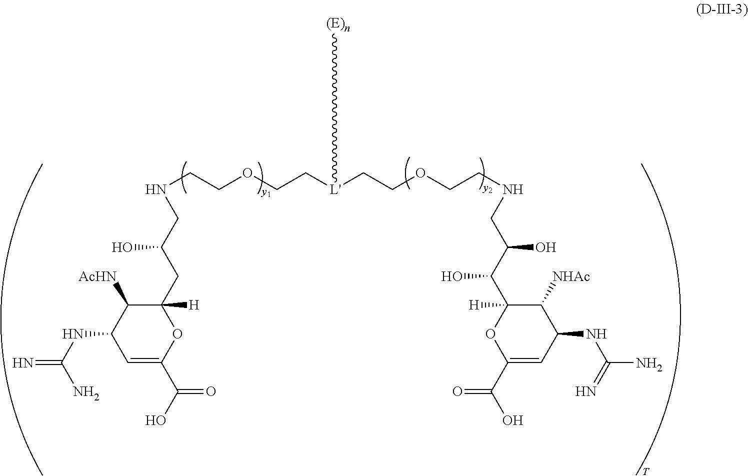

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-III-4):

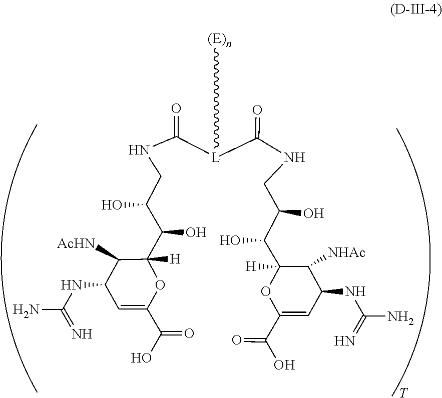

or a pharmaceutically acceptable salt thereof.

In some embodiments, the conjugate is described by formula (D-III-5):

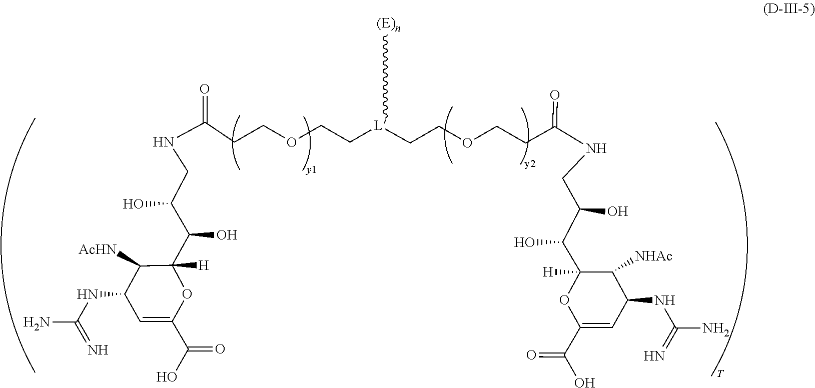

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments. L′ is a nitrogen atom.

In some embodiments, the conjugate is described by formula (D-III-6):

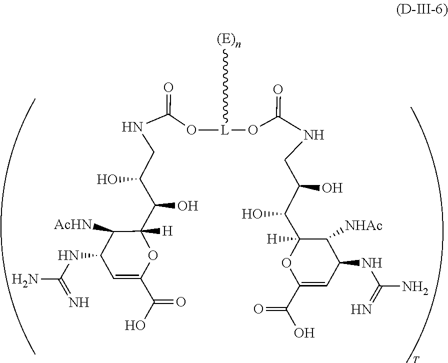

or a pharmaceutically acceptable salt thereof.

In some embodiments, the conjugate is described by formula (D-III-7):

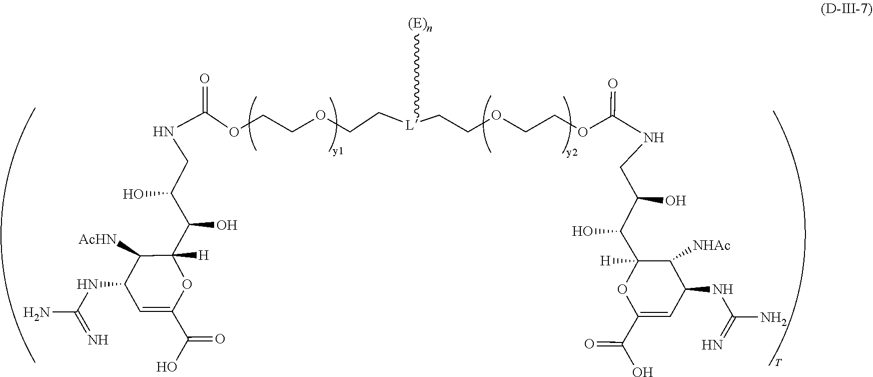

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-III-8):

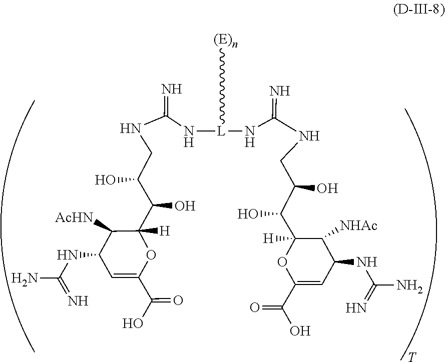

or a pharmaceutically acceptable salt thereof.

In some embodiments, the conjugate is described by formula (D-III-9):

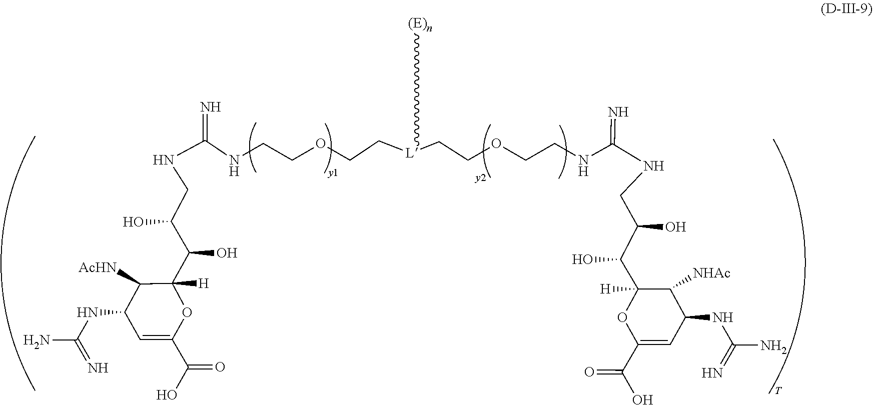

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate is described by formula (D-IV):

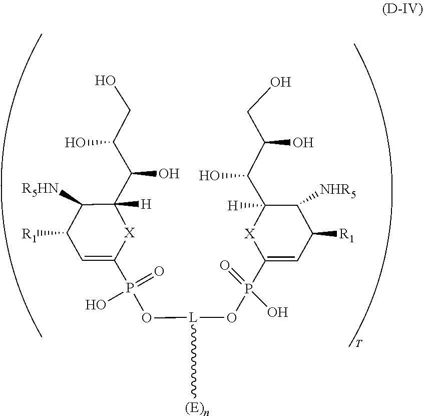

or a pharmaceutically acceptable salt thereof.

In some embodiments, the conjugate is described by formula (D-IV-1):

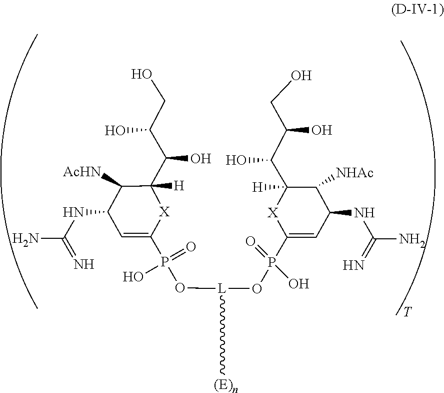

or a pharmaceutically acceptable salt thereof.

In some embodiments, the conjugate is described by formula (D-IV-2):

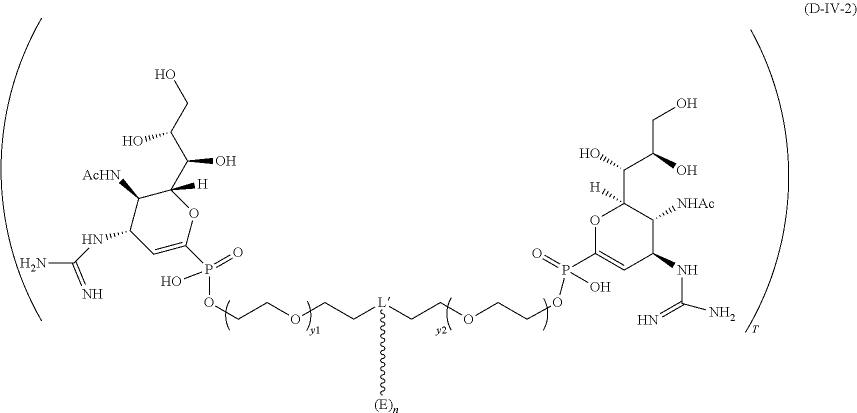

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments. L′ is a nitrogen atom.

In some embodiments, the conjugate is described by formula (D-V):

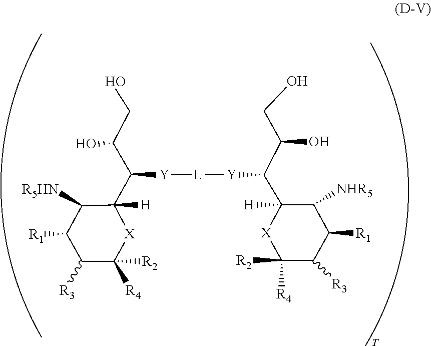

or a pharmaceutically acceptable salt thereof.

In some embodiments, the conjugate is described by formula (D-V-1):

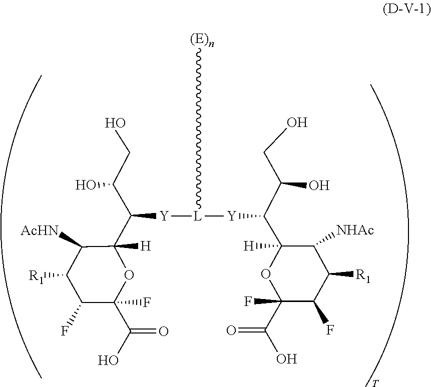

or a pharmaceutically acceptable salt thereof.

In some embodiments the conjugate is described formula (D-V-2:

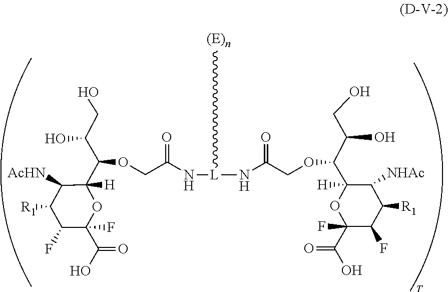

or a pharmaceutically acceptable salt thereof.

In some embodiments, the conjugate is described by formula (D-V-3):

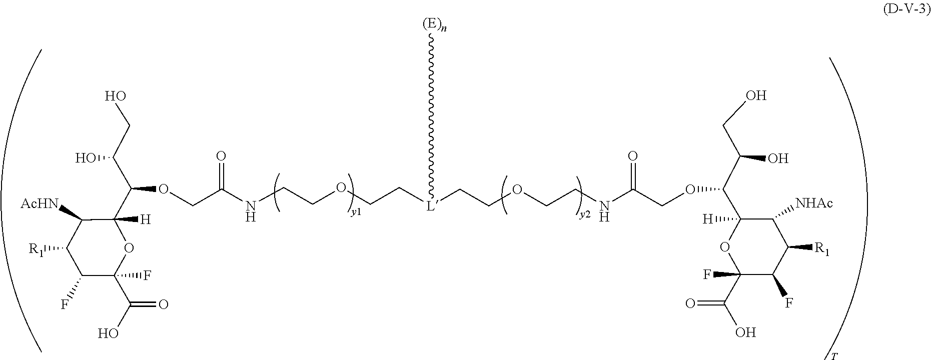

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom. In some embodiments, y₁ and y₂ are each 1, y₁ and y₂ are each 2, or y₁ and y₂ are each 3.

In some embodiments, the conjugate is described by formula (D-V-4):

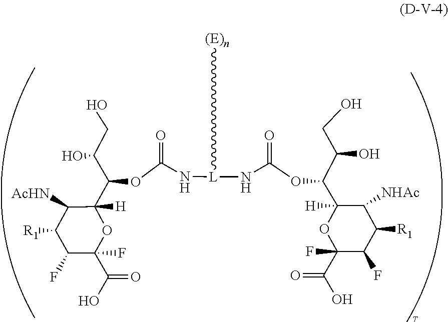

or a pharmaceutically acceptable salt thereof.

In some embodiments, the conjugate is described by formula (D-V-5):

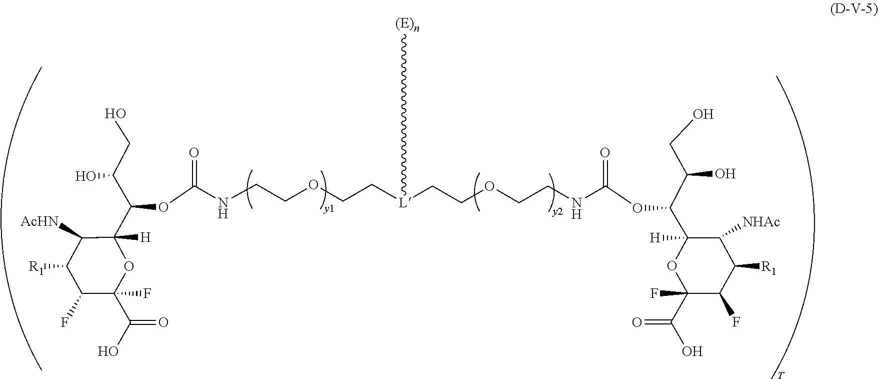

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom. In some embodiments, y₁ and y₂ are each 1, y₁ and y₂ are each 2, or y₁ and y₂ are each 3.

In some embodiments, the conjugate is described by formula (D-V-6):

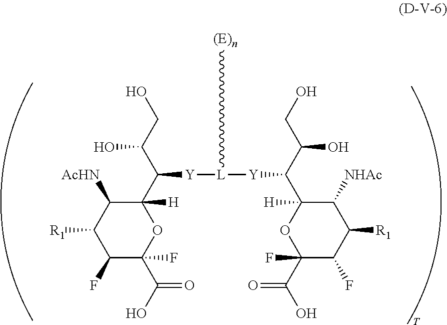

or a pharmaceutically acceptable salt thereof.

In some embodiments, the conjugate is described by formula (D-V-7):

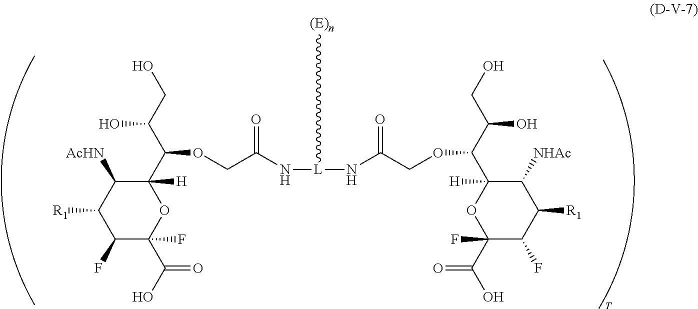

or a pharmaceutically acceptable salt thereof.

In some embodiments, the conjugate is described by formula (D-V-8):

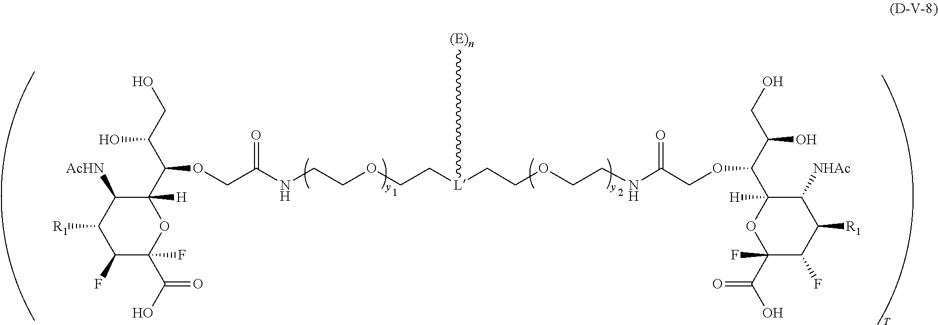

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom. In some embodiments, y₁ and y₂ are each 1, y₁ and y₂ are each 2, or y₁ and y₂ are each 3.

In some embodiments, the conjugate is described by formula (D-V-9):

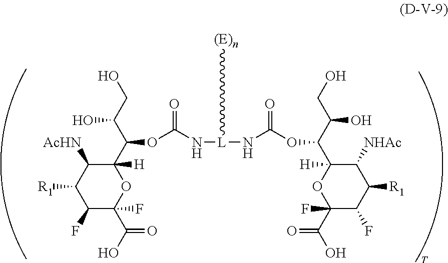

or a pharmaceutically acceptable salt thereof.

In some embodiments, the conjugate is described by formula (D-V-10):

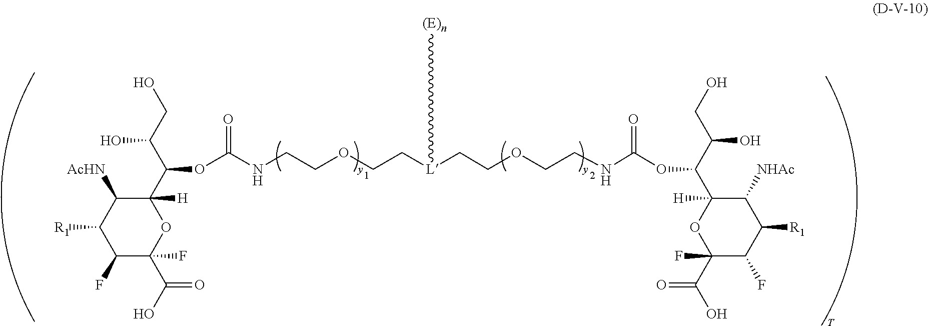

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments. L′ is a nitrogen atom. In some embodiments, y₁ and y₂ are each 1, y₁ and y₂ are each 2, or y₁ and y₂ are each 3.

In some embodiments, the conjugate is described by formula (D-VI):

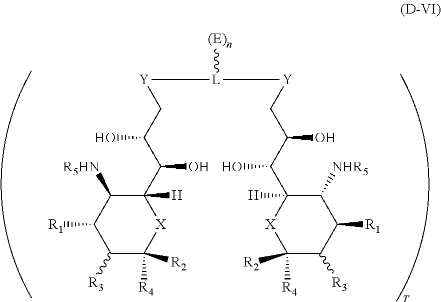

or a pharmaceutically acceptable salt thereof.

In some embodiments, the conjugate is described by formula (D-VI-1):

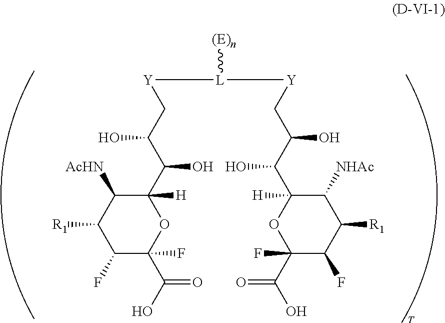

or a pharmaceutically acceptable salt thereof.

In some embodiments, the conjugate is described by formula (D-VI-2):

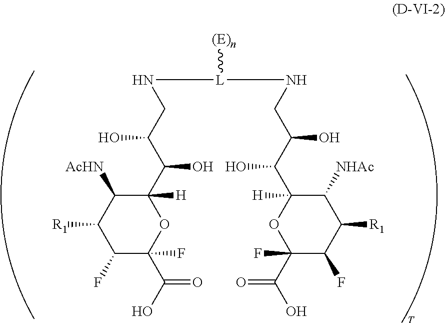

or a pharmaceutically acceptable salt thereof.

In some embodiments, the conjugate is described by formula (D-VI-3):

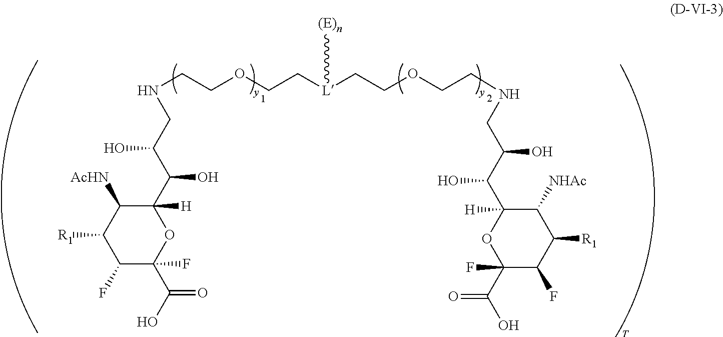

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments. L′ is a nitrogen atom. In some embodiments, y₁ and y₂ are each 1, y₁ and y₂ are each 2, or y₁ and y₂ are each 3.

In some embodiments, the conjugate is described by formula (D-VI-4):

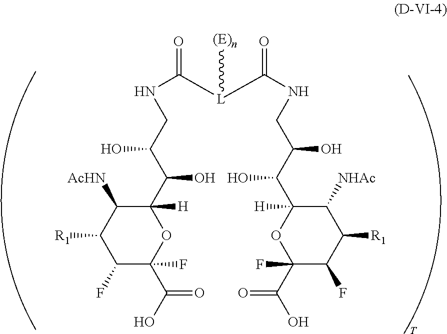

or a pharmaceutically acceptable salt thereof.

In some embodiments, the conjugate is described by formula (D-VI-5):

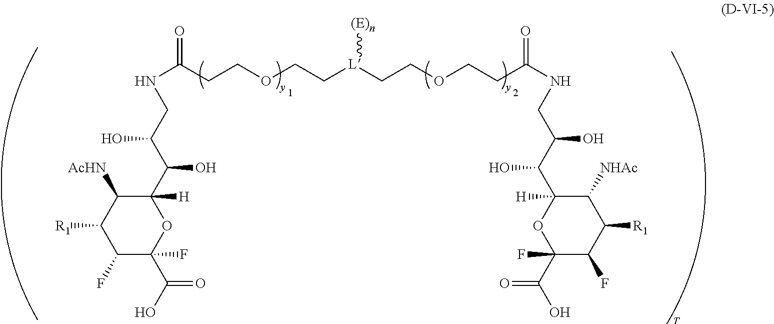

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom. In some embodiments, y₁ and y₂ are each 1, y₁ and y₂ are each 2, or y₁ and y₂ are each 3.

In some embodiments, the conjugate is described by formula (D-VI-6):

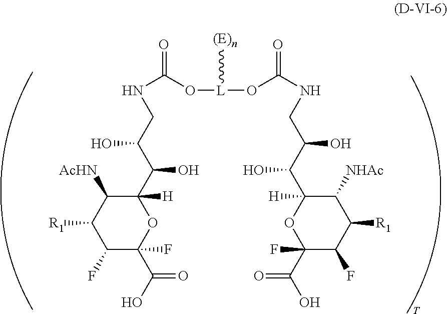

or a pharmaceutically acceptable salt thereof.

In some embodiments, the conjugate is described by formula (D-VI-7):

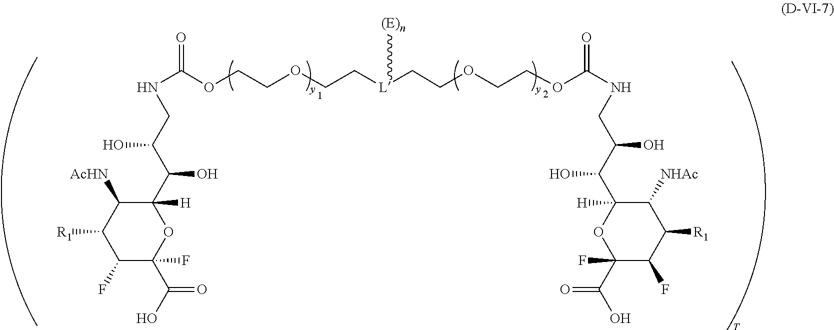

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom. In some embodiments, y₁ and y₂ are each 1, y₁ and y₂ are each 2, or y₁ and y₂ are each 3.

In some embodiments, the conjugate is described by formula (D-VI-8):

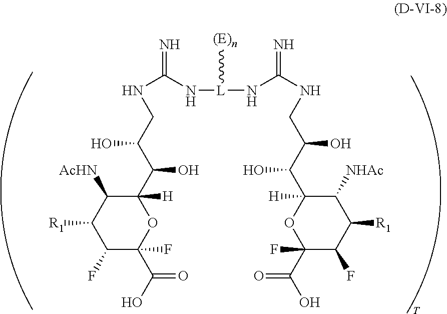

or a pharmaceutically acceptable salt thereof.

In some embodiments, the conjugate is described by formula (D-VI-9):

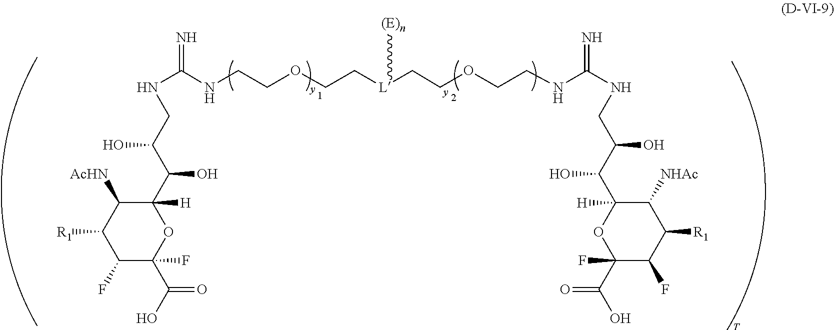

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom. In some embodiments, y₁ and y₂ are each 1, y₁ and y₂ are each 2, or y₁ and y₂ are each 3.

In some embodiments, the conjugate is described by formula (D-VII):

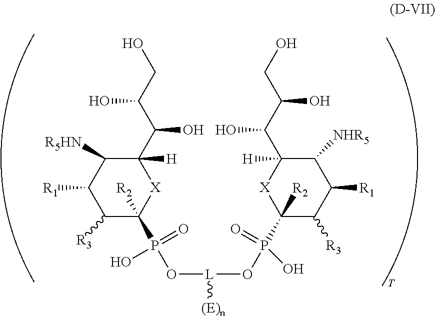

or a pharmaceutically acceptable salt thereof.

In some embodiments, the conjugate is described by formula (D-VIII):

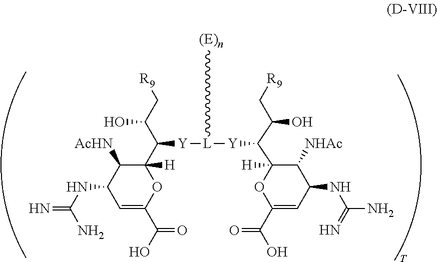

or a pharmaceutically acceptable salt thereof.

In some embodiments, the conjugate is described by formula (D-VIII-1):

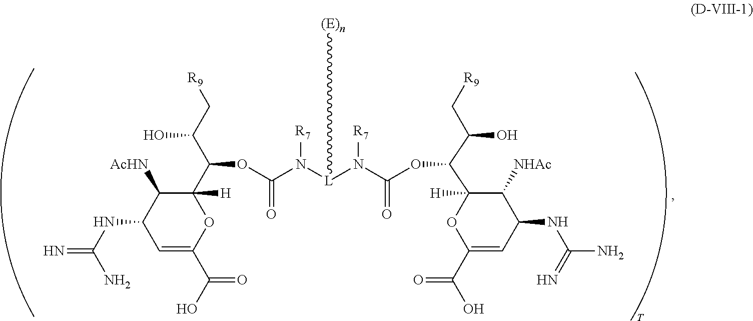

wherein R₇ is selected from H, C1-C20 alkyl, C3-C20 cycloalkyl, C3-C20 heterocycloalkyl; C5-C15 aryl, and C2-C15 heteroaryl; or a pharmaceutically acceptable salt thereof. In some embodiments, R₇ is selected from C1-C20 alkyl, C3-C20 cycloalkyl, C3-C20 heterocycloalkyl; C5-C15 aryl, and C2-C15 heteroaryl. In some embodiments, R₇ is methyl, ethyl, propyl, or butyl.

In some embodiments of any of the aspects described herein. R₁ is OH. In some embodiments of any of the aspects described herein, R₁ is NH₂. In some embodiments of any of the aspects described herein, R₁ is —NHC(═NH)NH₂. In some embodiments of any of the aspects described herein, R₂ is —F. In some embodiments of any of the aspects described herein, R₃ is —F. In some embodiments of any of the aspects described herein, R₄ is —CO₂H. In some embodiments of any of the aspects described herein. R₅ is —COCH₃.

In some embodiments of any of the aspects described herein, L or L′ includes one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments of any of the aspects described herein, L or L′ is oxo substituted. In some embodiments, the backbone of L or L′ comprises no more than 250 atoms. In some embodiments, L or L′ is capable of forming an amide, a carbamate, a sulfonyl, or a urea linkage. In some embodiments L or L′ is a bond. In some embodiments, L or L′ is an atom. In some embodiments, L or L′ is a nitrogen atom.

In some embodiments of any of the aspects described herein, each L is described by formula (D-L-I):

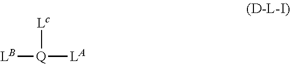

wherein L^{A} is described by formula G^{A1}-(Z^{A1})_{g1}—(Y^{A1})ₕ₁—(Z^{A2})ᵢ₁—(Y^{A2})ⱼ₁—(Z^{A3})ₖ₁—(Y^{A3})ₗ₁—(Z^{A4})ₘ₁—(Y^{A4})ₙ₁—(Z^{A5})o₁-G^{A2}; L^{B} is described by formula G^{B1}-(Z^{B1})_{g2}—(Y^{B1})ₕ₂—(Z^{B2})ᵢ₂—(Y^{B2})ⱼ₂—(Z^{B3})ₖ₂—(Y^{B3})ₗ₂—(Z^{B4})ₘ₂—(Y^{B4})ₙ₂—(Z^{B5})o₂-G^{B2}; L^{C} is described by formula G^{C1}-(Z^{C1})_{g3}—(Y^{C1})ₕ₃—(Z^{C2})ᵢ₃—(Y^{C2})ⱼ₃—(Z^{C3})ₖ₃—(Y^{C3})ₗ₃—(Z^{C4})ₘ₃—(Y^{C4})ₙ₃—(Z^{C5})o₃-G^{C2}; G^{A1} is a bond attached to Q; G^{A2} is a bond attached to A1; G^{B1} is a bond attached to Q); G^{B2} is a bond attached to A2; G^{C1} is a bond attached to Q; G^{C2} is a bond attached to E or a functional group capable of reacting with a functional group conjugated to E (e.g., maleimide and cysteine, amine and activated carboxylic acid, thiol and maleimide, activated sulfonic acid and amine, isocyanate and amine, azide and alkyne, and alkene and tetrazine); each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4} and Z^{C5} is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, Y^{B4}, Y^{C1}, Y^{C2}, Y^{C3}, and Y^{C4} is, independently, O, S, NRⁱ, P, carbonyl, thiocarbonyl, sulfonyl, phosphate, phosphoryl, or imino; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, o2, g3, h3, i3, j3, k3, 13, m3, n3, and 03 is, independently, 0 or 1; Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

In some embodiments. L^{C} may have two points of attachment to the Fc domain (e.g., two G^{C2}).

In some embodiments of any of the aspects described herein, L includes a polyethylene glycol (PEG) linker. A PEG linker includes a linker having the repeating unit structure (—CH₂CH₂O—)ₙ, wherein n is an integer from 2 to 100. A polyethylene glycol linker may covalently join a first neuraminidase inhibitor and a second neuraminidase inhibitor (e.g., in a conjugate of any one of formulas (D-I)-(D-VIII)). A polyethylene glycol linker may covalently join a neuraminidase inhibitor dimer and E (e.g., in a conjugate of any one of formulas (D-I)-(D-VIII)). A polyethylene glycol linker may be selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀). In some embodiments, L^{c} includes a PEG linker, where L^{c} is covalently attached to each of Q and E.

Intermediates of Table 1a may be conjugated to an Fc domain or Fc domain monomer (e.g., by way of a linker) by any suitable methods known to those of skill in the art, including any of the methods described or exemplified herein. In some embodiments, one or more nitrogen atoms of one or more surface exposed lysine residues of E or one or more sulfur atoms of one or more surface exposed cysteines in E is covalently conjugated to a linker (e.g., a PEG₂-PEG₂₀ linker). The linker conjugated to E may be functionalized such that it may react to form a covalent bond with any of the Ints described herein (e.g., an Int of Table 1a). In some embodiments, E is conjugated to a linker functionalized with an azido group and the Int (e.g., an Int of Table 1a) is functionalized with an alkyne group. Conjugation (e.g., by click chemistry) of the linker-azido of E and linker-alkyne of the Int forms a conjugate of the disclosure, for example a conjugate described by formula (5). In yet other embodiments, E is conjugated to a linker functionalized with an alkyne group and the Int (e.g., an Int of Table 1a) is functionalized with an azido group. Conjugation of the linker-alkyne of E and linker-azido of the Int forms a conjugate of the disclosure, for example a conjugate described by any one of formulas (D-I)-(D-VIII). In yet other embodiments, the Int (e.g., an Int of Table 1a) is functionalized with a phenyl ester group (e.g., a trifluorophenyl ester group or a tetrafluorophenyl ester group). Conjugation (e.g., by acylation) of E and the linker-phenyl ester (e.g., trifluorophenyl ester or tetrafluorophenyl ester) of the Int forms a conjugate of the invention, for example a conjugate described by any one of formulas (D-I)-(D-VIII). Conjugation (e.g., by acylation) of E and the linker-phenyl ester (e.g., trifluorophenyl ester or tetrafluorophenyl ester) of the Int is conducted. e.g., by methods described herein.

TABLE 1a
Intermediates

| Intermediate | Structure |
|---|---|
| Int-1 | 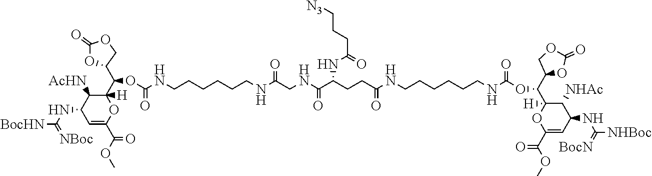 |
| Int-2 | 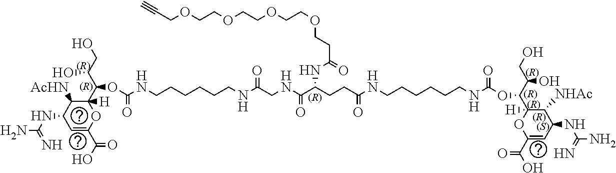 |
| Int-3 | 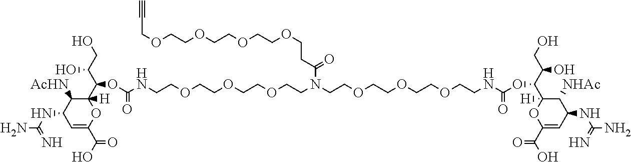 [SMILES: C#CCOCCOCCOCCOCCC(=O)N(CCOCCOCCOCCNC(=O)O[C@@H]([C@@H]1OC(C(=O)O)=C[C@H](NC(=N)N)[C@H]1NC(C)=O)[C@H](O)CO)CCOCCOCCOCCNC(=O)O[C@@H]([C@@H]1OC(C(=O)O)=C[C@H](NC(=N)N)[C@H]1NC(C)=O)[C@H](O)CO] |
| Int-7 | 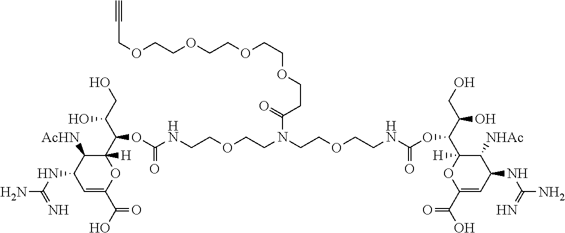 [SMILES: C#CCOCCOCCOCCOCCC(=O)N(CCOCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| Int-9 | 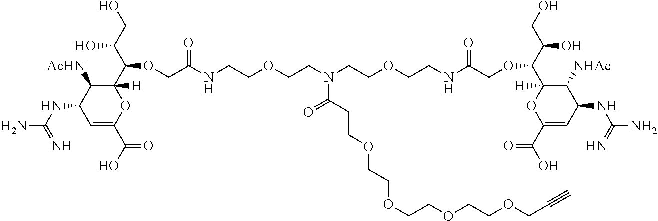 [SMILES: C#CCOCCOCCOCCOCCC(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| Int-12 | 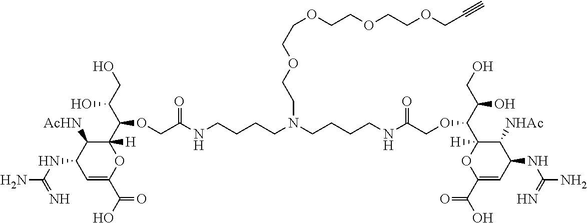 [SMILES: C#CCOCCOCCOCCOCCN(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| Int-17 | 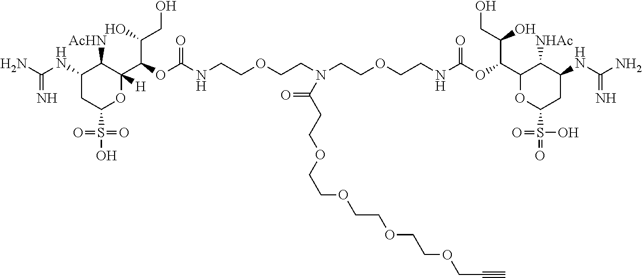 [SMILES: C#CCOCCOCCOCCOCCC(=O)N(CCOCCNC(=O)O[C@@H]([C@@H]1O[C@H](S(=O)(=O)O)C[C@H](NC(=N)N)[C@H]1NC(C)=O)[C@H](O)CO)CCOCCNC(=O)O[C@@H](C1O[C@@H](S(=O)(=O)O)C[C@H](NC(=N)N)[C@H]1NC(C)=O)[C@H](O)CO] |
| Int-18 | 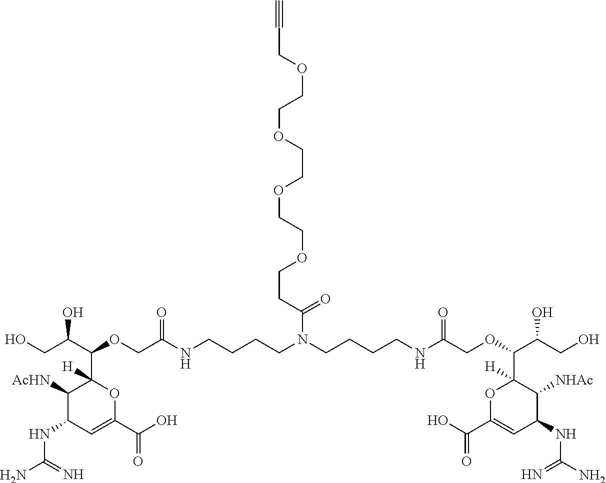 [SMILES: C#CCOCCOCCOCCOCCC(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| Int-19 | 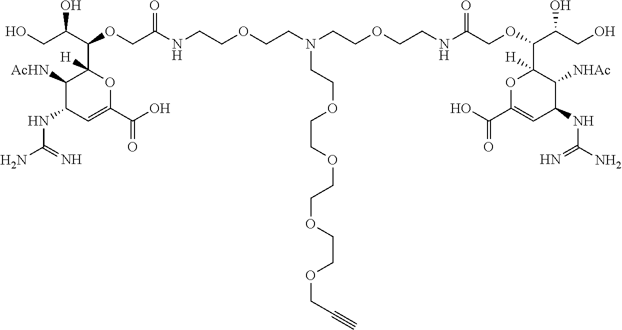 [SMILES: C#CCOCCOCCOCCOCCN(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| Int-20 | 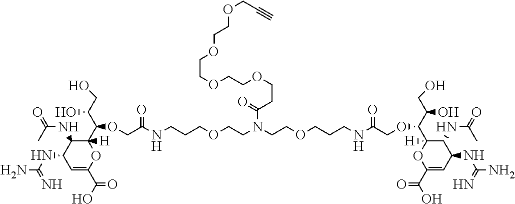 [SMILES: C#CCOCCOCCOCCOCCC(=O)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| Int-21 | 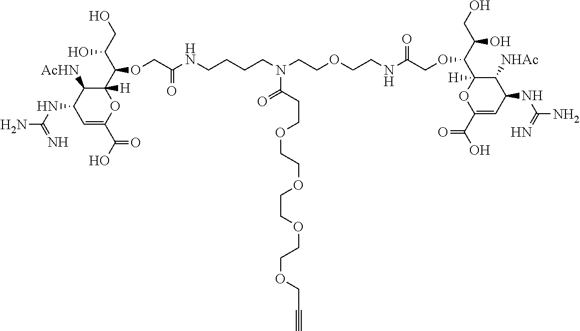 [SMILES: C#CCOCCOCCOCCOCCC(=O)N(CCCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| Int-23 | 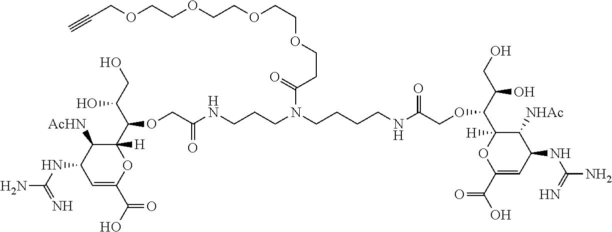 [SMILES: C#CCOCCOCCOCCOCCC(=O)N(CCCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| Int-24 | 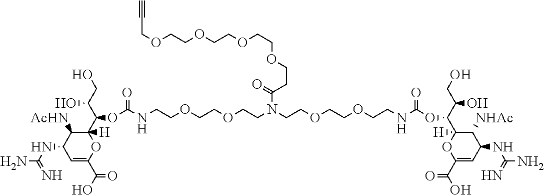 [SMILES: C#CCOCCOCCOCCOCCC(=O)N(CCOCCOCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| Int-25 | 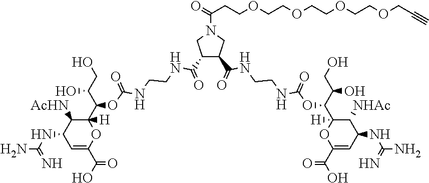 [SMILES: C#CCOCCOCCOCCOCCC(=O)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| Int-26 | 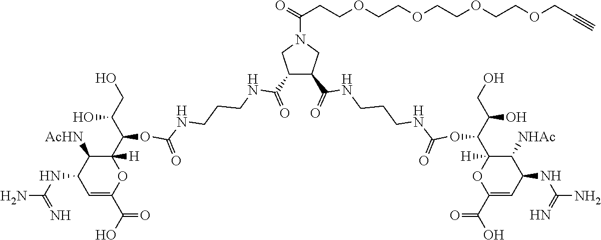 [SMILES: C#CCOCCOCCOCCOCCC(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@H]([C@@H]2OC(C(=O)O)=C[C@H](NC(=N)N)[C@H]2NC(C)=O)[C@H](O)CO)C1] |
| Int-27 | 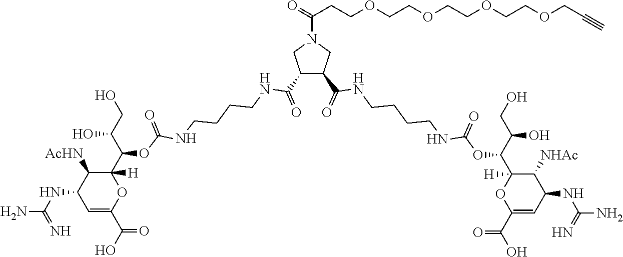 [SMILES: C#CCOCCOCCOCCOCCC(=O)N1C[C@@H](C(=O)NCCCCNC(=O)O[C@@H]([C@@H]2OC(C(=O)O)=C[C@H](NC(=N)N)[C@H]2NC(C)=O)[C@H](O)CO)[C@H](C(=O)NCCCCNC(=O)O[C@@H]([C@@H]2OC(C(=O)O)=C[C@H](NC(=N)N)[C@H]2NC(C)=O)[C@H](O)CO)C1] |
| Int-28 | 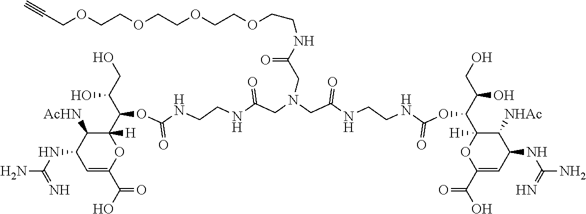 [SMILES: C#CCOCCOCCOCCOCCNC(=O)CN(CC(=O)NCCNC(=O)O[C@@H]([C@@H]1OC(C(=O)O)=C[C@H](NC(=N)N)[C@H]1NC(C)=O)[C@H](O)CO)CC(=O)NCCNC(=O)O[C@@H]([C@@H]1OC(C(=O)O)=C[C@H](NC(=N)N)[C@H]1NC(C)=O)[C@H](O)CO] |
| Int-31 | 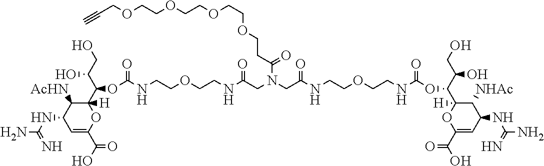 [SMILES: C#CCOCCOCCOCCOCCC(=O)N(CC(=O)NCCOCCNC(=O)O[C@@H]([C@@H]1OC(C(=O)O)=C[C@H](NC(=N)N)[C@H]1NC(C)=O)[C@H](O)CO)CC(=O)NCCOCCNC(=O)O[C@@H]([C@@H]1OC(C(=O)O)=C[C@H](NC(=N)N)[C@H]1NC(C)=O)[C@H](O)CO] |
| Int-34 | 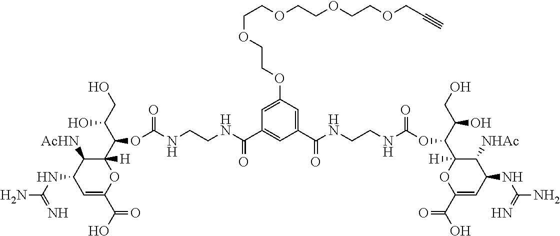 [SMILES: C#CCOCCOCCOCCOCCOc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| Int-38 | 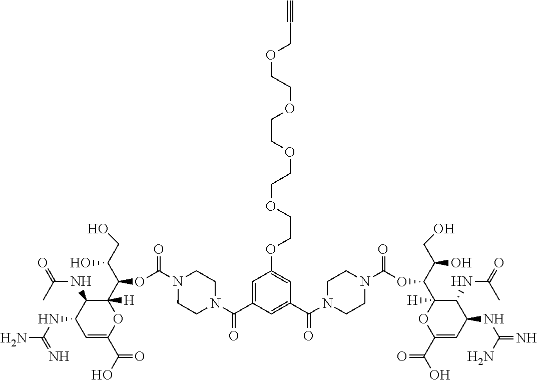 [SMILES: C#CCOCCOCCOCCOCCOc1cc(C(=O)N2CCN(C(=O)O[C@@H]([C@@H]3OC(C(=O)O)=C[C@H](NC(=N)N)[C@H]3NC(C)=O)[C@H](O)CO)CC2)cc(C(=O)N2CCN(C(=O)O[C@@H]([C@@H]3OC(C(=O)O)=C[C@H](NC(=N)N)[C@H]3NC(C)=O)[C@H](O)CO)CC2)c1] |
| Int-39 | 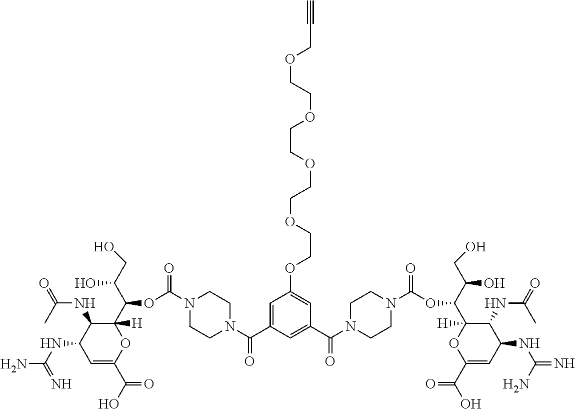 [SMILES: C#CCOCCOCCOCCOCCOc1cc(C(=O)N2CCN(C(=O)O[C@@H]([C@@H]3OC(C(=O)O)=C[C@H](NC(=N)N)[C@H]3NC(C)=O)[C@H](O)CO)CC2)cc(C(=O)N2CCN(C(=O)O[C@@H]([C@@H]3OC(C(=O)O)=C[C@H](NC(=N)N)[C@H]3NC(C)=O)[C@H](O)CO)CC2)c1] |
| Int-42 | 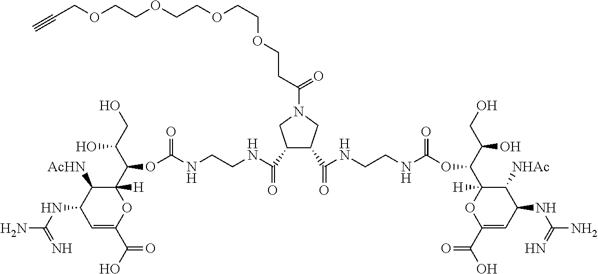 [SMILES: C#CCOCCOCCOCCOCCC(=O)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| Int-43 | 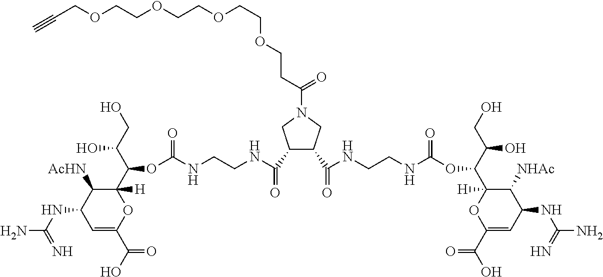 [SMILES: C#CCOCCOCCOCCOCCC(=O)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| Int-44 | 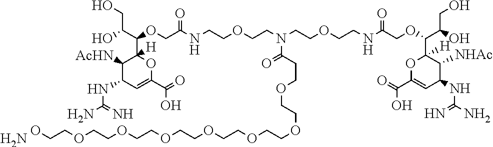 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCOCCOCCON)[C@H](O)CO)OC(C(=O)O)=C[C@@H]1NC(=N)N] |
| Int-45 | 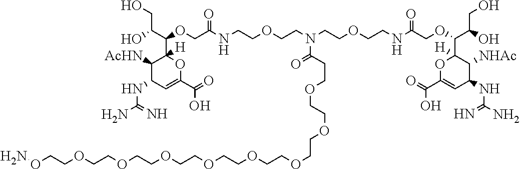 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCOCCOCCOCCON)[C@H](O)CO)OC(C(=O)O)=C[C@@H]1NC(=N)N] |
| Int-46 | 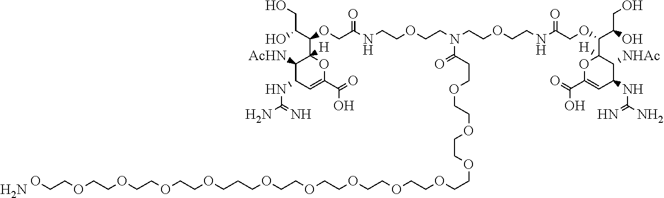 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCOCCOCCOCCCOCCOCCOCCOCCON)[C@H](O)CO)OC(C(=O)O)=C[C@@H]1NC(=N)N] |
| Int-47 | 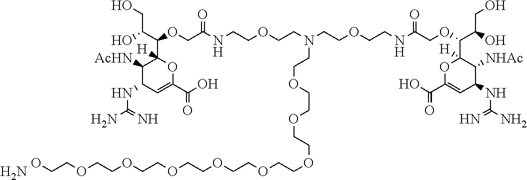 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)CCOCCOCCOCCOCCOCCOCCOCCOCCON)[C@H](O)CO)OC(C(=O)O)=C[C@@H]1NC(=N)N] |
| Int-48 | 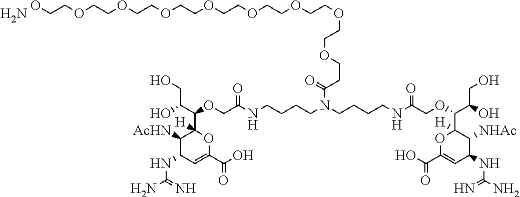 [SMILES: CC(=O)N[C@H]1[C@H]([C@H](OCC(=O)NCCCCN(CCCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCOCCOCCOCCON)[C@H](O)CO)OC(C(=O)O)=C[C@@H]1NC(=N)N] |
| Int-49 | 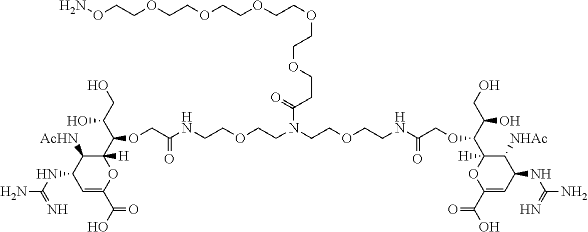 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCON)[C@H](O)CO)OC(C(=O)O)=C[C@@H]1NC(=N)N] |
| Int-50 | 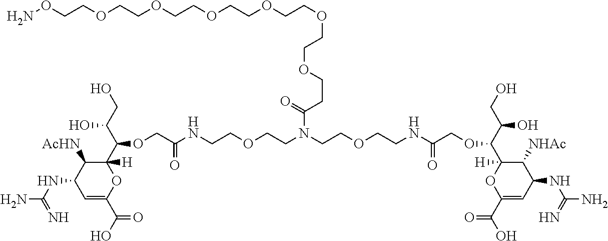 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCOCCON)[C@H](O)CO)OC(C(=O)O)=C[C@@H]1NC(=N)N] |
| Int-52 | 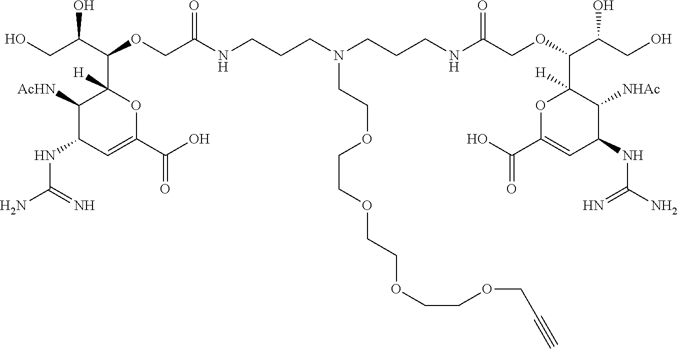 [SMILES: C#CCOCCOCCOCCOCCN(CCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| Int-53 | 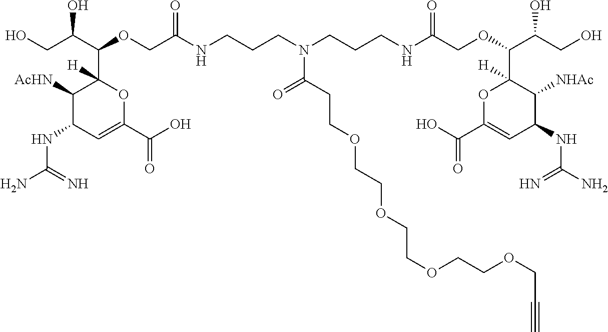 [SMILES: C#CCOCCOCCOCCOCCC(=O)N(CCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| Int-54 | 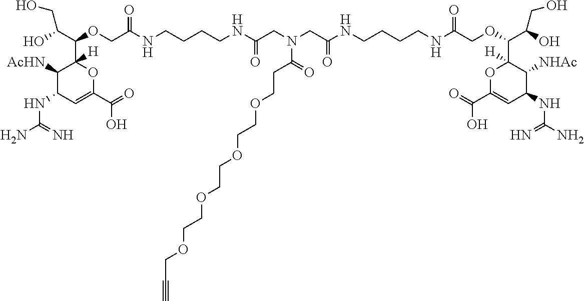 [SMILES: C#CCOCCOCCOCCOCCC(=O)N(CC(=O)NCCCCNC(=O)CO[C@@H]([C@@H]1OC(C(=O)O)=C[C@H](NC(=N)N)[C@H]1NC(C)=O)[C@H](O)CO)CC(=O)NCCCCNC(=O)CO[C@@H]([C@@H]1OC(C(=O)O)=C[C@H](NC(=N)N)[C@H]1NC(C)=O)[C@H](O)CO] |
| Int-55 | 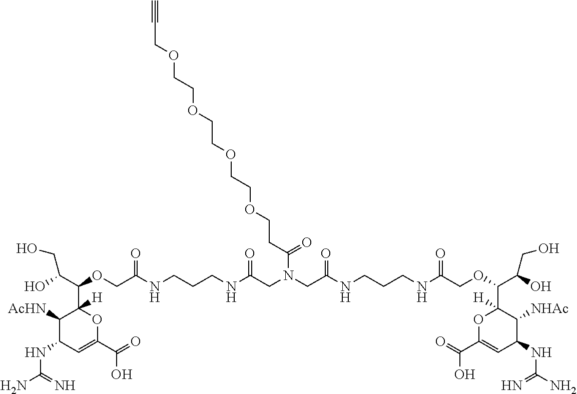 [SMILES: C#CCOCCOCCOCCOCCC(=O)N(CC(=O)NCCCNC(=O)CO[C@@H]([C@@H]1OC(C(=O)O)=C[C@H](NC(=N)N)[C@H]1NC(C)=O)[C@H](O)CO)CC(=O)NCCCNC(=O)CO[C@@H]([C@@H]1OC(C(=O)O)=C[C@H](NC(=N)N)[C@H]1NC(C)=O)[C@H](O)CO] |
| Int-57 | 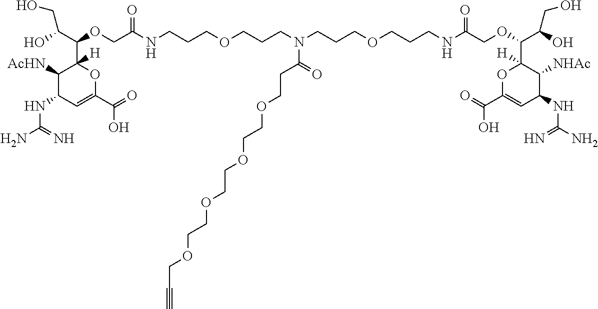 [SMILES: C#CCOCCOCCOCCOCCC(=O)N(CCCOCCCNC(=O)CO[C@@H]([C@@H]1OC(C(=O)O)=C[C@H](NC(=N)N)[C@H]1NC(C)=O)[C@H](O)CO)CCCOCCCNC(=O)CO[C@@H]([C@@H]1OC(C(=O)O)=C[C@H](NC(=N)N)[C@H]1NC(C)=O)[C@H](O)CO] |
| Int-58 | 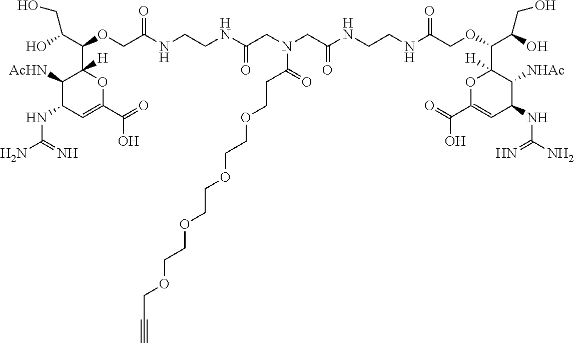 [SMILES: C#CCOCCOCCOCCOCCC(=O)N(CC(=O)NCCNC(=O)CO[C@@H]([C@@H]1OC(C(=O)O)=C[C@H](NC(=N)N)[C@H]1NC(C)=O)[C@H](O)CO)CC(=O)NCCNC(=O)CO[C@@H]([C@@H]1OC(C(=O)O)=C[C@H](NC(=N)N)[C@H]1NC(C)=O)[C@H](O)CO] |
| Int-59 | 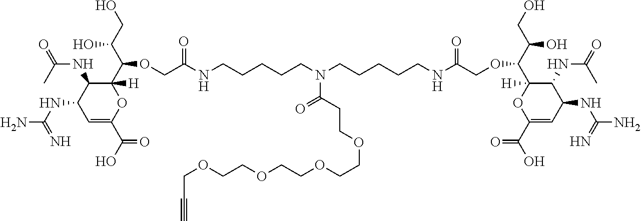 [SMILES: C#CCOCCOCCOCCOCCC(=O)N(CCCCCNC(=O)CO[C@@H]([C@@H]1OC(C(=O)O)=C[C@H](NC(=N)N)[C@H]1NC(C)=O)[C@H](O)CO)CCCCCNC(=O)CO[C@@H]([C@@H]1OC(C(=O)O)=C[C@H](NC(=N)N)[C@H]1NC(C)=O)[C@H](O)CO] |
| Int-60 | 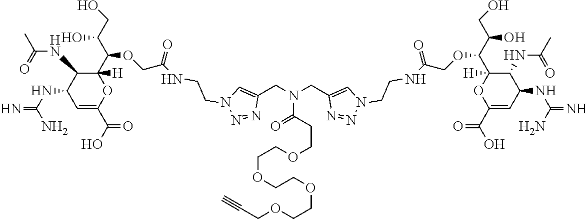 [SMILES: C#CCOCCOCCOCCOCCC(=O)N(Cc1cn(CCNC(=O)CO[C@@H]([C@@H]2OC(C(=O)O)=C[C@H](NC(=N)N)[C@H]2NC(C)=O)[C@H](O)CO)nn1)Cc1cn(CCNC(=O)CO[C@@H]([C@@H]2OC(C(=O)O)=C[C@H](NC(=N)N)[C@H]2NC(C)=O)[C@H](O)CO)nn1] |
| Int-61 | 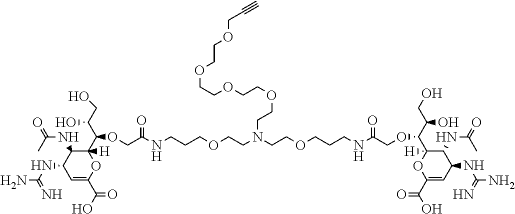 [SMILES: C#CCOCCOCCOCCOCCN(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| Int-62 | 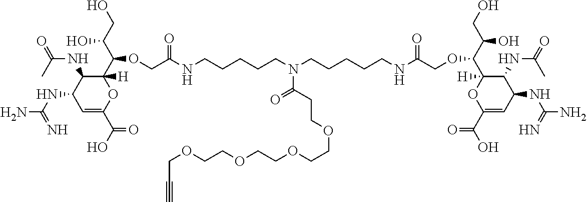 [SMILES: C#CCOCCOCCOCCOCCC(=O)N(CCCCCNC(=O)CO[C@@H]([C@@H]1OC(C(=O)O)=C[C@H](NC(=N)N)[C@H]1NC(C)=O)[C@H](O)CO)CCCCCNC(=O)CO[C@@H]([C@@H]1OC(C(=O)O)=C[C@H](NC(=N)N)[C@H]1NC(C)=O)[C@H](O)CO] |
| Int-63 | 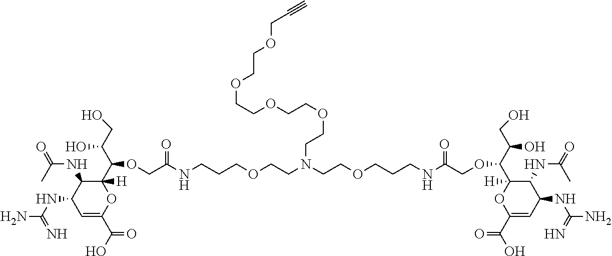 [SMILES: C#CCOCCOCCOCCOCCN(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| Int-64 | 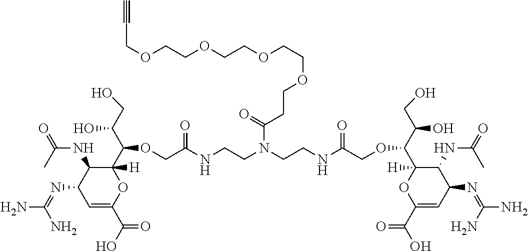 [SMILES: C#CCOCCOCCOCCOCCC(=O)N(CCNC(=O)CO[C@@H]([C@@H]1OC(C(=O)O)=C[C@H](N=C(N)N)[C@H]1NC(C)=O)[C@H](O)CO)CCNC(=O)CO[C@@H]([C@@H]1OC(C(=O)O)=C[C@H](N=C(N)N)[C@H]1NC(C)=O)[C@H](O)CO] |
| Int-65 | 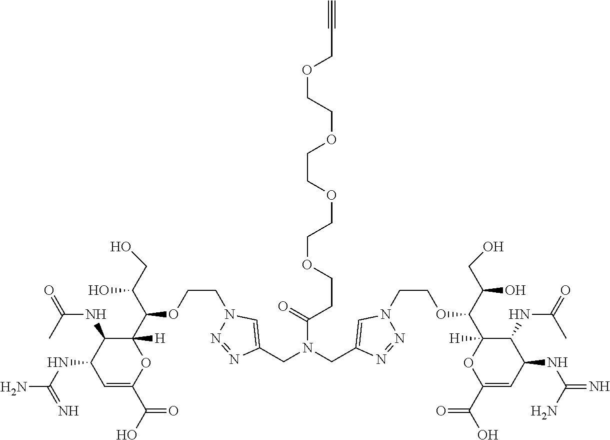 [SMILES: C#CCOCCOCCOCCOCCC(=O)N(Cc1cn(CCO[C@@H]([C@@H]2OC(C(=O)O)=C[C@H](NC(=N)N)[C@H]2NC(C)=O)[C@H](O)CO)nn1)Cc1cn(CCO[C@@H]([C@@H]2OC(C(=O)O)=C[C@H](NC(=N)N)[C@H]2NC(C)=O)[C@H](O)CO)nn1] |
| Int-69 | 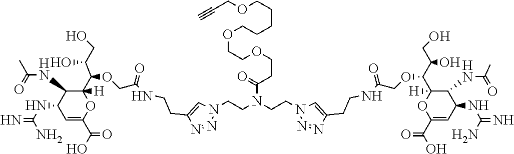 [SMILES: C#CCOCCCCCOCCOCCC(=O)N(CCn1cc(CCNC(=O)CO[C@@H]([C@@H]2OC(C(=O)O)=C[C@H](NC(=N)N)[C@H]2NC(C)=O)[C@H](O)CO)nn1)CCn1cc(CCNC(=O)CO[C@@H]([C@@H]2OC(C(=O)O)=C[C@H](NC(=N)N)[C@H]2NC(C)=O)[C@H](O)CO)nn1] |
| Int-70 | 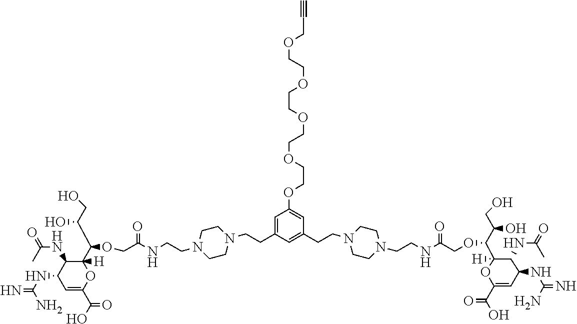 [SMILES: C#CCOCCOCCOCCOCCOc1cc(CCN2CCN(CCNC(=O)CO[C@@H]([C@@H]3OC(C(=O)O)=C[C@H](NC(=N)N)[C@H]3NC(C)=O)[C@H](O)CO)CC2)cc(CCN2CCN(CCNC(=O)CO[C@@H]([C@@H]3OC(C(=O)O)=C[C@H](NC(=N)N)[C@H]3NC(C)=O)[C@H](O)CO)CC2)c1] |
| Int-71 | 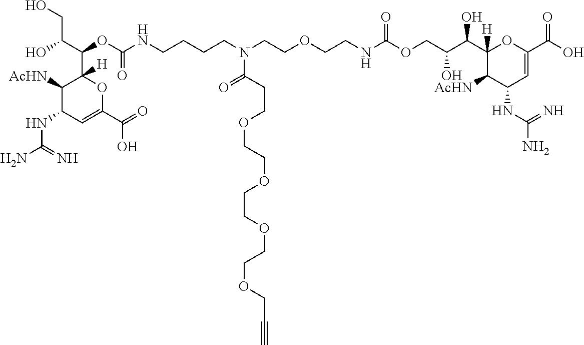 [SMILES: C#CCOCCOCCOCCOCCC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)OC[C@@H](O)[C@@H](O)[C@@H]1OC(C(=O)O)=C[C@H](NC(=N)N)[C@H]1NC(C)=O] |
| Int-72 | 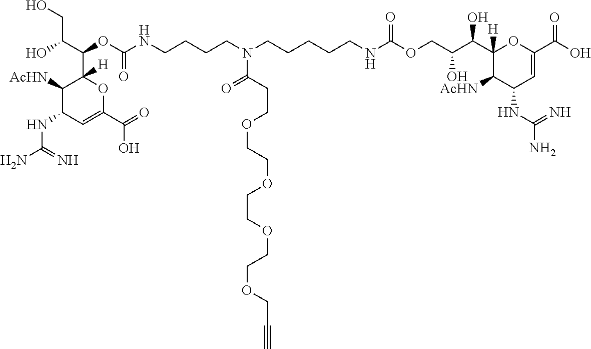 [SMILES: C#CCOCCOCCOCCOCCC(=O)N(CCCCCNC(=O)OC[C@@H](O)[C@@H](O)[C@@H]1OC(C(=O)O)=C[C@H](NC(=N)N)[C@H]1NC(C)=O)CCCCNC(=O)O[C@@H]([C@@H]1OC(C(=O)O)=C[C@H](NC(=N)N)[C@H]1NC(C)=O)[C@H](O)CO] |
| Int-73 | 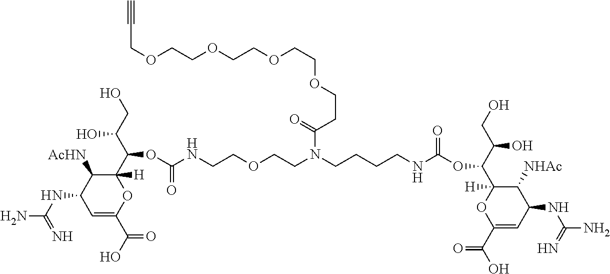 [SMILES: C#CCOCCOCCOCCOCCC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| Int-74 | 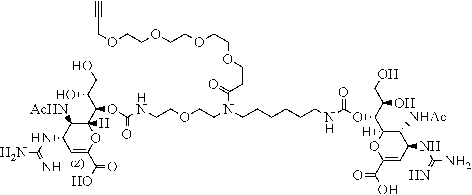 [SMILES: C#CCOCCOCCOCCOCCC(=O)N(CCCCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| Int-75 | 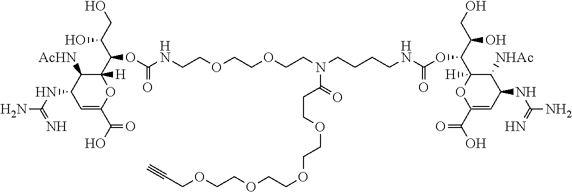 [SMILES: C#CCOCCOCCOCCOCCC(=O)N(CCCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| Int-76 | 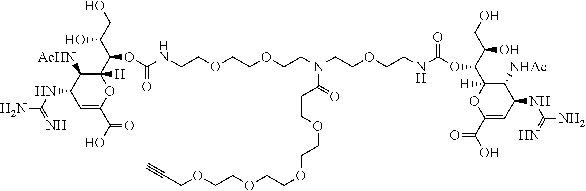 [SMILES: C#CCOCCOCCOCCOCCC(=O)N(CCOCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| Int-77 | 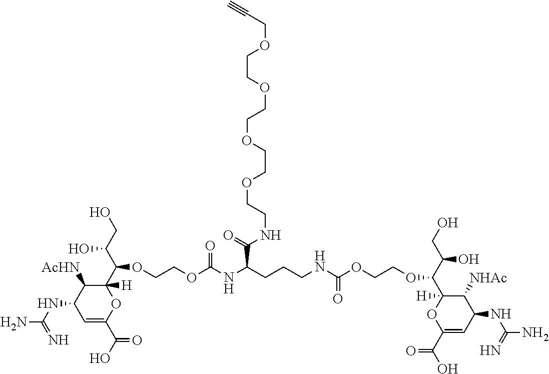 [SMILES: C#CCOCCOCCOCCOCCNC(=O)[C@@H](CCCNC(=O)OCCO[C@@H]([C@@H]1OC(C(=O)O)=C[C@H](NC(=N)N)[C@H]1NC(C)=O)[C@H](O)CO)NC(=O)OCCO[C@@H]([C@@H]1OC(C(=O)O)=C[C@H](NC(=N)N)[C@H]1NC(C)=O)[C@H](O)CO] |
| Int-78 | 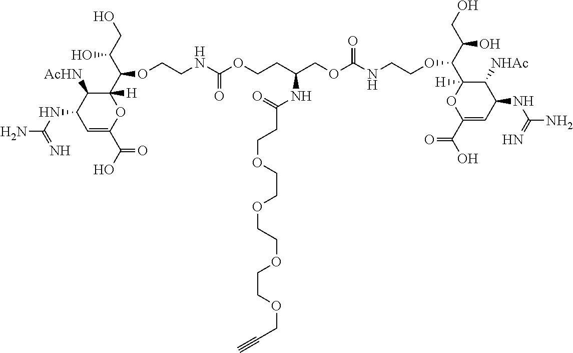 [SMILES: C#CCOCCOCCOCCOCCC(=O)N[C@@H](CCOC(=O)NCCO[C@@H]([C@@H]1OC(C(=O)O)=C[C@H](NC(=N)N)[C@H]1NC(C)=O)[C@H](O)CO)COC(=O)NCCO[C@@H]([C@@H]1OC(C(=O)O)=C[C@H](NC(=N)N)[C@H]1NC(C)=O)[C@H](O)CO] |
| Int-79 | 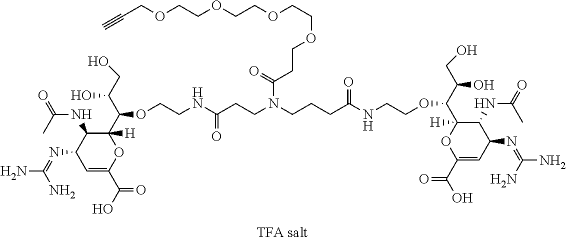 [SMILES: C#CCOCCOCCOCCOCCC(=O)N(CCCC(=O)NCCO[C@@H]([C@@H]1OC(C(=O)O)=C[C@H](N=C(N)N)[C@H]1NC(C)=O)[C@H](O)CO)CCC(=O)NCCO[C@@H]([C@@H]1OC(C(=O)O)=C[C@H](N=C(N)N)[C@H]1NC(C)=O)[C@H](O)CO] |
| Int-81 | 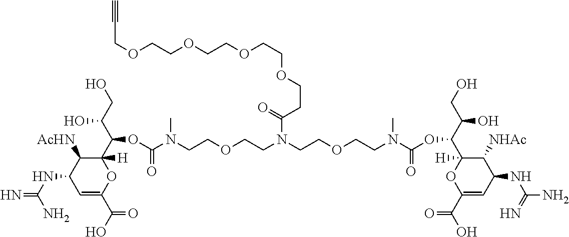 [SMILES: C#CCOCCOCCOCCOCCC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-82 | 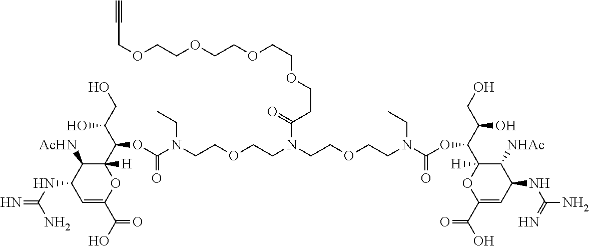 [SMILES: C#CCOCCOCCOCCOCCC(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| Int-83 | 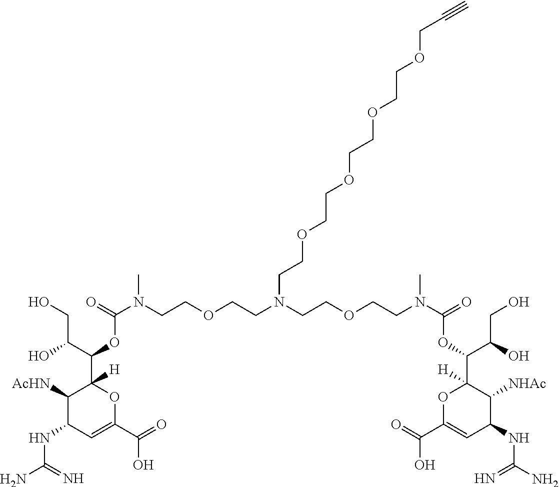 [SMILES: C#CCOCCOCCOCCOCCN(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-84 | 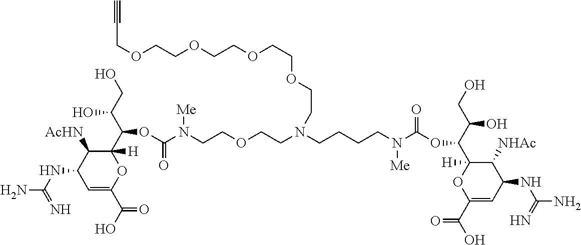 [SMILES: C#CCOCCOCCOCCOCCN(CCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-85 | 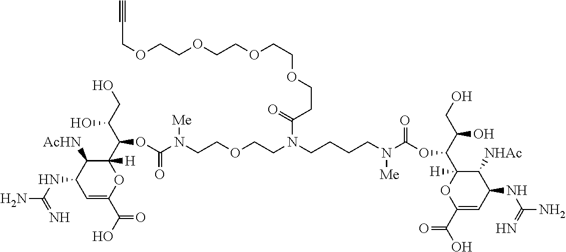 [SMILES: C#CCOCCOCCOCCOCCC(=O)N(CCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-86 | 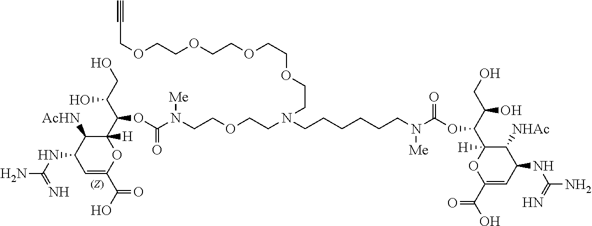 [SMILES: C#CCOCCOCCOCCOCCN(CCCCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-87 | 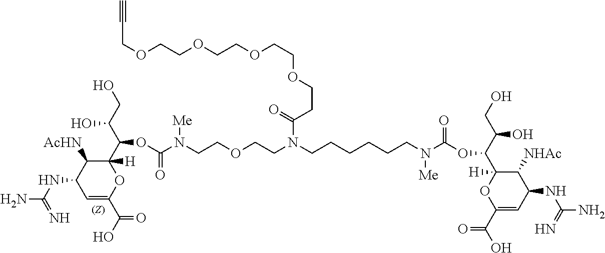 [SMILES: C#CCOCCOCCOCCOCCC(=O)N(CCCCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-88 | 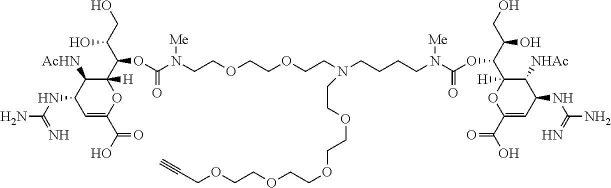 [SMILES: C#CCOCCOCCOCCOCCN(CCCCN(C)C(=O)O[C@@H]([C@@H]1OC(C(=O)O)=C[C@H](NC(=N)N)[C@H]1NC(C)=O)[C@H](O)CO)CCOCCOCCN(C)C(=O)O[C@@H]([C@@H]1OC(C(=O)O)=C[C@H](NC(=N)N)[C@H]1NC(C)=O)[C@H](O)CO] |
| Int-89 | 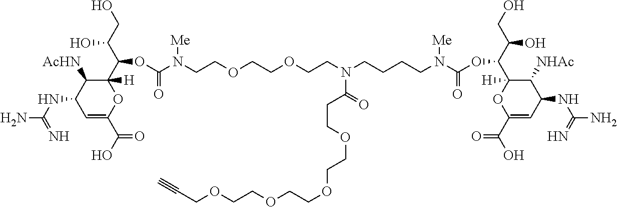 [SMILES: C#CCOCCOCCOCCOCCC(=O)N(CCCCN(C)C(=O)O[C@@H]([C@@H]1OC(C(=O)O)=C[C@H](NC(=N)N)[C@H]1NC(C)=O)[C@H](O)CO)CCOCCOCCN(C)C(=O)O[C@@H]([C@@H]1OC(C(=O)O)=C[C@H](NC(=N)N)[C@H]1NC(C)=O)[C@H](O)CO] |
| Int-90 | 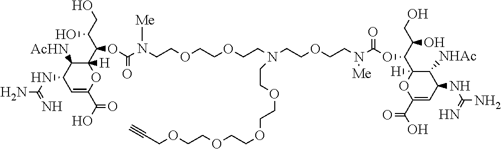 [SMILES: C#CCOCCOCCOCCOCCN(CCOCCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-93 | 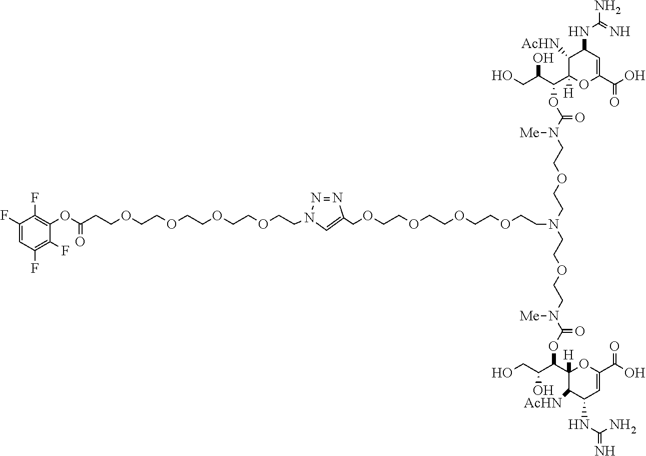 |
| Int-94 | 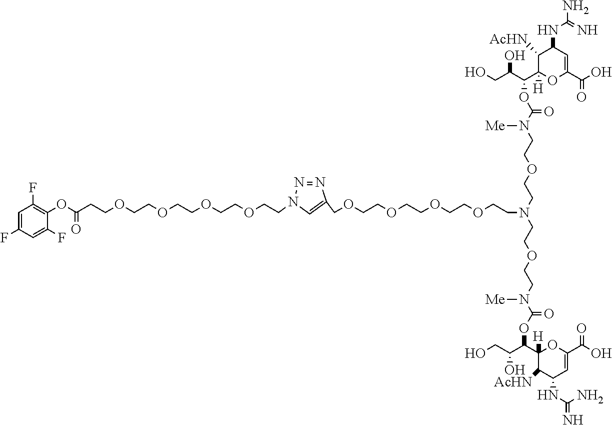 |

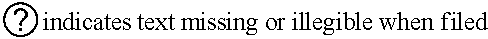

In another aspect, the disclosure features a conjugate of Table 1b. Conjugates of Table 1b include conjugates formed by the covalent reaction of an Int of Table 1a with a linker which is in turn conjugated to E (e.g., an Fc domain monomer). In some embodiments, the reactive moiety of the Int (e.g., the alkyne, azido, or amine group) reacts with a corresponding reactive group (e.g., an alkyne, azido, or phenyl ester group) of a linker (represented by L) covalently attached to E, such that an Int of Table 1a is covalently attached to E. As represented in Table 1b, L′ corresponds to the remainder of L (e.g., L′ is a linker that covalently joins the Int and E). For example, L′ may include a triazole (formed by the click chemistry reaction between the Int and a linker conjugated to E) and a linker (e.g., a PEG₂-PEG₂₀ linker) which in turn is conjugated to an amino acid side chain of E.

In some embodiments the conjugate of Table 1b, n is 1 or 2. When n is 1, each E includes an Fc domain monomer (e.g., an Fc domain monomer having the sequence of any one of SEQ ID NOs: 1-29). When n is 2, each E includes an Fc domain monomer (e.g., an Fc domain monomer having the sequence of any one of SEQ ID NOs: 1-29), and the Fc domain monomers dimerize to form an Fc domain.

In some embodiments of any conjugate of Table 1b, T is an integer from 1 to 20 (e.g., 1, 2, 3, 4, 5, 6, 7, 8, 9, 10, 11, 12, 13, 14, 15, 16, 17, 18, 19, or 20). The disclosure also provides a population of any of the conjugates of Table 1b wherein the average value of T is 1 to 20 (e.g., the average value of T is 1 to 2, 1 to 3, 1 to 4, 1 to 5, 5 to 10, 10 to 15, 15 to 20, 1.5 to 3.5, 2.5 to 4.5, 3.5 to 5.5, 4.5 to 6.5, 5.5 to 7.5, 6.5 to 8.5, 7.5 to 9.5, or 8.5 to 10.5).

The squiggly line in the conjugates of Table 1b indicates that each L′-Int is covalently attached to an amino acid side chain in E (e.g., the nitrogen atom of a surface exposed lysine or the sulfur atom of a surface exposed cysteine in E).

TABLE 1b
Conjugates Corresponding to Intermediates of Table 1a

| Intermediate | |
|---|---|
| of Table 1a | Conjugate Structure |
| Int-1 | 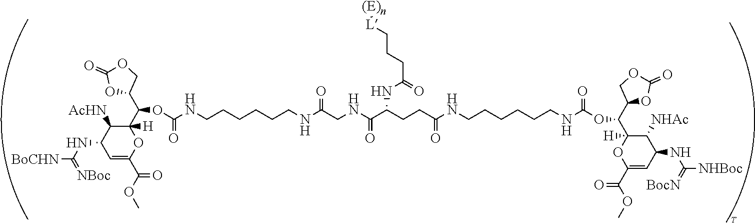 |
| Int-2 | 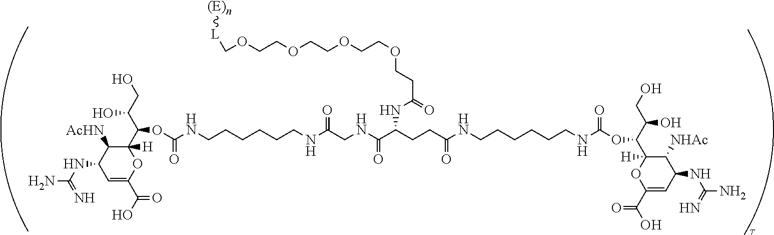 |
| Int-3 | 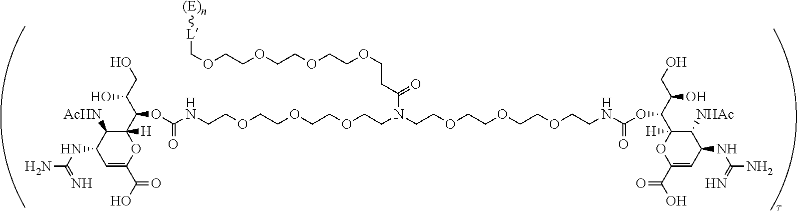 |
| Int-7 | 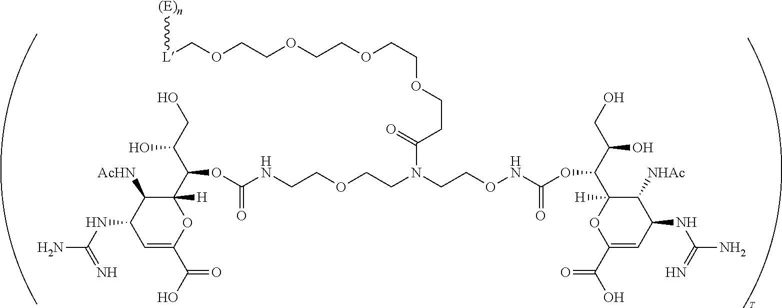 |
| Int-9 | 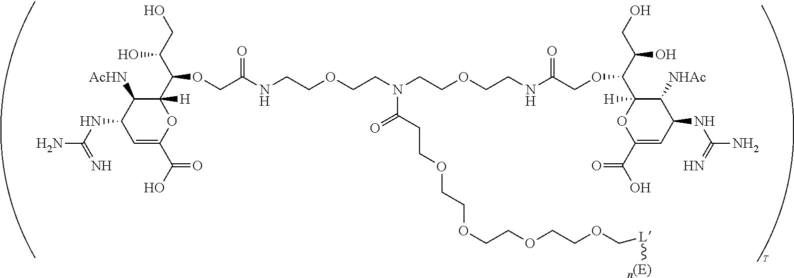 |
| Int-12 | 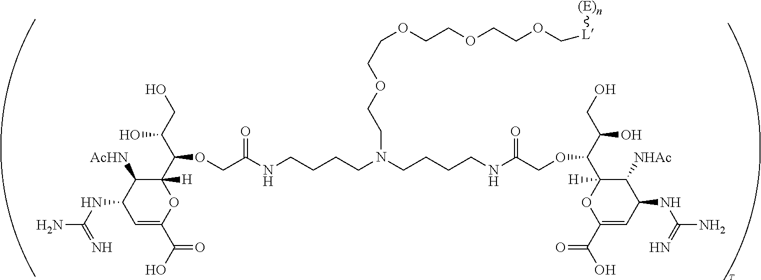 |
| Int-17 | 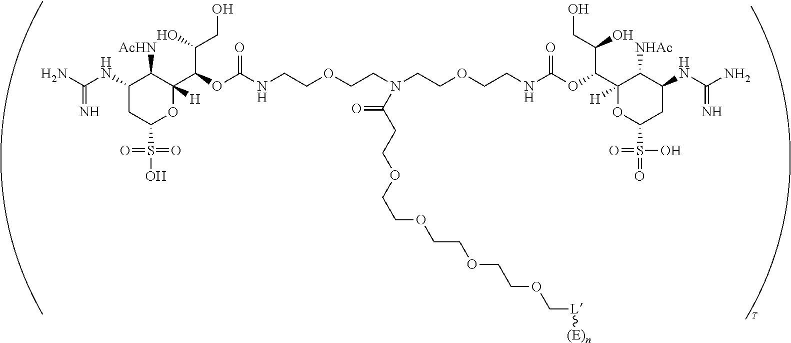 |
| Int-18 | 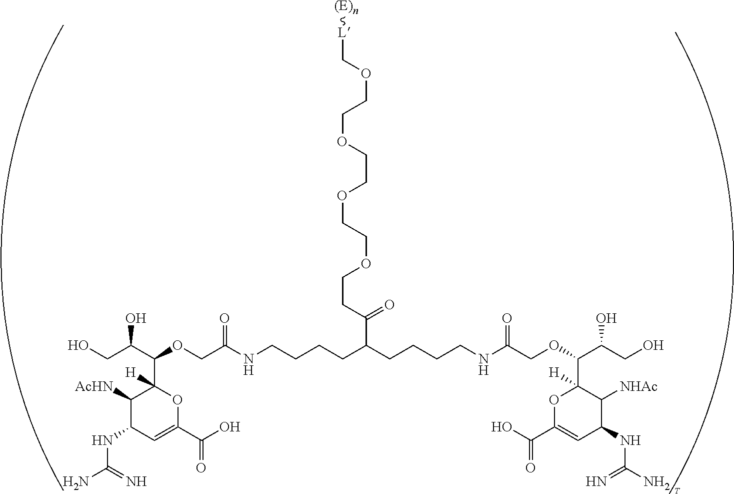 |
| Int-19 | 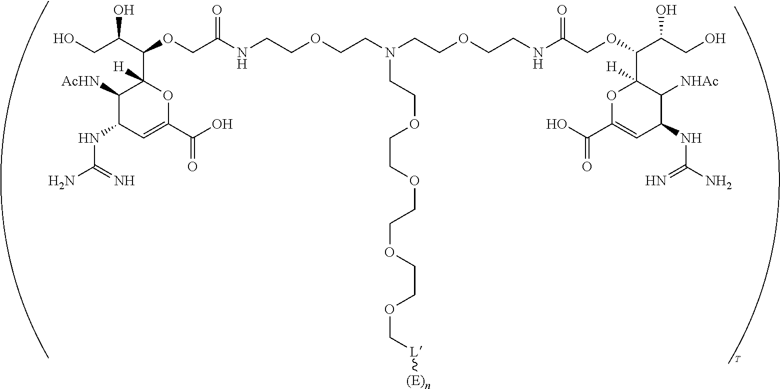 |
| Int-20 | 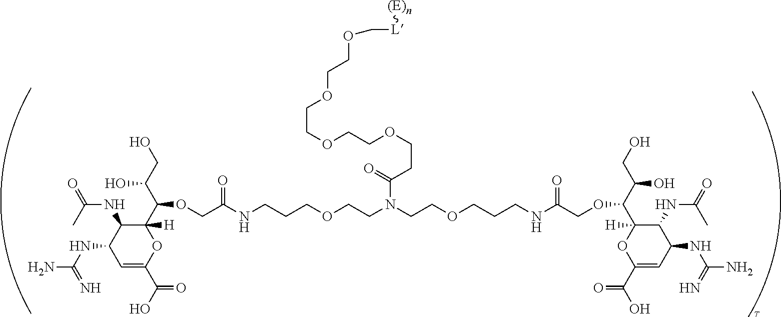 |
| Int-21 | 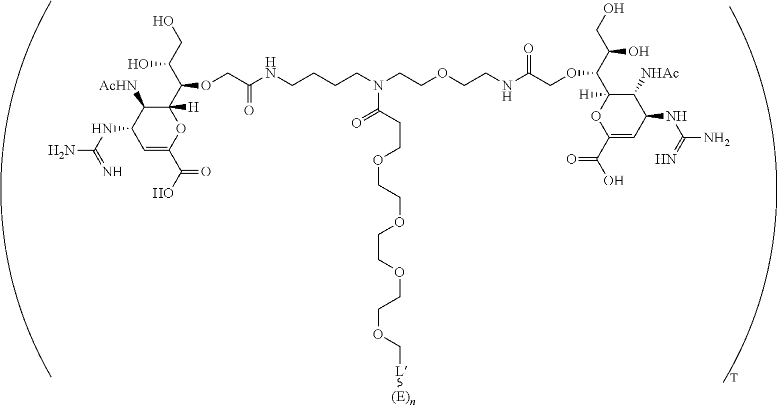 |
| Int-23 | 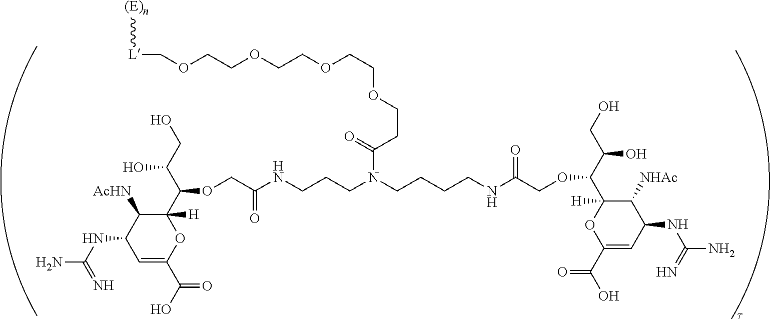 |
| Int-24 | 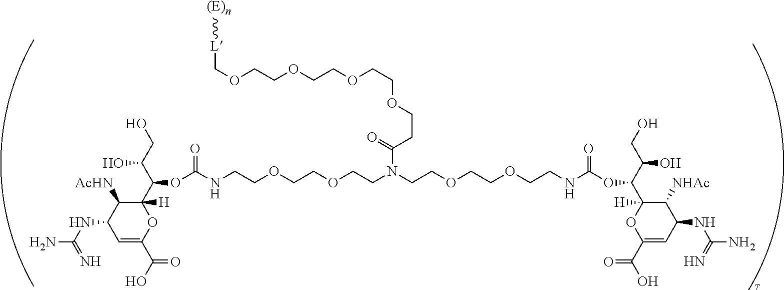 |
| Int-25 | 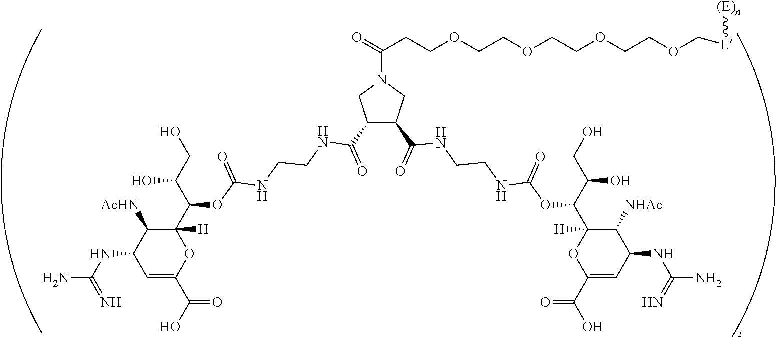 |
| Int-26 | 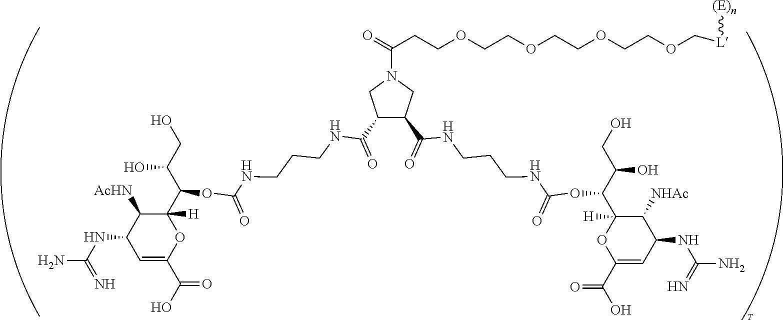 |
| Int-27 | 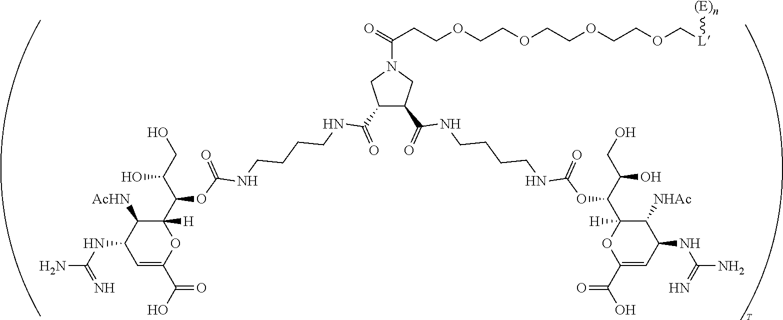 |
| Int-28 | 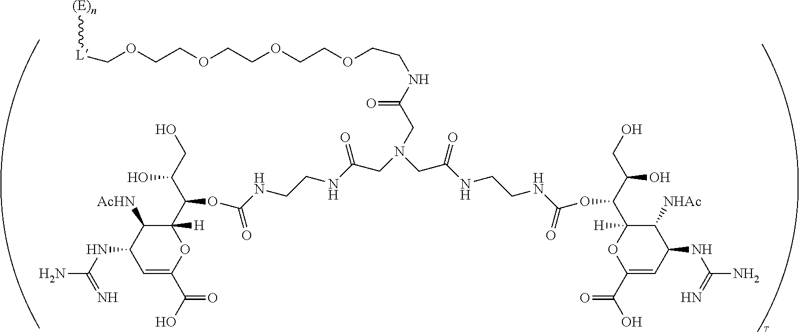 |
| Int-31 | 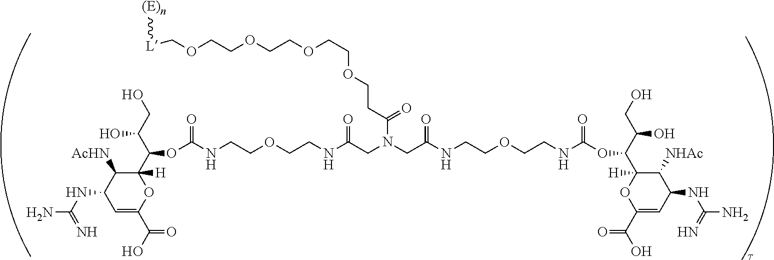 |
| Int-34 | 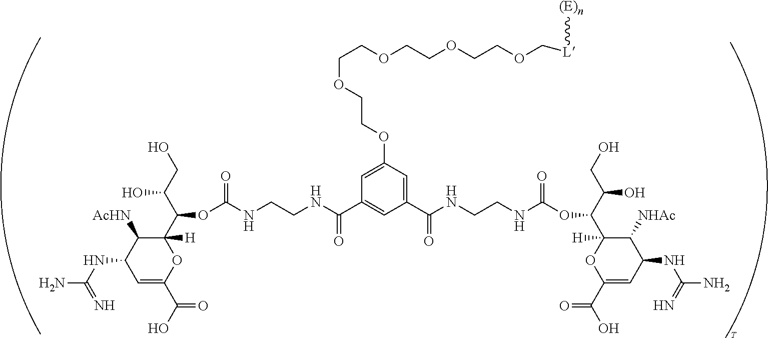 |
| Int-38 | 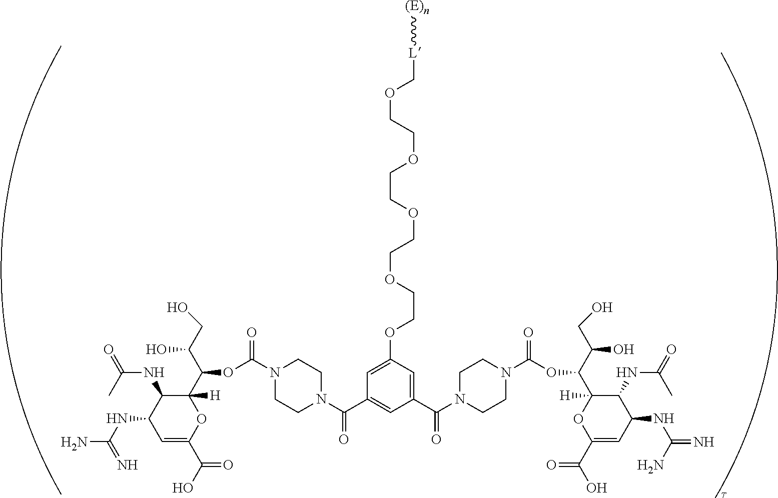 |
| Int-39 | 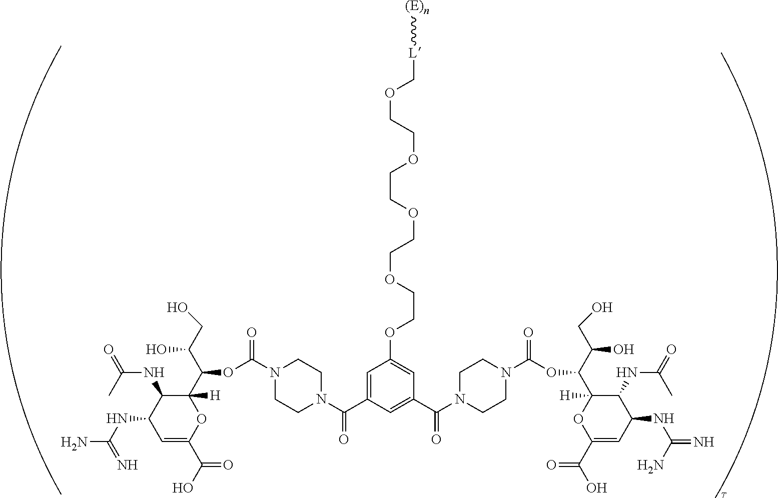 |
| Int-42 | 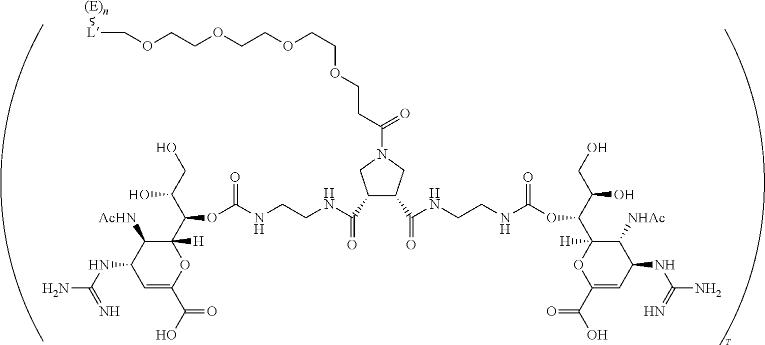 |
| Int-43 | 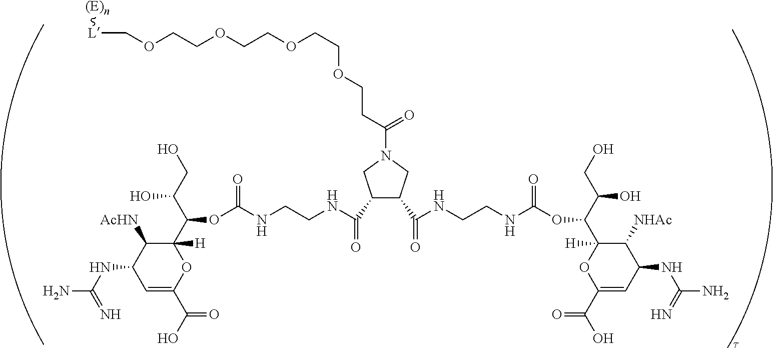 |
| Int-44 | 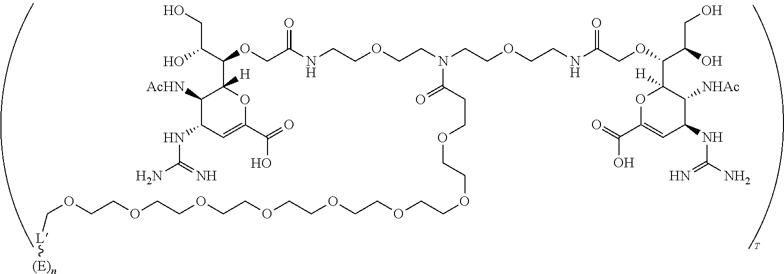 |
| Int-45 | 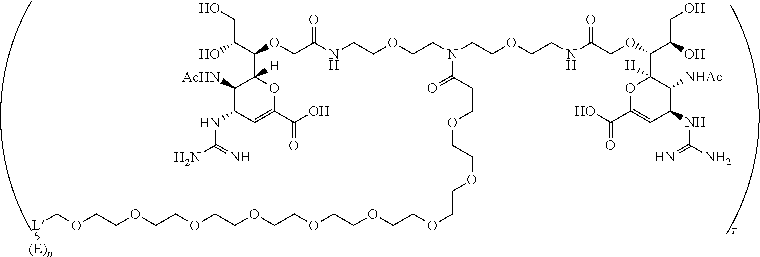 |
| Int-46 | 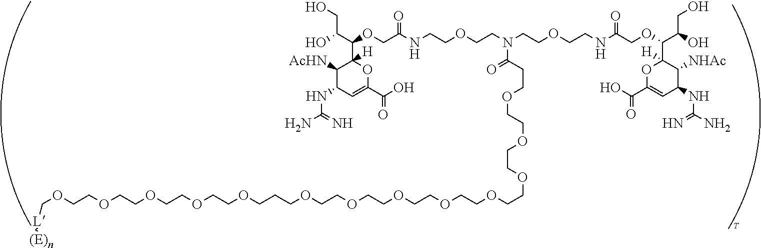 |
| Int-47 | 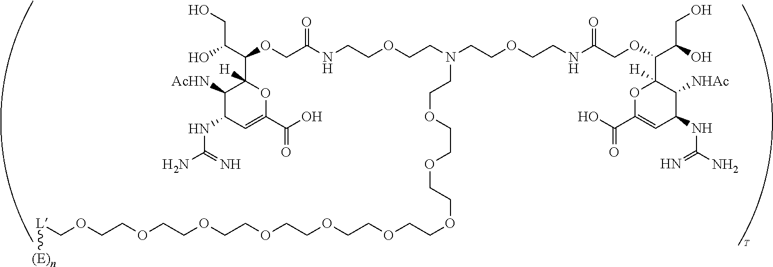 |
| Int-48 | 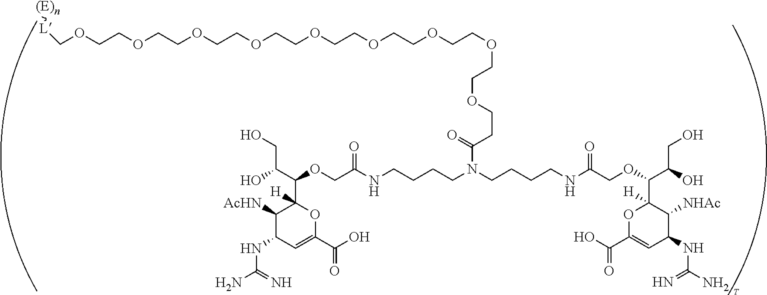 |
| Int-49 | 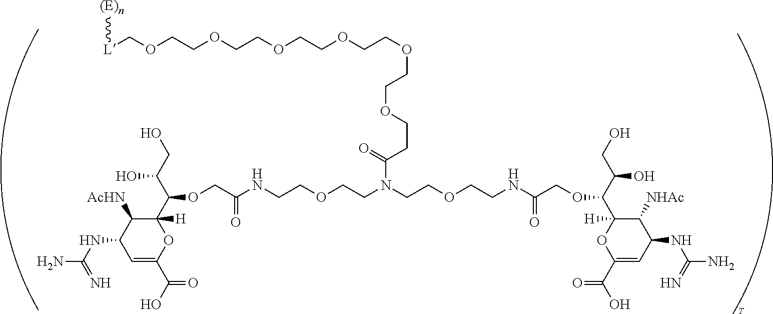 |
| Int-50 | 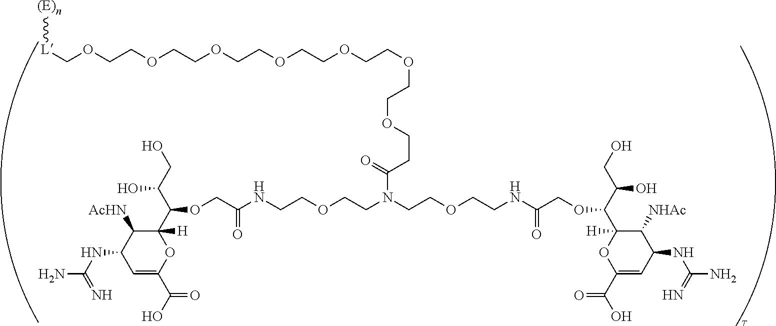 |
| Int-52 | 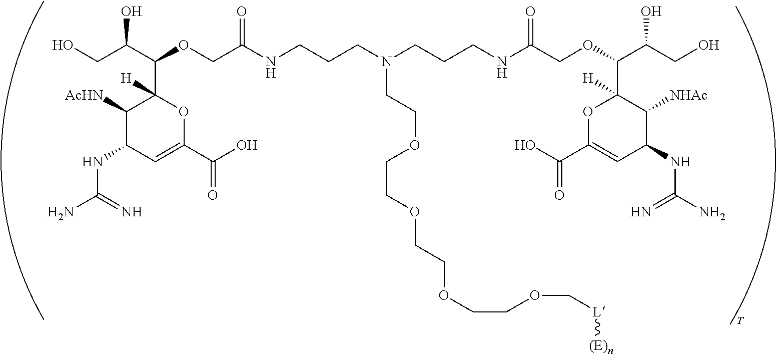 |
| Int-53 | 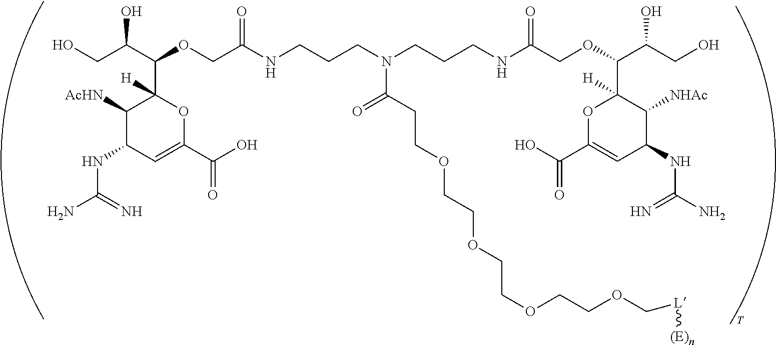 |
| Int-54 | 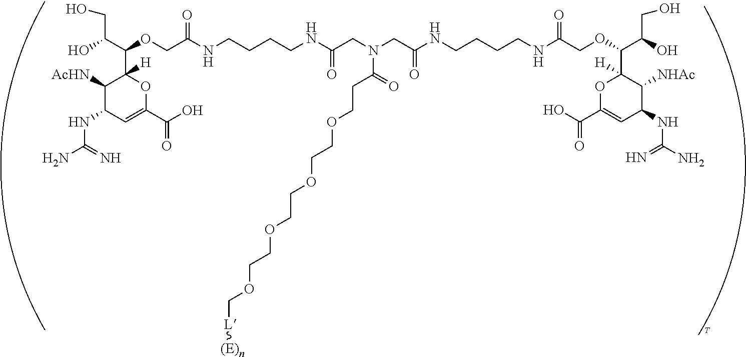 |
| Int-55 | 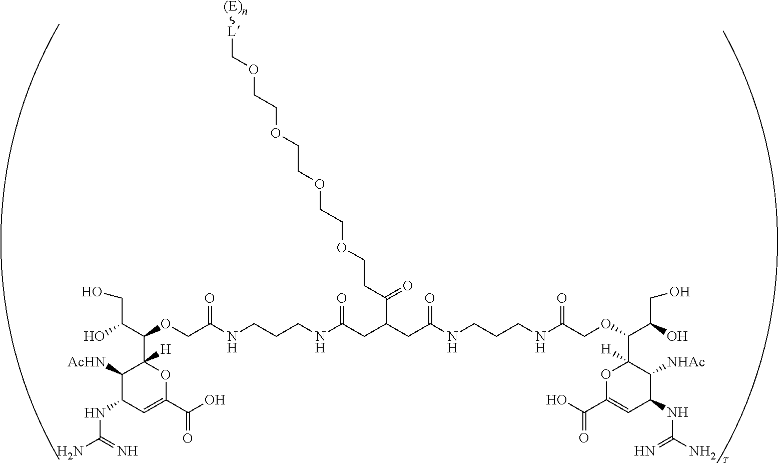 |
| Int-57 | 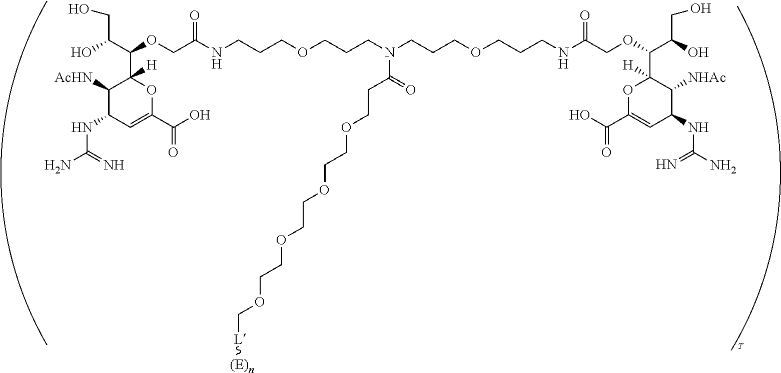 |
| Int-58 | 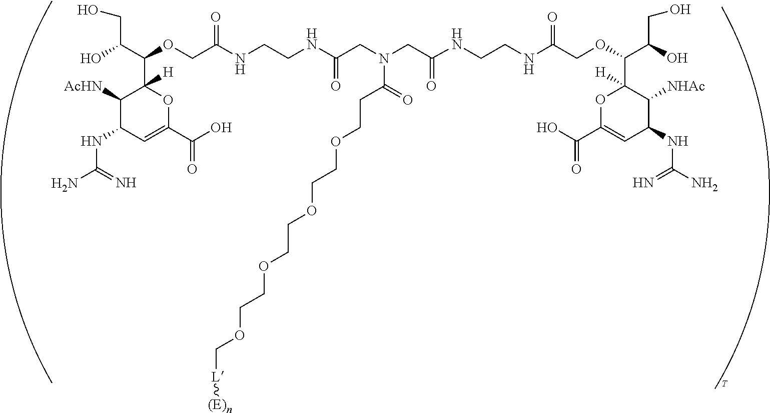 |
| Int-59 | 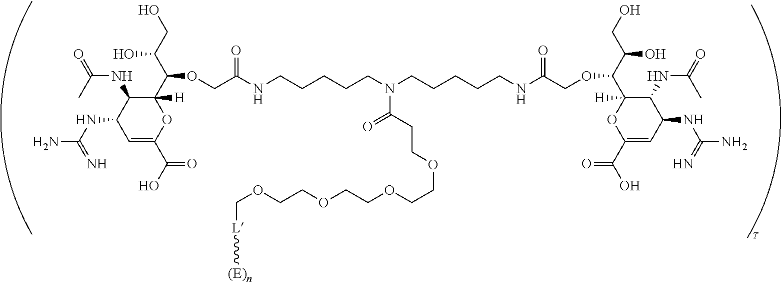 |
| Int-60 | 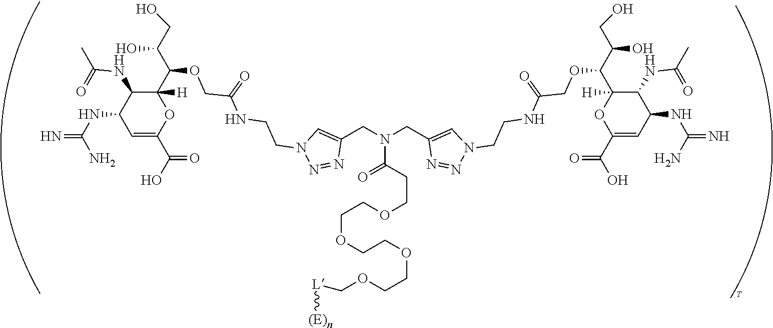 |
| Int-61 | 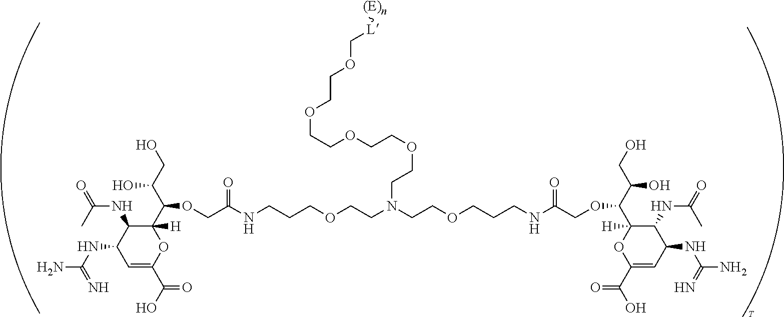 |
| Int-62 | 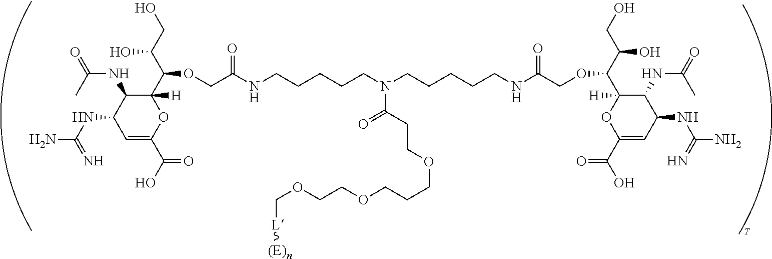 |
| Int-63 | 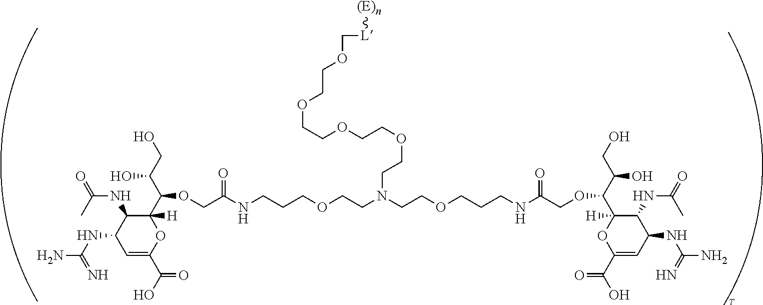 |
| Int-64 | 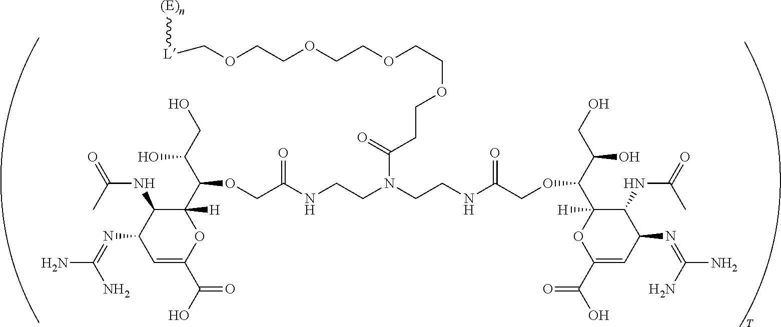 |
| Int-65 | 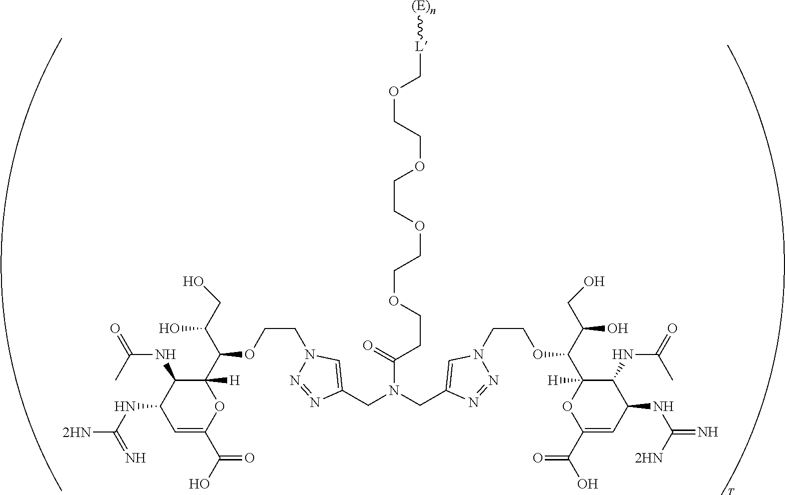 |
| Int-67 | 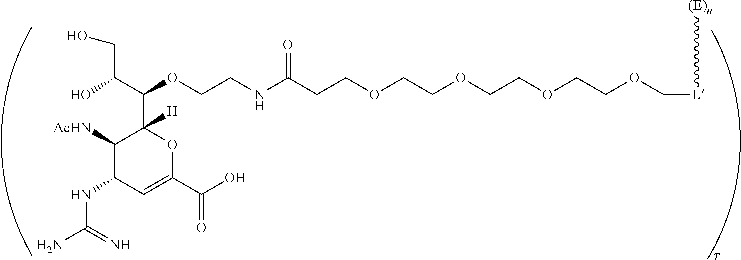 |
| Int-68 | 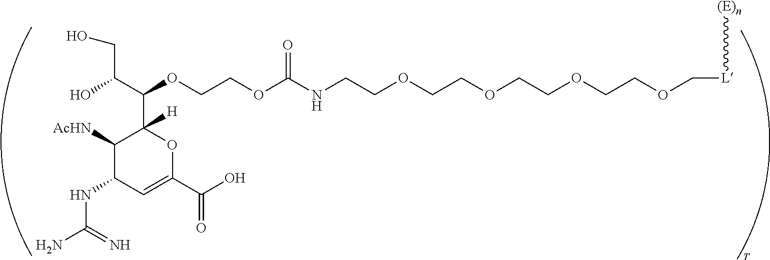 |
| Int-69 | 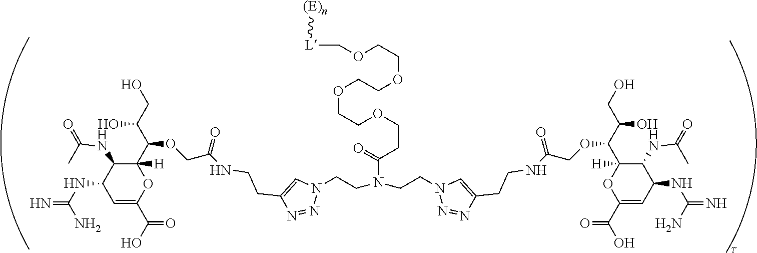 |
| Int-70 | 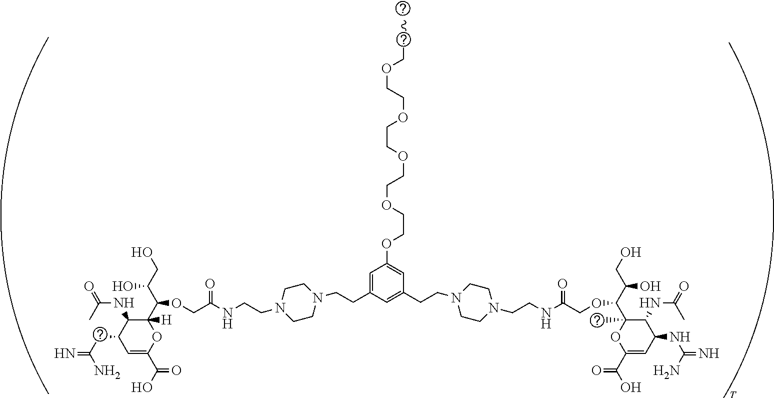 |
| Int-71 | 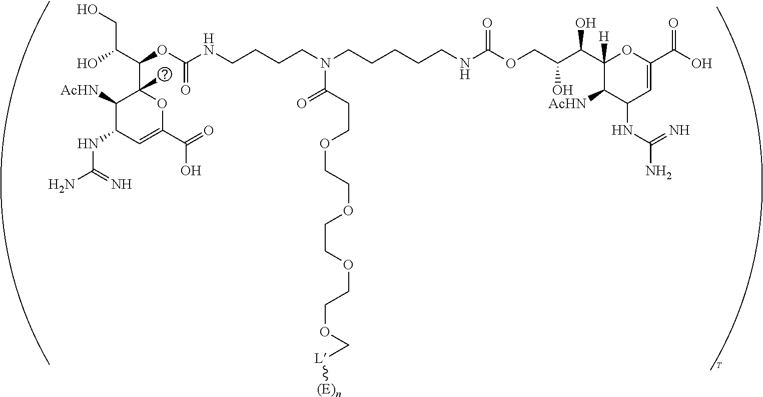 |
| Int-72 | 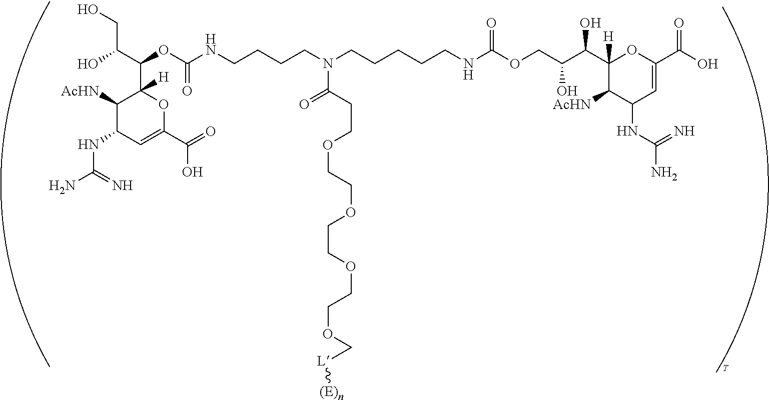 |
| Int-73 | 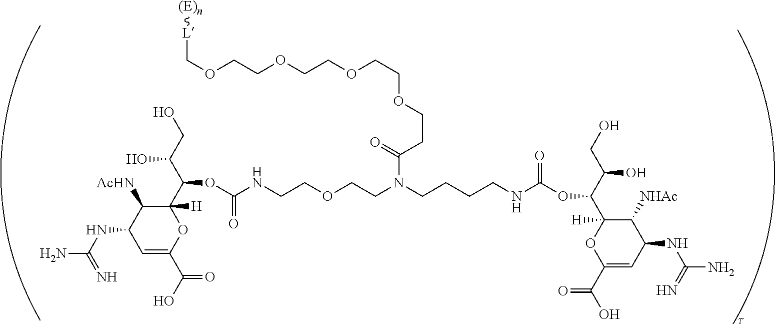 |
| Int-74 | 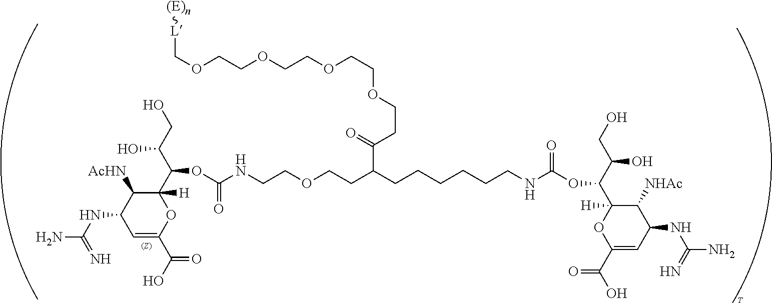 |
| Int-75 | 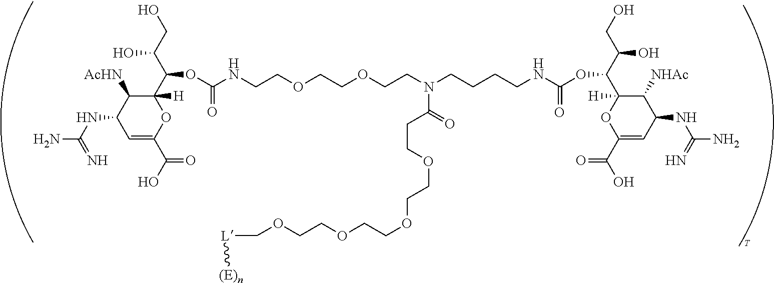 |
| Int-76 | 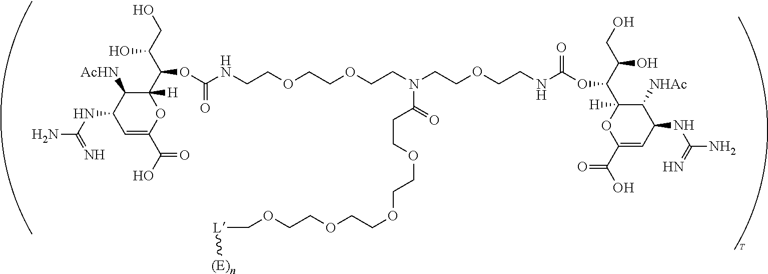 |
| Int-77 | 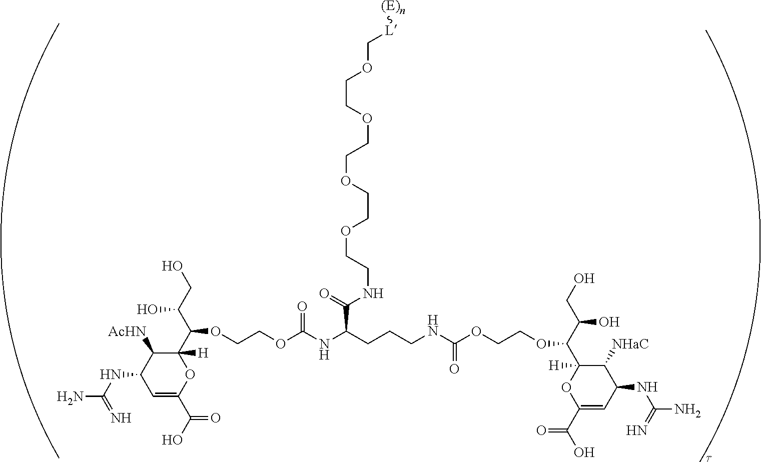 |
| Int-78 | 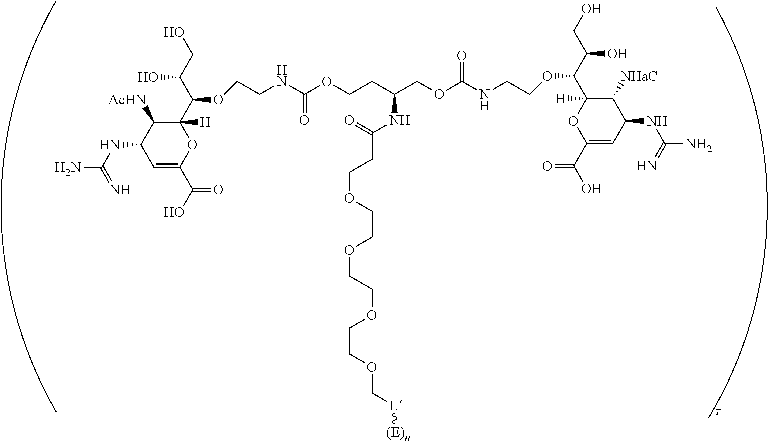 |
| Int-79 | 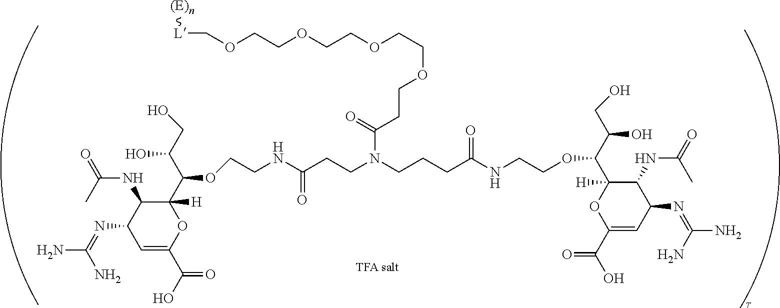 |
| Int-81 | 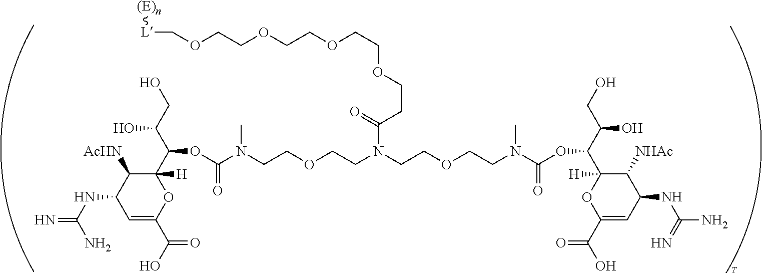 |
| Int-82 | 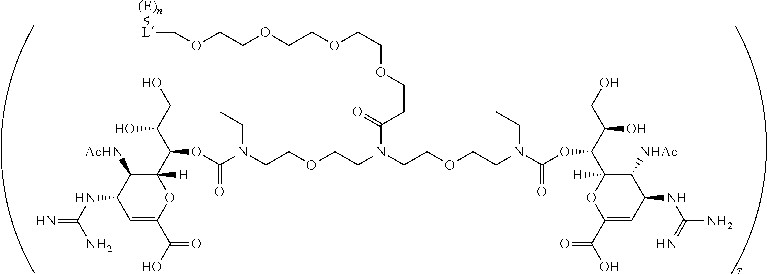 |
| Int-83 | 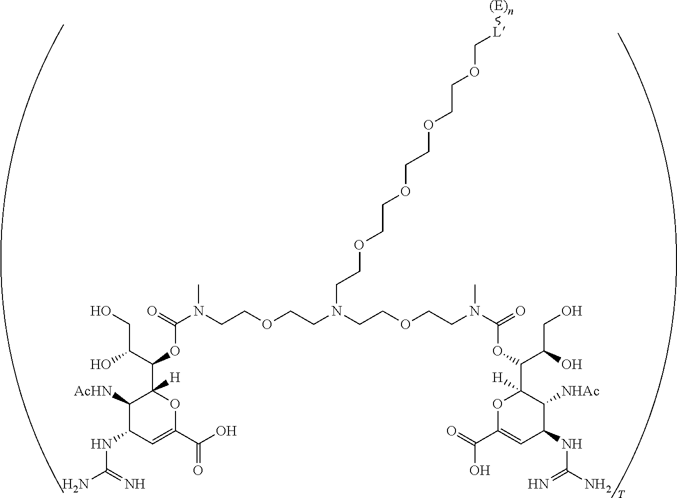 |
| Int-84 | 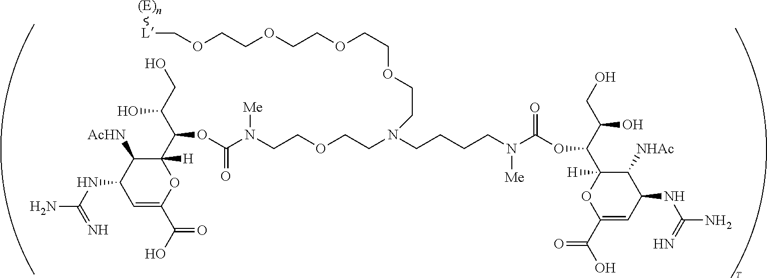 |
| Int-85 | 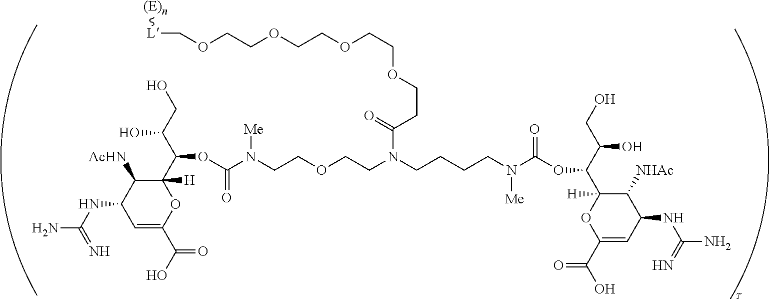 |
| Int-86 | 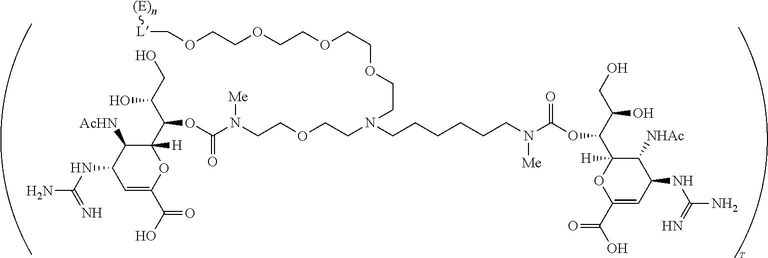 |
| Int-87 | 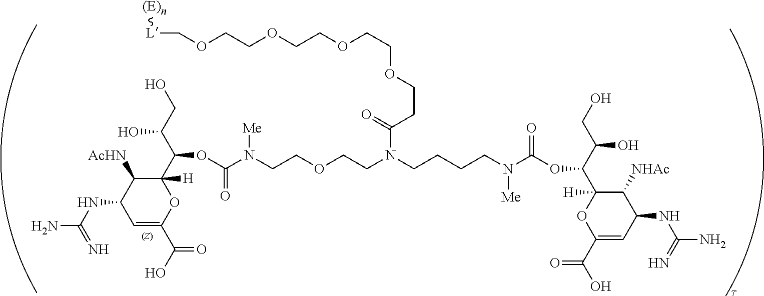 |
| Int-88 | 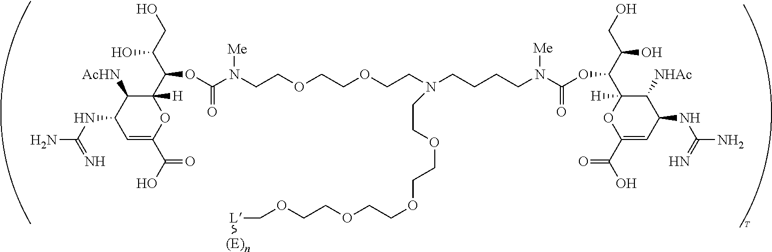 |
| Int-89 | 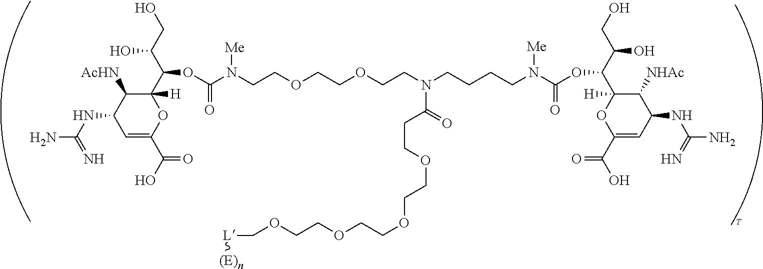 |
| Int-90 | 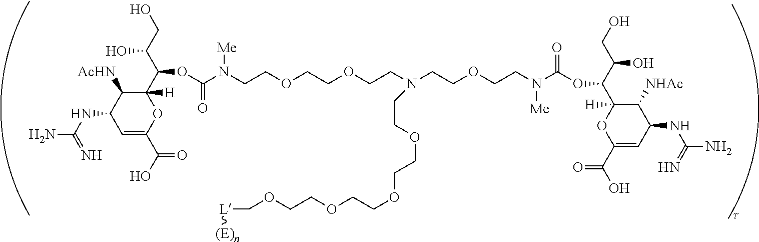 |
| Int-93, Int-94 | 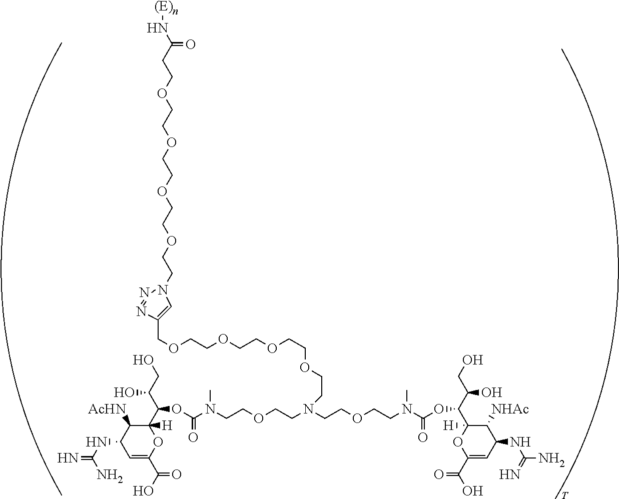 |

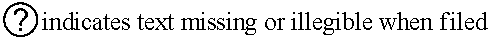

In some embodiments, each E includes an Fc domain monomer having a sequence at least 95% identical to the amino acid sequence of any one of SEQ ID NOs: 1-29. In some embodiments, each E includes an Fc domain monomer having the sequence of any one of SEQ ID NOs: 1-29.

In some embodiments, each E includes an Fc domain monomer including amino acid substitutions at positions (i) 252, 254, and 258, (ii) 309, 311, and 434, or (ii) 428 and 434, and wherein the substitution at position 252 is a tyrosine, the substitution at position at position 254 is a threonine, the substitution at position 258 is a glutamic acid, the substitution at position 309 is an aspartic acid, the substitution at position at position 311 is a histidine, the substitution at positions 428 is a leucine, and the substitution at position 434 is a serine.

In some embodiments, each E includes an Fc domain monomer including an amino acid that is not lysine at position 246; a tyrosine at position 252; a threonine at position 254; and a glutamic acid at position 256. In some embodiments, each E includes an Fc domain monomer includes an amino acid that is not lysine at position 248; an aspartic acid at position 309; a histidine at position 311; and a serine at position 434. In some embodiments, each E includes an Fc domain monomer includes an amino acid that is not lysine at position 248; a methionine at position 428; and a serine at position 434.

In some embodiments, each E includes an Fc domain monomer having an amino acid substitution at position 248 selected from serine, glycine, alanine, threonine, asparagine, glutamine, arginine, histidine, glutamic acid, or aspartic acid. In some embodiments, the amino acid at position 248 is a serine.

In some embodiments, each E includes an Fc domain monomer having a substitution at position 220. In some embodiments, the amino acid at position 220 is a serine.

In some embodiments, each E includes an Fc domain monomer including an aspartic acid at position 356 and a leucine at position 358. In some embodiments, each E includes an Fc domain monomer including a glutamic acid at position 356 and a methionine at position 358.

In some embodiments, each E includes an Fc domain monomer including a substitution at position 297, wherein position 297 is not an asparagine. In some embodiments, the amino acid at position 297 is an alanine.

In some embodiments, the Fc domain monomer is a variant of human IgG1 or human IgG2.

In some embodiments, the Fc domain monomer includes less than about 300 amino acid residues (e.g., less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, or less than about 220 amino acid residues). In some embodiments, the Fc domain monomer is less than about 40 kDa (e.g., less than about 35 kDa, less than about 30 kDa, less than about 25 kDa).

In some embodiments, the Fc domain monomer includes at least 200 amino acid residues (e.g., at least 210, at least 220, at least 230, at least 240, at least 250, at least 260, at least 270, at least 280, at least 290, or at least 300 amino residues). In some embodiments, the Fc domain monomer is at least 20 kDa (e.g., at least 25 kDa, at least 30 kDa, or at least 35 kDa).

In some embodiments, the Fc domain monomer includes 200 to 400 amino acid residues (e.g., 200 to 250, 250 to 300, 300 to 350, 350 to 400, 200 to 300, 250 to 350, or 300 to 400 amino acid residues). In some embodiments, the Fc domain monomer is between 200 and 300 amino acid residues (e.g., between 210 and 300, between 230 and 300, between 250 and 300, between 270 and 300, between 290 and 300, between 210 and 290, between 220 and 280, between 230 and 270, between 240 and 260, or between 245 and 255 amino acid residues) in length. In particular embodiments, the Fc domain monomer is between 240 and 255 amino acid residues (e.g., 241 amino acid residues, 242 amino acid residues, 243 amino acid residues, 244 amino acid residues, 245 amino acid residues, 246 amino acid residues, 247 amino acid residues, 248 amino acid residues, 249 amino acid residues, 250 amino acid residues, 251 amino acid residues, 252 amino acid residues, 253 amino acid residues, or 254 amino acid residues). In even more particular embodiments, the Fc domain monomer is 246 amino acid residues in length. In some embodiments, the Fc domain monomer is 20 to 40 kDa (e.g., 20 to 25 kDa, 25 to 30 kDa, 35 to 40 kDa, 20 to 30 kDa, 25 to 35 kDa, or 30 to 40 KDa). In some embodiments, the Fc domain monomer is between about 20 kDa and about 40 kDa (e.g., 20 kDa to 25 kDa, 25 k Da to 30 k Da, 30 k Da to 35 k Da, 35 k Da to 40 kDa) in mass.

In some embodiments, the N-terminus of the Fc domain monomer includes between 10 and 20 residues (e.g., 11, 12, 13, 14, 15, 16, 17, 18, or 19 residues) of the Fab domain.

In some embodiments, the N-terminus of the Fc domain monomer is any one of amino acid residues 198-205. In some embodiments, the N-terminus of the Fc domain monomer is amino acid residue 201 (e.g., Asn 201). In some embodiments, the N-terminus of the Fc domain monomer is amino acid residue 202 (e.g., Val 202).

In some embodiments, the C-terminus of the Fc domain monomer is any one of amino acid residues 437-447. In some embodiments, the C-terminus of the Fc domain monomer is amino acid residue 446 (e.g., Gly 446). In some embodiments, the C-terminus of the Fc domain monomer is amino acid residue 447 (e.g. Lys 447).

In some embodiments, E is an Fc domain monomer. In some embodiments, n is 2 and each E dimerizes to form an Fc domain.

In some embodiments of any of the aspects described herein, wherein E includes an Fc domain monomer, the Fc domain monomer (e.g., the Fc domain monomer having the sequence of any one of SEQ ID NOs: 1-29) includes a triple mutation corresponding to M252Y/S254T/T256E (YTE). As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-29 may be mutated to include a YTE mutation.

In some embodiments of any of the aspects described herein, wherein E includes an Fc domain monomer, the Fc domain monomer (e.g., the Fc domain monomer having the sequence of any one of SEQ ID NOs: 1-29) includes a double mutant corresponding to M428L/N434S (LS). As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-29 may be mutated to include a LS mutation.

In some embodiments of any of the aspects described herein, wherein E includes an Fc domain monomer, the Fc domain monomer (e.g., the Fc domain monomer having the sequence of any one of SEQ ID NOs: 1-29) includes a mutant corresponding to N434H. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-29 may be mutated to include an N434H mutation.

In some embodiments of any of the aspects described herein, wherein E includes an Fc domain monomer, the Fc domain monomer (e.g., the Fc domain monomer having the sequence of any one of SEQ ID NOs: 1-29) includes a mutant corresponding to C220S. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-29 may be mutated to include a C220S mutation.

In some embodiments of any of the aspects described herein, the Fc domain monomer (e.g., the Fc domain monomer having the sequence of any one of SEQ ID NOs: 1-29) includes a triple mutation corresponding to V309D/Q311H/N434S (DHS). As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-29 may be mutated to include a DHS mutation.

In some embodiments, the squiggly line connected to E indicates that the L of each A₁-L-A₂ is covalently attached to a nitrogen atom of a solvent-exposed lysine of E.

In some embodiments, the squiggly line connected to E indicates that the L of each A₁-L-A₂ L is covalently attached to a sulfur atom of a solvent-exposed cysteine of E.

In some embodiments of any of the aspects described herein, the squiggly line of any one of formulas (D-I)-(D-VIII) may represent a covalent bond between E and the L of A₁-L or A₂-L-A₁. In some embodiments of any of the aspects described herein, the squiggly line of any one of formulas (D-I)-(D-VIII) may represent that one or more amino acid side chains of E (e.g., one or more nitrogen atoms of one or more surface exposed lysine residues of E or one or more sulfur atoms of one or more surface exposed cysteines in E) have been conjugated to a linker (e.g., a PEG₂-PEG₂₀ linker) wherein the linker has been functionalized with a reactive moiety, such that the reactive moiety forms a covalent bond with the L of any A₁-L or any A₂-L-A₁ described herein.

In some embodiments of any of the aspects described herein. T is an integer from 1 to 20 (e.g., 1, 2, 3, 4, 5, 6, 7, 8, 9, 10, 11, 12, 13, 14, 15, 16, 17, 18, 19, or 20). In some embodiments of any of the aspects described herein, T is 1, 2, 3, 4, or 5. When T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected.

In another aspect, the disclosure provides a population of conjugates having the structure of any of the conjugates described herein (e.g., a population of conjugates having the formula of any one of formulas (D-I)-(D-VIII)), wherein the average value of T is 1 to 20 (e.g., the average value of T is 1 to 2, 1 to 3, 1 to 4, 1 to 5, 5 to 10, 10 to 15, 15 to 20, 1.5 to 3.5, 2.5 to 4.5, 3.5 to 5.5, 4.5 to 6.5, 5.5 to 7.5, 6.5 to 8.5, 7.5 to 9.5, or 8.5 to 10.5).

In another aspect, the disclosure provides a pharmaceutical composition comprising any of the conjugates described herein (e.g., a conjugate of any one of formulas (D-I)-(D-VIII)), or a pharmaceutically acceptable salt thereof, and a pharmaceutically acceptable excipient.

In another aspect, the disclosure provides a method for the treatment of a subject having a viral infection or presumed to have a viral infection, the method comprising administering to the subject an effective amount of any of the conjugates or compositions described herein (e.g., a conjugate of any one of formulas (D-I)-(D-VIII)).

In another aspect, the disclosure provides a method for the prophylactic treatment of a viral infection in a subject in need thereof, the method comprising administering to the subject an effective amount of any of the conjugates or compositions described herein (e.g., a conjugate of any one of formulas (D-I)-(D-VIII)).

In some embodiments, the viral infection is caused by influenza virus or parainfluenza virus. In some embodiments, the viral infection is influenza virus A, B, or C, or parainfluenza virus.

In some embodiments, the subject is immunocompromised. In some embodiments, the subject has been diagnosed with humoral immune deficiency, T cell deficiency, neutropenia, asplenia, or complement deficiency. In some embodiments, the subject is being treated or is about to be treated with an immunosuppressive therapy. In some embodiments, the subject has been diagnosed with a disease which causes immunosuppression. In some embodiments, the disease is cancer or acquired immunodeficiency syndrome. In some embodiments, the cancer is leukemia, lymphoma, or multiple myeloma. In some embodiments, the subject has undergone or is about to undergo hematopoietic stem cell transplantation. In some embodiments, the subject has undergone or is about to undergo an organ transplant.

In some embodiments, the subject has or is at risk of developing a secondary infection. In some embodiments, the secondary infection is a bacterial infection (e.g., methicillin-resistant Staphylococcus aureus (MRSA), Streptococcus pneumoniae, Pseudomonas aeruginosa, and/or Haemophilus influenzae), a viral infection, or a fungal infection. In particular embodiments, the secondary infection is MRSA. In certain embodiments, the secondary infection is S. pneumoniae. In some embodiments, the secondary infection is a respiratory infection (e.g., an infection of the respiratory tract). In some embodiments, the secondary infection is associated with (e.g., causes) pneumonia (e.g., bacterial or viral pneumonia). In some embodiments, the subject has or is at risk of developing pneumonia.

In another aspect, the disclosure features a method of preventing a secondary infection in a subject diagnosed with an influenza infection, wherein the method includes administering to the subject a conjugate or composition described herein.

In some embodiments, administering a conjugate or composition of the present disclosure to a subject diagnosed with an influenza infection decreases the likelihood of developing a secondary infection, e.g., by 10%, 20%, 30%, 40%, 50%, 60%, 70%, 80%, 90%, 100%, 200%, 300%, 400%, 500% or more (e.g., as compared to a subject suffering from influenza not treated with the conjugate or composition). For example, administering a conjugate or composition of the present disclosure to a subject diagnosed with an influenza infection decreases the likelihood of developing a secondary bacterial infection (e.g., MRSA, Streptococcus pneumoniae, Pseudomonas aeruginosa, and/or Haemophilus influenzae), e.g., by 10%, 20%, 30%, 40%, 50%, 60%, 70%, 80%, 90%, 100%, 200%, 300%, 400%, 500% or more.

In some embodiments, the conjugate or composition is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularily, mucosally, intrapericardially, intraumbilically, intraocularally, orally, locally, by inhalation, by injection, or by infusion.

In some embodiments, the subject is treated with a second therapeutic agent. In some embodiments, the second therapeutic agent is an antiviral agent. In some embodiments, the antiviral agent is selected from pimovidir, oseltamivir, zanamivir, peramivir, laninamivir, amantadine, or rimantadine. In particular embodiments, the second therapeutic agent is pimovidir. In some embodiments, the second therapeutic agent is a viral vaccine. In some embodiments, the viral vaccine elicits an immune response in the subject against influenza virus A, B, or C, or parainfluenza virus.

In some embodiments, the conjugate is administered in combination with an antiviral agent, where the antiviral agent is baloxavir. In certain embodiments, the conjugate is described by formula (D-II-6). In other embodiments, the conjugate is described by formula (D-II-7). In certain embodiments, the conjugate and baloxavir are administered sequentially. In other embodiments, the conjugate and baloxavir are administered simultaneously.

In one aspect, the disclosure provides a method for treating or preventing a viral infection in subject by administering to the subject: a) an effective amount of a conjugate or composition described herein; and b) a second therapeutic agent. In certain embodiments, the conjugate is administered to the subject after the subject has a viral infection, is presumed to have a viral infection, or has been exposed to a virus. In some embodiments, the conjugate is administered to the subject prophylactically. In certain embodiments, the second therapeutic agent is administered to the subject after the subject has a viral infection, is presumed to have a viral infection, or has been exposed to the virus. In some embodiments, the second therapeutic agent is administered to the subject prophylactically. In some embodiments, the second therapeutic agent is administered within 30 days, within 14 days, within 7 days, within 2 days, or within 24 hours days of the conjugate. In particular embodiments, the second therapeutic agent is administered within 2 days of the conjugate. In certain embodiments, the second therapeutic agent is an antiviral agent (e.g., pimovidir, oseltamivir, zanamivir, peramivir, laninamivir, amantadine, baloxavir marboxil, baloxavir acid, rimantadine, or a pharmaceutically acceptable salt thereof). In particular embodiments, the antiviral agent is baloxavir marboxil, baloxavir acid, or a pharmaceutically acceptable salt thereof. In certain embodiments, the baloxavir marboxil is administered in an amount between 20 mg and 90 mg (e.g., between 25 mg and 50 mg, between 45 mg and 70 mg, or between 65 and 90 mg). In some embodiments, the baloxavir marboxil is administered orally. In certain embodiments, the baloxavir marboxil is administered as a single dose. In other embodiments, the baloxavir marboxil is administered as more than one dose. In particular embodiments, the baloxavir marboxil is administered in an amount between 20 mg and 40 mg. In other embodiments, the baloxavir marboxil is administered in an amount between 30 and 80 mg.

In some embodiments of any of the aspects described herein, A₁ and/or A₂ have the structure described by (A-I):

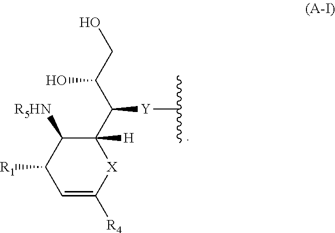

In preferred embodiments, wherein A₁ and/or A₂ have the structure described by (A-I): R₁ is —NHC(═NH)NH₂, R₄ is —CO₂H, R₅ is —COCH₃, and/or X is —O—. In preferred embodiments, A₁ and/or A₂ have the structure of zanamivir described by:

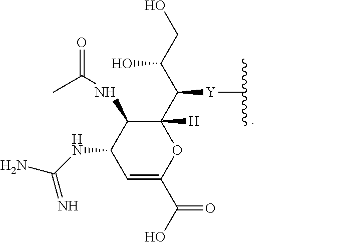

In some embodiments of any of the aspects described herein, A₁ and/or A₂ have the structure described by (A-II):

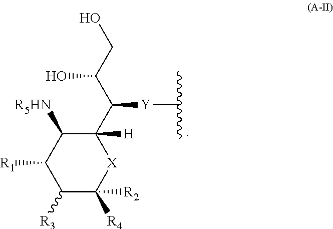

In preferred embodiments, wherein A₁ and/or A₂ have the structure described by (A-II): R₁ is —NHC(═NH)NH₂. R₂ is H or F, R₃ is H or F, R₄ is —CO₂H, R₅ is —COCH₃, and/or X is —O—. In preferred embodiments, A₁ and/or A₂ have the structure described by:

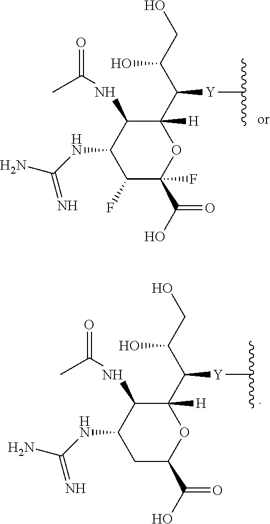

In some embodiments of any of the aspects described herein, A₁ and/or A₂ have the structure described by (A-III):

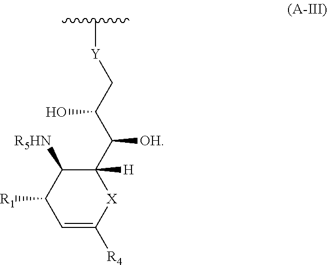

In preferred embodiments, wherein A₁ and/or A₂ have the structure described by (A-III): R₁ is —NHC(═NH)NH₂. R₄ is —CO₂H, R₅ is —COCH₃, and/or X is —O—. In preferred embodiments, A₁ and/or A₂ have the structure of zanamivir described by:

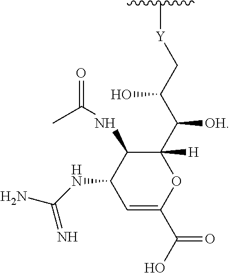

In some embodiments of any of the aspects described herein, A₁ and/or A₂ have the structure described by (A-IV):

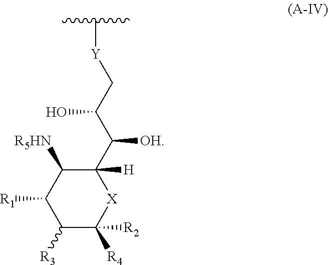

In preferred embodiments, wherein A₁ and/or A₂ have the structure described by (A-IV): R₁ is —NHC(═NH)NH₂, R₂ is H or F, R₃ is H or F, R₄ is —CO₂H, R₅ is —COCH₃, and/or X is —O—. In preferred embodiments, A₁ and/or A₂ have the structure described by:

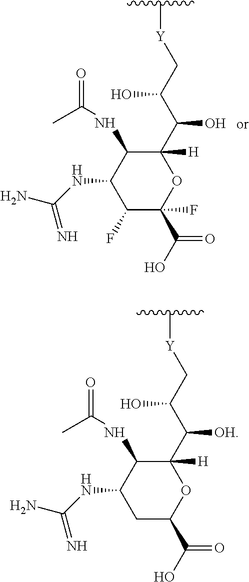

In some embodiments of any of the aspects described herein, A₁ and/or A₂ have the structure described by (A-V):

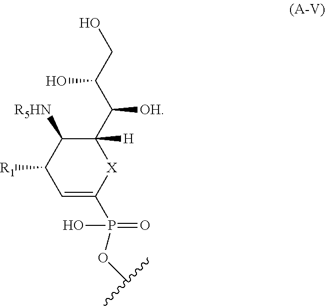

In preferred embodiments, wherein A₁ and/or A₂ have the structure described by (A-V): R₁ is —NHC(═NH)NH₂, R₅ is —COCH₃, and/or X is —O—. In preferred embodiments, A₁ and/or A₂ have the structure described by:

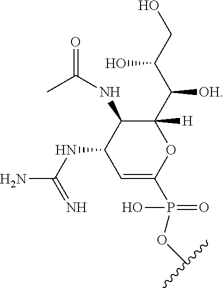 [SMILES: *OP(=O)(O)C1=C[C@H](NC(=N)N)[C@@H](NC(C)=O)[C@H]([C@H](O)[C@H](O)CO)O1]

In some embodiments of any of the aspects described herein, A₁ and/or A₂ have the structure described by (A-VI):

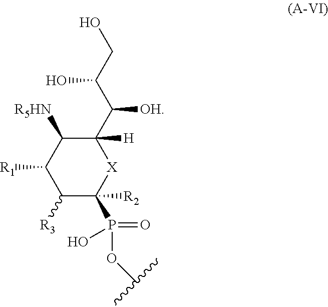

In preferred embodiments, wherein A₁ and/or A₂ have the structure described by (A-VI): R₁ is —NHC(═NH)NH₂, R₂ is H or F, R₃ is H or F, R₅ is —COCH₃, and/or X is —O—. In preferred embodiments. A₁ and/or A₂ have the structure described by:

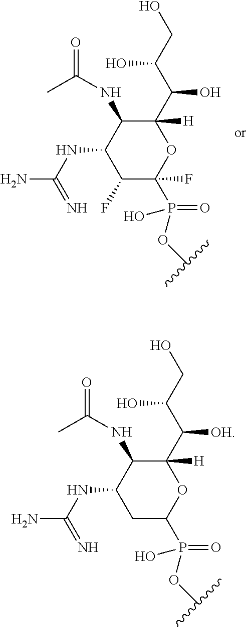 [SMILES: *OP(=O)(O)[C@]1(F)O[C@@H]([C@H](O)[C@H](O)CO)[C@H](NC(C)=O)[C@@H](NC(=N)N)[C@H]1F ; *OP(=O)(O)C1C[C@H](NC(=N)N)[C@@H](NC(C)=O)[C@H]([C@H](O)[C@H](O)CO)O1]

In some embodiments of any of the aspects described herein, A₁ and/or A₂ have the structure described by (A-VII):

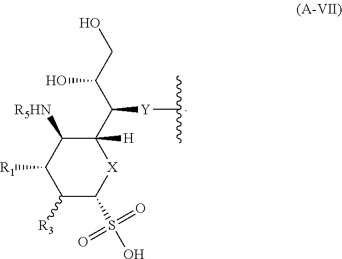

In preferred embodiments, wherein A₁ and/or A₂ have the structure described by (A-VII): R₁ is —NHC(═NH)NH₂, R₃ is H, R₅ is —COCH₃, and/or X is —O—. In preferred embodiments, A₁ and/or A₂ have the structure of sulfozanamivir described by:

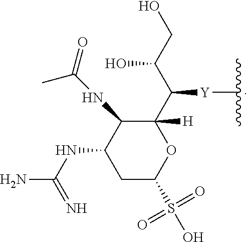

In some embodiments of any of the aspects described herein, A₁ and/or A₂ have the structure described by (A-VIII):

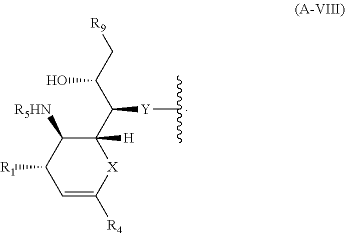

In some embodiments, each A₁ and each A₂ is described by formula (A-VIII-1):

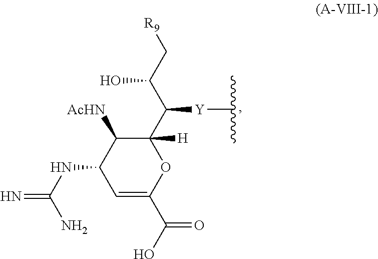

In some embodiments, each A₁ and each A₂ is independently selected from any one of formulas (A-VIII-1a)-(A-VIII-1d):

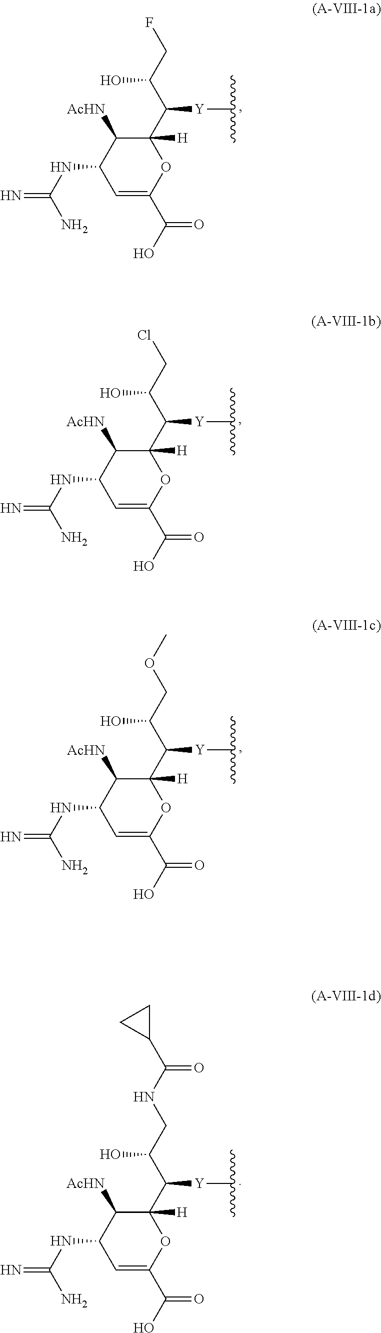

In an aspect, the disclosure features a method of synthesizing a conjugate of formula (D-I):

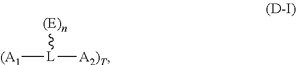

wherein each A, and each A₂ is independently selected from any one of formulas (A-I)-(A-VIII) as described herein;
n is 1 or 2;
each E includes an Fc domain monomer (e.g., an Fc domain monomer having the sequence of any one of SEQ ID NOs: 1-29);
L is a linker covalently attached to E and to Y of each of A₁ and A₂;
T is an integer from 1 to 20; and
each squiggly line in formula (D-I) indicates that L is covalently attached (e.g., by way of a covalent bond or linker) to each E,
the method including the steps of:
(a) providing a first composition including E;
(b) providing a second composition including a compound of formula (DF-I) or salt thereof:

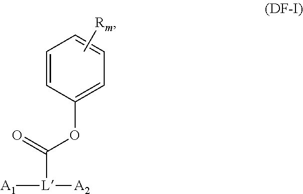

wherein
L′ is the remainder of L;
m is 0, 1, 2, 3, or 4; and
each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

In some embodiments, the first composition including E is an Fc domain (e.g., n is 2, each E is an Fc domain monomer, and the Fc domain monomers dimerize to form an Fc domain).

In some embodiments, L′ includes G, wherein G is optionally substituted C1-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ cycloalkylene, optionally substituted C₂-C₁₀ heterocycloalkylene, optionally substituted C₆-C₁₀ arylene, or optionally substituted C₂-C₁₀ heteroarylene.

In some embodiments, a compound of formula (DF-I) or salt thereof has the structure of any Int described herein (e.g., an Int of Table 1a, for example, Int-93 or Int-94).

In some embodiments, a compound of formula (DF-I) or salt thereof includes the structure of any Int described herein (e.g., an Int of Table 1a, for example, Int-93 or Int-94).

In some embodiments, a compound of formula (DF-I) or salt thereof is synthesized from the structure of any Int described herein (e.g., an Int of Table 1a, for example, Int-93 or Int-94).

In some embodiments, a compound of formula (DF-I), where each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-I) where m is 3, provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-I) where m is 3 and each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In an aspect, the disclosure features a method of synthesizing a conjugate of formula (D-I):

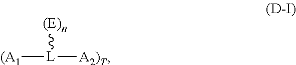

wherein each A₁ and each A₂ is independently selected from any one of formulas (A-I)-(A-VIII) as described herein;
n is 1 or 2;
each E includes an Fc domain monomer (e.g., an Fc domain monomer having the sequence of any one of SEQ ID NOs: 1-29);
L is a linker covalently attached to E and to Y of each of A₁ and A₂;
T is an integer from 1 to 20; and
each squiggly line in formula (D-I) indicates that L is covalently attached (e.g., by way of a covalent bond or linker) to each E,
the method including the steps of:
(a) providing a first composition including E;
(b) providing a second composition including a compound of formula (DF-II) or salt thereof:

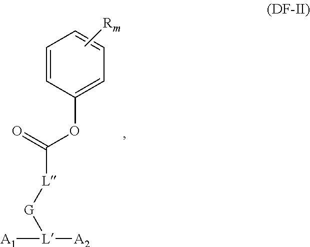

wherein G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ cycloalkylene, optionally substituted C₂-C₁₀ heterocycloalkylene, optionally substituted C₆-C₁₀ arylene, or optionally substituted C₂-C₁₀ heteroarylene;
L′-G-L″ is the remainder of L;
m is 0, 1, 2, 3, or 4; and
each R is, independently, halo, cyano, nitro, optionally substituted C₁-C₆ alkyl group, or optionally substituted C₁-C₆ heteroalkyl group;
and
(c) combining the first composition, the second composition, and a buffer to form a mixture.

In some embodiments, the first composition including E is an Fc domain (e.g., n is 2, each E is an Fc domain monomer, and the Fc domain monomers dimerize to form an Fc domain.

In some embodiments, G is optionally substituted C₁-C₆ heteroalkylene or optionally substituted C₂-C₁₀ heteroarylene. In some embodiments, G is optionally substituted C₁-C₆ heteroalkylene.

In some embodiments. G is

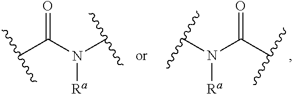

here R^{a} is H, optionally substituted C₁-C₂₀ alkylene (e.g., optionally substituted C₁-C₆ alkylene), or optionally substituted C₁-C₂₀ heteroalkylene (e.g., optionally substituted C₁-C₆ heteroalkylene).

In some embodiments, G is optionally substituted C₂-C₁₀ heteroarylene. In some embodiments, G is optionally substituted C₂-C₅ heteroarylene. In some embodiments, G is a 5-membered or 6-membered optionally substituted C₂-C₅ heteroarylene. In some embodiments, G is a triazolylene.

In some embodiments, the conjugate of formula (D-I) has the structure of:

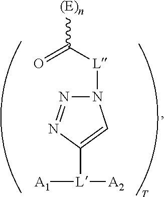

and the method includes the steps of:
(a) providing a first composition including E;
(b) providing a second composition including a compound of formula (DF-II-A) or salt thereof:

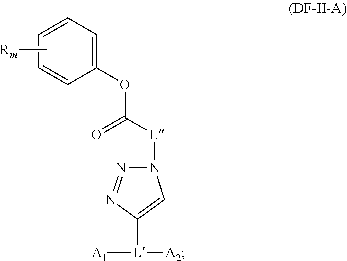

and
(c) combining the first composition, the second composition, and a buffer to form a mixture.

In some embodiments, the synthesis of compound of formula (DF-II-A) includes:
(d) providing a third composition including formula (D-G1-A) or salt thereof:

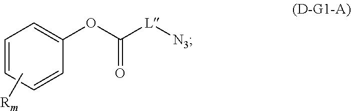

(e) providing a fourth composition including formula (D-G1-B) or salt thereof:

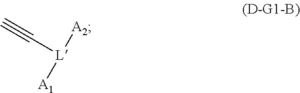

and
(f) combining the third composition and the fourth composition to form a mixture.

In some embodiments, the conjugate of formula (D-I) has the structure of

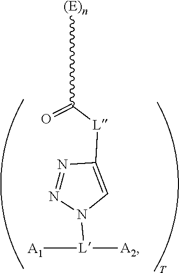

and the method includes the steps of:
(a) providing a first composition including E;
(b) providing a second composition including a compound of formula (DF-II-B) or salt thereof:

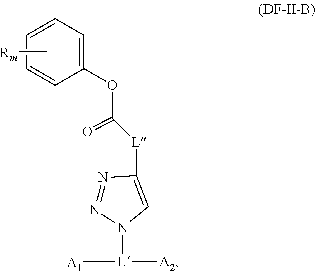

and
(c) combining the first composition, the second composition, and a buffer to form a mixture.

In some embodiments, the synthesis of compound of formula (DF-II-B) includes:
(d) providing a third composition including formula (D-2-A) or salt hereof:

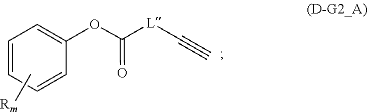

(e) providing a fourth composition including formula (D-G2-B) or salt thereof:

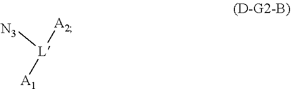

and
(f) combining the third composition and the fourth composition to form a mixture.

In some embodiments, step (f) includes the use of a Cu(I) source.

In another aspect, the disclosure features a method of synthesizing a conjugate of formula (D-I):

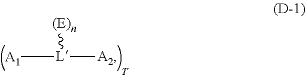

wherein each A₁ and each A₂ is independently selected from any one of formulas (A-I)-(A-VIII) as described herein;
n is 1 or 2;
each E includes an Fc domain monomer (e.g., an Fc domain monomer having the sequence of any one of SEQ ID NOs: 1-29);
L is a linker covalently attached to E and to Y of each of A₁ and A₂;
T is an integer from 1 to 20; and
each squiggly line in formula (D-I) indicates that L is covalently attached (e.g., by way of a covalent bond or linker) to each E.
the method including the steps of:
(a) providing a first composition including formula (D-G3-A) or a salt thereof:

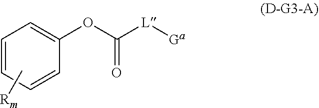

where G^{a} is a functional group that reacts with G^{b} to form G;
(b) providing a second composition including formula (D-G3-B) or a salt thereof:

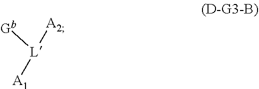

where G^{b} is a functional group that reacts with G^{a} to form G; and
(c) combining the first composition and the second composition to form a first mixture,
where m is 0, 1, 2, 3, or 4; and each R is, independently, halo, cyano, nitro, optionally substituted C₁-C₆ alkyl group, or optionally substituted C₁-C₆ heteroalkyl group.

In some embodiments, step (c) includes the use of a Cu(I) source.

In some embodiments, the method further includes:
(d) providing a third composition including E; and
(e) combining the third composition, the first mixture, and a buffer to form a second mixture.

In some embodiments, G^{a} includes optionally substituted amino. In some embodiments, G^{b} includes a carbonyl.

In some embodiments, G^{a} includes a carbonyl. In some embodiments, G^{b} includes optionally substituted amino.

In some embodiments, G^{a} includes an azido group. In some embodiments, G^{b} includes an alknyl group.

In some embodiments. G^{a} includes an alkynyl group. In some embodiments, G^{b} includes an azido group.

In some embodiments of any of the aspects described herein, a compound of formula (DF-II) or salt thereof has the structure of any Int described herein (e.g., an Int of Table 1a, for example, Int-93 or Int-94).

In some embodiments of any of the aspects described herein, a compound of formula (DF-II) or salt thereof includes the structure of any Int described herein (e.g., an Int of Table 1a, for example, Int-93 or Int-94).

In some embodiments of any of the aspects described herein, a compound of formula (DF-II) or salt thereof is synthesized from the structure of any Int described herein (e.g., an Int of Table 1a, for example, Int-93 or Int-94).

In some embodiments, a compound of formula (DF-II) (e.g., a compound of formula (DF-II-A) or (DF-II-B) and/or a compound of formula (D-G1-A) or (D-G2-A), where each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-II) (e.g., a compound of formula (DF-II-A) or (DF-II-B) and/or a compound of formula (D-G1-A) or (D-G2-A), where m is 3, provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-II) (e.g., a compound of formula (DF-II-A) or (DF-II-B) and/or a compound of formula (D-G1-A) or (D-G2-A), where m is 3 and each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments of any of the aspects described herein, E includes at least one lysine residue. In some embodiments, the squiggly line in formula (D-I) is covalently bound to a lysine residue of each E.

In some embodiments of any of the aspects described herein, E includes at least one cysteine residue. In some embodiments, the squiggly line in formula (D-I) is covalently bound to a cysteine residue of each E.

In some embodiments of any of the aspects described herein, each R is, independently, halo, cyano, nitro, haloalkyl, or

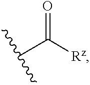

where R^{z} is optionally substituted C₁-C₅ alkyl group or optionally substituted C₁-C₅ heteroalkyl group. In some embodiments, each R is, independently, halo, cyano, nitro, or haloalkyl.

In some embodiments, each R is, independently, F, Cl, Br, or I.

In some embodiments, each R is F.

In some embodiments, m is 3 or 4. In some embodiments, m is 3. In some embodiments, m is 4.

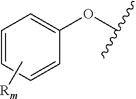

In some embodiments,

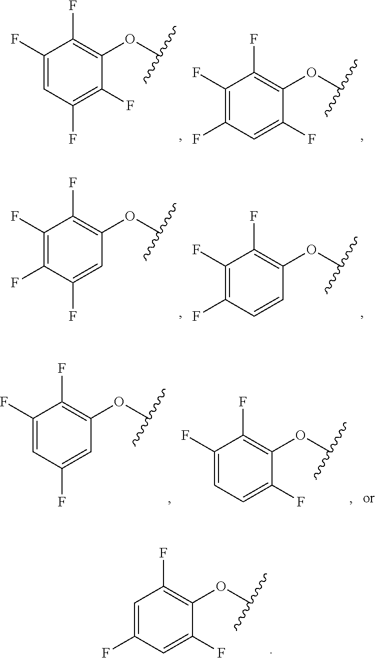

In some embodiments,

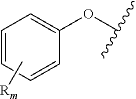

is

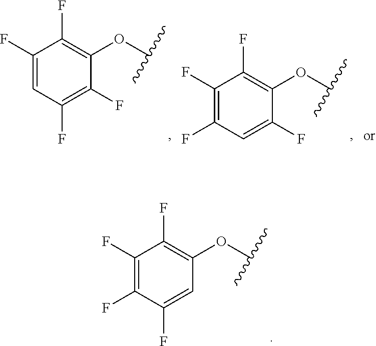

In some embodiments,

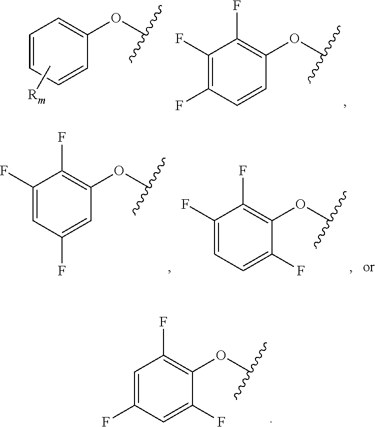

In some embodiments

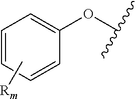

is

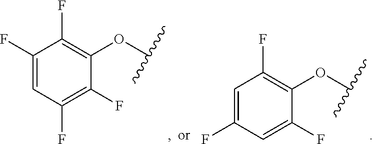 [SMILES: *Oc1c(F)c(F)cc(F)c1F ; *Oc1c(F)cc(F)cc1F]

In some embodiments,

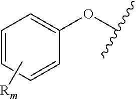

is

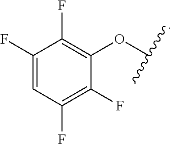 [SMILES: *Oc1c(F)c(F)cc(F)c1F]

In some embodiments

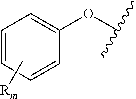

is

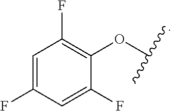 [SMILES: *Oc1c(F)cc(F)cc1F]

In some embodiments of any of the aspects described herein, the buffer includes borate or carbonate. In some embodiments, the buffer includes borate. In some embodiments, the buffer includes carbonate.

In some embodiments, the buffer has a pH of about 7.0 to 10.0 (e.g., about 7.0 to 7.5, 7.5 to 8.0, 8.0 to 8.5, 8.5 to 9.0, 9.0 to 9.5, 9.5 to 10.0, 7.0 to 8.0, 7.5 to 8.5, 8.0 to 9.0, 8.5 to 9.5, 9.0 to 10.0, 7.0 to 9.0, 7.5 to 9.5, or 8.0 to 10.0).

In some embodiments, the buffer has a pH of about 7.0. In some embodiments, the buffer has a pH of about 7.1. In some embodiments, the buffer has a pH of about 7.2. In some embodiments, the buffer has a pH of about 7.3. In some embodiments, the buffer has a pH of about 7.4. In some embodiments, the buffer has a pH of about 7.5. In some embodiments, the buffer has a pH of about 7.6. In some embodiments, the buffer has a pH of about 7.7. In some embodiments, the buffer has a pH of about 7.8. In some embodiments, the buffer has a pH of about 7.9. In some embodiments, the buffer has a pH of about 8.0. In some embodiments, the buffer has a pH of about 8.1. In some embodiments, the buffer has a pH of about 8.2. In some embodiments, the buffer has a pH of about 8.3. In some embodiments, the buffer has a pH of about 8.4. In some embodiments, the buffer has a pH of about 8.5. In some embodiments, the buffer has a pH of about 8.6. In some embodiments, the buffer has a pH of about 8.7. In some embodiments, the buffer has a pH of about 8.8. In some embodiments, the buffer has a pH of about 8.9. In some embodiments, the buffer has a pH of about 9.0. In some embodiments, the buffer has a pH of about 9.5. In some embodiments, the buffer has a pH of about 9.6. In some embodiments, the buffer has a pH of about 9.7. In some embodiments, the buffer has a pH of about 9.8. In some embodiments, the buffer has a pH of about 9.9. In some embodiments, the buffer has a pH of about 10.0.

In some embodiments of any of the aspects described herein, step (c) or step (e) is conducted at a temperature of 5 to 50° C., such as 20 to 30° C. (e.g., 20 to 25, 21 to 26, 22 to 27, 23 to 28, 24 to 29, or 25 to 30° C.).

In some embodiments, step (c) or step (e) is conducted at a temperature of about 25° C.

In some embodiments, step (c) or step (e) is conducted for about 1 to 24 hours, such as 1 to 12 hours (e.g., 1 to 2, 1 to 5, 2 to 3, 2 to 5, 2 to 10, 2 to 12, 3 to 4, 4 to 5, 1 to 3, 2 to 4, or 3 to 5 hours).

In some embodiments, step (c) or step (e) is conducted for about 2 hours. In some embodiments, step (c) or step (e) is conducted for about 3 hours. In some embodiments, step (c) or step (e) is conducted for about 4 hours. In some embodiments, step (c) or step (e) is conducted for about 5 hours. In some embodiments, step (c) or step (e) is conducted for about 6 hours. In some embodiments, step (c) or step (e) is conducted for about 7 hours. In some embodiments, step (c) or step (e) is conducted for about 8 hours. In some embodiments, step (c) or step (e) is conducted for about 9 hours. In some embodiments, step (c) or step (e) is conducted for about 10 hours. In some embodiments, step (c) or step (e) is conducted for about 11 hours. In some embodiments, step (c) or step (e) is conducted for about 12 hours.

In some embodiments, the first composition or third composition includes phosphate-buffered saline buffer.

In some embodiments, the buffer has a pH of about 7.0 to 8.0 (e.g., about 7.0 to 7.5, 7.5 to 8.0, 7.0 to 7.2, 7.2 to 7.4, 7.4 to 7.6, 7.6 to 7.8, or 7.8 to 8.0).

In some embodiments, the buffer has a pH of about 7.5.

In some embodiments, the second composition or the first mixture includes DMF.

In some embodiments, the method further includes a purification step. In some embodiments, the purification step includes dialysis in arginine buffer. In some embodiments, the purification step includes a buffer exchange.

In some embodiments. T is an integer from 1 to 20 (e.g., 1, 2, 3, 4, 5, 6, 7, 8, 9, 10, 11, 12, 13, 14, 15, 16, 17, 18, 19, or 20). In some embodiments, the average value of T is 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20. In certain embodiments, the average T is 1 to 10 (e.g., 1.5, 2, 2.5, 3, 3.5, 4, 4.5, 5, 5.5, 6, 6.5, 7, 7.5, 8, 8.5, 9, 9.5, or 10). In certain embodiments, the average T is 1 to 5 (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5). In some embodiment, the average T is 5 to 10 (e.g., 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10). In some embodiments, the average T is 2.5 to 7.5 (e.g., 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5).

### Definitions

To facilitate the understanding of this disclosure, a number of terms are defined below. Terms defined herein have meanings as commonly understood by a person of ordinary skill in the areas relevant to the present disclosure. Terms such as “a”, “an,” and “the” are not intended to refer to only a singular entity, but include the general class of which a specific example may be used for illustration. The terminology herein is used to describe specific embodiments of the disclosure, but their usage does not delimit the disclosure, except as outlined in the claims.

The term “neuraminidase inhibitor” or ““viral neuraminidase inhibitor,” as used herein, refers to compounds that decreases the activity of the enzyme influenza virus neuraminidase (e.g., from influenza virus A, B, or C). A neuraminidase inhibitor may be identified by methods known to those of skill in the art, for example, by reduction of viral replication in an influenza viral plaque reduction assay, e.g., at concentrations less than 20 μM (e.g., less than 10 μM, 5 μM, 2 μM, 1 μM, 500 nM or 100 nM). Viral neuraminidase inhibitors known to those of skill in the art include zanamivir, sulfozanamivir, and analogs thereof (see, for example, Hadházi et al. A sulfozanamivir analogue has potent anti-influenza virus activity. Chem Med Chem Comm. 13:785-789 (2018)). In particular, zanamivir and analogs thereof include viral neuraminidase inhibitors of formulas (A-I)-(A-VIII).

The term “inhibits neuraminidase activity,” as used herein refers to an IC₅₀ of less than or equal to 1,000 nM, for example, as measured in accordance with the neuraminidase inhibition assay in Example 2 of WO 2021/046549. Specifically, the IC₅₀ represents the concentration of the influenza virus neuraminidase inhibitor that is required for 50% inhibition in vitro. In some aspects, an IC₅₀ of less than or equal to 100 nM or less than or equal to 10 nM in accordance with neuraminidase inhibition assay is indicative of a compound inhibiting neuraminidase activity.

By “viral infection” is meant the pathogenic growth of a virus (e.g., the influenza virus) in a host organism (e.g., a human subject). A viral infection can be any situation in which the presence of a viral population(s) is damaging to a host body. Thus, a subject is “suffering” from a viral infection when an excessive amount of a viral population is present in or on the subject's body, or when the presence of a viral population(s) is damaging the cells or other tissue of the subject.

As used herein, the term “Fc domain monomer” refers to a polypeptide chain that includes at least a hinge domain and second and third antibody constant domains (C_{H}2 and C_{H}3) or functional fragments thereof (e.g., fragments that that capable of (i) dimerizing with another Fc domain monomer to form an Fc domain, and (ii) binding to an Fc receptor. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., IgG1). An Fc domain monomer does not include any portion of an immunoglobulin that is capable of acting as an antigen-recognition region, e.g., a variable domain or a complementarity determining region (CDR). Fc domain monomers in the conjugates as described herein can contain one or more changes from a wild-type Fc domain monomer sequence (e.g., 1-10, 1-8, 1-6, 1-4 amino acid substitutions, additions, or deletions) that alter the interaction between an Fc domain and an Fc receptor. Examples of suitable changes are known in the art. In some embodiments, the N-terminus of the Fc domain monomer is any one of amino acid residues 198-205. In some embodiments, the N-terminus of the Fc domain monomer is amino acid residue 201 (e.g., Asn 201). In some embodiments, the N-terminus of the Fc domain monomer is amino acid residue 202 (e.g., Val 202). In some embodiments, the C-terminus of the Fc domain monomer is any one of amino acid residues 437-447. In some embodiments, the C-terminus of the Fc domain monomer is amino acid residue 446 (e.g., Gly 446). In some embodiments, the C-terminus of the Fc domain monomer is amino acid residue 447 (e.g. Lys 447). C-terminal Lys447 of the Fc region may or may not be present, without affecting the structure or stability of the Fc region. C-terminal Lys 447 may be proteolytically cleaved upon expression of the polypeptide. In some embodiments of any of the Fc domain monomers described herein, C-terminal Lys 447 is optionally present or absent. The N-terminal N (Asn) of the Fc region may or may not be present, without affecting the structure of stability of the Fc region. N-terminal Asn may be deamidated upon expression of the polypeptide. In some embodiments of any of the Fc domain monomers described herein, N-terminal Asn is optionally present or absent. Unless otherwise specified herein, numbering of amino acid residues in the IgG or Fc domain monomer is according to the EU numbering system for antibodies, also called the Kabat EU index, as described, for example, in Kabat et al., Sequences of Proteins of Immunological Interest, 5th Ed. Public Health Service, National Institutes of Health, Bethesda, M D, 1991.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers. The term “covalently attached” refers to two parts of a conjugate that are linked to each other by a covalent bond formed between two atoms in the two parts of the conjugate.

As used-herein, a “surface exposed amino acid” or “solvent-exposed amino acid,” such as a surface exposed cysteine or a surface exposed lysine refers to an amino acid that is accessible to the solvent surrounding the protein. A surface exposed amino acid may be a naturally-occurring or an engineered variant (e.g., a substitution or insertion) of the protein. In some embodiments, a surface exposed amino acid is an amino acid that when substituted does not substantially change the three-dimensional structure of the protein.

The terms “linker,” “L,” and “L′,” as used herein, refer to a covalent linkage or connection between two or more components in a conjugate (e.g., between two neuraminidase inhibitors in a conjugate described herein, between a neuraminidase inhibitor and an Fc domain in a conjugate described herein, and between a dimer of two neuraminidase inhibitors and an Fc domain in a conjugate described herein). In some embodiments, a conjugate described herein may contain a linker that has a trivalent structure (e.g., a trivalent linker). A trivalent linker has three arms, in which each arm is covalently linked to a component of the conjugate (e.g., a first arm conjugated to a first neuraminidase inhibitor, a second arm conjugated to a second neuraminidase inhibitor, and a third arm conjugated to an Fc domain).

Molecules that may be used as linkers include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. The first functional group may form a covalent linkage with a first component in the conjugate and the second functional group may form a covalent linkage with the second component in the conjugate. In some embodiments of a trivalent linker, two arms of a linker may contain two dicarboxylic acids, in which the first carboxylic acid may form a covalent linkage with the first neuraminidase inhibitor in the conjugate and the second carboxylic acid may form a covalent linkage with the second neuraminidase inhibitor in the conjugate, and the third arm of the linker may for a covalent linkage with an Fc domain in the conjugate. Examples of dicarboxylic acids are described further herein. In some embodiments, a molecule containing one or more maleimide groups may be used as a linker, in which the maleimide group may form a carbon-sulfur linkage with a cysteine in a component (e.g., an Fc domain) in the conjugate. In some embodiments, a molecule containing one or more alkyne groups may be used as a linker, in which the alkyne group may form a 1,2,3-triazole linkage with an azide in a component (e.g., an Fc domain) in the conjugate. In some embodiments, a molecule containing one or more azide groups may be used as a linker, in which the azide group may form a 1,2,3-triazole linkage with an alkyne in a component (e.g., an Fc domain) in the conjugate. In some embodiments, a molecule containing one or more bis-sulfone groups may be used as a linker, in which the bis-sulfone group may form a linkage with an amine group a component (e.g., an Fc domain) in the conjugate. In some embodiments, a molecule containing one or more sulfonic acid groups may be used as a linker, in which the sulfonic acid group may form a sulfonamide linkage with a component in the conjugate. In some embodiments, a molecule containing one or more isocyanate groups may be used as a linker, in which the isocyanate group may form a urea linkage with a component in the conjugate. In some embodiments, a molecule containing one or more haloalkyl groups may be used as a linker, in which the haloalkyl group may form a covalent linkage, e.g., C—N and C—O linkages, with a component in the conjugate.

In some embodiments, a linker provides space, rigidity, and/or flexibility between the two or more components. In some embodiments, a linker may be a bond, e.g., a covalent bond. The term “bond” refers to a chemical bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms. In some embodiments, a linker includes no more than 250 non-hydrogen atoms. In some embodiments, the backbone of a linker includes no more than 250 atoms. The “backbone” of a linker refers to the atoms in the linker that together form the shortest path from one part of a conjugate to another part of the conjugate (e.g., the shortest path linking a first neuraminidase inhibitor and a second neuraminidase inhibitor). The atoms in the backbone of the linker are directly involved in linking one part of a conjugate to another part of the conjugate (e.g., linking a first neuraminidase inhibitor and a second neuraminidase inhibitor). For examples, hydrogen atoms attached to carbons in the backbone of the linker are not considered as directly involved in linking one part of the conjugate to another part of the conjugate.

In some embodiments, a linker may comprise a synthetic group derived from, e.g., a synthetic polymer (e.g., a polyethylene glycol (PEG) polymer). In some embodiments, a linker may comprise one or more amino acid residues, such as D- or L-amino acid residues. In some embodiments, a linker may be a residue of an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may comprise one or more, e.g., 1-100, 1-50, 1-25, 1-10, 1-5, or 1-3, optionally substituted alkylene, optionally substituted heteroalkylene (e.g., a PEG unit), optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted cycloalkenylene, optionally substituted heterocycloalkenylene, optionally substituted cycloalkynylene, optionally substituted heterocycloalkynylene, optionally substituted arylene, optionally substituted heteroarylene (e.g., pyridine), O, S, NRⁱ (Rⁱ is H, optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted heterocycloalkyl, optionally substituted cycloalkenyl, optionally substituted heterocycloalkenyl, optionally substituted cycloalkynyl, optionally substituted heterocycloalkynyl, optionally substituted aryl, or optionally substituted heteroaryl), P, carbonyl, thiocarbonyl, sulfonyl, phosphate, phosphoryl, or imino. For example, a linker may comprise one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

The terms “alkyl,” “alkenyl,” and “alkynyl,” as used herein, include straight-chain and branched-chain monovalent substituents, as well as combinations of these, containing only C and H when unsubstituted. When the alkyl group includes at least one carbon-carbon double bond or carbon-carbon triple bond, the alkyl group can be referred to as an “alkenyl” or “alkynyl” group respectively. The monovalency of an alkyl, alkenyl, or alkynyl group does not include the optional substituents on the alkyl, alkenyl, or alkynyl group. For example, if an alkyl, alkenyl, or alkynyl group is attached to a compound, monovalency of the alkyl, alkenyl, or alkynyl group refers to its attachment to the compound and does not include any additional substituents that may be present on the alkyl, alkenyl, or alkynyl group. In some embodiments, the alkyl or heteroalkyl group may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, the alkenyl, heteroalkenyl, alkynyl, or heteroalkynyl group may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12, C2-C10, C2-C8, C2-C6, or C2-C4). Examples include, but are not limited to, methyl, ethyl, isobutyl, sec-butyl, tert-butyl, 2-propenyl, and 3-butynyl.

The term “cycloalkyl,” as used herein, represents a monovalent saturated or unsaturated non-aromatic cyclic alkyl group. A cycloalkyl may have, e.g., three to twenty carbons (e.g., a C3-C7, C3-C8, C3-C9, C3-C10, C3-C11, C3-C12. C3-C14, C3-C16, C3-C18, or C3-C20 cycloalkyl). Examples of cycloalkyls include, but are not limited to, cyclopropyl, cyclobutyl, cyclopentyl, cyclohexyl, and cycloheptyl. When the cycloalkyl group includes at least one carbon-carbon double bond, the cycloalkyl group can be referred to as a “cycloalkenyl” group. A cycloalkenyl may have, e.g., four to twenty carbons (e.g., a C4-C7, C4-C8, C4-C9, C4-C10, C4-C11, C4-C12, C4-C14, C4-C16, C4-C18, or C4-C20 cycloalkenyl). Exemplary cycloalkenyl groups include, but are not limited to, cyclopentenyl, cyclohexenyl, and cycloheptenyl. When the cycloalkyl group includes at least one carbon-carbon triple bond, the cycloalkyl group can be referred to as a “cycloalkynyl” group. A cycloalkynyl may have, e.g., eight to twenty carbons (e.g., a C8-C9, C8-C10, C8-C11, C8-C12, C8-C14, C8-C16, C8-C18, or C8-C20 cycloalkynyl). The term “cycloalkyl” also includes a cyclic compound having a bridged multicyclic structure in which one or more carbons bridges two non-adjacent members of a monocyclic ring, e.g., bicyclo[2.2.1.]heptyl and adamantane. The term “cycloalkyl” also includes bicyclic, tricyclic, and tetracyclic fused ring structures, e.g., decalin and spiro cyclic compounds.

The term “aryl,” as used herein, refers to any monocyclic or fused ring bicyclic or tricyclic system which has the characteristics of aromaticity in terms of electron distribution throughout the ring system, e.g., phenyl, naphthyl, or phenanthrene. In some embodiments, a ring system contains 5-15 ring member atoms or 5-10 ring member atoms. An aryl group may have, e.g., five to fifteen carbons (e.g., a C5-C6, C5-C7, C5-C8, C5-C9, C5-C10, C5-C11, C5-C12, C5-C13, C5-C14, or C5-C15 aryl). The term “heteroaryl” also refers to such monocyclic or fused bicyclic ring systems containing one or more, e.g., 1-4, 1-3, 1, 2, 3, or 4, heteroatoms selected from O, S and N. A heteroaryl group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroaryl). The inclusion of a heteroatom permits inclusion of 5-membered rings to be considered aromatic as well as 8-membered rings. Thus, typical heteroaryl systems include, e.g., pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, and imidazolyl. Because tautomers are possible, a group such as phthalimido is also considered heteroaryl. In some embodiments, the aryl or heteroaryl group is a 5- or 6-membered aromatic rings system optionally containing 1-2 nitrogen atoms. In some embodiments, the aryl or heteroaryl group is an optionally substituted phenyl, pyridyl, indolyl, pyrimidyl, pyridazinyl, benzothiazolyl, benzimidazolyl, pyrazolyl, imidazolyl, isoxazolyl, thiazolyl, or imidazopyridinyl. In some embodiments, the aryl group is phenyl. In some embodiments, an aryl group may be optionally substituted with a substituent such an aryl substituent, e.g., biphenyl.

The term “alkaryl,” refers to an aryl group that is connected to an alkylene, alkenylene, or alkynylene group. In general, if a compound is attached to an alkaryl group, the alkylene, alkenylene, or alkynylene portion of the alkaryl is attached to the compound. In some embodiments, an alkaryl is C6-C35 alkaryl (e.g., C6-C16, C6-C14, C6-C12, C6-C10, C6-C9, C6-C8, C7, or C6 alkaryl), in which the number of carbons indicates the total number of carbons in both the aryl portion and the alkylene, alkenylene, or alkynylene portion of the alkaryl. Examples of alkaryls include, but are not limited to, (C1-C8)alkylene(C8-C12)aryl, (C2-C8)alkenylene(C6-C12)aryl, or (C2-C8)alkynylene(C6-C12)aryl. In some embodiments, an alkaryl is benzyl or phenethyl. In a heteroalkaryl, one or more heteroatoms selected from N, O, and S may be present in the alkylene, alkenylene, or alkynylene portion of the alkaryl group and/or may be present in the aryl portion of the alkaryl group. In an optionally substituted alkaryl, the substituent may be present on the alkylene, alkenylene, or alkynylene portion of the alkaryl group and/or may be present on the aryl portion of the alkaryl group.

The term “amino,” as used herein, represents —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino group is —NH₂.

The term “alkamino,” as used herein, refers to an amino group, described herein, that is attached to an alkylene (e.g., C1-C5 alkylene), alkenylene (e.g., C2-C5 alkenylene), or alkynylene group (e.g., C2-C5 alkenylene). In general, if a compound is attached to an alkamino group, the alkylene, alkenylene, or alkynylene portion of the alkamino is attached to the compound. The amino portion of an alkamino refers to —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino portion of an alkamino is —NH₂. An example of an alkamino group is C1-C5 alkamino, e.g., C2 alkamino (e.g., CH₂CH₂NH₂ or CH₂CH₂N(CH₃)₂). In a heteroalkamino group, one or more, e.g., 1-4, 1-3, 1, 2, 3, or 4, heteroatoms selected from N, O, and S may be present in the alkylene, alkenylene, or alkynylene portion of the heteroalkamino group. In some embodiments, an alkamino group may be optionally substituted. In a substituted alkamino group, the substituent may be present on the alkylene, alkenylene, or alkynylene portion of the alkamino group and/or may be present on the amino portion of the alkamino group. The term “alkamide,” as used herein, refers to an amide group that is attached to an alkylene (e.g., C1-C5 alkylene), alkenylene (e.g., C2-C5 alkenylene), or alkynylene (e.g., C2-C5 alkenylene) group. In general, if a compound is attached to an alkamide group, the alkylene, alkenylene, or alkynylene portion of the alkamide is attached to the compound. The amide portion of an alkamide refers to —C(O)—N(R^{x})₂, where each R^{x} is, independently, H, alkyl, alkenyl, alkynyl, aryl, alkaryl, cycloalkyl, or two R^{x} combine to form a heterocycloalkyl. In some embodiment, the amide portion of an alkamide is —C(O)NH₂. An alkamide group may be —(CH₂)₂—C(O)NH₂ or —CH₂—C(O)NH₂. In a heteroalkamide group, one or more, e.g., 1-4, 1-3, 1, 2, 3, or 4, heteroatoms selected from N, O, and S may be present in the alkylene, alkenylene, or alkynylene portion of the heteroalkamide group. In some embodiments, an alkamide group may be optionally substituted. In a substituted alkamide group, the substituent may be present on the alkylene, alkenylene, or alkynylene portion of the alkamide group and/or may be present on the amide portion of the alkamide group.

The terms “alkylene,” “alkenylene,” and “alkynylene,” as used herein, refer to divalent groups having a specified size. In some embodiments, an alkylene may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, an alkenylene or alkynylene may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12. C2-C10, C2-C8, C2-C6, or C2-C4). Alkylene, alkenylene, and/or alkynylene includes straight-chain and branched-chain forms, as well as combinations of these. The divalency of an alkylene, alkenylene, or alkynylene group does not include the optional substituents on the alkylene, alkenylene, or alkynylene group. For example, two neuraminidase inhibitors may be attached to each other by way of a linker that includes alkylene, alkenylene, and/or alkynylene, or combinations thereof. Each of the alkylene, alkenylene, and/or alkynylene groups in the linker is considered divalent with respect to the two attachments on either end of alkylene, alkenylene, and/or alkynylene group. For example, if a linker includes -(optionally substituted alkylene)-(optionally substituted alkenylene)-(optionally substituted alkylene)-, the alkenylene is considered divalent with respect to its attachments to the two alkylenes at the ends of the linker. The optional substituents on the alkenylene are not included in the divalency of the alkenylene. The divalent nature of an alkylene, alkenylene, or alkynylene group (e.g., an alkylene, alkenylene, or alkynylene group in a linker) refers to both of the ends of the group and does not include optional substituents that may be present in an alkylene, alkenylene, or alkynylene group. Because they are divalent, they can link together multiple (e.g., two) parts of a conjugate, e.g., a first neuraminidase inhibitor and a second neuraminidase inhibitor. Alkylene, alkenylene, and/or alkynylene groups can be substituted by the groups typically suitable as substituents for alkyl, alkenyl and alkynyl groups as set forth herein. For example, C═O is a C1 alkylene that is substituted by an oxo (═O). For example, —HCR—C≡C— may be considered as an optionally substituted alkynylene and is considered a divalent group even though it has an optional substituent, R. Heteroalkylene, heteroalkenylene, and/or heteroalkynylene groups refer to alkylene, alkenylene, and/or alkynylene groups including one or more, e.g., 1-4, 1-3, 1, 2, 3, or 4, heteroatoms, e.g., N, O, and S. For example, a polyethylene glycol (PEG) polymer or a PEG unit —(CH₂)₂—O— in a PEG polymer is considered a heteroalkylene containing one or more oxygen atoms.

As used herein, a “combination therapy” or “administered in combination” means that a conjugate described herein (e.g., a conjugate of any one of formulas (D-I)-(D-VIII)) and one (or more) different agents or treatments are administered to a subject as part of a defined treatment regimen for a viral infection. The treatment regimen defines the doses and periodicity of administration of each agent such that the effects of the separate agents on the subject overlap. In some embodiments, the delivery of the conjugate and the one or more agents is simultaneous or concurrent and the conjugate and the one or more agents may be co-formulated. In some embodiments, the conjugate and the one or more agents are not co-formulated and are administered in a sequential manner as part of a prescribed regimen. In some embodiments, administration of the conjugate and the one or more agents or treatments in combination is such that the reduction in a symptom, or other parameter related to the viral infection, is greater than what would be observed with one agent or treatment delivered alone or in the absence of the other. The effect of the conjugate and the one or more agents can be partially additive, wholly additive, or greater than additive (e.g., synergistic). Sequential or substantially simultaneous administration of each therapeutic agent can be by any appropriate route including, but not limited to, oral routes, intravenous routes, intramuscular routes, and direct absorption through mucous membrane tissues. The therapeutic agents can be administered by the same route or by different routes. For example, a conjugate described herein may be administered by intravenous injection while a second therapeutic agent of the combination may be administered orally.

The term “cycloalkylene,” as used herein, refers to a divalent cyclic group linking together two parts of a compound. For example, one carbon within the cycloalkylene group may be linked to one part of the compound, while another carbon within the cycloalkylene group may be linked to another part of the compound. A cycloalkylene group may include saturated or unsaturated non-aromatic cyclic groups. A cycloalkylene may have, e.g., three to twenty carbons in the cyclic portion of the cycloalkylene (e.g., a C3-C7, C3-C8, C3-C9, C3-C10, C3-C11, C3-C12. C3-C14, C3-C16, C3-C18, or C3-C20 cycloalkylene). When the cycloalkylene group includes at least one carbon-carbon double bond, the cycloalkylene group can be referred to as a “cycloalkenylene” group. A cycloalkenylene may have, e.g., four to twenty carbons in the cyclic portion of the cycloalkenylene (e.g., a C4-C7, C4-C8, C4-C9. C4-C10, C4-C11. C4-C12, C4-C14, C4-C16, C4-C18, or C4-C20 cycloalkenylene). When the cycloalkylene group includes at least one carbon-carbon triple bond, the cycloalkylene group can be referred to as a “cycloalkynylene” group. A cycloalkynylene may have, e.g., four to twenty carbons in the cyclic portion of the cycloalkynylene (e.g., a C4-C7, C4-C8, C4-C9. C4-C10, C4-C11, C4-C12, C4-C14, C4-C16, C4-C18, or C8-C20 cycloalkynylene). A cycloalkylene group can be substituted by the groups typically suitable as substituents for alkyl, alkenyl and alkynyl groups as set forth herein. Heterocycloalkylene refers to a cycloalkylene group including one or more, e.g., 1-4, 1-3, 1, 2, 3, or 4, heteroatoms, e.g., N, O, and S. Examples of cycloalkylenes include, but are not limited to, cyclopropylene and cyclobutylene. A tetrahydrofuran may be considered as a heterocycloalkylene.

The term “arylene,” as used herein, refers to a multivalent (e.g., divalent or trivalent) aryl group linking together multiple (e.g., two or three) parts of a compound. For example, one carbon within the arylene group may be linked to one part of the compound, while another carbon within the arylene group may be linked to another part of the compound. An arylene may have, e.g., five to fifteen carbons in the aryl portion of the arylene (e.g., a C5-C6, C5-C7, C5-C8, C5-C9. C5-C10, C5-C11, C5-C12, C5-C13, C5-C14, or C5-C15 arylene). An arylene group can be substituted by the groups typically suitable as substituents for alkyl, alkenyl and alkynyl groups as set forth herein. Heteroarylene refers to an aromatic group including one or more, e.g., 1-4, 1-3, 1, 2, 3, or 4, heteroatoms, e.g., N, O, and S. A heteroarylene group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroarylene).

The term “optionally substituted,” as used herein, refers to having 0, 1, or more substituents, such as 0-25, 0-20, 0-10 or 0-5 substituents. Substituents include, but are not limited to, alkyl, alkenyl, alkynyl, aryl, alkaryl, acyl, heteroaryl, heteroalkyl, heteroalkenyl, heteroalkynyl, heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described above, and hetero versions of any of the groups or moieties described above. Substituents include, but are not limited to, F, Cl, methyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroalkyl, heteroalkenyl, or heteroaryl, and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members.

An optionally substituted group or moiety refers to a group or moiety (e.g., any one of the groups or moieties described above) in which one of the atoms (e.g., a hydrogen atom) is optionally replaced with another substituent. For example, an optionally substituted alkyl may be an optionally substituted methyl, in which a hydrogen atom of the methyl group is replaced by, e.g., OH. As another example, a substituent on a heteroalkyl or its divalent counterpart, heteroalkylene, may replace a hydrogen on a carbon or a hydrogen on a heteroatom such as N. For example, the hydrogen atom in the group —R—NH—R— may be substituted with an alkamide substituent, e.g., —R—N[(CH₂C(O)N(CH₃)₂]—R. Generally, an optional substituent is a noninterfering substituent. A “noninterfering substituent” refers to a substituent that leaves the ability of the conjugates described herein (e.g., conjugates of any one of formulas (D-I)-(D-VIII)) to either bind to viral neuraminidase or to inhibit the proliferation of influenza virus. Thus, in some embodiments, the substituent may alter the degree of such activity. However, as long as the conjugate retains the ability to bind to viral neuraminidase or to inhibitor viral proliferation, the substituent will be classified as “noninterfering.” For example, the noninterfering substituent would leave the ability of the compound to provide antiviral efficacy based on an IC50 value of 10 μM or less in a viral plaque reduction assay, such as in Example 2 of WO 2021/046549 based on an IC50 value against influenza virus neuraminidase of less than 500 nM. Thus, the substituent may alter the degree of inhibition based on plaque reduction or influenza virus neuraminidase inhibition. However, as long as the compounds herein such as compounds of formulas (A-I)-(A-VIII) retain the ability to inhibit influenza virus neuraminidase activity, the substituent will be classified as “noninterfering.” A number of assays for determining viral plaque reduction or the ability of any compound to inhibit influenza virus neuraminidase are available in the art, and some are exemplified in the Examples below.

The term “hetero,” when used to describe a chemical group or moiety, refers to having at least one heteroatom that is not a carbon or a hydrogen, e.g., N, O, and S. Any one of the groups or moieties described above may be referred to as hetero if it contains at least one heteroatom. For example, a heterocycloalkyl, heterocycloalkenyl, or heterocycloalkynyl group refers to a cycloalkyl, cycloalkenyl, or cycloalkynyl group that has one or more heteroatoms independently selected from, e.g., N, O, and S. An example of a heterocycloalkenyl group is a maleimido. For example, a heteroaryl group refers to an aromatic group that has one or more heteroatoms independently selected from, e.g., N, O, and S. One or more heteroatoms may also be included in a substituent that replaced a hydrogen atom in a group or moiety as described herein. For example, in an optionally substituted heteroaryl group, if one of the hydrogen atoms in the heteroaryl group is replaced with a substituent (e.g., methyl), the substituent may also contain one or more heteroatoms (e.g., methanol).

The term “acyl,” as used herein, refers to a group having the structure:

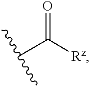

wherein R^{z} is an optionally substituted alkyl, alkenyl, alkynyl, cycloalkyl, cycloalkenyl, cycloalkynyl, aryl, alkaryl, alkamino, heteroalkyl, heteroalkenyl, heteroalkynyl, heterocycloalkyl, heterocycloalkenyl, heterocycloalkynyl, heteroaryl, heteroalkaryl, or heteroalkamino.

The term “halo” or “halogen,” as used herein, refers to any halogen atom, e.g., F, Cl, Br, or I. Any one of the groups or moieties described herein may be referred to as a “halo moiety” if it contains at least one halogen atom, such as haloalkyl.

The term “hydroxyl,” as used herein, represents an —OH group.

The term “oxo,” as used herein, refers to a substituent having the structure ═O, where there is a double bond between an atom and an oxygen atom.

The term “carbonyl,” as used herein, refers to a group having the structure:

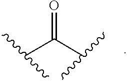 [SMILES: *C(*)=O]

The term “thiocarbonyl,” as used herein, refers to a group having the structure:

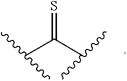 [SMILES: *C(*)=S]

The term “phosphate,” as used herein, represents the group having the structure:

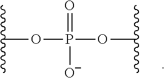 [SMILES: *OP(=O)([O-])O*]

The term “phosphoryl,” as used herein, represents the group having the structure:

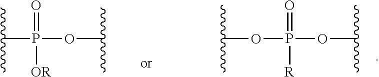

The term “sulfonyl,” as used herein, represents the group having the structure:

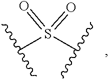 [SMILES: *S(*)(=O)=O]

The term “imino,” as used herein, represents the group having the structure:

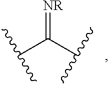

wherein R is an optional substituent.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 5th Edition (John Wiley & Sons, New York, 2014), which is incorporated herein by reference. N-protecting groups include, e.g., acyl, aryloyl, and carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, carboxybenzyl (CBz), 4-chlorobenzoyl, 4-bromobenzoyl, 4-nitrobenzoyl, and chiral auxiliaries such as protected or unprotected D, L or D, L-amino acid residues such as alanine, leucine, phenylalanine; sulfonyl-containing groups such as benzenesulfonyl and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl (BOC), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl (Fmoc), cyclopentyloxycarbonyl, adamantyloxycarbonyl, cyclohexyloxycarbonyl, and phenylthiocarbonyl; alkaryl groups such as benzyl, triphenylmethyl, and benzyloxymethyl; and silyl groups such as trimethylsilyl.

The term “amino acid,” as used herein, means naturally occurring amino acids and non-naturally occurring amino acids.

The term “naturally occurring amino acids,” as used herein, means amino acids including Ala, Arg, Asn, Asp, Cys, Gln, Glu, Gly, His, Ile, Leu, Lys, Met, Phe, Pro, Ser, Thr, Trp, Tyr, and Val.

The term “non-naturally occurring amino acid,” as used herein, means an alpha amino acid that is not naturally produced or found in a mammal.

As used herein, the term “percent (%) identity” refers to the percentage of amino acid residues of a candidate sequence, e.g., an Fc-IgG, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity of a given candidate sequence to, with, or against a given reference sequence (which can alternatively be phrased as a given candidate sequence that has or includes a certain percent amino acid sequence identity to, with, or against a given reference sequence) is calculated as follows:

$$100 \times ( fraction of A / B )$$

where A is the number of amino acid residues scored as identical in the alignment of the candidate sequence and the reference sequence, and where B is the total number of amino acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

Two polynucleotide or polypeptide sequences are said to be “identical” if the sequence of nucleotides or amino acids in the two sequences is the same when aligned for maximum correspondence as described above. Comparisons between two sequences are typically performed by comparing the sequences over a comparison window to identify and compare local regions of sequence similarity. A “comparison window” as used herein, refers to a segment of at least about 15 contiguous positions, about 20 contiguous positions, about 25 contiguous positions, or more (e.g., about 30 to about 75 contiguous positions, or about 40 to about 50 contiguous positions), in which a sequence may be compared to a reference sequence of the same number of contiguous positions after the two sequences are optimally aligned.

The term “treating” or “to treat,” as used herein, refers to a therapeutic treatment of a viral infection in a subject. In some embodiments, a therapeutic treatment may slow the progression of the viral infection, improve the subject's outcome, and/or eliminate the infection. In some embodiments, a therapeutic treatment of a viral infection in a subject may alleviate or ameliorate of one or more symptoms or conditions associated with the viral infection, diminish the extent of the viral, stabilize (i.e., not worsening) the state of the viral infection, prevent the spread of the viral infection, and/or delay or slow the progress of the viral infection, as compare the state and/or the condition of the viral infection in the absence of the therapeutic treatment.

The term “average value of T,” as used herein, refers to the mean number of dimers of neuraminidase inhibitors conjugated to an Fc domain within a population of conjugates. In some embodiments, within a population of conjugates, the average number of dimers of neuraminidase inhibitors conjugated to an Fc domain monomer may be from 1 to 20 (e.g., the average value of T is 1 to 2, 1 to 3, 1 to 4, 1 to 5, 5 to 10, 10 to 15, 15 to 20, 1.5 to 3.5, 2.5 to 4.5, 3.5 to 5.5, 4.5 to 6.5, 5.5 to 7.5, 6.5 to 8.5, 7.5 to 9.5, or 8.5 to 10.5). In some embodiments, the average value of T is 1, 2, 3, 4, 5, 6, 7, 8, 9, 10, 11, 12, 13, 14, 15, 16, 17, 18, 19, or 20.

The term “subject,” as used herein, can be a human, non-human primate, or other animal, such as but not limited to dog, cat, horse, cow, pig, turkey, goat, fish, monkey, chicken, rat, mouse, and sheep.

The term “therapeutically effective amount,” as used herein, refers to an amount, e.g., pharmaceutical dose, effective in inducing a desired effect in a subject or in treating a subject having a condition or disorder described herein (e.g., a viral infection, such as an influenza infection). It is also to be understood herein that a “therapeutically effective amount” may be interpreted as an amount giving a desired therapeutic and/or preventative effect, taken in one or more doses or in any dosage or route, and/or taken alone or in combination with other therapeutic agents (e.g., an antiviral agent described herein). For example, in the context of administering a conjugate described herein (e.g., a conjugate of any one of formulas (D-I)-(D-VIII)) that is used for the treatment of a viral infection, an effective amount of a conjugate is, for example, an amount sufficient to prevent, slow down, or reverse the progression of the viral infection as compared to the response obtained without administration of the conjugate.

As used herein, the term “pharmaceutical composition” refers to a medicinal or pharmaceutical formulation that contains at least one active ingredient (e.g., a conjugate of any one of formulas (D-I)-(D-VIII)) as well as one or more excipients and diluents to enable the active ingredient suitable for the method of administration. The pharmaceutical composition of the present disclosure includes pharmaceutically acceptable components that are compatible with a conjugate described herein (e.g., a conjugate of any one of formulas (D-I)-(D-VIII)).

As used herein, the term “pharmaceutically acceptable carrier” refers to an excipient or diluent in a pharmaceutical composition. For example, a pharmaceutically acceptable carrier may be a vehicle capable of suspending or dissolving the active conjugate (e.g., a conjugate of any one of formulas (D-I)-(D-VIII)). The pharmaceutically acceptable carrier must be compatible with the other ingredients of the formulation and not deleterious to the recipient. In the present disclosure, the pharmaceutically acceptable carrier must provide adequate pharmaceutical stability to a conjugate described herein. The nature of the carrier differs with the mode of administration. For example, for oral administration, a solid carrier is preferred; for intravenous administration, an aqueous solution carrier (e.g., WFI, and/or a buffered solution) is generally used.

The term “pharmaceutically acceptable salt,” as used herein, represents salts of the conjugates described herein (e.g., conjugates of any one of formulas (D-I)-(D-VIII)) that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

The term “drug-to-antibody ratio” or “DAR” refers to the average number of small molecule drug moieties (e.g., the average number of small molecule drug dimers) conjugated to an antibody or Fc domain, described herein. In some embodiments described herein, the DAR is represented by ‘T’ (e.g., in formulas (D-I)-(D-VIII)). As used herein, each dimer moiety (e.g., each zanamivir dimer) conjugated to the Fc domain or antibody corresponds to a DAR or “T” value of 1.0. DAR values may affect the efficacy, potency, pharmacokinetics, or toxicity of the drug.

The term “secondary infection,” as used herein, refers to an infection that occurs in a subject during or after another (referred to as primary) infection in that subject (e.g., during or after a primary influenza infection). A secondary infections may be caused by the primary infection or may be caused by treatment of the primary infection. In some cases, primary infections alter the immune system making the subject more susceptible to a secondary infection. In some cases, treatment of the primary infection makes the subject more susceptible to a secondary infection. For example, the influenza virus has been associated with secondary infections (e.g., increased risk of developing a secondary infection), such as bacterial secondary infections, for example of the respiratory tract. Secondary infections associated with influenza infection increase the morbidity and mortality of influenza. Secondary infections include co-infections. The terms “secondary infection” and “co-infection” are used interchangeably herein.

The term “about,” as used herein, indicates a deviation of up to ±5%. For example, about 10% refers to from 9.5% to 10.5%.

Any values provided in a range of values include both the upper and lower bounds, and any values contained within the upper and lower bounds.

The term “(D-I)-(D-VIII)”, as used herein, represents the formulas of any one of (D-I), (D-II), (D-II-1), (D-I-2), (D-I-3), (D-II-4), (D-I-5), (D-II-6), (D-II-7), (D-I-8), (D-II-9), (D-II-10), (D-III), (D-III-1), (D-III-2), (D-III-3), (D-III-4), (D-III-5), (D-III-6), (D-III-7), (D-III-8), (D-III-9), (D-IV), (D-IV-1), (D-IV-2), (D-V), (D-V-1), (D-V-2), (D-V-3), (D-V-4), (D-V-5), (D-V-8), (D-V-7), (D-V-8), (D-V-9), (D-V-10), (D-VI), (D-VI-1), (D-VI-2), (D-VI-3), (D-VI-4), (D-VI-5). (D-VI-6), (D-VI-7), (D-VI-8), (D-VI-9), (D-VII), (D-VIII), (D-VIII-1).

Other features and advantages of the conjugates described herein will be apparent from the following Detailed Description and the claims.

### DESCRIPTION OF THE DRAWINGS

FIG. 1 is an image showing a portion of the crystal structure of the Fc domain of human IgG1 (PDB ID 4W4N), showing the positions of the K246 side-chain, and M252, S254, and T256, which may be mutated to Y. T, and E, respectively, in an engineered Fc variant that demonstrates enhanced binding to the human FcRn receptor. The terminal nitrogen atom of the K248 lysine side-chain is in close proximity to the side-chain atoms of residues 252, 254 and 256 in the FcRn binding-site, (approximately 10-14 Angstroms). Large chemical groups conjugated to K246 may interfere with FcRn binding.

FIG. 2 is an image of a non-reducing (NR) and reducing (R) SDS-PAGE of an Fc domain formed from Fc domain monomers having the sequence of SEQ ID NO: 12.

FIG. 3 is an image showing an exemplary conjugate of the disclosure.

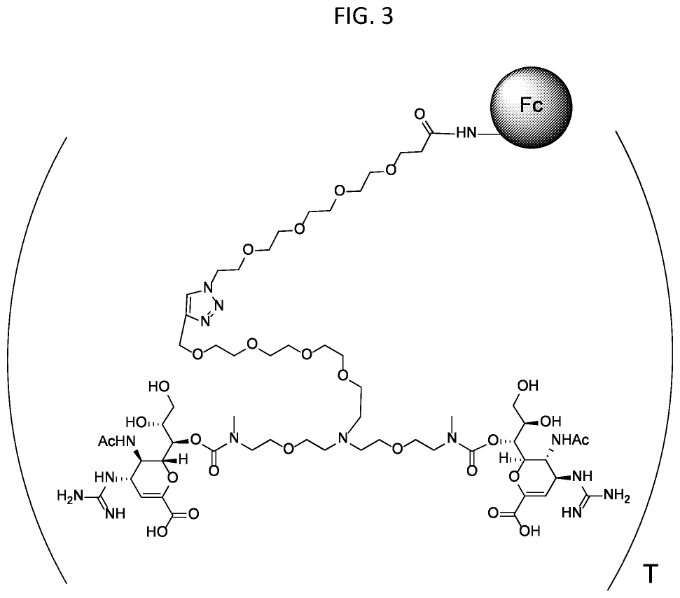

### DETAILED DESCRIPTION

The disclosure features conjugates, compositions, and methods for the treatment of viral infections (e.g., influenza viral infections). The conjugates disclosed herein include dimers of viral neuraminidase inhibitors (e.g., zanamivir or analogs thereof) conjugated to Fc monomers or Fc domains. The neuraminidase inhibitor (e.g., zanamivir or analogs thereof) in the conjugates targets neuraminidase on the surface of the viral particle. The Fc monomers or Fc domains in the conjugates bind to FcγRs (e.g., FcRn, FcγRI, FcγRIIa, FcγRIIc, FcγRIIIa, and FcγRIIIb) on immune cells. e.g., neutrophils, to activate phagocytosis and effector functions, such as antibody-dependent cell-mediated cytotoxicity (ADCC), thus leading to the engulfment and destruction of viral particles by immune cells and further enhancing the antiviral activity of the conjugates. Such compositions are useful in methods for the inhibition of viral growth and in methods for the treatment of viral infections, such as those caused by an influenza virus A, influenza virus B and influenza virus C.

### I. Viral Infections

The compounds and pharmaceutical compositions described herein (e.g., a conjugate of any one of formulas (D-I)-(D-VIII)) can be used to treat a viral infection (e.g., an influenza viral infection, such as influenza A, B, C, or parainfluenza).

Viral infection refers to the pathogenic growth of a virus (e.g., the influenza virus) in a host organism (e.g., a human subject). A viral infection can be any situation in which the presence of a viral population(s) is damaging to a host body. Thus, a subject is suffering from a viral infection when an excessive amount of a viral population is present in or on the subject's body, or when the presence of a viral population(s) is damaging the cells or other tissue of the subject.

Influenza, commonly known as “the flu”, is an infectious disease caused by an influenza virus. Symptoms can be mild to severe. The most common symptoms include: a high fever, runny nose, sore throat, muscle pains, headache, coughing, and feeling tired. These symptoms typically begin two days after exposure to the virus and most last less than a week. The cough, however, may last for more than two weeks. In children, there may be nausea and vomiting, but these are less common in adults. Complications of influenza may include viral pneumonia, secondary bacterial pneumonia, sinus infections, and worsening of previous health problems such as asthma or heart failure. Severe complications may occur in subjects having weakened immune systems, such as the young, the old, those with illnesses that weaken the immune system, and those undergoing therapy treatment resulting in a weakening of the immune system.

Subjects infected with influenza are also at increased risk of developing secondary infections (e.g., secondary bacterial, viral, or fungal infections), in particular, bacterial infections such as methicillin-resistant Staphylococcus aureus (MRSA), Streptococcus pneumoniae, Pseudomonas aeruginosa, and/or Haemophilus influenzae. Bacterial secondary infections further increase morbidity and mortality of influenza infection.

Three types of influenza viruses affect human subjects, namely Type A, Type B. and Type C. Usually, the virus is spread through the air from coughs or sneezes. This is believed to occur mostly over relatively short distances. It can also be spread by touching surfaces contaminated by the virus and then touching the mouth or eyes. A person may be infectious to others both before and during the time they are showing symptoms. The infection may be confirmed by testing the throat, sputum, or nose for the virus. A number of rapid tests are available; however, people may still have the infection if the results are negative. A type of polymerase chain reaction that detects the virus's RNA may be used to diagnose influenza infection.

### II. Conjugates of the Disclosure

Provided herein are synthetic conjugates useful in the treatment of viral infections (e.g., influenza infections). The conjugates disclosed herein include an Fc domain conjugated to one or more dimers of two neuraminidase inhibitors (e.g., neuraminidase inhibitors selected from zanamivir or analogs thereof). The dimers of two neuraminidase inhibitors include a first neuraminidase inhibitor which is zanamivir or an analog thereof (e.g., of formula (A-I)-(A-VIII)) and a second neuraminidase inhibitor which is zanamivir or an analog thereof (e.g., of formula (A-I)-(A-VIII)). The first and second neuraminidase inhibitors are linked to each other by way of a linker.

Without being bound by theory, in some aspects, conjugates described herein bind to the surface of a viral particle (e.g., bind to viral neuraminidase enzyme on the surface on an influenza viral particle) through the interactions between the neuraminidase inhibitor moieties in the conjugates and proteins on the surface of the viral particle. The neuraminidase inhibitor disrupts neuraminidase, an envelope glycoprotein that cleaves sialic acids, i.e., terminal neuraminic acid residues, from glycan structures on the surface of infected host cells, releasing progeny viruses and allowing the spread of the virus from the host cell to uninfected surrounding cells.

Conjugates of the disclosure include neuraminidase inhibitor dimers conjugated to an Fc domain or Fc monomer. The Fc domain in the conjugates described herein binds to the FcγRs (e.g., FcRn, FcγRI, FcγRIIa, FcγRIIc, FcγRIIIa, and FcγRIIIb) on immune cells. The binding of the Fc domain in the conjugates described herein to the FcγRs on immune cells activates phagocytosis and effector functions, such as antibody-dependent cell-mediated cytotoxicity (ADCC), thus leading to the engulfment and destruction of viral particles by immune cells and further enhancing the antiviral activity of the conjugates.

Conjugates provided herein are described by any one of formulas (D-I)-(D-VIII). In some embodiments, the conjugates described herein include one or more dimers of neuraminidase inhibitors conjugated to an Fc domain. In some embodiments, when n is 2, E (an Fc domain monomer) dimerizes to form an Fc domain.

Conjugates described herein may be synthesized using available chemical synthesis techniques in the art. In cases where a functional group is not available for conjugation, a molecule may be derivatized using conventional chemical synthesis techniques that are well known in the art. In some embodiments, the conjugates described herein contain one or more chiral centers. The conjugates include each of the isolated stereoisomeric forms as well as mixtures of stereoisomers in varying degrees of chiral purity, including racemic mixtures. It also encompasses the various diastereomers, enantiomers, and tautomers that can be formed.

### Neuraminidase Inhibitors

A component of the conjugates described herein is an influenza virus neuraminidase inhibitor moiety. An influenza virus neuraminidase inhibitor disrupts neuraminidase, an envelope glycoprotein that cleaves sialic acids, i.e., terminal neuraminic acid residues, from glycan structures on the surface of infected host cells, releasing progeny viruses and allowing the spread of the virus from the host cell to uninfected surrounding cells. Examples of an influenza virus neuraminidase inhibitor include zanamivir (Relenza), sulfozanamivir, and analogs thereof that retain neuraminidase inhibitor binding and/or activity.

Viral neuraminidase inhibitors of the disclosure include zanamivir, sulfozanamivir, and analogs thereof, such as the viral neuraminidase inhibitors of formulas (A-I)-(A-VIII).

Preferably the viral neuraminidase inhibitor is selected from zanamivir or sulfozanamivir.

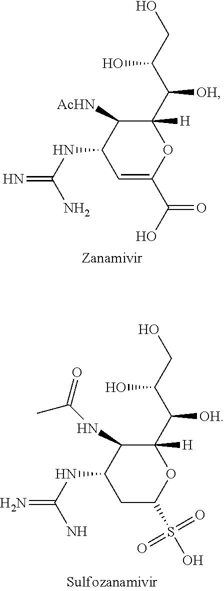

### Conjugates of Dimers of Neuraminidase Inhibitors Linked to an Fc Domain

The conjugates described herein include an Fc domain or an Fc monomer covalently linked to one or more dimers of neuraminidase inhibitors. The dimers of two neuraminidase inhibitors include a first neuraminidase inhibitor (e.g., a first viral neuraminidase inhibitor of formulas (A-I)-(A-VIII)) and a second neuraminidase inhibitor (e.g., a second viral neuraminidase inhibitor of formulas (A-I)-(A-VIII)). The first and second neuraminidase inhibitors are linked to each other by way of a linker, such as a linker described herein. In some embodiments of the dimers of neuraminidase inhibitors, the first and second neuraminidase inhibitors are the same. In some embodiments, the first and second neuraminidase inhibitors are different.

In some embodiments, the disclosure provides a conjugate, or a pharmaceutically acceptable salt thereof, described by any one of formulae (D-I), (D-II), (D-III), (D-IV), (D-V), (D-VI), (D-VII), or (D-VIII).

In the conjugates described herein, the squiggly line connected to E indicates that one or more (e.g., 1, 2, 3, 4, 5, 6, 7, 8, 9, 10, 11, 12, 13, 14, 15, 16, 17, 18, 19, or 20) dimers of neuraminidase inhibitors may be attached to an Fc domain monomer or Fc domain. In some embodiments, when n is 1, one or more (e.g., 1, 2, 3, 4, 5, 6, 7, 8, 9, or 10) dimers of neuraminidase inhibitors may be attached to an Fc domain monomer or Fc domain. In some embodiments, when n is 2, one or more (e.g., 1, 2, 3, 4, 5, 6, 7, 8, 9, 10, 11, 12, 13, 14, 15, 16, 17, 18, 19, or 20) dimers of neuraminidase inhibitors may be attached to an Fc domain. The squiggly line in the conjugates described herein is not to be construed as a single bond between one or more dimers of neuraminidase inhibitors and an atom in the Fc domain. In some embodiments, when T is 1, one dimer of neuraminidase inhibitors may be attached to an atom in the Fc domain monomer or Fc domain. In some embodiments, when T is 2, two dimers of neuraminidase inhibitors may be attached to an atom in the Fc domain monomer or Fc domain.

As described further herein, a linker in a conjugate described herein (e.g., L or L) may be a branched structure. As described further herein, a linker in a conjugate described herein (e.g., L or L) may be a multivalent structure, e.g., a divalent or trivalent structure having two or three arms, respectively. In some embodiments when the linker has three arms, two of the arms may be attached to the first and second neuraminidase inhibitors and the third arm may be attached to the Fc domain monomer or Fc domain.

In conjugates having an Fc domain covalently linked to one or more dimers of neuraminidase inhibitors, as represented by the formulae above, when n is 2, two Fc domain monomers (each Fc domain monomer is represented by E) dimerize to form an Fc domain.

### Regioisomers of Conjugates Including Zanamivir or Analogs Thereof

Conjugates may be produced as a mixture or regioisomers. A particular regioisomer or mixture of regioisomers may be preferred for reasons such as ease of synthesis, thermostability, oxidative stability, pharmacokinetics (e.g., metabolic stability or bioavailability), effector binding, or therapeutic efficacy.

In some embodiments, a conjugate of the disclosure includes zanamivir or an analog thereof (e.g., any of (A-I)-(A-VIII)). Zanamivir or an analog thereof may be conjugated to an Fc domain (e.g., by way of a linker) through, for example, the C7 position (see, e.g., (A-I), (A-II), (A-VII), or (A-VIII)) or through the C9 position (see, e.g., (A-III) or (A-IV)):

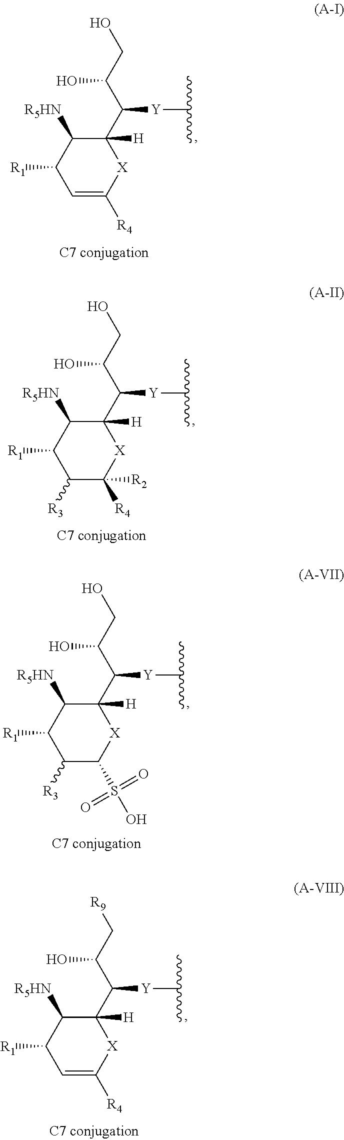

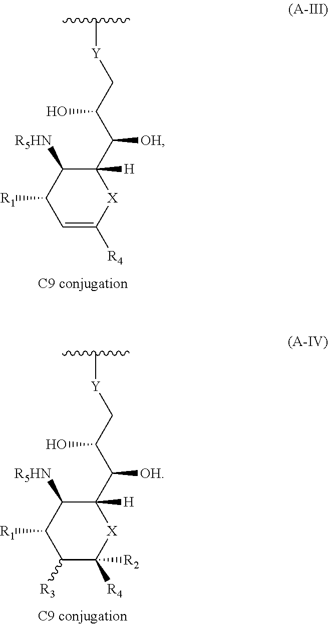

The present disclosure includes a population of conjugates (e.g., a population of conjugates of any one of formulas (D-I)-(D-VIII)) wherein the population of conjugates includes any of the dimeric conjugates described herein and one or more of its corresponding regioisomers. For example, a population of conjugates may include a (1) a C7-C7 dimer (e.g., both zanamivir or analog thereof moieties of the dimer are conjugated (e.g., by way of a linker) at their respective C7 positions to an Fc domain), (2) a C9-C9 dimer (e.g., both zanamivir or analog thereof moieties of the dimer are conjugated (e.g., by way of a linker) at their respective C9 positions to an Fc domain), and/or (3) a C7-C9 dimer (e.g., one zanamivir or analog thereof moiety is conjugated (e.g., by way of a linker) to and Fc domain through its C7 position and the other zanamivir or analog thereof moiety is conjugated (e.g., by way of a linker) to an Fc domain through its C9 position).

The population of dimeric conjugates may have a specified ratio of C7-C7 linked conjugate to C7-C9 linked conjugate to C9-C9 linked conjugate. For example, the population of conjugates may have substantially 100% C7-C7 linked conjugate, and substantially 0% C7-C9 or C9-C9 linked conjugate. The population of conjugates may have substantially 100% C9-C9 linked conjugate, and substantially 0% C7-C7 or C7-C9 linked conjugate. The population of conjugates may have substantially 100% C7-C9 linked conjugate, and substantially 0% C7-C7 or C9-C9 linked conjugate.

The population of conjugates may have greater than 99%, 98%, 97%, 96%, 95%, 90%, 85%, 80%, 75%, 70%, 60%, 65%, 60%, 55%, or 50% C7-C7 linked conjugate.

The population of conjugates may have less than 50%, 40%, 30%, 25%, 20%, 15%, 10%, 5%, 4%, 3%, 2%, or 1% C9-C9 linked conjugate.

The population of conjugates may have less than 50%, 40%, 30%, 25%, 20%, 15%, 10%, 5%, 4%, 3%, 2%, or 1% C7-C9 linked conjugate.

For any of the above-described populations of regioisomers, A₁ and/or A₂ may be selected from zanamivir or any of the zanamivir analogs described herein (e.g., any of (A-I)-(A-VIII)). In particular, the C7-linked zanamivir or analogs thereof is described by (A-I), (A-II), (A-VII), and (A-VIII), and C9-linked zanamivir or analogs thereof is described by (A-III) or (A-IV). Exemplary methods for preparing regioisomers, e.g., C7, C9, C7-C7, C7-C9, and C9-C9 linked regioisomers, are described in Examples 100-103, 123 and 124 of WO 2021/046549. In some instances, it may be preferable to have 95% or more, 96% or more, 97% or more, 98% or more, 99% or more, or substantially 100% C7-C7 linked dimer conjugates. In these instances, it may be preferable to prepare the intermediate with a method that forms substantially C7-C7 linked dimer intermediates, such as the methods described, for example, in Examples 103 and 123 WO 2021/046549. The method of Example 103 of WO 2021/046549 is exemplary of methods used to achieve primarily the C7 or C7-C7 linked intermediate and may be used to prepare any intermediate described herein.

Zanamivir analogs having a modification (e.g., a substituent other than OH) at position C9 (e.g., zanamivir analogs described by (A-XIII)) may increase the ratio of C7-linked zanamivir to C9-linked zanamivir by preventing the migration from C7-linked zanamivir to C9-linked zanamivir. Exemplary C9-modified zanamivir analogs are described herein (see, e.g., conjugates described by D-XI or M-XI).

In preferred embodiments, the conjugate is a conjugate of any one of formulas (D-I)-(D-VIII), wherein A₁ and/or A₂ are described by formula (A-I), (A-II), (A-VII), or (A-VIII) and Y is

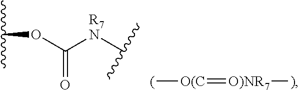

wherein R₇ is selected from H, C1-C20 alkyl, C3-C20 cycloalkyl, C3-C20 heterocycloalkyl; C5-C15 aryl, and C2-C15 heteroaryl. In preferred embodiments, A₁ and/or A₂ are described by formula (A-I) (e.g., zanamivir). In preferred embodiment F is, R₇ is C1-C20 alkyl (e.g., —CH₃, —CH₂CH₃, —CH₂CH₂CH₃). Such conjugates have been shown to exhibit increased stability of the C7-linkage, resulting in less C7 to C9 migration (see, e.g., conjugates described by D-II-6 or D-II-7). The resulting product is therefore expected to be more homogenous and exhibit increased efficacy. The preferred conjugate is more homogenous, has an increased proportion (e.g., substantially pure, such as greater than 95%, 96%, 97%, 98%, or 99% pure) C7-linked zanamivir, and retains efficacy against influenza.

### III. Fc Domain Monomers and Fc Domains

An Fc domain monomer includes a hinge domain, a C_{H}2 antibody constant domain, and a C_{H}3 antibody constant domain. In some embodiments, the Fc domain includes an amino acid substitution at position 246 (e.g., K246X where X is any amino acid that is not Lys, such as K246S, K246G, K246A, K246T, K246N, K246Q, K246R, K246H, K246E, or K246DC220S). In some embodiments, the Fc domain monomer includes at least the following mutations K246X, M252Y, S254T, and T256E, where X is not Lys. In some embodiments, the Fc domain monomer includes at least the following mutations K246X, V309D, Q311H, and N434S, where X is not Lys. In some embodiments, the Fc domain monomer includes at least the following mutations K246X, M428L, and N434S, where X is not Lys. In some embodiments, the Fc domain further includes a mutation of position 220, e.g., a C220S mutation. Amino acid substitutions are relative to a wild-type Fc monomer amino acid sequence, e.g., wild-type human IgG1 or IgG2.

The Fc domain monomer can be of immunoglobulin antibody isotype IgG, IgE, IgM, IgA, or IgD. The Fc domain monomer can also be of any immunoglobulin antibody isotype (e.g., IgG1, IgG2a, IgG2b, IgG3, or IgG4). The Fc domain monomer can be of any immunoglobulin antibody allotype (e.g., IGHG1*01 (i.e., G1m(za)), IGHG1*07 (i.e., G1m(zax)), IGHG1*04 (i.e., G1m(zav)), IGHG1*03 (G1m(f)), IGHG1*08 (i.e., G1m(fa)), IGHG2*01, IGHG2*06, IGHG2*02, IGHG3*01, IGHG3*05, IGHG3*10, IGHG3*04, IGHG3*09, IGHG3*11, IGHG3*12, IGHG3*06, IGHG3*07, IGHG3*08, IGHG3*13, IGHG3*03, IGHG3*14, IGHG3*15. IGHG3*16, IGHG3*17, IGHG3*18, IGHG3*19, IGHG2*04, IGHG4*01, IGHG4*03, or IGHG4*02) (as described in, for example, in Vidarsson et al. IgG subclasses and allotypes: from structure to effector function. Frontiers in Immunology. 5(520):1-17 (2014)). The Fc domain monomer can also be of any species, e.g., human, murine, or mouse. A dimer of Fc domain monomers is an Fc domain that can bind to an Fc receptor, which is a receptor located on the surface of leukocytes. In some embodiments, an Fc domain monomer in the conjugates described herein may contain one or more amino acid substitutions, additions, and/or deletion relative to an Fc domain monomer having a sequence of any one of SEQ ID NOs: 1-29. In some embodiments, an Asn in an Fc domain monomer in the conjugates as described herein may be replaced by Ala in order to prevent N-linked glycosylation. In some embodiments, an Fc domain monomer in the conjugates described herein may also containing additional Cys additions.

In some embodiments, an Fc domain monomer in the conjugates as described herein includes an additional moiety, e.g., an albumin-binding peptide, a purification peptide (e.g., a hexa-histidine peptide), or a signal sequence (e.g., IL2 signal sequence) attached to the N- or C-terminus of the Fc domain monomer. In some embodiments, an Fc domain monomer in the conjugate does not contain any type of antibody variable region, e.g., V_{H}, V_{L}, a complementarity determining region (CDR), or a hypervariable region (HVR).

In some embodiments, an Fc domain monomer in the conjugates as described herein may have a sequence that is at least 95% identical (e.g., 97%, 99%, or 99.5% identical) to the sequence of any one of SEQ ID NOs: 1-29 shown below. In some embodiments, an Fc domain monomer in the conjugates as described herein may have a sequence of any one of SEQ ID NOs: 1-29 shown below.

In some embodiments, an Fc domain monomer includes at least the following mutations K246X, M252Y, S254T, and T256E, where X is not Lys. For example, the Fc domain monomer has a sequence that is at least 95% identical (e.g., 97%, 99%, or 99.5% identical) to the sequence of any one of SEQ ID NOs: 9-15 shown below. In some embodiments, an Fc domain monomer has the sequence of any one of SEQ ID Nos: 9-15 shown below.

In some embodiments, an Fc domain monomer includes at least the following mutations K246X, V309D, Q311H, and N434S, where X is not Lys. For example, the Fc domain monomer has a sequence that is at least 95% identical (e.g., 97%, 99%, or 99.5% identical) to the sequence of any one of SEQ ID Nos: 16-22 shown below. In some embodiments, an Fc domain monomer has the sequence of any one of SEQ ID NOs: 16-22 shown below.

In some embodiments, an Fc domain monomer includes at least the following mutations K246X, M428L, and N434S, where X is not Lys. For example, the Fc domain monomer has a sequence that is at least 95% identical (e.g., 97%, 99%, or 99.5% identical) to the sequence of any one of SEQ ID NOs: 23-28 shown below. In some embodiments, an Fc domain monomer has the sequence of any one of SEQ ID NOs: 23-29 shown below.

SEQ ID NO: 1: mature human IgG1 Fc; X₁ (position 201) is Asn or absent; X₂ (position 220) is Cys or Ser; X₃ (position 248) is Ser, Gly, Ala, Thr, Asn, Gln, Arg, His, Glu, or Asp; X₄ (position 252) is Met or Tyr, X₅ (position 254) is Ser or Thr; X₆ (position 256) is Thr or Glu; X₇ (position 297) is Asn or Ala; X₈ (position 309) is Leu or Asp; X₉ (position 311) is Gln or His; X₁₀ (position 356) is Asp or Glu; and X₁₁ (position 358) is Leu or Met; X₁₂ (position 428) is Met or Leu; X₁₃ (position 434) is Asn or Ser; X₁₄ (position 447) is Lys or absent; N-terminal Fab residues are underlined; hinge residues are italicized

X₁VNHKPSNTKVDKKVEPKSX₂DKTHTCPPCPAPELLGGPSVFLFPPX₃P
KDTLX₄IX₅RX₆PEVTCVVVDVSHEDPEVKFNWYVDGVEVHNAKTKPREE
QYX₇STYRVVSVLTVX₈HX₉DWLNGKEYKCKVSNKALPAPIEKTISKAKG
QPREPQVYTLPPSRX₁₀EX₁₁TKNQVSLTCLVKGFYPSDIAVEWESNGQP
ENNYKTTPPVLDSDGSFFLYSKLTVDKSRWQQGNVFSCSVX₁₂HEALH
X₁₃HYTQKSLSLSPGX₁₄

SEQ ID NO: 2: mature human IgG1 Fc; Cys to Ser substitution (#); X₁ is Asn or absent; X₂ is Ser, Gly, Ala, Thr, Asn, Gln, Arg, His, Glu, or Asp; X₃ is Asn or Ala; X₄ is Asp or Glu; and X₅ is Leu or Met; X₆ is Lys or absent; N-terminal Fab residues are underlined; hinge residues are italicized

X₁VNHKPSNTKVDKKVEPKSS(#)DKTHTCPPCPAPELLGGPSVFLFPP
X₂PKDTLMISRTPEVTCVVVDVSHEDPEVKFNWYVDGVEVHNAKTKPREE
QYX₃STYRVVSVLTVLHQDWLNGKEYKCKVSNKALPAPIEKTISKAKGQP
REPQVYTLPPSRX₄EX₅TKNQVSLTCLVKGFYPSDIAVEWESNGQPENNY
KTTPPVLDSDGSFFLYSKLTVDKSRWQQGNVFSCSVMHEALHNHYTQKSL
SLSPGX₆

In some embodiments, the Fc domain monomer includes the amino acid sequence of SEQ ID NO: 2 where X₂ is Ser. In some embodiments, the Fc domain monomer includes the amino acid sequence of SEQ ID NO: 2 where X₂ is Gly. In some embodiments, the Fc domain monomer includes the amino acid sequence of SEQ ID NO: 2 where X₂ is Ala. In some embodiments, the Fc domain monomer includes the amino acid sequence of SEQ ID NO: 2 where X₂ is Thr. In some embodiments, the Fc domain monomer includes the amino acid sequence of SEQ ID NO: 2 where X₂ is Asn. In some embodiments, the Fc domain monomer includes the amino acid sequence of SEQ ID NO: 2 where X₂ is Gln. In some embodiments, the Fc domain monomer includes the amino acid sequence of SEQ ID NO: 2 where X₂ is Arg. In some embodiments, the Fc domain monomer includes the amino acid sequence of SEQ ID NO: 2 where X₂ is Glu. In some embodiments, the Fc domain monomer includes the amino acid sequence of SEQ ID NO: 2 where X₂ is Asp.

In some embodiments, the Fc domain monomer includes the amino acid sequence of SEQ ID NO: 2 where X₄ is Asp and X₅ is Leu (corresponding to Fc allotype G1m(fa)). In some embodiments, the Fc domain monomer includes the amino acid sequence of SEQ ID NO: 2 where X₄ is Glu and X₅ is Met (corresponding to Fc allotype G1m(f)).

SEQ ID NO: 3: mature human IgG1 Fc; Cys to Ser substitution (#); X₁ is Ser, Gly, Ala, Thr, Asn, Gln, Arg, His, Glu, or Asp; X₂ is Asn or Ala; X₃ is Asp or Glu; and X₄ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLMISRTPEVTCVVVDVSHEDPEVKFNWYVDGVEVHNAKTKPREEQY
X₂STYRVVSVLTVLHQDWLNGKEYKCKVSNKALPAPIEKTISKAKGQPRE
PQVYTLPPSRX₃EX₄TKNQVSLTCLVKGFYPSDIAVEWESNGQPENNYKT
TPPVLDSDGSFFLYSKLTVDKSRWQQGNVFSCSVMHEALHNHYTQKSLSL
SPG

In some embodiments, the Fc domain monomer includes the amino acid sequence of SEQ ID NO: 3 where X₁ is Ser. In some embodiments, the Fc domain monomer includes the amino acid sequence of SEQ ID NO: 3 where X₁ is Gly. In some embodiments, the Fc domain monomer includes the amino acid sequence of SEQ ID NO: 3 where X₁ is Ala. In some embodiments, the Fc domain monomer includes the amino acid sequence of SEQ ID NO: 3 where X₁ is Thr. In some embodiments, the Fc domain monomer includes the amino acid sequence of SEQ ID NO: 3 where X₁ is Asn. In some embodiments, the Fc domain monomer includes the amino acid sequence of SEQ ID NO: 3 where X₁ is Gln. In some embodiments, the Fc domain monomer includes the amino acid sequence of SEQ ID NO: 3 where X₁ is Arg. In some embodiments, the Fc domain monomer includes the amino acid sequence of SEQ ID NO: 3 where X₁ is Glu. In some embodiments, the Fc domain monomer includes the amino acid sequence of SEQ ID NO: 3 where X₁ is Asp.

In some embodiments, the Fc domain monomer includes the amino acid sequence of SEQ ID NO: 3 where X₃ is Asp and X₄ is Leu (corresponding to Fc allotype G1 m(fa)). In some embodiments, the Fc domain monomer includes the amino acid sequence of SEQ ID NO: 3 where X₃ is Glu and X₄ is Met (corresponding to Fc allotype G1m(f)).

SEQ ID NO: 4: mature human IgG1 Fc; Cys to Ser substitution (#); Lys to Ser substitution(*); X₁ is Asp or Glu; and X₂ is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRX₁EX₂TKNQVSLTCLVKGFYPSDIAVEWESNGQPENNY
KTTPPVLDSDGSFFLYSKLTVDKSRWQQGNVFSCSVMHEALHNHYTQKSL
SLSPG

SEQ ID NO: 5: mature human IgG1 Fc; Cys to Ser substitution (#); Lys to Ser substitution(*); allotype G1m(fa) (bold italics); N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSL
SPG

SEQ ID NO: 6: mature human IgG1 Fc; Cys to Ser substitution (#); Lys to Ser substitution(*); YTE triple mutation (bold and underlined); allotype G1m(f) (bold italics); N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPG

SEQ ID NO: 7: mature human IgG1 Fc; Cys to Ser substitution (#); Asn to Ala substitution (\hat{ }); X₁ is Ser, Gly, Ala, Thr. Asn, Gln, Arg, His, Glu, or Asp; X₂ is Asp or Glu; and X₃ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLMISRTPEVTCVVVDVSHEDPEVKFNWYVDGVEVHNAKTKPREEQY
A(^{∧})STYRVVSVLTVLHQDWLNGKEYKCKVSNKALPAPIEKTISKAKGQP
REPQVYTLPPSRX₂EX₃TKNQVSLTCLVKGFYPSDIAVEWESNGQPENNY
KTTPPVLDSDGSFFLYSKLTVDKSRWQQGNVFSCSVMHEALHNHYTQKSL
SLSPG

SEQ ID NO: 8: mature human IgG1 Fc; Cys to Ser substitution (#); Lys to Ser substitution(*); Asn to Ala substitution (\hat{ }); X₁ is Asp or Glu; and X₂ is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYA(^{∧})STYRVVSVLTVLHQDWLNGKEYKCKVSNKALPAPIEKTISKAK
GQPREPQVYTLPPSRX₁EX₂TKNQVSLTCLVKGFYPSDIAVEWESNGQPE
NNYKTTPPVLDSDGSFFLYSKLTVDKSRWQQGNVFSCSVMHEALHNHYTQ
KSLSLSPG

SEQ ID NO: 9 mature human IgG1 Fc; Cys to Ser substitution (#); YTE triple mutation (bold and underlined); X₁ is Asn or absent; X₂ is Ser, Gly, Ala, Thr, Asn, Gln, Arg, His, Glu, or Asp; X₃ is Asn or Ala; X₄ is Asp or Glu; and X₅ is Leu or Met; X₅ is Lys or absent; N-terminal Fab residues are underlined; hinge residues are italicized

X₁VNHKPSNTKVDKKVEPKSS(#)DKTHTCPPCPAPELLGGPSVFLFPP
X₂PKDTLYITREPEVTCVVVDVSHEDPEVKFNWYVDGVEVHNAKTKPREE
QYX₃STYRVVSVLTVLHQDWLNGKEYKCKVSNKALPAPIEKTISKAKGQP
REPQVYTLPPSRX₄EX₅TKNQVSLTCLVKGFYPSDIAVEWESNGQPENNY
KTTPPVLDSDGSFFLYSKLTVDKSRWQQGNVFSCSVMHEALHNHYTQKSL
SLSPGX₆

In some embodiments, the Fc domain monomer includes the amino acid sequence of SEQ ID NO: 9 where X₂ is Ser. In some embodiments, the Fc domain monomer includes the amino acid sequence of SEQ ID NO: 9 where X₂ is Gly. In some embodiments, the Fc domain monomer includes the amino acid sequence of SEQ ID NO: 9 where X₂ is Ala. In some embodiments, the Fc domain monomer includes the amino acid sequence of SEQ ID NO: 9 where X₂ is Thr. In some embodiments, the Fc domain monomer includes the amino acid sequence of SEQ ID NO: 9 where X₂ is Asn. In some embodiments, the Fc domain monomer includes the amino acid sequence of SEQ ID NO: 9 where X₂ is Gln. In some embodiments, the Fc domain monomer includes the amino acid sequence of SEQ ID NO: 9 where X₂ is Arg. In some embodiments, the Fc domain monomer includes the amino acid sequence of SEQ ID NO: 9 where X₂ is Glu. In some embodiments, the Fc domain monomer includes the amino acid sequence of SEQ ID NO: 9 where X₂ is Asp.

In some embodiments, the Fc domain monomer includes the amino acid sequence of SEQ ID NO: 9 where X₄ is Asp and X₅ is Leu (corresponding to Fc allotype G1m(fa)). In some embodiments, the Fc domain monomer includes the amino acid sequence of SEQ ID NO: 9 where X₄ is Glu and X₅ is Met (corresponding to Fc allotype G1m(f)).

SEQ ID NO: 10: mature human IgG1 Fc; Cys to Ser substitution (#); YTE triple mutation (bold and underlined); X₁ is Ser. Gly, Ala, Thr, Asn, Gln, Arg, His, Glu, or Asp; X₂ is Asn or Ala; X₃ is Asp or Glu; and X₄ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLYITREPEVTCVVVDVSHEDPEVKFNWYVDGVEVHNAKTKPREEQY
X₂STYRVVSVLTVLHQDWLNGKEYKCKVSNKALPAPIEKTISKAKGQPRE
PQVYTLPPSRX₃EX₄TKNQVSLTCLVKGFYPSDIAVEWESNGQPENNYKT
TPPVLDSDGSFFLYSKLTVDKSRWQQGNVFSCSVMHEALHNHYTQKSLSL
SPG

In some embodiments, the Fc domain monomer includes the amino acid sequence of SEQ ID NO: 10 where X₁ is Ser. In some embodiments, the Fc domain monomer includes the amino acid sequence of SEQ ID NO: 10 where X₁ is Gly. In some embodiments, the Fc domain monomer includes the amino acid sequence of SEQ ID NO: 10 where X₁ is Ala. In some embodiments, the Fc domain monomer includes the amino acid sequence of SEQ ID NO: 10 where X₁ is Thr. In some embodiments, the Fc domain monomer includes the amino acid sequence of SEQ ID NO: 10 where X₁ is Asn. In some embodiments, the Fc domain monomer includes the amino acid sequence of SEQ ID NO: 10 where X₁ is Gln. In some embodiments, the Fc domain monomer includes the amino acid sequence of SEQ ID NO: 10 where X₁ is Arg. In some embodiments, the Fc domain monomer includes the amino acid sequence of SEQ ID NO: 10 where X₁ is Glu. In some embodiments, the Fc domain monomer includes the amino acid sequence of SEQ ID NO: 10 where X₁ is Asp.

In some embodiments, the Fc domain monomer includes the amino acid sequence of SEQ ID NO: 10 where X₃ is Asp and X₄ is Leu (corresponding to Fc allotype G1m(fa)). In some embodiments, the Fc domain monomer includes the amino acid sequence of SEQ ID NO: 10 where X₃ is Glu and X₄ is Met (corresponding to Fc allotype G1m(f)).

SEQ ID NO: 11: mature human IgG1 Fc; Cys to Ser substitution (#); Lys to Ser substitution(*); YTE triple mutation (bold and underlined); X₁ is Asp or Glu; and X₂ is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLYITREPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRX₁EX₂TKNQVSLTCLVKGFYPSDIAVEWESNGQPENNY
KTTPPVLDSDGSFFLYSKLTVDKSRWQQGNVFSCSVMHEALHNHYTQKSL
SLSPG

SEQ ID NO: 12: mature human IgG1 Fc; Cys to Ser substitution (#); Lys to Ser substitution(*); YTE triple mutation (bold and underlined); allotype G1m(fa) (bold italics); N-terminal Fab residues are underlined; hinge residues are italicized

NVNHKPSNTKVDKKVEPKSS(#)DKTHTCPPCPAPELLGGPSVFLFPPS
(*)PKDTLYITREPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSL
SPG

SEQ ID NO: 13: mature human IgG1 Fc; Cys to Ser substitution (#); Lys to Ser substitution(*); YTE triple mutation (bold and underlined); allotype G1m(t) (bold Italics); N-terminal Fab residues are underlined; hinge residues are italicized

NVNHKPSNTKVDKKVEPKSS(#)DKTHTCPPCPAPELLGGPSVFLFPPS
(*)PKDTLYITREPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPG

SEQ ID NO: 14: mature human IgG1 Fc; Cys to Ser substitution (#); Asn to Ala substitution (\hat{ }); YTE triple mutation (bold and underlined); X₁ is Ser, Gly, Ala, Thr, Asn, Gln, Arg, His, Glu, or Asp; Xz is Asp or Glu; and X₃ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLYITREPEVTCVVVDVSHEDPEVKFNWYVDGVEVHNAKTKPREEQY
A(^{∧})STYRVVSVLTVLHQDWLNGKEYKCKVSNKALPAPIEKTISKAKGQP
REPQVYTLPPSRX₂EX₃TKNQVSLTCLVKGFYPSDIAVEWESNGQPENNY
KTTPPVLDSDGSFFLYSKLTVDKSRWQQGNVFSCSVMHEALHNHYTQKSL
SLSPG

SEQ ID NO: 15: mature human IgG1 Fc; Cys to Ser substitution (#); Lys to Ser substitution(*); Asn to Ala substitution (\hat{ }); YTE triple mutation (bold and underlined); X₁ is Asp or Glu; and X₂ is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLYITREPEVTCVVVDVSHEDPEVKFNWYVDGVEVHNAKTKPRE
EQYA(^{∧})STYRVVSVLTVLHQDWLNGKEYKCKVSNKALPAPIEKTISKAK
GQPREPQVYTLPPSRX₁EX₂TKNQVSLTCLVKGFYPSDIAVEWESNGQPE
NNYKTTPPVLDSDGSFFLYSKLTVDKSRWQQGNVFSCSVMHEALHNHYTQ
KSLSLSPG

SEQ ID NO: 16: mature human IgG1 Fc; Cys to Ser substitution (#); DHS triple mutation (bold and underlined); X₁ is Asn or absent; Xx is Ser, Gly, Ala, Thr, Asn, Gln, Arg, His, Glu, or Asp; X₃ is Asn or Ala; X₄ is Asp or Glu; and X₅ is Leu or Met; X₅ is Lys or absent; N-terminal Fab residues are underlined; hinge residues are italicized

X₁VNHKPSNTKVDKKVEPKSS(#)DKTHTCPPCPAPELLGGPSVFLFPP
X₂PKDTLMISRTPEVTCVVVDVSHEDPEVKFNWYVDGVEVHNAKTKPREE
QYX₃STYRVVSVLTVDHHDWLNGKEYKCKVSNKALPAPIEKTISKAKGQP
REPQVYTLPPSRX₄EX₅TKNQVSLTCLVKGFYPSDIAVEWESNGQPENNY
KTTPPVLDSDGSFFLYSKLTVDKSRWQQGNVFSCSVMHEALHSHYTQKSL
SLSPGX₆

In some embodiments, the Fc domain monomer includes the amino acid sequence of SEQ ID NO: 16 where X₂ is Ser. In some embodiments, the Fc domain monomer includes the amino acid sequence of SEQ ID NO: 16 where X₂ is Gly. In some embodiments, the Fc domain monomer includes the amino acid sequence of SEQ ID NO: 16 where X₂ is Ala. In some embodiments, the Fc domain monomer includes the amino acid sequence of SEQ ID NO: 16 where X₂ is Thr. In some embodiments, the Fc domain monomer includes the amino acid sequence of SEQ ID NO: 16 where X₂ is Asn. In some embodiments, the Fc domain monomer includes the amino acid sequence of SEQ ID NO: 16 where X₂ is Gln. In some embodiments, the Fc domain monomer includes the amino acid sequence of SEQ ID NO: 16 where X₂ is Arg. In some embodiments, the Fc domain monomer includes the amino acid sequence of SEQ ID NO: 16 where X₂ is Glu. In some embodiments, the Fc domain monomer includes the amino acid sequence of SEQ ID NO: 1 where X₂ is Asp.

In some embodiments, the Fc domain monomer includes the amino acid sequence of SEQ ID NO: 16 where X₄ is Asp and X₅ is Leu (corresponding to Fc allotype G1m(fa)). In some embodiments, the Fc domain monomer includes the amino acid sequence of SEQ ID NO: 16 where X₄ is Glu and X₅ is Met (corresponding to Fc allotype G1m(f)).

SEQ ID NO: 17: mature human IgG1 Fc; Cys to Ser substitution (#); DHS triple mutation (bold and underlined); X₁ is Ser, Gly, Ala, Thr, Asn, Gln, Arg, His, Glu, or Asp; X₂ is Asn or Ala; X₃ is Asp or Glu; and X₄ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLMISRTPEVTCVVVDVSHEDPEVKFNWYVDGVEVHNAKTKPREEQY
X₂STYRVVSVLTVDHHDWLNGKEYKCKVSNKALPAPIEKTISKAKGQPRE
PQVYTLPPSRX₃EX₄TKNQVSLTCLVKGFYPSDIAVEWESNGQPENNYKT
TPPVLDSDGSFFLYSKLTVDKSRWQQGNVFSCSVMHEALHSHYTQKSLSL
SPG

In some embodiments, the Fc domain monomer includes the amino acid sequence of SEQ ID NO: 17 where X₁ is Ser. In some embodiments, the Fc domain monomer includes the amino acid sequence of SEQ ID NO: 17 where X₁ is Gly. In some embodiments, the Fc domain monomer includes the amino acid sequence of SEQ ID NO: 17 where X₁ is Ala. In some embodiments, the Fc domain monomer includes the amino acid sequence of SEQ ID NO: 17 where X₁ is Thr. In some embodiments, the Fc domain monomer includes the amino acid sequence of SEQ ID NO: 17 where X₁ is Asn. In some embodiments, the Fc domain monomer includes the amino acid sequence of SEQ ID NO: 17 where X₁ is Gln. In some embodiments, the Fc domain monomer includes the amino acid sequence of SEQ ID NO: 17 where X₁ is Arg. In some embodiments, the Fc domain monomer includes the amino acid sequence of SEQ ID NO: 17 where X₁ is Glu. In some embodiments, the Fc domain monomer includes the amino acid sequence of SEQ ID NO: 17 where X₁ is Asp.

In some embodiments, the Fc domain monomer includes the amino acid sequence of SEQ ID NO: 17 where X₃ is Asp and X₄ is Leu (corresponding to Fc allotype G1m(fa)). In some embodiments, the Fc domain monomer includes the amino acid sequence of SEQ ID NO: 17 where X₃ is Glu and X₄ is Met (corresponding to Fc allotype G1m(f)).

SEQ ID NO: 18: mature human IgG1 Fc; Cys to Ser substitution (#); Lys to Ser substitution(*); DHS triple mutation (bold and underlined); X₁ is Asp or Glu; and Xz is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVDHHDWLNGKEYKCKVSNKALPAPIEKTISKAKGQP
REPQVYTLPPSRX₁EX₂TKNQVSLTCLVKGFYPSDIAVEWESNGQPENNY
KTTPPVLDSDGSFFLYSKLTVDKSRWQQGNVFSCSVMHEALHSHYTQKSL
SLSPG

SEQ ID NO: 19: mature human IgG1 Fc; Cys to Ser substitution (#); Lys to Ser substitution(*); DHS triple mutation (bold and underlined); allotype G1m(fa) (bold italics); N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVDHHDWLNGKEYKCKVSNKALPAPIEKTISKAKGQP
REPQVYTLPPSRDELTKNQVSLTCLVKGFYPSDIAVEWESNGQPENNYKT
TPPVLDSDGSFFLYSKLTVDKSRWQQGNVFSCSVMHEALHSHYTQKSLSL
SPG

SEQ ID NO: 20: mature human IgG1 Fc; Cys to Ser substitution (#); Lys to Ser substitution(*); DHS triple mutation (bold and underlined); allotype G1m(t) (bold italics); N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVDHHDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHSHYTQKSLSL
SPG

SEQ ID NO: 21: mature human IgG1 Fc; Cys to Ser substitution (#); Asn to Ala substitution (\hat{ }); DHS triple mutation (bold and underlined); X₁ is Ser, Gly, Ala, Thr, Asn, Gln, Arg, His, Glu, or Asp; X₂ is Asp or Glu; and X₃ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLMISRTPEVTCVVVDVSHEDPEVKFNWYVDGVEVHNAKTKPREEQY
A(^{∧})STYRVVSVLTVDHHDWLNGKEYKCKVSNKALPAPIEKTISKAKGQP
REPQVYTLPPSRX₂EX₃TKNQVSLTCLVKGFYPSDIAVEWESNGQPENNY
KTTPPVLDSDGSFFLYSKLTVDKSRWQQGNVFSCSVMHEALHSHYTQKSL
SLSPG

SEQ ID NO: 22: mature human IgG1 Fc; Cys to Ser substitution (#); Lys to Ser substitution(*); Asn to Ala substitution (\hat{ }); DHS triple mutation (bold and underlined); X₁ is Asp or Glu; and X₂ is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYA(^{∧})STYRVVSVLTVDHHDWLNGKEYKCKVSNKALPAPIEKTISKAK
GQPREPQVYTLPPSRX₁EX₂TKNQVSLTCLVKGFYPSDIAVEWESNGQPE
NNYKTTPPVLDSDGSFFLYSKLTVDKSRWQQGNVFSCSVMHEALHSHYTQ
KSLSLSPG

SEQ ID NO: 23: mature human IgG1 Fc; Cys to Ser substitution (#); LS double mutation (bold and underlined); X₁ is Asn or absent; X₂ is Ser, Gly, Ala, Thr, Asn, Gln, Arg, His, Glu, or Asp; X₃ is Asn or Ala; X₄ is Asp or Glu; and X₅ is Leu or Met; X₆ is Lys or absent; N-terminal Fab residues are underlined; hinge residues are italicized

X₁VNHKPSNTKVDKKVEPKSS(#)DKTHTCPPCPAPELLGGPSVFLFPP
X₂PKDTLMISRTPEVTCVVVDVSHEDPEVKFNWYVDGVEVHNAKTKPREE
QYX₃STYRVVSVLTVLHQDWLNGKEYKCKVSNKALPAPIEKTISKAKGQP
REPQVYTLPPSRX₄EX₅TKNQVSLTCLVKGFYPSDIAVEWESNGQPENNY
KTTPPVLDSDGSFFLYSKLTVDKSRWQQGNVFSCSVLHEALHSHYTQKSL
SLSPGX₆

In some embodiments, the Fc domain monomer includes the amino acid sequence of SEQ ID NO: 23 where X₂ is Ser. In some embodiments, the Fc domain monomer includes the amino acid sequence of SEQ ID NO: 23 where X₂ is Gly. In some embodiments, the Fc domain monomer includes the amino acid sequence of SEQ ID NO: 23 where X₂ is Ala. In some embodiments, the Fc domain monomer includes the amino acid sequence of SEQ ID NO: 23 where X₂ is Thr. In some embodiments, the Fc domain monomer includes the amino acid sequence of SEQ ID NO: 23 where X₂ is Asn. In some embodiments, the Fc domain monomer includes the amino acid sequence of SEQ ID NO: 23 where X₂ is Gln. In some embodiments, the Fc domain monomer includes the amino acid sequence of SEQ ID NO: 23 where X₂ is Arg. In some embodiments, the Fc domain monomer includes the amino acid sequence of SEQ ID NO: 23 where X₂ is Glu. In some embodiments, the Fc domain monomer includes the amino acid sequence of SEQ ID NO: 23 where X₂ is Asp.

In some embodiments, the Fc domain monomer includes the amino acid sequence of SEQ ID NO: 23 where X₄ is Asp and X₅ is Leu (corresponding to Fc allotype G1m(fa)). In some embodiments, the Fc domain monomer includes the amino acid sequence of SEQ ID NO: 23 where X₄ is Glu and X₅ is Met (corresponding to Fc allotype G1m(f)).

SEQ ID NO: 24: mature human IgG1 Fc; Cys to Ser substitution (#); LS double mutation (bold and underlined); X₁ is Ser, Gly, Ala, Thr, Asn, Gln, Arg, His, Glu, or Asp; X₂ is Asn or Ala; X₃ is Asp or Glu; and X₄ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLMISRTPEVTCVVVDVSHEDPEVKFNWYVDGVEVHNAKTKPREEQY
X₂STYRVVSVLTVLHQDWLNGKEYKCKVSNKALPAPIEKTISKAKGQPRE
PQVYTLPPSRX₃EX₄TKNQVSLTCLVKGFYPSDIAVEWESNGQPENNYKT
TPPVLDSDGSFFLYSKLTVDKSRWQQGNVFSCSVLHEALHSHYTQKSLSL
SPG

In some embodiments, the Fc domain monomer includes the amino acid sequence of SEQ ID NO: 24 where X₁ is Ser. In some embodiments, the Fc domain monomer includes the amino acid sequence of SEQ ID NO: 24 where X₁ is Gly. In some embodiments, the Fc domain monomer includes the amino acid sequence of SEQ ID NO: 24 where X₁ is Ala. In some embodiments, the Fc domain monomer includes the amino acid sequence of SEQ ID NO: 24 where X₁ is Thr. In some embodiments, the Fc domain monomer includes the amino acid sequence of SEQ ID NO: 24 where X₁ is Asn. In some embodiments, the Fc domain monomer includes the amino acid sequence of SEQ ID NO: 24 where X₁ is Gln. In some embodiments, the Fc domain monomer includes the amino acid sequence of SEQ ID NO: 24 where X₁ is Arg. In some embodiments, the Fc domain monomer includes the amino acid sequence of SEQ ID NO: 24 where X₁ is Glu. In some embodiments, the Fc domain monomer includes the amino acid sequence of SEQ ID NO: 24 where X₁ is Asp.

In some embodiments, the Fc domain monomer includes the amino acid sequence of SEQ ID NO: 24 where X₃ is Asp and X₄ is Leu (corresponding to Fc allotype G1 m(fa)). In some embodiments, the Fc domain monomer includes the amino acid sequence of SEQ ID NO: 24 where X₃ is Glu and X₄ is Met (corresponding to Fc allotype G1m(o)).

SEQ ID NO: 25: mature human IgG1 Fc; Cys to Ser substitution (#); Lys to Ser substitution(*); LS double mutation (bold and underlined); X₁ is Asp or Glu; and X₂ is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRX₁EX₂TKNQVSLTCLVKGFYPSDIAVEWESNGQPENNY
KTTPPVLDSDGSFFLYSKLTVDKSRWQQGNVFSCSVLHEALHSHYTQKSL
SLSPG

SEQ ID NO: 26: mature human IgG1 Fc; Cys to Ser substitution (#); Lys to Ser substitution(*); LS double mutation (bold and underlined); allotype G1 m(fa) (bold italics); N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKT
TPPVLDSDGSFFLYSKLTVDKSRWQQGNVFSCSVLHEALHSHYTQKSLSL
SPG

SEQ ID NO: 27: mature human IgG1 Fc; Cys to Ser substitution (#); Lys to Ser substitution(*); LS double mutation (bold and underlined); allotype G1m(t) (bold italics); N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVLHEALHSHYTQKSLSL
SPG

SEQ ID NO: 28: mature human IgG1 Fc; Cys to Ser substitution (#); Asn to Ala substitution (\hat{ }); LS double mutation (bold and underlined); X₁ is Ser, Gly, Ala, Thr, Asn, Gln, Arg, His, Glu, or Asp; X₂ is Asp or Glu; and X₃ is Leu or Met; N-terminal Fab residues are underlined; hinge residues are italicized

NVNHKPSNTKVDKKVEPKSS(#)DKTHTCPPCPAPELLGGPSVFLFPPX₁
PKDTLMISRTPEVTCVVVDVSHEDPEVKFNWYVDGVEVHNAKTKPREEQY
A(^{∧})STYRVVSVLTVLHQDWLNGKEYKCKVSNKALPAPIEKTISKAKGQP
REPQVYTLPPSRX₂EX₃TKNQVSLTCLVKGFYPSDIAVEWESNGQPENNY
KTTPPVLDSDGSFFLYSKLTVDKSRWQQGNVFSCSVLHEALHSHYTQKSL
SLSPG

SEQ ID NO: 29: mature human IgG1 Fc; Cys to Ser substitution (#); Lys to Ser substitution(*); Asn to Ala substitution (\hat{ }); LS double mutation (bold and underlined); X₁ is Asp or Glu; and X₂ is Leu or Met; N-terminal Fab residues are underlined; hinge residues are italicized

NVNHKPSNTKVDKKVEPKSS(#)DKTHTCPPCPAPELLGGPSVFLFPPS
(*)PKDTLMISRTPEVTCVVVDVSHEDPEVKFNWYVDGVEVHNAKTKPRE
EQYA(^{∧})STYRVVSVLTVLHQDWLNGKEYKCKVSNKALPAPIEKTISKAK
GQPREPQVYTLPPSRX₁EX₂TKNQVSLTCLVKGFYPSDIAVEWESNGQPE
NNYKTTPPVLDSDGSFFLYSKLTVDKSRWQQGNVFSCSVLHEALHSHYTQ
KSLSLSPG

As defined herein, an Fc domain includes two Fc domain monomers that are dimerized by the interaction between the C_{H}3 antibody constant domains, as well as one or more disulfide bonds that form between the hinge domains of the two dimerizing Fc domain monomers. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present disclosure binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the Fc domain monomer or Fc domain of the disclosure is an aglycosylated Fc domain monomer or Fc domain (e.g., an Fc domain monomer or and Fc domain that maintains engagement to an Fc receptor (e.g., FcRn). For example, the Fc domain is an aglycosylated IgG1 variants that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105(51):20167-20172, which is incorporated herein in its entirety.

In some embodiments, the Fc domain or Fc domain monomer of the disclosure is engineered to enhance binding to the neonatal Fc receptor (FcRn). For example, the Fc domain may include the triple mutation corresponding to M252Y/S254T/T256E (YTE) (e.g., an IgG1, such as a human or humanized IgG1 having a YTE mutation). The Fc domain may include the double mutant corresponding to M428L/N434S (LS) (e.g., an IgG1, such as a human or humanized IgG1 having an LS mutation). The Fc domain may include the single mutant corresponding to N434H (e.g., an IgG1, such as a human or humanized IgG1 having an N434H mutation). The Fc domain may include the single mutant corresponding to C220S (e.g., and IgG1, such as a human or humanized IgG1 having a C220S mutation). The Fc domain may include a quadruple mutant corresponding to C220S/L309D/Q311H/N434S (CDHS) (e.g., an IgG1, such as a human or humanized IgG1 having a CDHS mutation). The Fc domain may include a triple mutant corresponding to L309D/Q211H/N434S (DHS) (e.g., an IgG1, such as a human or humanized IgG1 having a DHS mutation).

The Fc domain may include a combination of one or more of the above-described mutations that enhance binding to the FcRn. Enhanced binding to the FcRn may increase the half-life Fc domain-containing conjugate. For example, incorporation of one or more amino acid mutations that increase binding to the FcRn (e.g., a YTE mutation, an LS mutation, or an N434H mutation) may increase the half life of the conjugate by 5%, 10%, 15%, 20%, 30%, 40%, 50%, 60%, 70%, 80%, 90%, 100%, 200%, 300%, 400%, 500% or more relative to a conjugate having an the corresponding Fc domain without the mutation that enhances FcRn binding. Exemplary Fc domains with enhanced binding to the FcRN and methods for making Fc domains having enhanced binding to the FcRN are known in the art, for example, as described in Maeda, A. et al., Identification of human IgG1 variant with enhanced FcRn binding and without increased binding to rheumatoid factor autoantibody, MABS, 2017, 9(5):844-853, which is incorporated herein in its entirety. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-29 may be mutated to include a YTE mutation, an LS mutation, and/or an N434H mutation by mutating the “corresponding residues” of the amino acid sequence. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-29 may be mutated to include a YTE mutation, an LS mutation, and/or an N434H mutation by mutating the “corresponding residues” of the amino acid sequence.

As used herein, a sulfur atom “corresponding to” a particular cysteine residue of a particular SEQ ID NO. should be understood to include the sulfur atom of any cysteine residue that one of skill in the art would understand to align to the particular cysteine of the particular sequence.

As used herein, a nitrogen atom “corresponding to” a particular lysine residue of a particular SEQ ID NO. should be understood to include the nitrogen atom of any lysine residue that one of skill in the art would understand to align to the particular lysine of the particular sequence.

### Activation of Immune Cells

Fc-gamma receptors (FcγRs) bind the Fc portion of immunoglobulin G (IgG) and play important roles in immune activation and regulation. For example, the IgG Fc domains in immune complexes (ICs) engage FcγRs with high avidity, thus triggering signaling cascades that regulate immune cell activation. The human FcγR family contains several activating receptors (FcγRI, FcγRIIa, FcγRIIc, FcγRIIIa, and FcγRIIIb) and one inhibitory receptor (FcγRIIb). FcγR signaling is mediated by intracellular domains that contain immune tyrosine activating motifs (ITAMs) for activating FcγRs and immune tyrosine inhibitory motifs (ITIM) for inhibitory receptor FcγRIIb. In some embodiments, FcγR binding by Fc domains results in ITAM phosphorylation by Src family kinases; this activates Syk family kinases and induces downstream signaling networks, which include PI3K and Ras pathways.

In the conjugates described herein, the portion of the conjugates including dimers of neuraminidase inhibitors bind to and inhibits viral neuraminidase leading to inhibition of viral replication, while the Fc domain portion of the conjugates bind to FcγRs (e.g., FcRn, FcγRI, FcγRIIa, FcγRIIc, FcγRIIIa, and FcγRIIIb) on immune cells and activate phagocytosis and effector functions, such as antibody-dependent cell-mediated cytotoxicity (ADCC), thus leading to the engulfment and destruction of viral particles by immune cells and further enhancing the antiviral activity of the conjugates. Examples of immune cells that may be activated by the conjugates described herein include, but are not limited to, macrophages, neutrophils, eosinophils, basophils, lymphocytes, follicular dendritic cells, natural killer cells, and mast cells.

### Half-Life

Biological half-life (t_{1/2}) is the time it takes a therapeutic to decrease its maximum concentration by half. Improvements in half-life for therapeutics can lower the efficacious dose. There are many variables that affect half-life from patient variables (e.g., age, blood circulation, diet, excessive fluids, low fluids, gender, history of drug use, kidney function, liver function, obesity, pre-existing conditions etc.) to therapeutic specific variables (e.g., therapeutic formulation, pharmacokinetics, administration method, drug clearance (e.g., kidney, liver, or lungs), tissue distribution and accumulation, therapeutic size, charge, pKa, etc.). For peptide therapeutics short plasma half-lives are commonly due to fast renal clearance as well as to enzymatic degradation occurring during systemic circulation. Modifications of the peptide or protein can lead to prolonged plasma half-life times. In some instances, the Fc domain or fusion protein are engineered to increase the half-life of the Fc domain monomer, conjugate, or fusion protein. In some embodiments, the Fc domain or Fc domain monomer of the disclosure is engineered to enhance binding to the neonatal Fc receptor (FcRn). Enhanced binding to the FcRn may increase the half-life Fc domain-containing conjugate or fusion protein, for example, the Fc domain monomer or Fc domain may increase the half-life of the conjugate by 5%, 10%, 15%, 20%, 30%, 40%, 50%, 60%, 70%, 80%, 90%, 100%, 200%, 300%, 400%, 500% or more relative to a conjugate having the corresponding Fc domain without a mutation, e.g., the K246X mutation, the K246X/M252Y/S254T/T256E mutations, the K246X/V309D/Q311H/N434S mutations, the K246X/M428L/N434S mutations, the C220S/K246X/M252Y/S254T/T256E mutations, the C220S/K246X/V309D/Q311H/N434S mutations, the C220S/K246X/M428L/N434S mutations, or further mutations that enhances FcRn binding. In some instances, the Fc domain monomer is engineered to include at least 220 residues.

### Renal Clearance

Many therapeutic peptides have short half-lives (minutes) in vivo due to their size. The rapid clearance and short half-life of peptides limit their development into successful drugs. One of the main causes of rapid clearance of peptides from systemic circulation is renal clearance. The glomeruli have a pore size of approximately 8 nm, and hydrophilic peptides with MW<2-25 kDa are susceptible to rapid filtration through the glomeruli of the kidney. In some embodiments, the Fc domain monomers and fusion proteins described herein are greater than 20 kDa. In some embodiments, the Fc domain monomers and fusion proteins of two conjugates or fusion proteins may dimerize to form a Fc domain. In some embodiments, the Fc domain monomer, the conjugate, or the fusion protein are engineered to decrease renal clearance. Decreased renal clearance may increase the half-life of the Fc domain monomer of a conjugate or fusion protein described herein, for example, the Fc domain may include at least about 200 amino acids (e.g., at least 200, at least 225, at least about 230, at least about 240, at least about 242, at least about 243, at least about 250, at least about 255, at least about 260, at least about 265, at least about 270, at least about 275, at least about 280, at least about 285, at least about 290, at least about 295, or at least about 300 amino acids).

### Tissue Distribution

After a therapeutic enters the systemic circulation, it is distributed to the body's tissues. Distribution is generally uneven because of different in blood perfusion, tissue binding, regional pH, and permeability of cell membranes. The entry rate of a drug into a tissue depends on the rate of blood flow to the tissue, tissue mass, and partition characteristics between blood and tissue. Distribution equilibrium (when the entry and exit rates are the same) between blood and tissue is reached more rapidly in richly vascularized areas, unless diffusion across cell membranes is the rate-limiting step. The size, shape, charge, target binding, FcRn and target binding mechanisms, route of administration, and formulation affect tissue distribution.

In some instances, the conjugates described herein may be optimized to distribute to lung tissue. In some instances, the conjugates have a concentration ratio of distribution in epithelial lining fluid of at least 30% the concentration of the conjugates in plasma within 2 hours after administration. In certain embodiments, ratio of the concentration is at least 45% within 2 hours after administration. In some embodiments, the ratio of concentration is at least 55% within 2 hours after administration. In particular, the ratio of concentration is at least 60% within 2 hours after administration. As shown in Example 190 WO 2021/046549, by 2 hours post injection, a conjugate having an Fc domain including a M252Y/S254T/T256E (YTE) mutation, ELF levels are surprisingly ˜60% of plasma exposure levels as measured by AUC across the rest of the time course indicating nearly immediate partitioning of the conjugate from plasma to the ELF in the lung. This demonstrates that an Fc containing conjugate rapidly distributes to lung, and maintains high concentrations in lung relative to levels in plasma.

### Boundaries of Fc Domain Monomer

The length (e.g., as determined by the N-terminal and C-terminal boundaries) of the Fc domain monomer may be optimized in order to prevent renal clearance and increase distribution to a desired tissue (e.g., lung tissue). Antibodies are divided into two domains: the Fc (effector) domain and the fragment antigen-binding (Fab) domain, the latter of which contains the antigen-binding regions. The present disclosure provides Fc domain monomers which include a portion of the Fab domain at the N-terminus of the Fc domain. Smaller Fc constructs (e.g., Fc constructs lacking a portion of the Fab domain) demonstrated a decreased half-life, likely due to renal elimination. To address this problem, the Fc constructs were iteratively lengthened by adding back in some of the Fab domain on the N-terminus, until further increases in size did not lead to improvements (e.g., in mouse pharmacokinetic experiments). The present disclosure provides Fc domain monomers which have been optimized (e.g., by length, mass, N-terminal, and/or C-terminal boundaries in addition to mutational variants) to achieve the desired increased half-life and/or tissue distribution.

In some embodiments, the N-terminus of the Fc domain monomer includes between 10 and 20 residues (e.g., 11, 12, 13, 14, 15, 16, 17, 18, or 19 residues) of the Fab domain. In certain embodiments, the N-terminus of the Fc domain monomer is any one of amino acid residues 198-205. In some embodiments, the N-terminus of the Fc domain monomer is amino acid residue 201 (e.g., Asn 201). In certain embodiments, the N-terminus of the Fc domain monomer is amino acid residue 202 (e.g., Val 202). In other embodiments, the C-terminus of the Fc domain monomer is any one of amino acid residues 437-447. In another embodiment, the C-terminus of the Fc domain monomer is amino acid residue 446 (e.g., Gly 446). In some embodiments, the C-terminus of the Fc domain monomer is amino acid residue 447 (e.g. Lys 447).

Lengthening the construct required the addition of a portion of the hinge region that contains a free cysteine residue (C220), which created issues with thiol mediated aggregation. C220 was mutated to a serine (C220S) to avert this problem.

### Therapeutic Agent Delivery

The large size of antibody molecules can make it difficult to transport targeting systems across cellular membranes. In some instances, large targeting systems can lead to slow elimination from the blood circulation, which can ultimately lead to myelotoxicity. In addition, in vivo use of antibody-based targeting systems is expensive and can lead to immunogenicity after repeated injections of such formulations. Antibody fragments which are smaller than whole antibodies have successfully been made but are still, in many instances, too large. Fragments can reach extracellular spaces more easily than whole antibodies. In some instances, the Fc domain monomers can be used in conjugates to deliver a therapeutic agent. In some instances, Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present disclosure binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn). Binding of the neonatal Fc receptor mediates internalization of the Fc domain monomer or conjugate of fusion protein thereof, thereby delivering a therapeutic agent to a cell. Upon internalization, an endocytic salvage pathway that prevents degradation of the Fc domain monomer or conjugate or fusion protein thereof. In some instances, the Fc domain monomer of Fc domain is engineered to increase neonatal Fc receptor binding.

### IV. Linkers

A linker refers to a linkage or connection between two or more components in a conjugate described herein (e.g., between two neuraminidase inhibitors in a conjugate described herein, between a neuraminidase inhibitor and an Fc domain in a conjugate described herein, and/or between a dimer of two neuraminidase inhibitors and an Fc domain in a conjugate described herein).

### Linkers in Conjugates Having an Fc Domain Covalently Linked to Dimers of Neuraminidase Inhibitors

In a conjugate containing an Fc domain monomer or an Fc domain covalently linked to one or more dimers of neuraminidase inhibitors as described herein, a linker in the conjugate (e.g., L or L′) may be a branched structure. As described further herein, a linker in a conjugate described herein (e.g., L or L′) may be a multivalent structure, e.g., a divalent or trivalent structure having two or three arms, respectively. In some embodiments when the linker has three arms, two of the arms may be attached to the first and second neuraminidase inhibitors and the third arm may be attached to the Fc domain monomer or an Fc domain. In some embodiments when the linker has two arms, one arm may be attached to an Fc domain and the other arm may be attached to one of the two neuraminidase inhibitors. In other embodiments, a linker with two arms may be used to attach the two neuraminidase inhibitors on a conjugate containing an Fc domain covalently linked to one or more dimers of neuraminidase inhibitors.

In some embodiments, a linker in a conjugate having an Fc domain covalently linked to one or more dimers of neuraminidase inhibitors is described by formula (D-L-I):

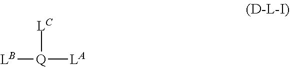

wherein L^{A} is described by formula G^{A1}-(Z^{A1})_{g1}—(Y^{A1})ₕ₁—(Z^{A2})ᵢ₁—(Y^{A2})ⱼ₁—(Z^{A3})ₖ₁—(Y^{A3})ₗ₁—(Z^{A4})ₘ₁—(Y^{A4})ₙ₁—(Z^{A5})o₁-G^{A2}; L^{B} is described by formula G^{B1}-(Z^{B1})_{g2}—(Y^{B1})ₕ₂—(Z^{B2})ᵢ₂—(Y^{B2})ⱼ₂—(Z^{B3})ₖ₂—(Y^{B3})ₗ₂—(Z^{B4})ₘ₂—(Y^{B4})ₙ₂—(Z^{B5})o₂-G^{B2}; L^{C} is described by formula G^{C1}-(Z^{C1})_{g3}—(Y^{C1})ₕ₃—(Z^{C2})ᵢ₃—(Y^{C2})ⱼ₃—(Z^{C3})ₖ₃—(Y^{C3})ₗ₃—(Z^{C4})ₘ₃—(Y^{C4})ₙ₃—(Z^{C5})o₃-G^{C2}; G^{A1} is a bond attached to Q in formula (D-L-I); G^{A2} is a bond attached to the first neuraminidase inhibitor (e.g., A₁); G^{B1} is a bond attached to Q in formula (D-L-I); G^{B2} is a bond attached to the second neuraminidase inhibitor (e.g., A₂); G^{C1} is a bond attached to Q in formula (D-L-I); G^{C2} is a bond attached to an Fc domain monomer, an Fc domain, or a functional group capable of reacting with a functional group conjugated to an Fc domain monomer or an Fc domain (e.g., maleimide and cysteine, amine and activated carboxylic acid, thiol and maleimide, activated sulfonic acid and amine, isocyanate and amine, azide and alkyne, and alkene and tetrazine); each of Z^{A1}, Z^{A2}, Z^{A3}, Z^{A4}, Z^{A5}, Z^{B1}, Z^{B2}, Z^{B3}, Z^{B4}, Z^{B5}, Z^{C1}, Z^{C2}, Z^{C3}, Z^{C4} and Z^{C5} is, independently, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, Y^{B4}, Y^{C1}, Y^{C2}, Y^{C3}, and Y^{C4} is, independently, O, S, NRⁱ, P, carbonyl, thiocarbonyl, sulfonyl, phosphate, phosphoryl, or imino; R₁ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, o2, g3, h3, i3, j3, k3, l3, m3, n3, and o3 is, independently, 0 or 1; Q is a nitrogen atom, optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C5-C20 cycloalkynylene, optionally substituted C8-C20 heterocycloalkynylene, optionally substituted C5-C15 arylene, or optionally substituted C2-C15 heteroarylene.

In some embodiments. L^{C} may have two points of attachment to the Fc domain (e.g., two G^{C2}).

In some embodiments, L includes a polyethylene glycol (PEG) linker. A PEG linker includes a linker having the repeating unit structure (—CH₂CH₂O—)ₙ, where n is an integer from 2 to 100. A polyethylene glycol linker may covalently join a first neuraminidase inhibitor and a second neuraminidase inhibitor (e.g., in a conjugate of any one of formulas (D-I)-(D-VIII)). A polyethylene glycol linker may covalently join a neuraminidase inhibitor dimer and E (e.g., in a conjugate of any one of formulas (D-I)-(D-VIII)). A polyethylene glycol linker may be selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀). In some embodiments, L; includes a PEG linker, where L^{C} is covalently attached to each of Q and E.

Linkers of formula (D-L-I) that may be used in conjugates described herein include, but are not limited to

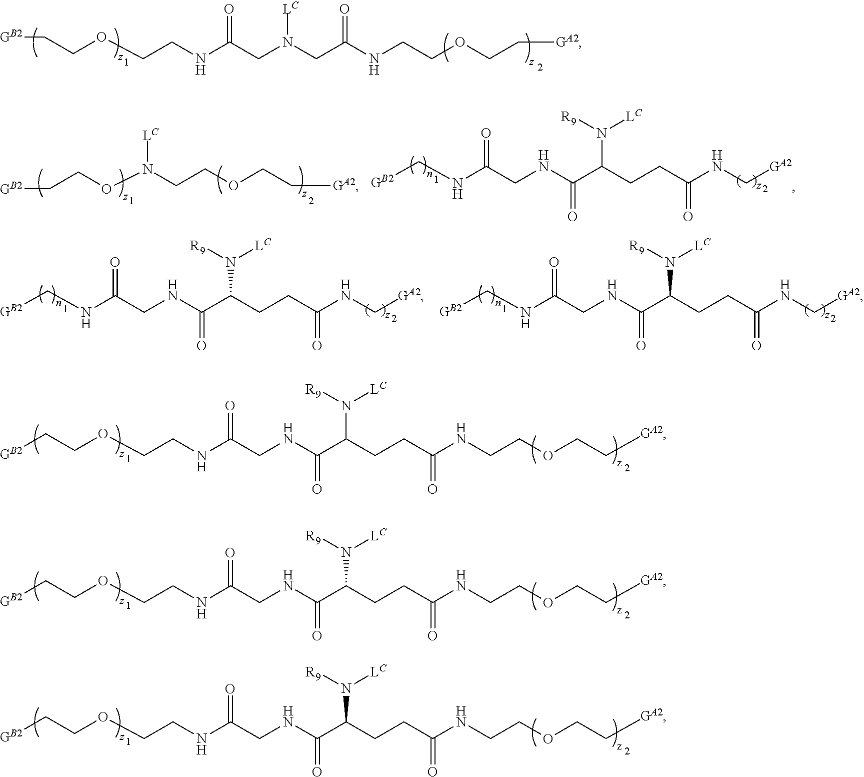

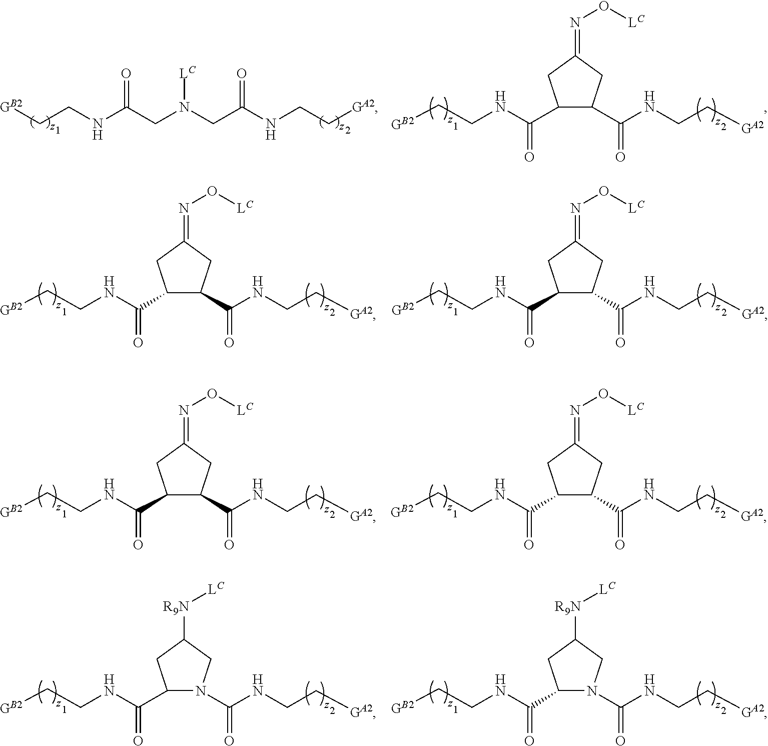

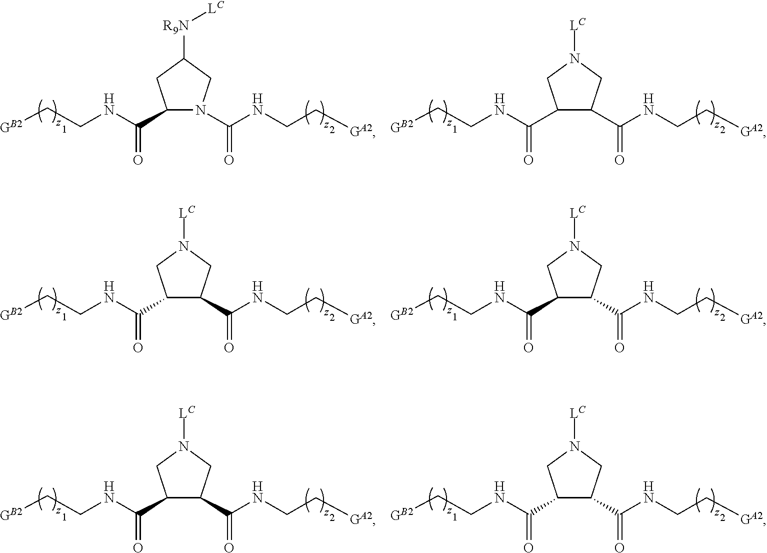

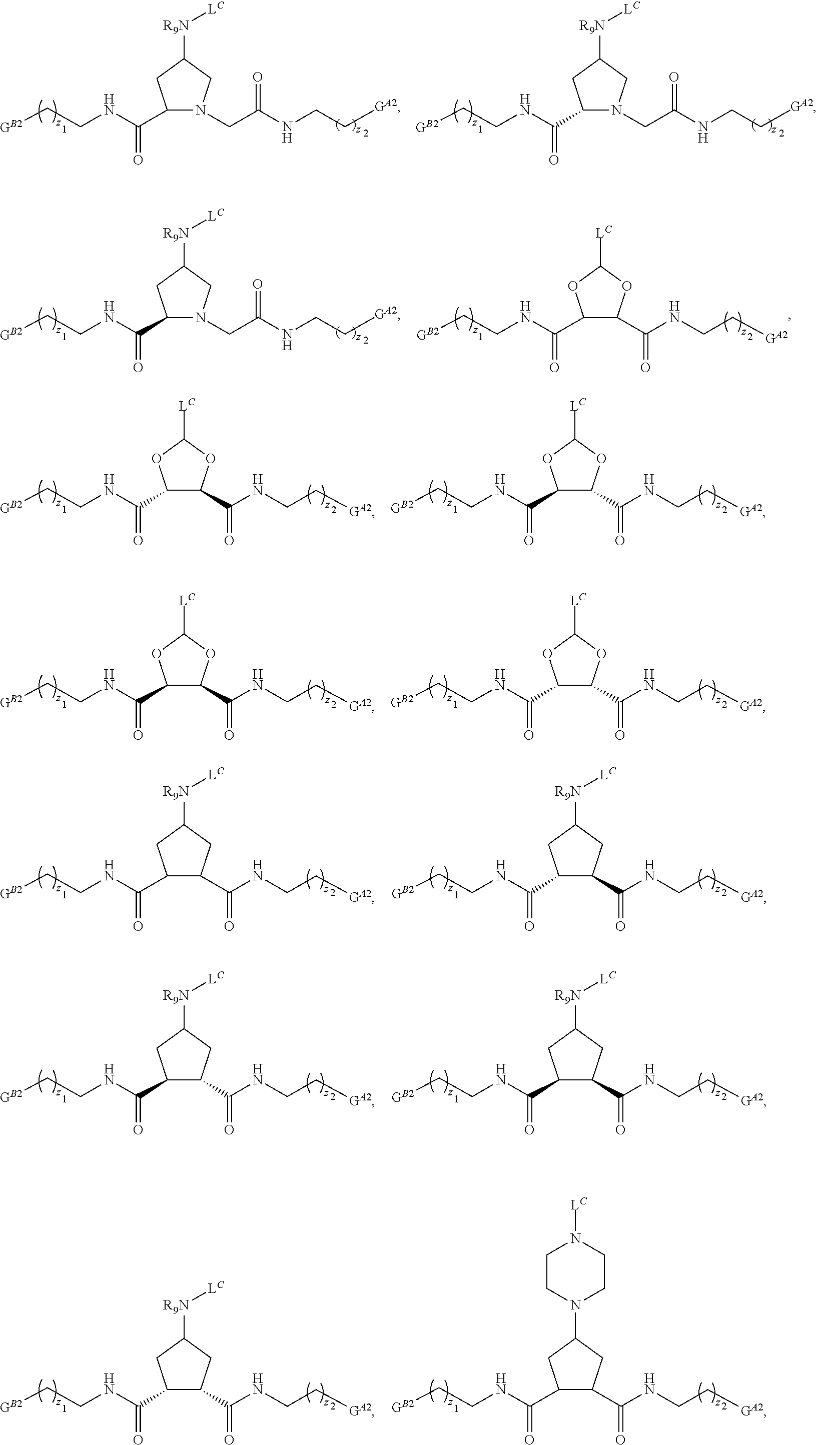

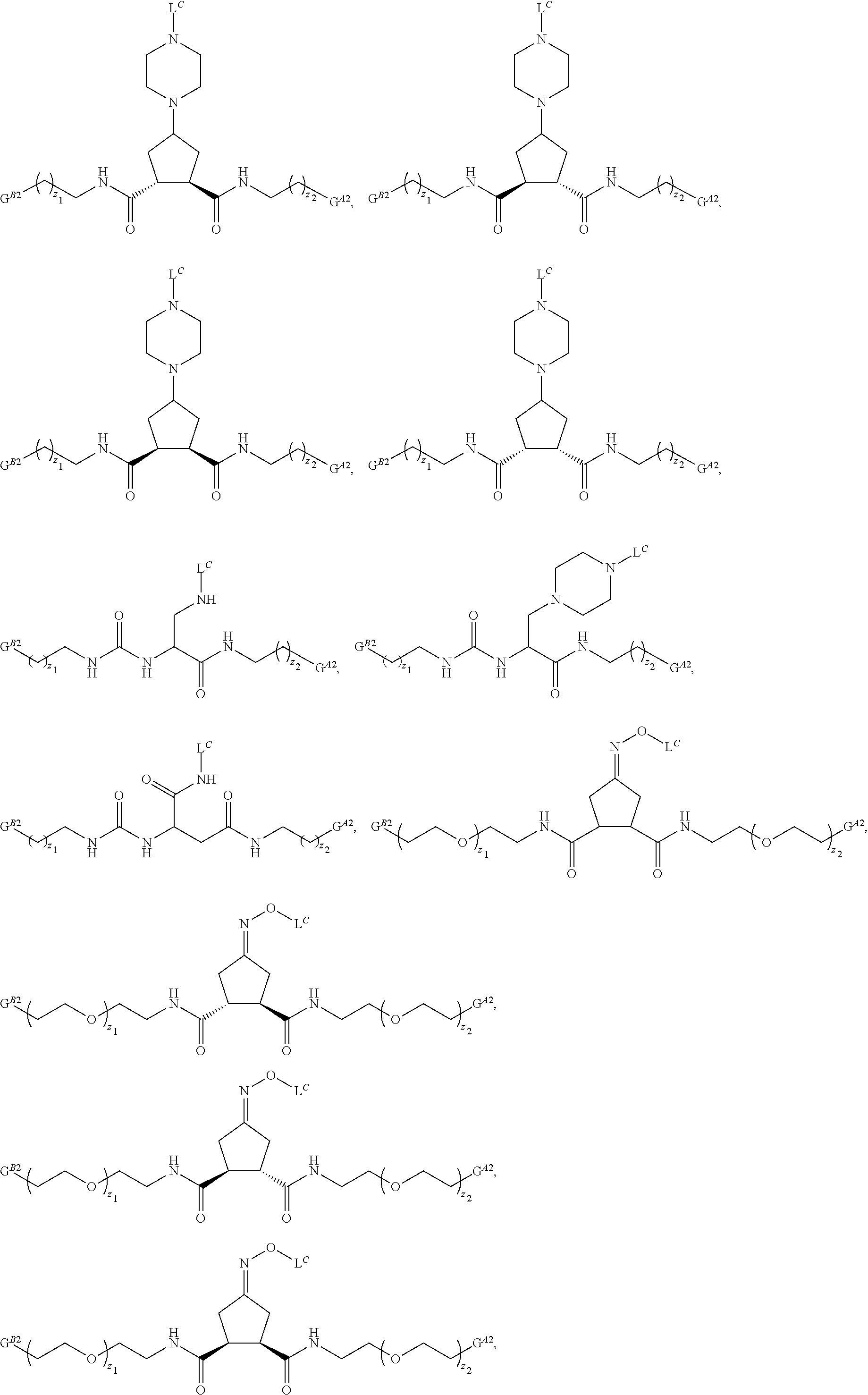

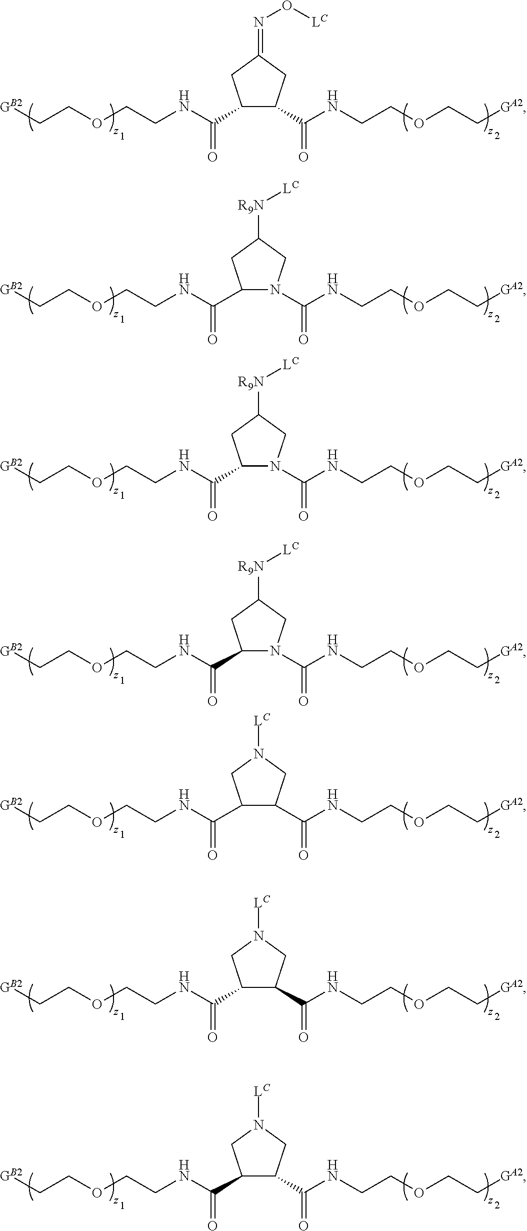

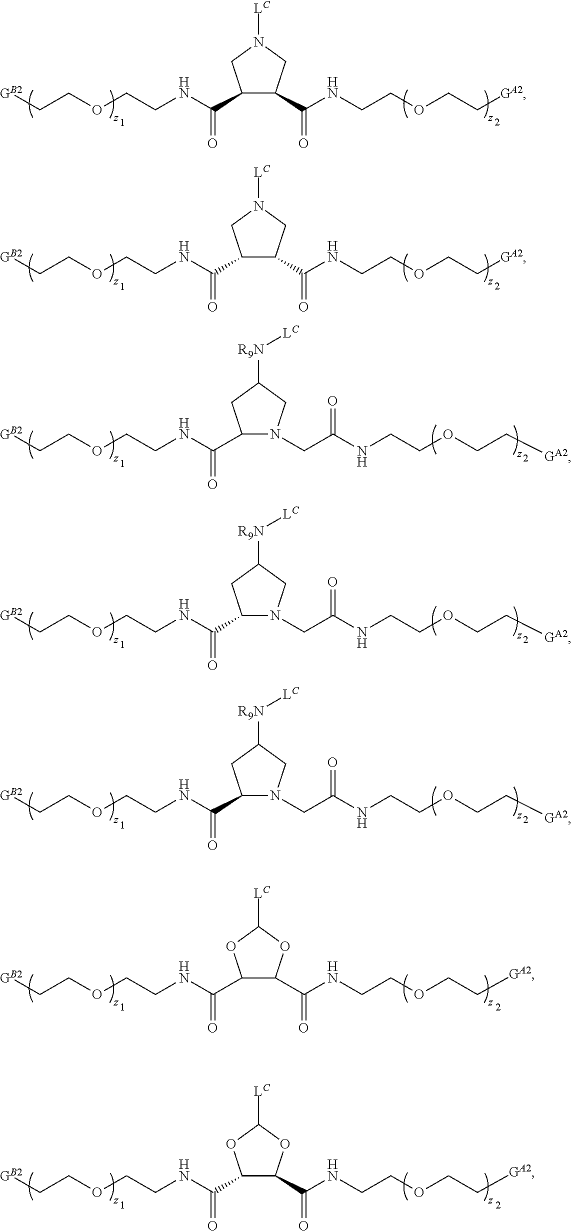

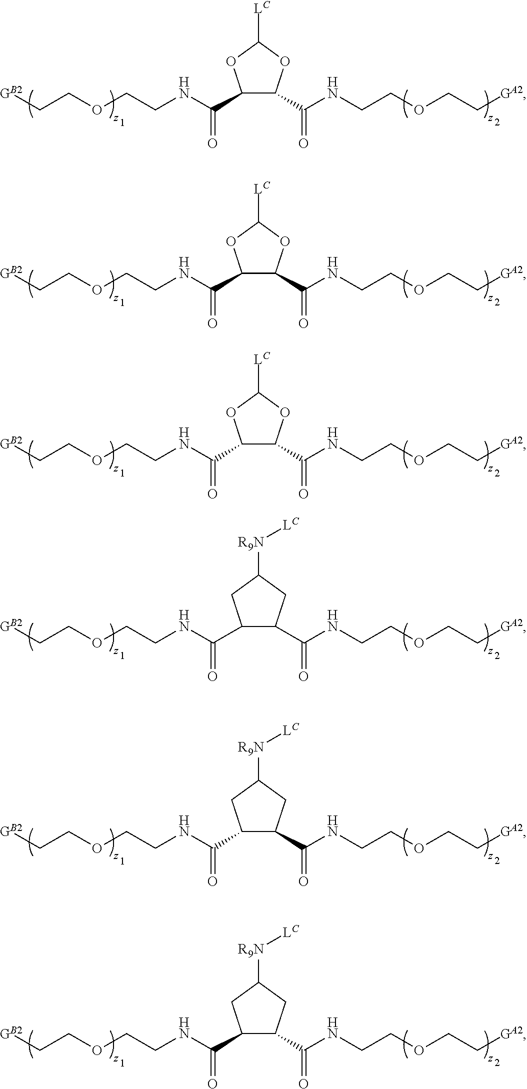

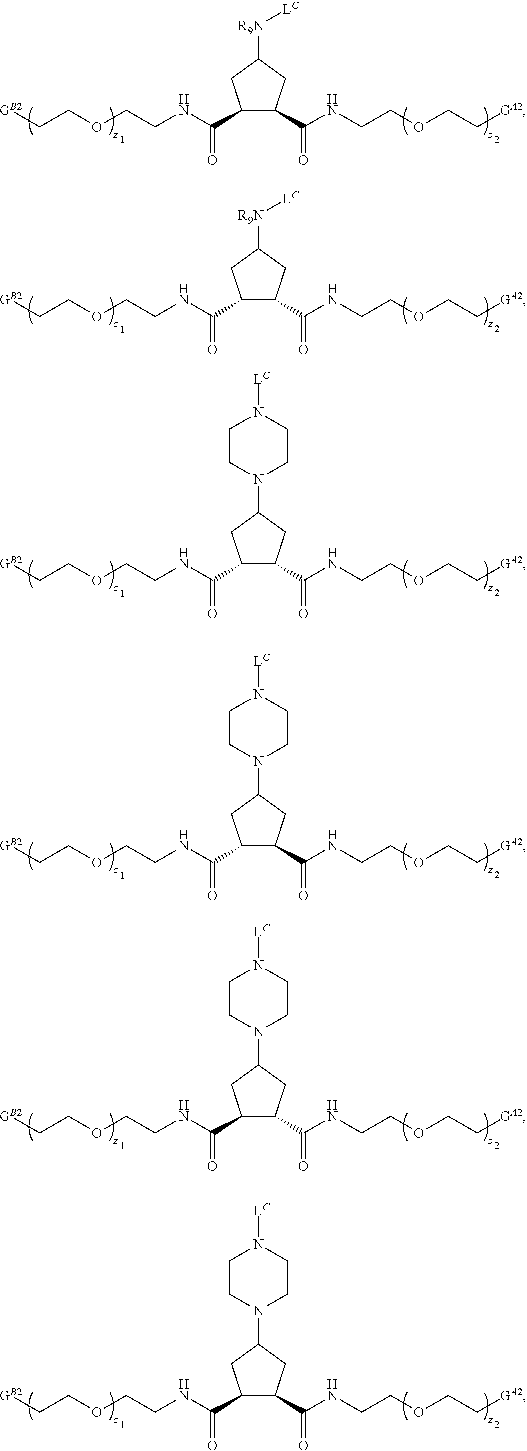

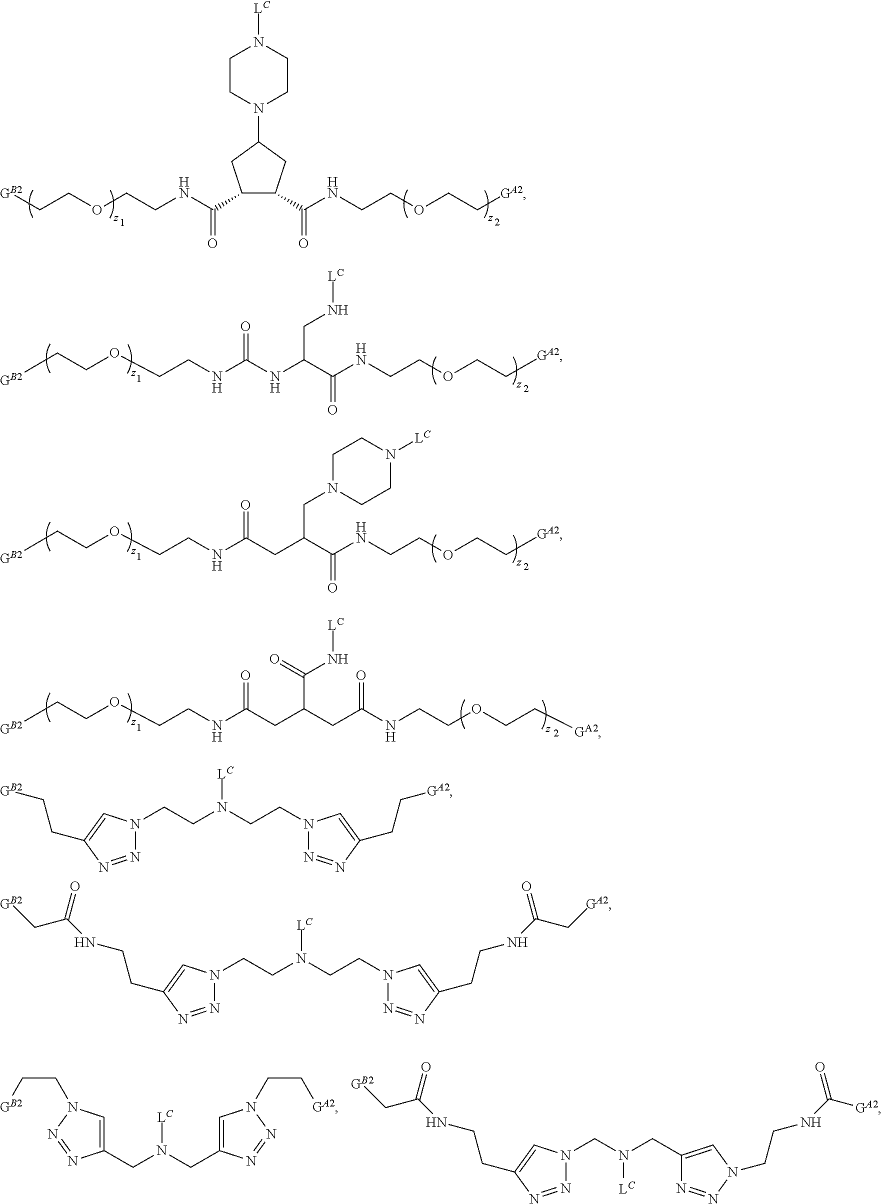

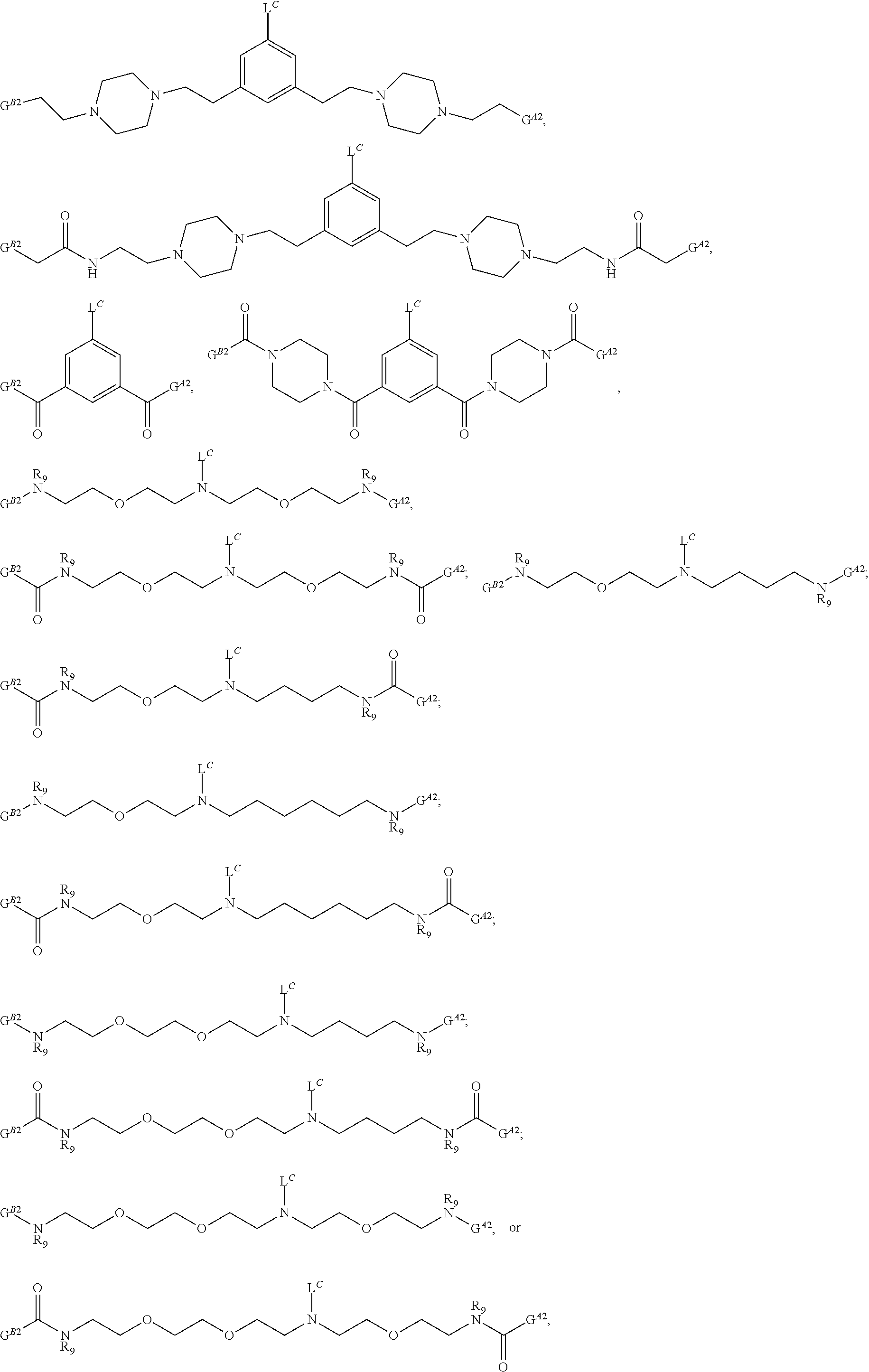

wherein z₁ and z₂ are each, independently, and integer from 1 to 20; and R₉ is selected from H, C1-C20 alkyl, C3-C20 cycloalkyl, C3-C20 heterocycloalkyl; C5-C15 aryl, and C2-C15 heteroaryl.

Linkers of the formula (D-L-I) may also include any of

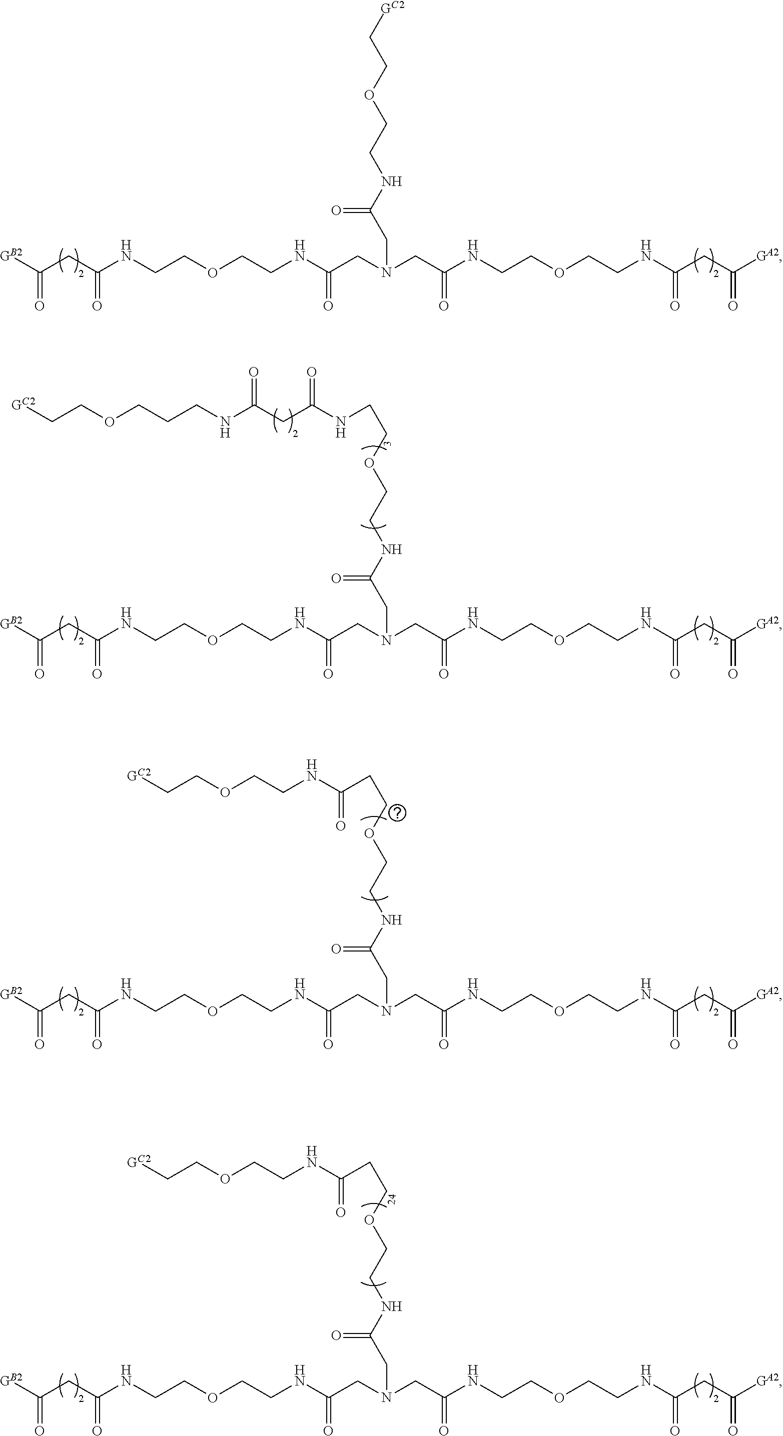

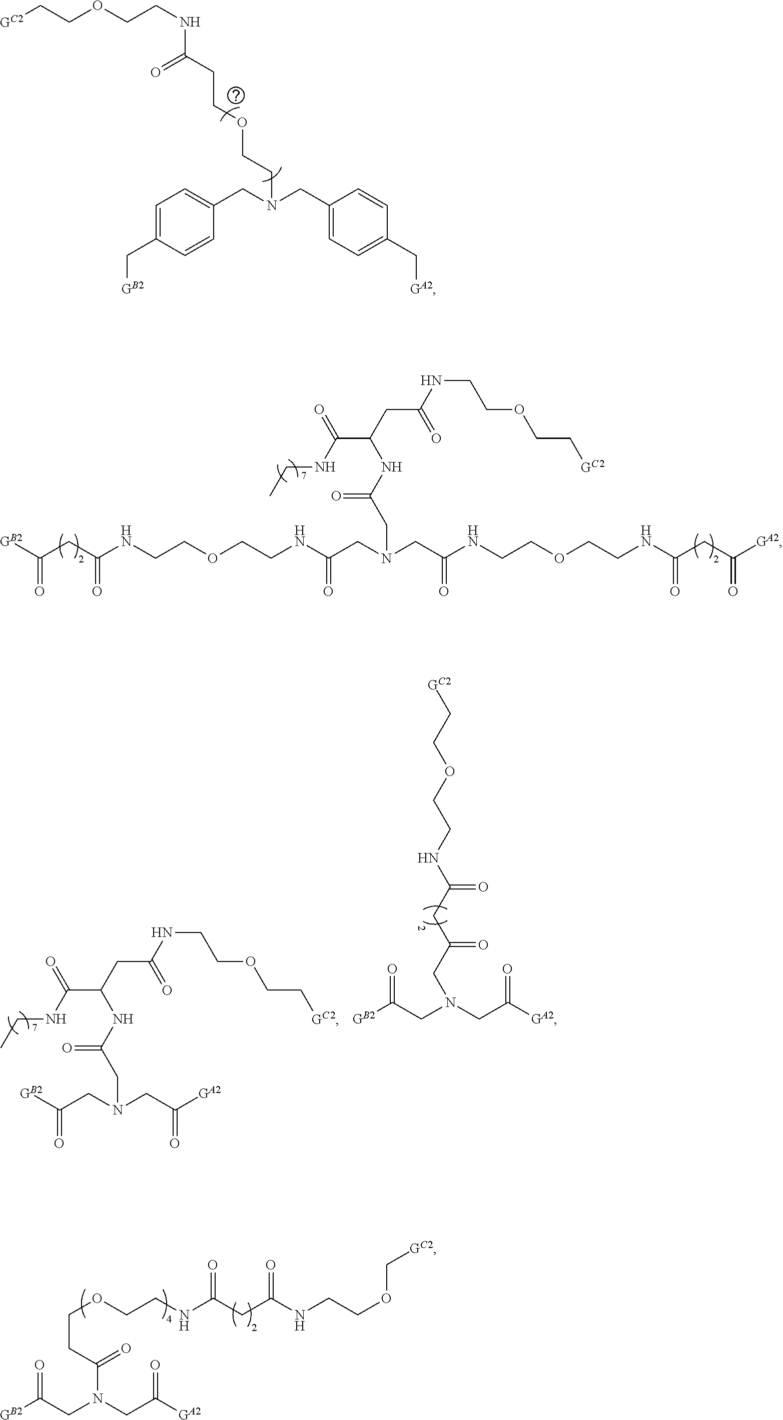

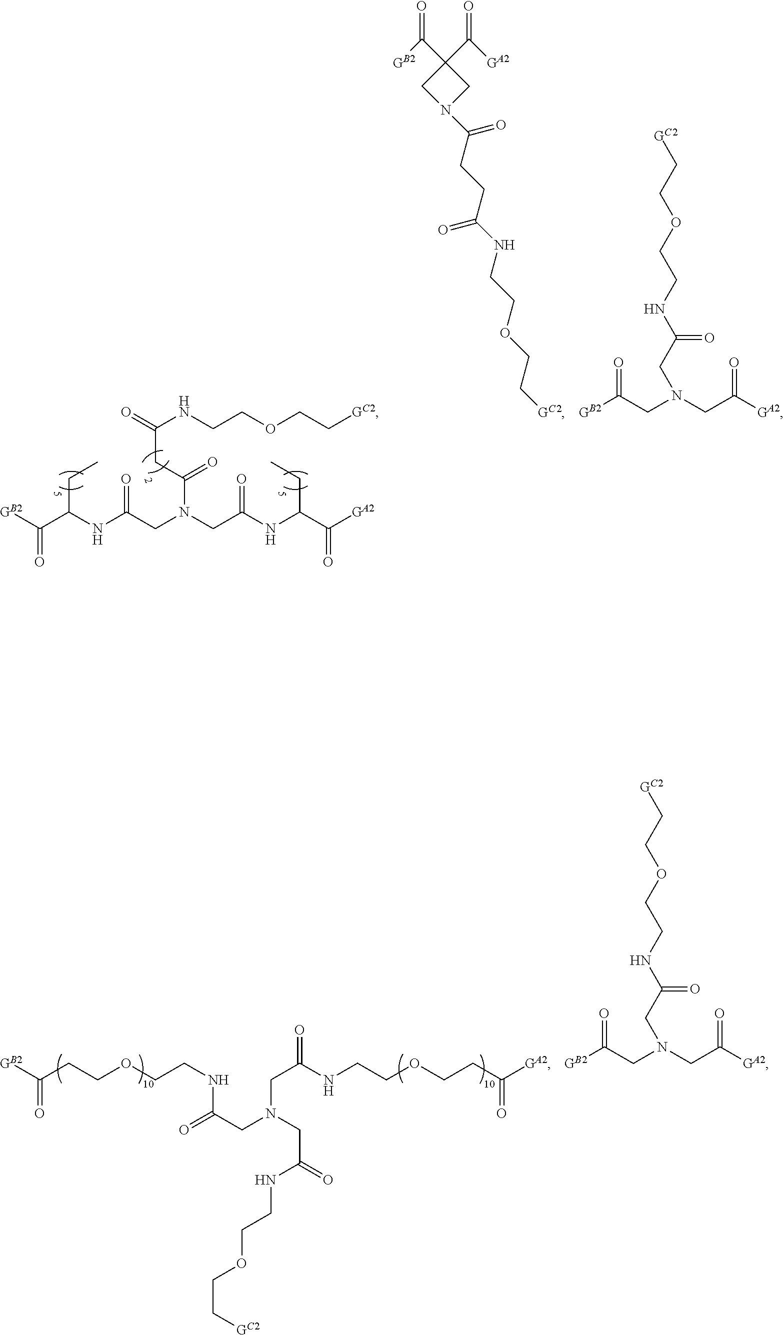

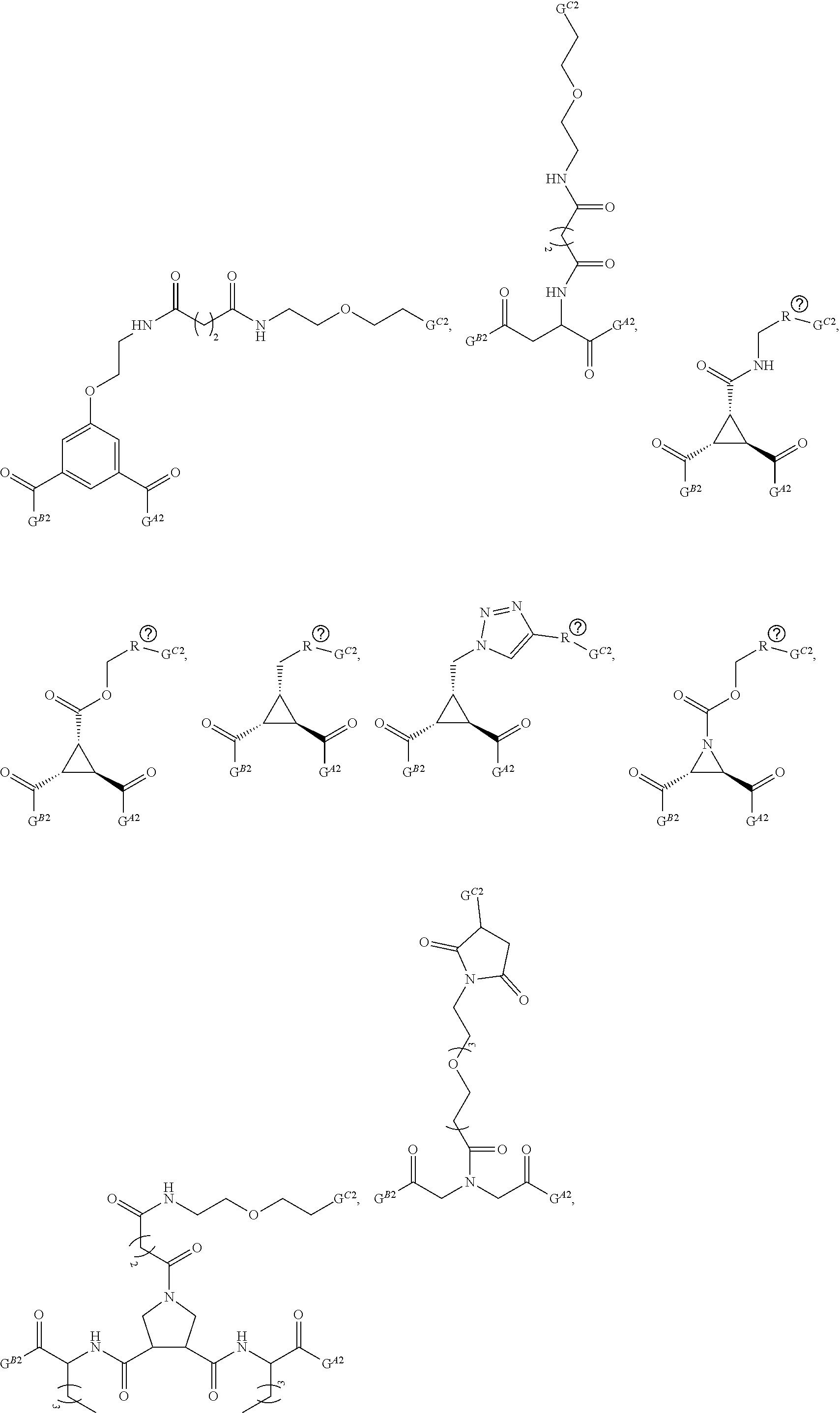

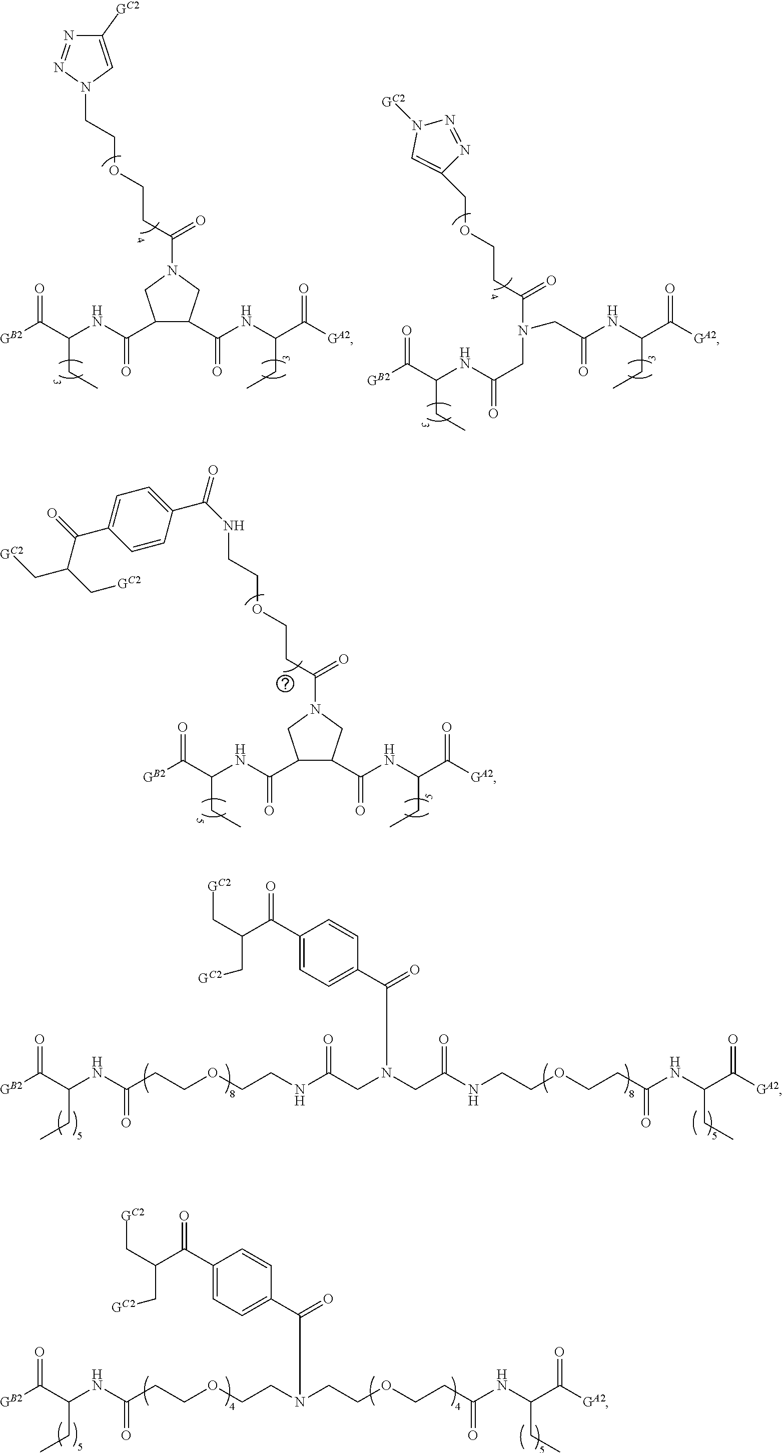

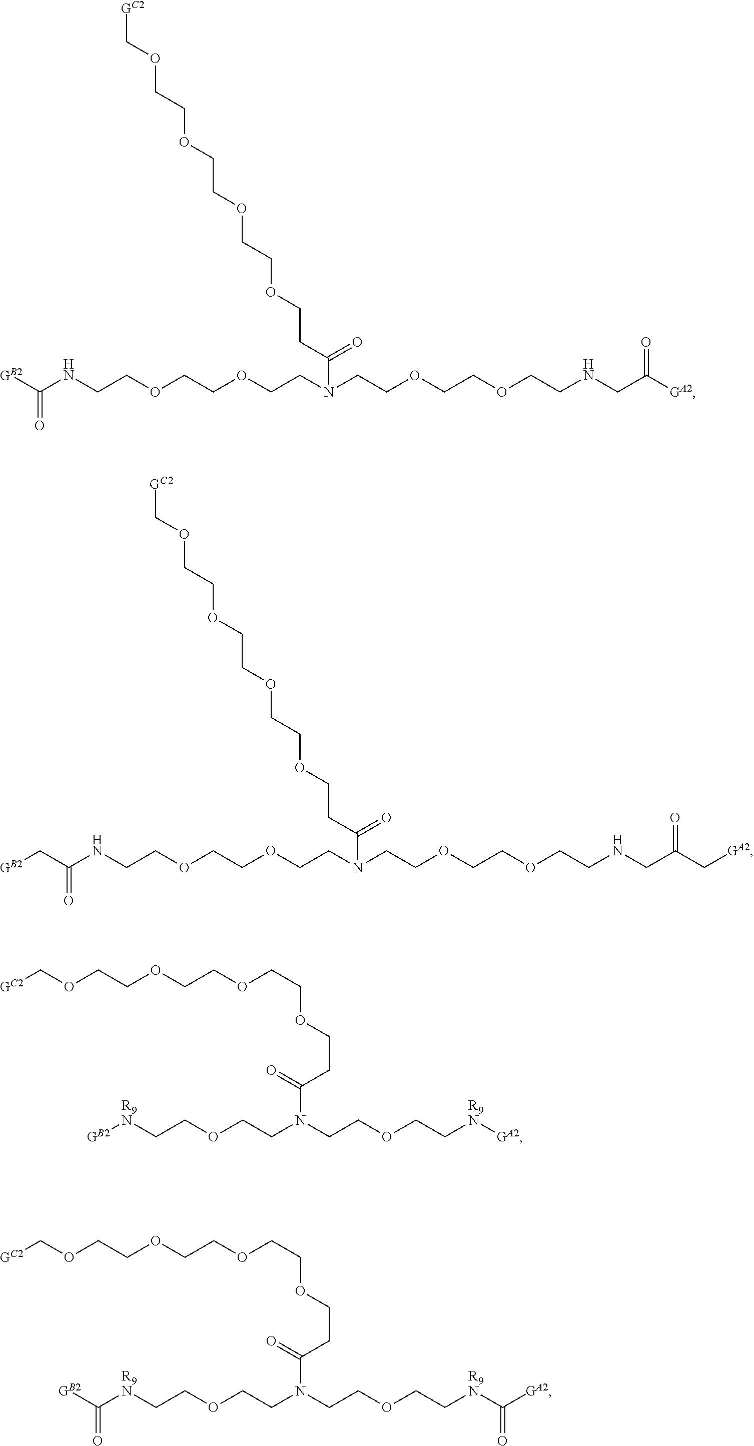

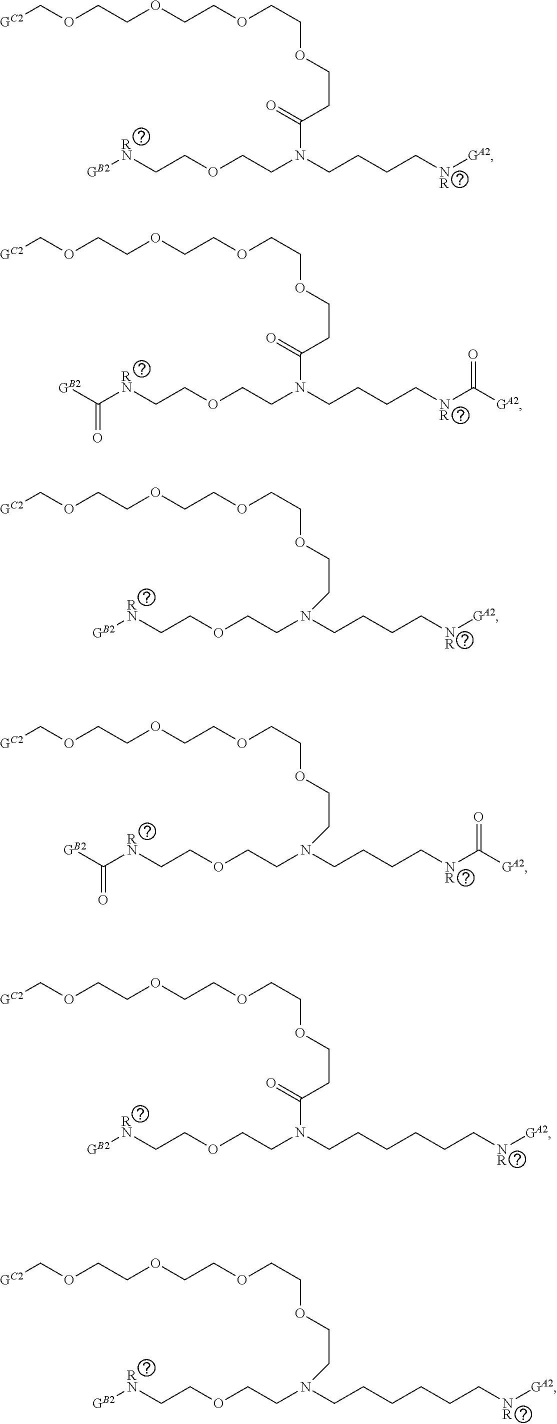

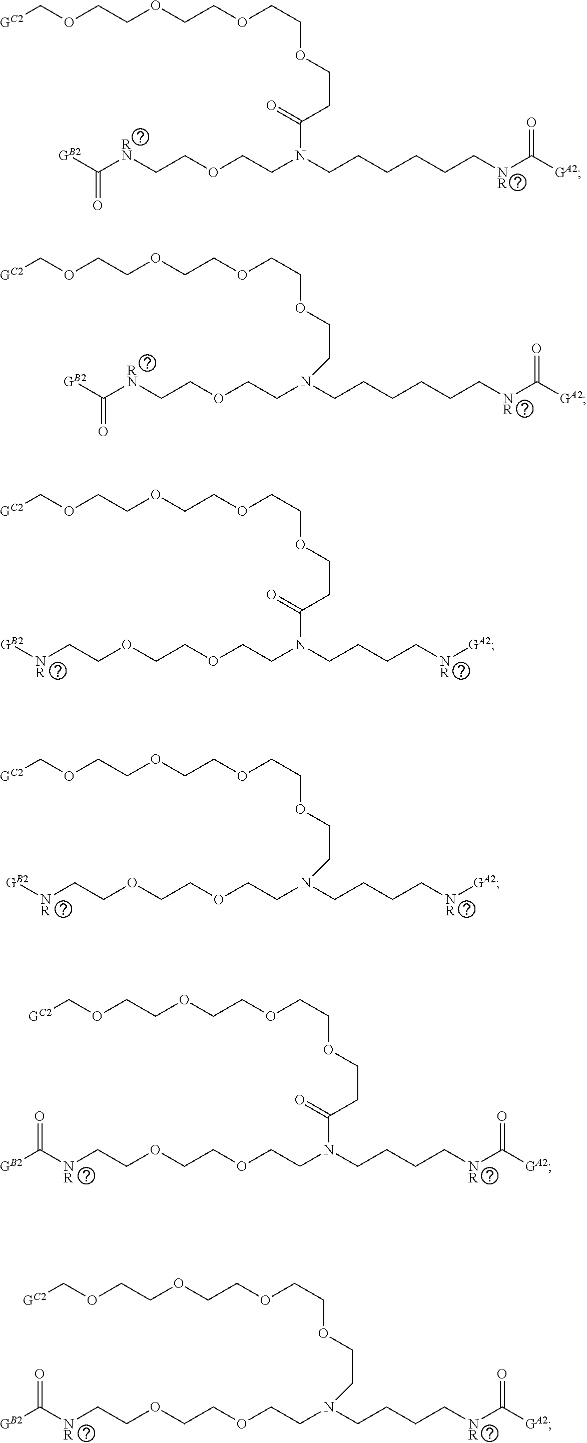

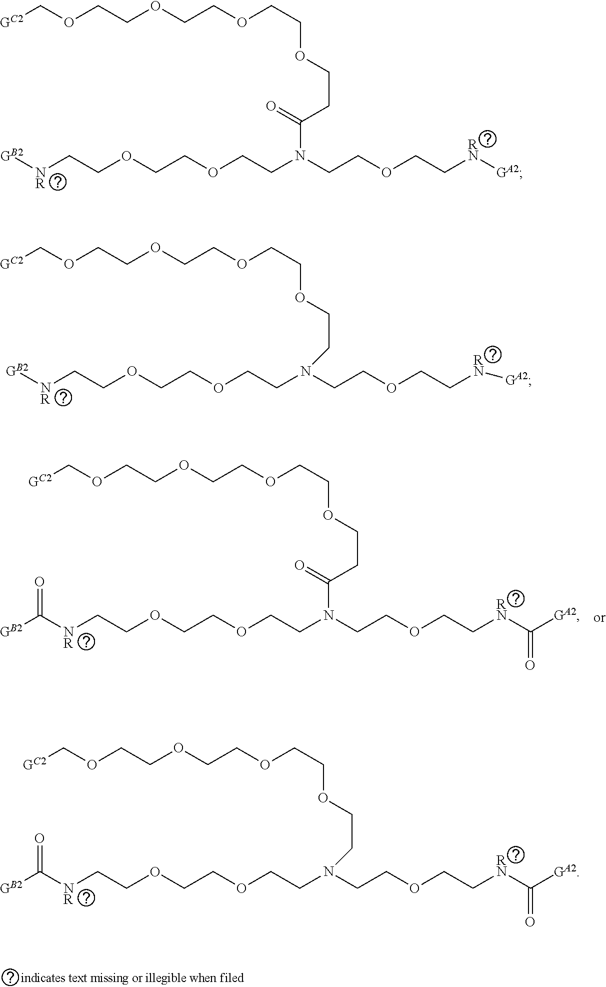

### Linking Groups

In some embodiments, a linker provides space, rigidity, and/or flexibility between the neuraminidase inhibitors and the Fc domain monomer or an Fc domain in the conjugates described here or between two neuraminidase inhibitors in the conjugates described herein. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker (L or L′ as shown in any one of formulas (D-I)-(D-VII)) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker (L or L) includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110,100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker (L or L) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For examples, hydrogen atoms attached to carbons in the backbone of the linker are not considered as directly involved in linking one part of the conjugate to another part of the conjugate.

Molecules that may be used to make linkers (L or L) include at least two functional groups, e.g., two carboxylic acid groups. In some embodiments of a trivalent linker, two arms of a linker may contain two dicarboxylic acids, in which the first carboxylic acid may form a covalent linkage with the first neuraminidase inhibitor in the conjugate and the second carboxylic acid may form a covalent linkage with the second neuraminidase inhibitor in the conjugate, and the third arm of the linker may for a covalent linkage (e.g., a C—O bond) with an Fc domain monomer or an Fc domain in the conjugate. In some embodiments of a divalent linker, the divalent linker may contain two carboxylic acids, in which the first carboxylic acid may form a covalent linkage with one component (e.g., a neuraminidase inhibitor) in the conjugate and the second carboxylic acid may form a covalent linkage (e.g., a C—S bond or a C—N bond) with another component (e.g., an Fc domain monomer or an Fc domain) in the conjugate.

In some embodiments, dicarboxylic acid molecules may be used as linkers (e.g., a dicarboxylic acid linker). For example, in a conjugate containing an Fc domain monomer or an Fc domain covalently linked to one or more dimers of neuraminidase inhibitors, the first carboxylic acid in a dicarboxylic acid molecule may form a covalent linkage with a hydroxyl or amine group of the first neuraminidase inhibitor and the second carboxylic acid may form a covalent linkage with a hydroxyl or amine group of the second neuraminidase inhibitor.

Examples of dicarboxylic acids molecules that may be used to form linkers include, but are not limited to,

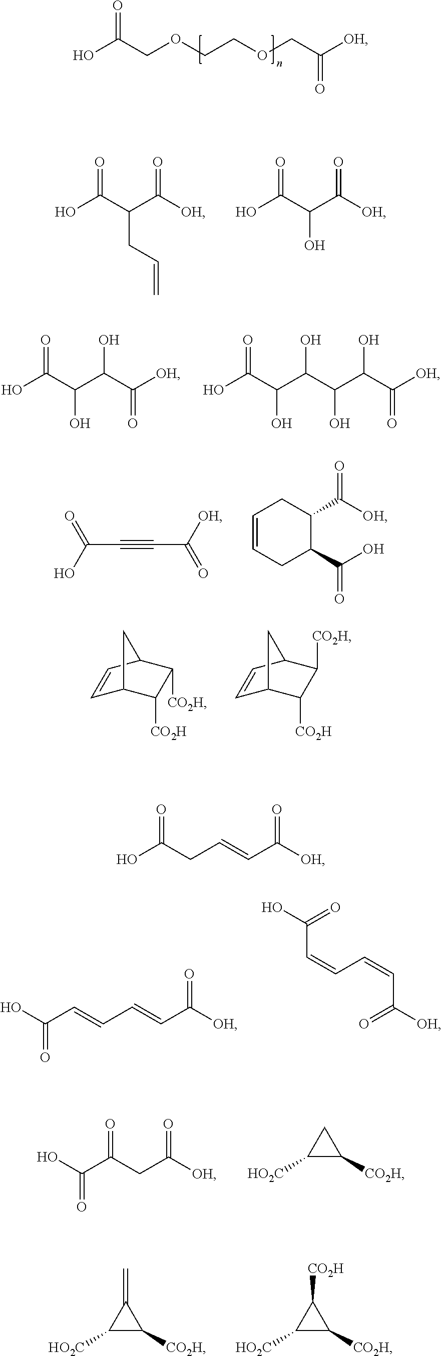

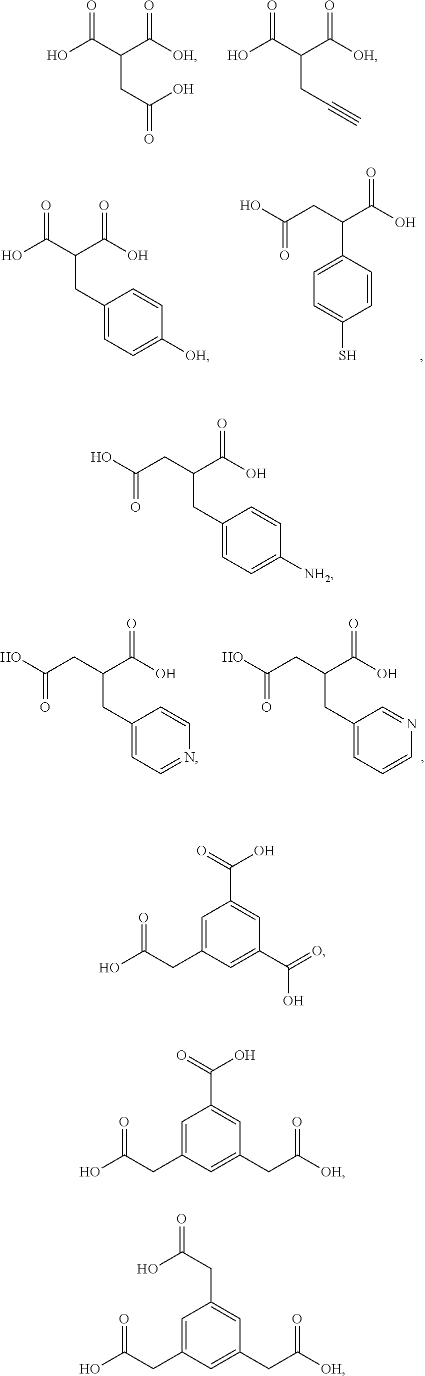

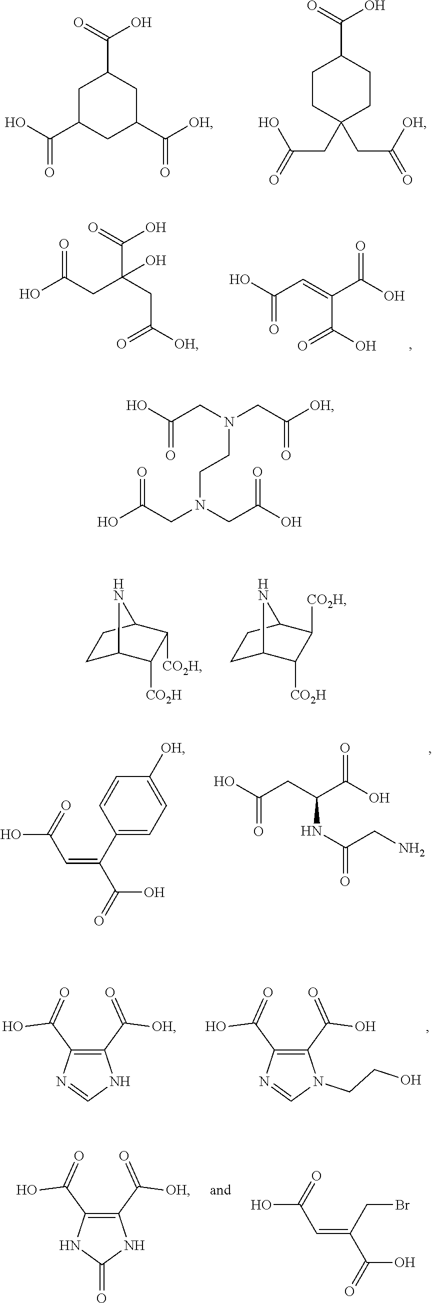

wherein n is an integer from 1 to 20 (e.g., n is 1, 2, 3, 4, 5, 6, 7, 8, 9, 10, 11, 12, 13, 14, 15, 16, 17, 18, 19, or 20).

Other examples of dicarboxylic acids molecules that may be used to form linkers include, but are not limited to,

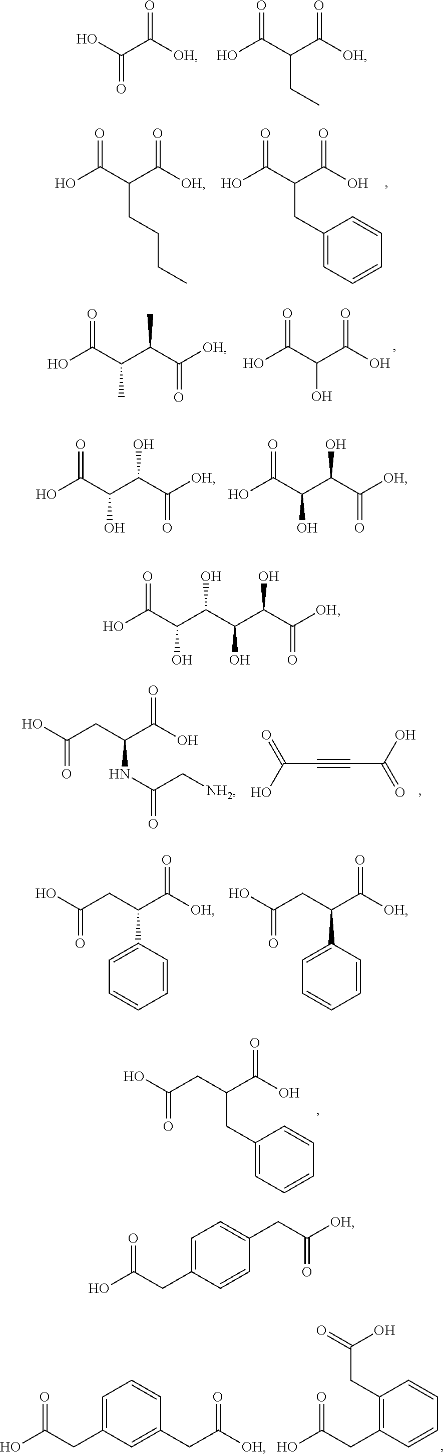

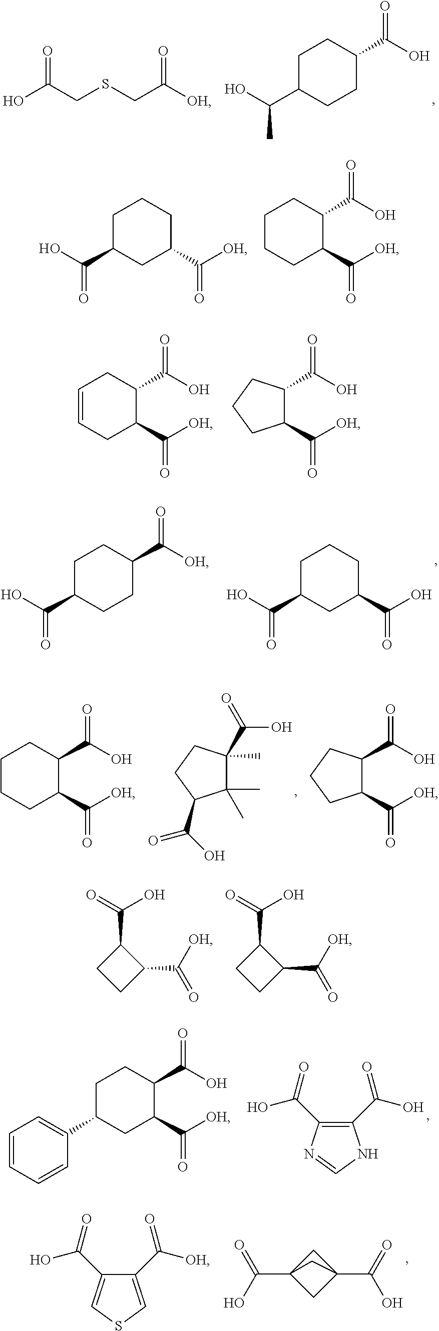

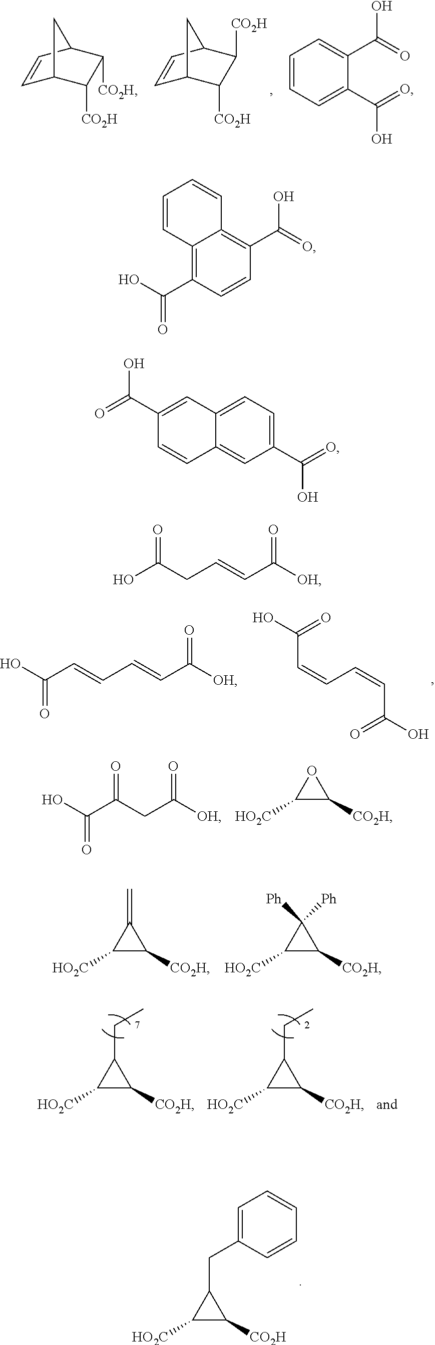

In some embodiments, dicarboxylic acid molecules, such as the ones described herein, may be further functionalized to contain one or more additional functional groups. Dicarboxylic acids may be further functionalized, for example, to provide an attachment point to an Fc domain monomer or an Fc domain (e.g., by way of a linker, such as a PEG linker).

In some embodiments, when the neuraminidase inhibitor is attached to Fc domain monomer or an Fc domain, the linking group may comprise a moiety comprising a carboxylic acid moiety and an amino moiety that are spaced by from 1 to 25 atoms. Examples of such linking groups include, but are not limited to,

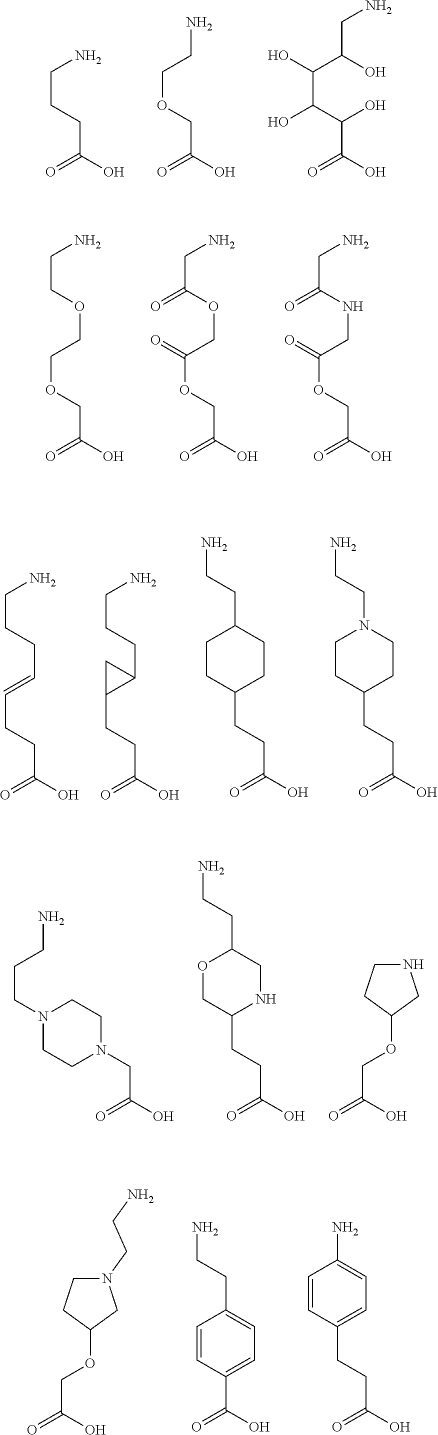

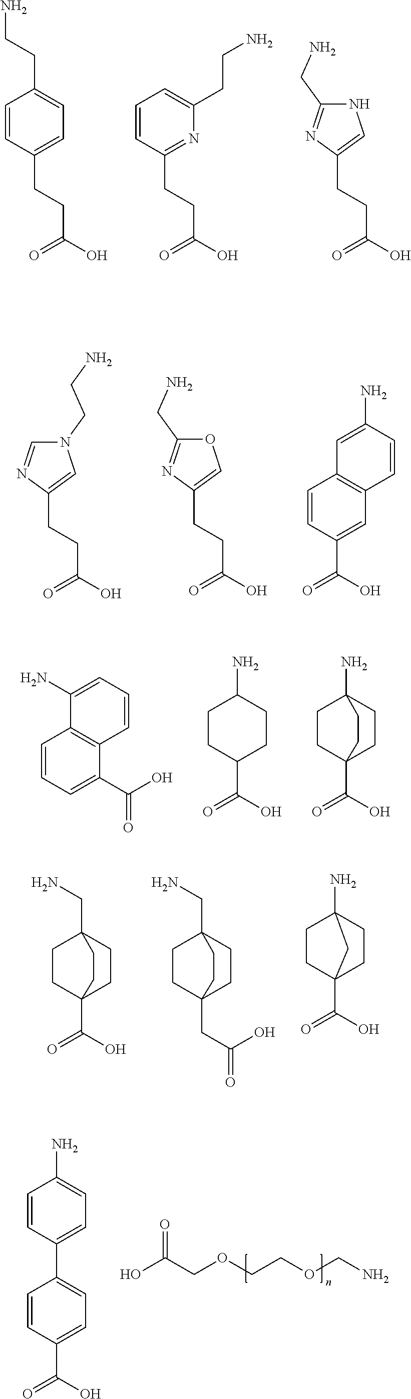

wherein n is an integer from 1 to 20 (e.g., n is 1, 2, 3, 4, 5, 6, 7, 8, 9, 10, 11, 12, 13, 14, 15, 16, 17, 18, 19, or 20).

In some embodiments, a linking group may include a moiety including a carboxylic acid moiety and an amino moiety, such as the ones described herein, may be further functionalized to contain one or more additional functional groups. Such linking groups may be further functionalized, for example, to provide an attachment point to an Fc domain monomer or an Fc domain (e.g., by way of a linker, such as a PEG linker).

In some embodiments, when the neuraminidase inhibitor is attached to Fc domain monomer or an Fc domain, the linking group may comprise a moiety comprising two or amino moieties (e.g., a diamino moiety) that are spaced by from 1 to 25 atoms. Examples of such linking groups include, but are not limited to,

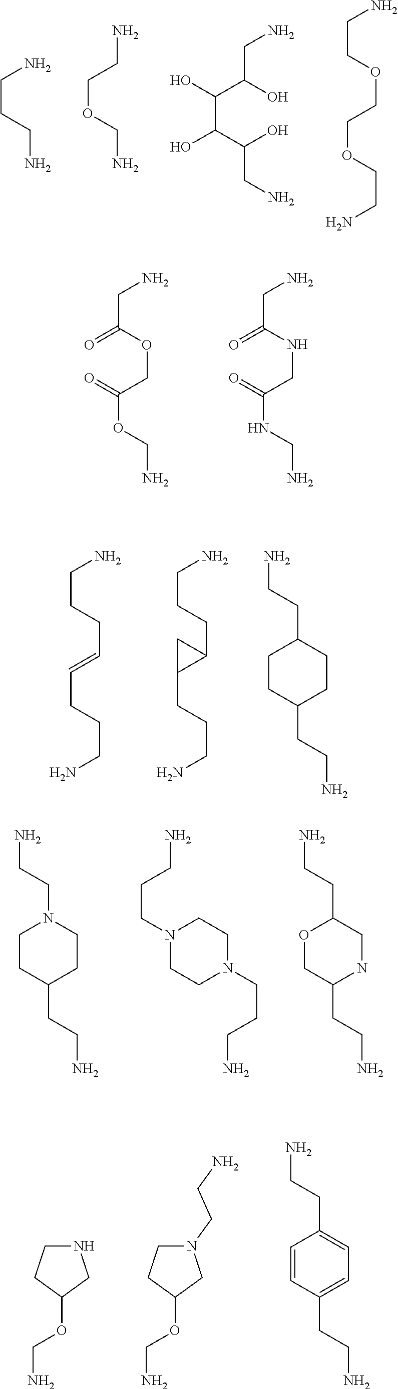

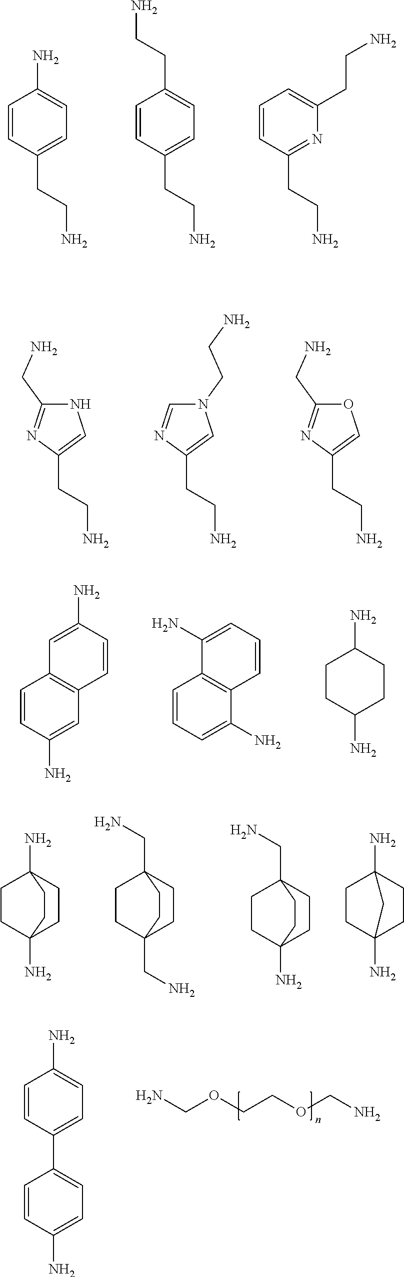

wherein n is an integer from 1 to 20 (e.g., n is 1, 2, 3, 4, 5, 6, 7, 8, 9, 10, 11, 12, 13, 14, 15, 16, 17, 18, 19, or 20).

In some embodiments, a linking group may include a diamino moiety, such as the ones described herein, may be further functionalized to contain one or more additional functional groups. Such diamino linking groups may be further functionalized, for example, to provide an attachment point to an Fc domain monomer or an Fc domain (e.g., by way of a linker, such as a PEG linker).

In some embodiments, a molecule containing an azide group may be used to form a linker, in which the azide group may undergo cycloaddition with an alkyne to form a 1,2,3-triazole linkage. In some embodiments, a molecule containing an alkyne group may be used to form a linker, in which the alkyne group may undergo cycloaddition with an azide to form a 1,2,3-triazole linkage. In some embodiments, a molecule containing a maleimide group may be used to form a linker, in which the maleimide group may react with a cysteine to form a C—S linkage. In some embodiments, a molecule containing one or more sulfonic acid groups may be used to form a linker, in which the sulfonic acid group may form a sulfonamide linkage with the linking nitrogen in a neuraminidase inhibitor. In some embodiments, a molecule containing one or more isocyanate groups may be used to form a linker, in which the isocyanate group may form a urea linkage with the linking nitrogen in a neuraminidase inhibitor. In some embodiments, a molecule containing one or more haloalkyl groups may be used to form a linker, in which the haloalkyl group may form a covalent linkage. e.g., C—N and C—O linkages, with a neuraminidase inhibitor.

In some embodiments, a linker (L or L) may comprise a synthetic group derived from, e.g., a synthetic polymer (e.g., a polyethylene glycol (PEG) polymer). In some embodiments, a linker may comprise one or more amino acid residues. In some embodiments, a linker may be an amino acid sequence (e.g., a 1-25 amino acid, 1-10 amino acid, 1-9 amino acid, 1-8 amino acid, 1-7 amino acid, 1-8 amino acid, 1-5 amino acid, 1-4 amino acid, 1-3 amino acid, 1-2 amino acid, or 1 amino acid sequence). In some embodiments, a linker (L or L′) may include one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rᵢ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

### Conjugation Chemistries

Neuraminidase inhibitor dimers (e.g., in a conjugate of any one of formulas (D-I)-(D-VIII)) may be conjugated to an Fc domain monomer or an Fc domain, e.g., by way of a linker, by any standard conjugation chemistries known to those of skill in the art. The following conjugation chemistries are specifically contemplated, e.g., for conjugation of a PEG linker (e.g., a functionalized PEG linker) to an Fc domain monomer or an Fc domain.

Covalent conjugation of two or more components in a conjugate using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Site-specific conjugation to a polypeptide (e.g., an Fc domain monomer or an Fc domain) may be accomplished using techniques known in the art. Exemplary techniques for site-specific conjugation of a small molecule to an Fc domain are provided in Agarwall. P., et al. Bioconjugate Chem. 26:176-192 (2015).

Other examples of functional groups capable of reacting with amino groups include, e.g., alkylating and acylating agents. Representative alkylating agents include: (i) an α-haloacetyl group, e.g., XCH₂CO— (where X=Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiffs base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sulfhydryl, and hydroxyl groups; (viii) an aziridine, which is reactive towards nucleophiles such as amino groups by ring opening; (ix) a squaric acid diethyl ester; and (x) an α-haloalkyl ether.

Examples of amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester, or derivatives thereof (e.g., azido-PEG₂-PEG₄₀-NHS ester); (v) an acid anhydride, e.g., a mixed, symmetrical, or N-carboxyanhydride; (vi) an acylazide; and (vii) an imidoester. Aldehydes and ketones may be reacted with amines to form Schiffs bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

In some embodiments, a linker of the disclosure (e.g., L or L′, such as L^{c} of D-L-I), is conjugated (e.g., by any of the methods described herein) to E (e.g., an Fc domain). In preferred embodiments of the disclosure, the linker is conjugated by way of: (a) a thiourea linkage (i.e., —NH(C═S)NH—) to a lysine of E; (b) a carbamate linkage (i.e., —NH(C═O)—O) to a lysine of E: (c) an amine linkage by reductive amination (i.e., —NHCH₂) between a lysine and E; (d) an amide (i.e., —NH—(C═O)CH₂) to a lysine of E; (e) a cysteine-maleimide conjugate between a maleimide of the linker to a cysteine of E; (f) an amine linkage by reductive amination (i.e., —NHCH₂) between the linker and a carbohydrate of E (e.g., a glycosyl group of an Fc domain monomer or an Fc domain); (g) a rebridged cysteine conjugate, wherein the linker is conjugated to two cysteines of E; (h) an oxime linkage between the linker and a carbohydrate of E (e.g., a glycosyl group of an Fc domain monomer or an Fc domain); (i) an oxime linkage between the linker and an amino acid residue of E; (j) an azido linkage between the linker and E; (k) direct acylation of a linker to E; or (l) a thioether linkage between the linker and E.

In some embodiments, a linker is conjugated to E, wherein the linkage includes the structure —NH(C═NH)X—, wherein X is O, HN, or a bond. In some embodiments, a linker is conjugated to E, wherein the linkage between the remainder of the linker and E includes the structure —NH(C═O)NH—. In some embodiments, a linker (e.g., an active ester, e.g., a nitrophenylester or N-hydroxysuccinimidyl ester, or derivatives thereof (e.g., a functionalized PEG linker (e.g., azido-PEG₂-PEG₄₀-NHS ester), is conjugated to E, with a T of (e.g., DAR) of between 0.5 and 10.0. In these instances, the E-(PEG₂-PEG₄₀)-azide can react with an Int having a terminal alkyne linker (e.g., L, or L′, such as L^{c} of D-L-I) through click conjugation. During click conjugation, the copper-catalyzed reaction of the an azide (e.g., the Fc-(PEG₂-PEG₄₀)-azide) with the alkyne (e.g., the Int having a terminal alkyne linker (e.g., L or L′, such as L^{c} of D-L-I) forming a 5-membered heteroatom ring. In some embodiments, the linker conjugated to E is a terminal alkyne and is conjugated to an Int having a terminal azide. Exemplary preparations of preparations of E-(PEG₂-PEG₄₀)-azide are described in Examples 7, 8, 61, 84, 88, and 124 of WO 2021/046549. Exemplary conjugates prepared through click conjugation are depicted in FIGS. 43, 61, and 102 of WO 2021/048549. The click chemistry conjugation procedure is depicted in FIG. 103 of WO 2021/048549. One of skill in the art would readily understand the final product from a click chemistry conjugation.

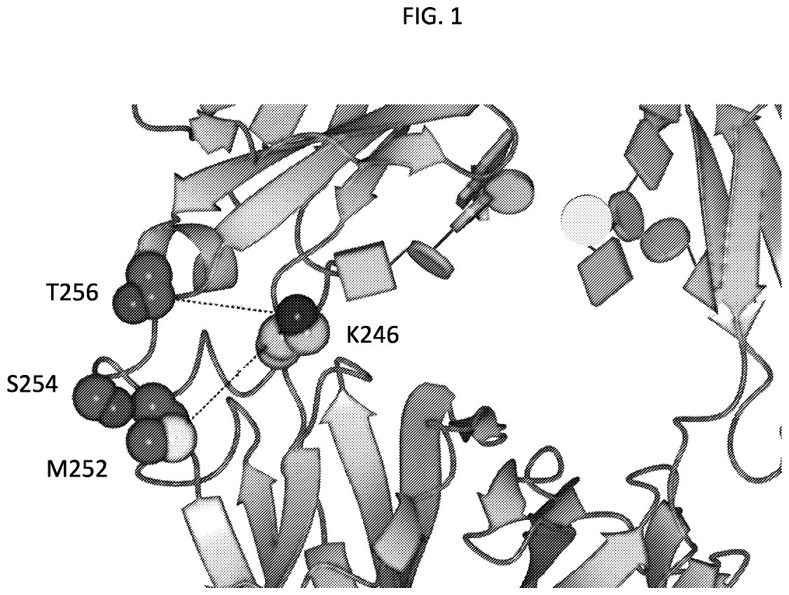

Exemplary linking strategies (e.g., methods for linking a dimer of a neuraminidase inhibitor to E, such as, by way of a linker) are further depicted in FIGS. 1, 28, 29, 30, 43, and 61 of WO 2021/046549.

### V. Combination Therapies

### Antiviral Agents

In some embodiments, one or more antiviral agents may be administered in combination (e.g., administered substantially simultaneously (e.g., in the same pharmaceutical composition or in separate pharmaceutical compositions) or administered separately at different times) with a conjugate described herein (e.g., a conjugate of any one of formulas (D-I)-(D-VIII)).

The antiviral agent may be administered substantially simultaneously (e.g., in the same pharmaceutical composition or in separate pharmaceutical compositions) as the conjugates, or may be administered prior to or following the conjugates (e.g., within a period of 1 day, 2, days, 5, days, 1 week, 2 weeks, 3 weeks, 1 month, 2 months, 6 months, or 12 months, or more). In some embodiments, the conjugate is administered by injection (e.g., intramuscularly, intradermally, intranasally, or subcutaneously), and the antiviral agent is administered orally. Most preferably, the conjugate is administered intravenously, and the antiviral agent is administered orally.

In some embodiments, the conjugate is administered prophylactically (e.g., prior to the subject coming into contact with the virus) and the antiviral agent is administered after the subject has a viral infection, is presumed to have a viral infection, or has been exposed to the virus. In some embodiments, the conjugate and the antiviral agent are both administered after the subject has a viral infection, is presumed to have a viral infection, or has been exposed to the virus. In some embodiments, the conjugate and the antiviral agent are both administered prophylactically.

The conjugate and the antiviral agent may be formulated in the same pharmaceutical composition or in separate pharmaceutical compositions. In preferred embodiments, the conjugate and the antiviral agent is formulated in separate pharmaceutical compositions (e.g., formulated for different routes of administration). In some embodiments, the conjugate and the antiviral agent are administered simultaneously (e.g., at substantially the same time, such as within 5 minutes, 30 minutes, 1-6 hours, 1-12 hours, or 1 day) or sequentially (e.g., at different times, such as more than 1 day apart). Provided the antiviral agent and the conjugate are administered sequentially, the antiviral agent is administered 1-50 (e.g., 1-15, 10-25, 20-35, 30-45, or 35-50) times after the administration of the conjugate (e.g., administrations 1 day, 2, days, 5, days, 1 week, 2 weeks, 3 weeks, 1 month, 2 months, 6 months, or 12 months, or more after the conjugate).

In some instances, an antiviral agent is administered to a subject in need thereof one or more times (e.g., 1-10 times or more: 1, 2, 3, 4, 5, 6, 7, 8, 9, or 10 times) daily, weekly, monthly, biannually, annually, or as medically necessary after the administration of a conjugate described herein (e.g., a conjugate of any one of formulas (D-I)-(D-VIII)).

In some embodiments, the antiviral agent is an antiviral agent for the treatment of influenza virus. For example, the antiviral agent may be an M2 ion channel blocker, a neuraminidase inhibitor (e.g., a long-acting neuraminidase inhibitor), a polymerase inhibitor, a hemagglutinin inhibitor, a fusion protein inhibitor, a COX-2 inhibitor, or a PPAR agonist. The antiviral agent may target either the virus or the host subject. The antiviral agent for the treatment of influenza virus used in combination with a conjugate described herein (e.g., a conjugate of any one of formulas (D-I)-(D-VIII)) may be selected from pimovidir, oseltamivir, zanamivir, peramivir, laninamivir, CS-8958, amantadine, rimantadine, cyanovirin-N, a cap-dependent endonuclease inhibitor (e.g., baloxavir marboxil), a polymerase inhibitor (e.g., T-705), a PB2 inhibitor (e.g., JNJ-63623872), a conjugated sialidase (e.g., DAS181), a thiazolide (e.g., nitazoxanide), a COX inhibitor, a PPAR agonist, a hemagglutinin-targeting antibody (e.g., a monoclonal antibody such as CR6261, CR8020, MED18852, MHAA4549A, or VIS410), or an siRNA targeting a host or viral gene, or prodrugs thereof, or pharmaceutically acceptable salts thereof.

Preferably, the antiviral agent is directed to a different therapeutic target than the conjugate, for example an M2 ion channel blocker, a polymerase inhibitor, a hemagglutinin inhibitor, a viral replication inhibitor (e.g., a cap-dependent endonuclease inhibitor), a fusion protein inhibitor, a COX-2 inhibitor, or a PPAR agonist. Most preferably, the antiviral agent is a cap-dependent endonuclease inhibitor (e.g., baloxavir marboxil). In some embodiments, the antiviral agent is administered in combination with a conjugate described by formula (D-II-6). In some embodiments, the antiviral agent is administered in combination with a conjugate described by formula (D-II-7). In preferred embodiments, an antiviral agent (e.g., baloxavir marboxil) is administered in combination with a conjugate described by formula (D-II-6). Most preferably, an antiviral agent (e.g., baloxavir marboxil) is administered in combination with a conjugate described formula (D-II-7).

### Baloxavir

In some embodiments, Baloxavir marboxil (BXM, prodrug form) or baloxavir acid (BXA, active form) or any salt thereof (Omoto et al. Scientific Reports. 8:9633, 2018; Japic CTI-153090; Japic CTI-163417; each of which are incorporated herein by reference in their entirety) may be administered in combination (e.g., administered substantially simultaneously (e.g., in the same pharmaceutical composition or in separate pharmaceutical compositions) or administered separately at different times) with a conjugate described herein (e.g., a conjugate of any one of formulas (D-I)-(D-VIII)).

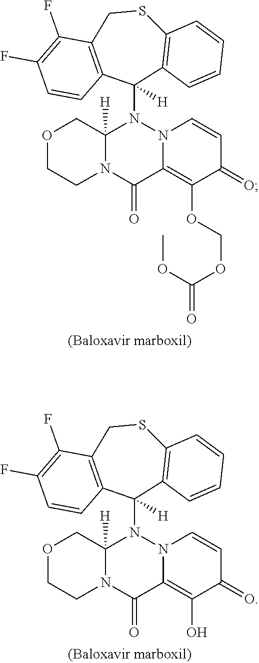 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

In some embodiments, Baloxavir marboxil, Baloxavir acid, or salt thereof is administered in a dosage ranging from about 0.1 mg to about 3000 mg, preferably about 0.1 mg to about 1000 mg, most preferable about 10 mg to about 100 mg (e.g., about 10 mg, about 20 mg, about 30 mg, about 40 mg, about 50 mg, about 60 mg, about 70 mg, about 80 mg, about 90 mg, or about 100 mg) per adult a day, if necessary, by division. In some embodiments, the Baloxavir marboxil, Baloxaviracid, or salt thereof is administered at a decreased dose or frequency compared to standard of care when administered in combination with the conjugate. The conjugate may be administered at a dose described herein.

In some embodiments, Baloxavir marboxil, Baloxavir acid, or a salt thereof is administered more frequently than the conjugate. For example, the conjugate may be administered once every 12 months, 6 months, 3 months, 2 months, 1 month, every 3 weeks, every 2 weeks, or weekly. The Baloxavir marboxil, Baloxavir acid, or salt thereof may be administered three times daily, twice daily, once daily, once every 2-6 days, once weekly, or once every two weeks. In some embodiments, Baloxavir marboxil, Baloxavir acid, or salts thereof, are administered one or more times (e.g., 1-10 times or more; 1, 2, 3, 4, 5, 8, 7, 8, 9, 10 times or more) after (e.g., within 6 months, 3 months, 2 months, 1 month, 3 weeks, 2 weeks, or 1 week) the administration of a conjugate described herein.

In some embodiments, Baloxavir marboxil. Baloxavir acid, or a salt thereof is administered orally.

In some embodiments, Baloxavir marboxil. Baloxavir acid, or a salt thereof is administered, e.g., orally, in a dosage ranging from about 0.01 mg to about 1000 mg, preferably about 0.05 mg to about 500 mg, per day. Dosage forms and strengths for Baloxavir marboxil (XOFLUZA™) are well known, with a single 40 mg oral dose for adults 40 to <80 kg and a single 80 mg oral dose for adults 280 kg. Dosage forms and strengths for Baloxavir marboxil (XOFLUZA™) for pediatric subjects (e.g., subjects 212 years old and 240 kg) is well known, with a single 40 mg oral dose for pediatric subjects 40 to <80 kg and a single 80 mg oral dose for pediatric subjects 280 kg.

When administered in combination with a conjugate of the present disclosure, the efficacy of baloxavir (e.g., baloxavir marboxil, baloxavir acid, or a salt thereof) may be enhanced, e.g., by a synergistic interaction of the baloxavir and the conjugate. This may permit the administration of baloxavir at a reduced dose (e.g., relative to the present clinical standard of care) without any loss of efficacy. This has the advantage of decreasing adverse events associate with administration of baloxavir. In some embodiments, Baloxavir marboxil, Baloxavir acid, or a salt thereof is administered in a reduced or subclinical dose (e.g., administered at a dose lower than without a conjugate described herein and/or lower than the present clinical standard of care (e.g., a dose lower than 40 mg oral dose (e.g., a dose ranging from 0.01 mg to 40 mg (e.g., 0.5 mg, 1.0 mg, 1.5 mg, 2.0 mg, 2.5 mg, 5.0 mg, 7.5 mg, 10 mg, 15 mg, 18 mg, 20 mg, 23 mg, 25 mg, 30 mg, 35 mg, or 38 mg oral dose))). Baloxavir marboxil (XOFLUZA™) may be provided in any amount sufficient to treat an influenza viral infection in a subject having previously been administered any conjugate described herein.

### Antiviral Vaccines

In some embodiments, any one of conjugates described herein (e.g., a conjugate of any one of formulas (D-I)-(D-VIII)) is administered in combination with an antiviral vaccine (e.g., a composition that elicits an immune response in a subject directed against a virus). The antiviral vaccine may be administered substantially simultaneously (e.g., in the same pharmaceutical composition or in separate pharmaceutical compositions) as the conjugates, or may be administered prior to or following the conjugates (e.g., within a period of 1 day, 2, days, 5, days, 1 week, 2 weeks, 3 weeks, 1 month, 2 months, 6 months, or 12 months, or more).

In some embodiments the viral vaccine comprises an immunogen that elicits an immune response in the subject against influenza virus A, B, C, or parainfluenza virus. In some embodiments the immunogen is an inactivated virus (e.g., the vaccine is a trivalent influenza vaccine that contains purified and inactivated material influenza virus A, B, C, or parainfluenza virus or any combination thereof). In some embodiments the vaccine is given as an intramuscular injection. In some embodiments, the vaccine is a live virus vaccine that contains live viruses that have been attenuated (weakened). In some embodiments the vaccine is administered as a nasal spray.

### VI. Methods

Methods described herein include, e.g., methods of protecting against or treating a viral infection (e.g., an influenza viral infection) in a subject and methods of preventing, stabilizing, or inhibiting the growth of viral particles. A method of treating a viral infection (e.g., an influenza viral infection) in a subject includes administering to the subject a conjugate described herein (e.g., a conjugate of any one of formulas (D-I)-(D-VIII)) or a pharmaceutical composition thereof. In some embodiments, the viral infection is cause by the influenza virus (e.g., influenza virus A, B, C, or parainfluenza virus). In some embodiments, the viral infection is caused by a resistant strain of virus. A method of preventing, stabilizing, or inhibiting the growth of viral particles or preventing the replication and spread of the virus includes contacting the virus or a site susceptible to viral growth with a conjugate described herein (e.g., a conjugate of any one of formulas (D-I)-(D-VIII)) or a pharmaceutical composition thereof.

The disclosure also provides a method of protecting against or treating a viral infection (e.g., an influenza viral infection) in a subject having or at risk of developing a secondary infection (e.g., a secondary bacterial infection, a secondary viral infection, or a secondary fungal infection), wherein the method includes administering to the subject a conjugate or composition described herein. The disclosure further provides a method of preventing a secondary infection in a subject diagnosed with an influenza infection, wherein the method includes administering to the subject a conjugate or composition described herein. In some embodiments, the secondary infection is a bacterial infection (e.g., methicillin-resistant Staphylococcus aureus (MRSA). Streptococcus pneumoniae, Pseudomonas aeruginosa, and/or Haemophilus influenzae), a viral infection, or a fungal infection. In particular embodiments, the secondary infection is MRSA. In certain embodiments, the secondary infection is S. pneumoniae. In some embodiments, the secondary infection is a respiratory infection (e.g., an infection of the respiratory tract). In some embodiments, the secondary infection is associated with (e.g., causes) pneumonia (e.g., bacterial or viral pneumonia). In some embodiments, the subject has or is at risk of developing pneumonia.

Moreover, methods described herein also include methods of protecting against or treating viral infection in a subject by administering to the subject a conjugate described herein (e.g., a conjugate of any one of formulas (D-I)-(D-VIII)). In some embodiments, the method further includes administering to the subject an antiviral agent or an antiviral vaccine.

Methods described herein also include methods of protecting against or treating a viral infection in a subject by administering to said subject (1) a conjugate described herein (e.g., a conjugate of any one of formulas (D-I)-(D-VIII)) and (2) an antiviral agent or an antiviral vaccine. Methods described herein also include methods of preventing, stabilizing, or inhibiting the growth of viral particles or preventing the replication or spread of a virus, by contacting the virus or a site susceptible to viral growth with (1) a conjugate described herein (e.g., a conjugate of any one of formulas (D-I)-(D-VIII)) and (2) an antiviral agent or an antiviral vaccine.

In some embodiments, the conjugate described herein (e.g., a conjugate of any one of formulas (D-I)-(D-VIII)) is administered first, followed by administering of the antiviral agent or antiviral vaccine alone. In some embodiments, the antiviral agent or antiviral vaccine is administered first, followed by administering of the conjugate described herein alone. In some embodiments, the conjugate described herein and the antiviral agent or antiviral vaccine are administered substantially simultaneously (e.g., in the same pharmaceutical composition or in separate pharmaceutical compositions). In some embodiments, the conjugate described herein or the antiviral agent or antiviral vaccine is administered first, followed by administering of the conjugate described herein and the antiviral agent or antiviral vaccine substantially simultaneously (e.g., in the same pharmaceutical composition or in separate pharmaceutical compositions). In some embodiments, the conjugate described herein and the antiviral agent or antiviral vaccine are administered first substantially simultaneously (e.g., in the same pharmaceutical composition or in separate pharmaceutical compositions), followed by administering of the conjugate described herein or the antiviral agent or antiviral vaccine alone. In some embodiments, when a conjugate described herein (e.g., a conjugate of any one of formulas (D-I)-(D-VIII)) and an antiviral agent or antiviral vaccine are administered together (e.g., substantially simultaneously in the same or separate pharmaceutical compositions, or separately in the same treatment regimen), inhibition of viral replication of each of the conjugate and the antiviral agent or antiviral vaccine may be greater (e.g., occur at a lower concentration) than inhibition of viral replication of each of the conjugate and the antiviral agent or antiviral vaccine when each is used alone in a treatment regimen.

### VII. Pharmaceutical Compositions and Preparations

A conjugate described herein may be formulated in a pharmaceutical composition for use in the methods described herein. In some embodiments, a conjugate described herein may be formulated in a pharmaceutical composition alone. In some embodiments, a conjugate described herein may be formulated in combination with an antiviral agent or antiviral vaccine in a pharmaceutical composition. In some embodiments, the pharmaceutical composition includes a conjugate described herein (e.g., a conjugate described by any one of formulas (D-I)-(D-VIII)) and pharmaceutically acceptable carriers and excipients.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acid residues such as glycine, glutamine, histidine, and lysine, and carbohydrates such as glucose, mannose, sucrose, and sorbitol.

Examples of other excipients include, but are not limited to, antiadherents, binders, coatings, compression aids, disintegrants, dyes, emollients, emulsifiers, fillers (diluents), film formers or coatings, flavors, fragrances, glidants (flow enhancers), lubricants, sorbents, suspensing or dispersing agents, or sweeteners. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The conjugates herein may have ionizable groups so as to be capable of preparation as pharmaceutically acceptable salts. These salts may be acid addition salts involving inorganic or organic acids or the salts may, in the case of acidic forms of the conjugates herein be prepared from inorganic or organic bases. Frequently, the conjugates are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include, but are not limited to, sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Depending on the route of administration and the dosage, a conjugate herein or a pharmaceutical composition thereof used in the methods described herein will be formulated into suitable pharmaceutical compositions to permit facile delivery. A conjugate (e.g., a conjugate of any one of formulas (D-I)-(D-VIII)) or a pharmaceutical composition thereof may be formulated to be administered intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularily, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). Depending on the route of administration, a conjugate herein or a pharmaceutical composition thereof may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

A conjugate described herein may be formulated in a variety of ways that are known in the art. For use as treatment of human and animal subjects, a conjugate described herein can be formulated as pharmaceutical or veterinary compositions. Depending on the subject (e.g., a human) to be treated, the mode of administration, and the type of treatment desired, e.g., prophylaxis or therapy, a conjugate described herein is formulated in ways consonant with these parameters. A summary of such techniques is found in Remington: The Science and Practice of Pharmacy, 22nd Edition, Lippincott Williams & Wilkins (2012); and Encyclopedia of Pharmaceutical Technology, 4th Edition, J. Swarbrick and J. C. Boylan, Marcel Dekker, New York (2013), each of which is incorporated herein by reference.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, and preservatives. The conjugates can be administered also in liposomal compositions or as microemulsions. Systemic administration may also include relatively noninvasive methods such as the use of suppositories, transdermal patches, transmucosal delivery and intranasal administration. Oral administration is also suitable for conjugates herein. Suitable forms include syrups, capsules, and tablets, as is understood in the art.

The pharmaceutical compositions can be administered parenterally in the form of an injectable formulation. Pharmaceutical compositions for injection can be formulated using a sterile solution or any pharmaceutically acceptable liquid as a vehicle. Formulations may be prepared as solid forms suitable for solution or suspension in liquid prior to injection or as emulsions. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Such injectable compositions may also contain amounts of nontoxic auxiliary substances such as wetting or emulsifying agents, pH buffering agents, such as sodium acetate and sorbitan monolaurate. Formulation methods are known in the art, see e.g., Pharmaceutical Preformulation and Formulation, 2nd Edition, M. Gibson, Taylor & Francis Group, CRC Press (2009).

The pharmaceutical compositions can be prepared in the form of an oral formulation. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Other pharmaceutically acceptable excipients for oral formulations include, but are not limited to, colorants, flavoring agents, plasticizers, humectants, and buffering agents. Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release of a conjugate described herein (e.g., a conjugate of any one of formulas (D-I)-(D-VIII)) or a pharmaceutical composition thereof can be achieved by appropriate coating of a tablet, capsule, pellet, or granulate formulation of the conjugate, or by incorporating the conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The pharmaceutical composition may be formed in a unit dose form as needed. The amount of active component, e.g., a conjugate described herein (e.g., a conjugate of any one of formulas (D-I)-(D-VIII)), included in the pharmaceutical compositions are such that a suitable dose within the designated range is provided (e.g., a dose within the range of 0.01-100 mg/kg of body weight).

### VIII. Routes of Administration and Dosages

In any of the methods described herein, conjugates herein may be administered by any appropriate route for treating or protecting against a viral infection (e.g., an influenza infection), or for preventing, stabilizing, or inhibiting the proliferation or spread of a virus (e.g., an influenza virus). Conjugates described herein may be administered to humans, domestic pets, livestock, or other animals with a pharmaceutically acceptable diluent, carrier, or excipient. In some embodiments, administering comprises administration of any of the conjugates described herein (e.g., conjugates of any one of formulas (D-I)-(D-VIII)) or compositions intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). In some embodiments, if an antiviral agent is also administered in addition to a conjugate described herein, the antiviral agent or a pharmaceutical composition thereof may also be administered in any of the routes of administration described herein.

The dosage of a conjugate described herein (e.g., a conjugate of any one of formulas (D-I)-(D-VIII)) or pharmaceutical compositions thereof depends on factors including the route of administration, the disease to be treated (e.g., the extent and/or condition of the viral infection), and physical characteristics, e.g., age, weight, general health, of the subject. Typically, the amount of the conjugate or the pharmaceutical composition thereof contained within a single dose may be an amount that effectively prevents, delays, or treats the viral infection without inducing significant toxicity. A pharmaceutical composition may include a dosage of a conjugate described herein ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 1 to about 30 mg/kg. In some embodiments, when a conjugate described herein (e.g., a conjugate of any one of formulas (D-I)-(D-VIII)) and an antiviral agent or antiviral vaccine are administered in combination (e.g., substantially simultaneously in the same or separate pharmaceutical compositions, or separately in the same treatment regimen), the dosage needed of the conjugate described herein may be lower than the dosage needed of the conjugate if the conjugate was used alone in a treatment regimen.

A conjugate described herein (e.g., a conjugate of any one of formulas (D-I)-(D-VIII)) or a pharmaceutical composition thereof may be administered to a subject in need thereof, for example, one or more times (e.g., 1-10 times or more; 1, 2, 3, 4, 5, 6, 7, 8, 9, or 10 times) daily, weekly, monthly, biannually, annually, or as medically necessary. Dosages may be provided in either a single or multiple dosage regimens. The timing between administrations may decrease as the medical condition improves or increase as the health of the patient declines. The dosage and frequency of administration may be adapted by the physician in accordance with conventional factors such as the extent of the infection and different parameters of the subject.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Expression of an Fc Domain Having a K246 Substitution Mutation

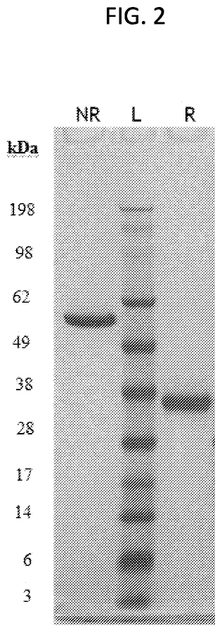

Reverse translations of the amino acids comprising an Fc domain monomer having a K246S substitution mutation (SEQ ID NO: 12) were synthesized by solid-phase synthesis and the oligonucleotide templates were cloned into pcDNA3.1(+) at the cloning sites HindIII and EcoRI (GenScript's GenSmart Gene Synthesis service). The construct included a signal sequence derived from the mouse Ig VH chain which is cleaved following expression. The pcDNA3.1(+) plasmids were transformed into Top10 E. coli cells (Invitrogen). DNA was amplified, extracted, and purified using the PURELINK® HiPure Plasmid Filter Maxiprep Kit (Invitrogen). The plasmid DNA is delivered, using the ExpiFectamine™ CHO Transfection Kit (Gibco), into ExpiCHO-S cells per the manufacturer's “maximum yield” protocol. Cells were centrifuged, filtered, and the supernatants were purified using MabSelect PrismA Resin (Cytiva). The purified molecule was analyzed using 4-12% Bis Tris SDS PAGE gels by loading 2 μg of each molecule into the gel, and staining using instant Blue staining. The gel included a molecular weight ladder with the indicated molecular weight standards. FIG. 2 shows non-reducing and reducing SDS-PAGE of the Fc domain formed from Fc domain monomers having the sequence of SEQ ID NO: 12. Reduced and non-reduced lanes are denoted by “R” and “NR”.

### Example 2. General Procedure for Synthesis of an Fc Conjugated to an Azido-Containing Linker at One or More Lysine Residues

Preparation of PEG4-azido NHS ester solution (0.050 M) in DMF/PBS: 16.75 mg of PEG4-azido NHS ester was dissolved in 0.100 mL of DMF at 0° C. and diluted to 0.837 mL by adding PBS 1× buffer at 0° C. This solution was used for preparing other PEG4-azido Fc with a variety of DAR values by adjusting the equivalents of this PEG4-azido NHS ester PBS solution.

Pretreatment of h-IgG1 Fc (107.2 mg in 8.800 mL of pH 7.4 PBS, MW˜57891 Da, 1.852 μmol): The Fc solution was transferred into four centrifugal concentrators (30,000 MWCO, 15 mL) and diluted to 15 mL with PBS×1 buffer and concentrated to a volume of ˜1.5 mL. The residue was diluted 1:10 in PBS pH 7.4, and concentrated again. This wash procedure was repeated for total of four times followed by dilution to 8.80 mL.

Preparation of PEG4-azido Fc: 0.050M PEG4-azidoNHS ester PBS buffer solution (0.593 mL, 29.6 μmol, 16 equivalents) was added to above solution of h-IgG1 Fc and the mixture was shaken rotated for 2 hours at ambient temperature. The solution was concentrated by using four centrifugal concentrators (30,000 MWCO, 15 mL) to a volume of ˜1.5 mL. The crude mixture was diluted 1:10 in PBS pH 7.4, and concentrated again. This wash procedure was repeated for total of three times. The concentrated Fc-PEG4-azide was diluted to 8.80 mL with pH 7.4 PBS buffer and ready for Click conjugation. The purified material was quantified using a NANODROP™ UV visible spectrophotometer (using a calculated extinction coefficient based on the amino acid sequence of h-IgG1). Yield was quantitative after purification.

### Example 3. General Procedure for Synthesis of a Conjugate Including an Fc Conjugated to One or More Small Molecules

Preparation of the Click reagent solution: 0.0050M CuSO₄ in PBS buffer solution: 10.0 mg CuSO₄ was dissolved in 12.53 mL PBS, then took 5.00 mL this CuSO₄ solution and added 43.1 mg BTTAA (CAS #1334179-85-9) and 247.5 mg sodium ascorbate to give the Click reagent solution (0.0050M CuSO4, 0.020M BTTAA and 0.25M sodium ascorbate).

To a solution of azido functionalized Fc (Example 1; 65.5 mg, 10.0 mL, 1.13 μmol) in a 15 mL centrifuge tube was added to an alkyne derivatized small molecule viral inhibitor (22.7 mg, 15.2 μmol, 3.0 equivalents per each azido of the Fc). After gently agitating to dissolve all solids, the mixture was treated with the Click reagent solution (1.80 mL). The resulting mixture was gently rotated for 12 hours at ambient temperature. It was purified by affinity chromatography over a protein A column, followed size exclusion chromatography.

### Example 4. Procedures for Making Intermediates (Ints) Including a Dimer of Zanamivir or an Analog Thereof

Exemplary methods for the production of Ints including two zanamivirs or analogs thereof joined by a trimeric linker, e.g., Ints described in Table 1a, are provided in WO 2021/046549, which is incorporated herein in its entirety. For example, methods of making Ints of Table 1a are provided in Examples 2-6, 11-15, 17, 19, 21, 22, 31, 45, 46, 49-52, 54, 56-58, 60, 73, 75, 77, 79, 81, 99-107, 109, 110, 112, 114-123, 138, 140, 143-146, 195, 197 of WO 2021/046549.

### Example 5. Procedures for Making Conjugates Including an Fc Domain Conjugated to One or More Ints

Exemplary methods for the production of conjugates of the present disclosure are provided in WO 2021/046549. For example, methods of making conjugates including an Fc domain conjugated to one or more dimers of zanamivir or an analog thereof are provided in Examples 7, 9, 10, 16, 18, 20, 32, 47, 50, 53, 55, 59, 61-63, 70-72, 74, 76, 78, 80, 82, 84-86, 88-92, 108, 111, 113, 124-129, 137, 139, 142, 148-151, 165, 196, 198, 207 of WO 2021/046549.

### Example 5. Characterization and Use of Conjugates

Exemplary methods for characterizing and using conjugates of the present disclosure, e.g., in the treatment of a viral infection such as an influenza infection, are provided in WO 2021/046549. For example, the characterization and use of conjugates including an Fc domain conjugated to one or more dimers of zanamivir or an analog thereof for viral inhibition are provided in Examples 23-30, 33-44, 48, 64-69, 83, 87, 93-98, 130-136, 141, 147, 152-155, 157-194, 199-206, 208-220 of WO 2021/046549.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. All publications, patents, and patent applications mentioned in the above specification are hereby incorporated by reference to the same extent as if each individual publication, patent or patent application was specifically and individually indicated to be incorporated by reference in its entirety.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

1. A conjugate described by formula (D-I): 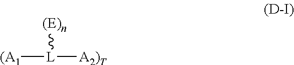
wherein A₁ and A₂ are zanamivir;
L is a inker;
E is an Fc domain monomer comprising an amino acid substitution at position 246, wherein the amino acid at position 246 is not a lysine, and wherein numbering is according to the EU index as in Kabat;
n is 1 or 2;
T is an integer from 1 to 20; and
the squiggly line indicates that L is covalently attached to E,
or a pharmaceutically acceptable salt thereof.

2-5. (canceled)

6. The conjugate of claim 1, wherein the conjugate is described by formula (D-II-6): 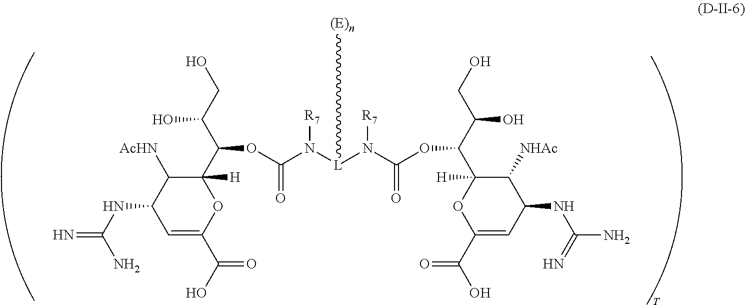
wherein R₇ is selected from H, C1-C20 alkyl, C3-C20 cycloalkyl, C3-C20 heterocycloalkyl; C5-C15 aryl, and C2-C15 heteroaryl;
or a pharmaceutically acceptable salt thereof.

7-8. (canceled)

9. The conjugate of claim 6, wherein the conjugate is described by formula (D-II-7): 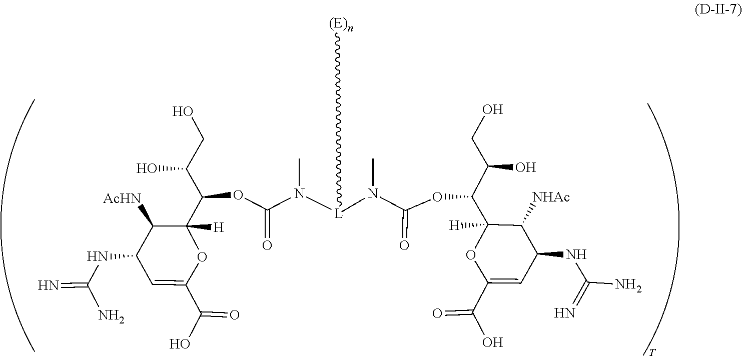
or a pharmaceutically acceptable salt thereof.

10. The conjugate of claim 9, wherein the conjugate is described by formula (D-II-8): 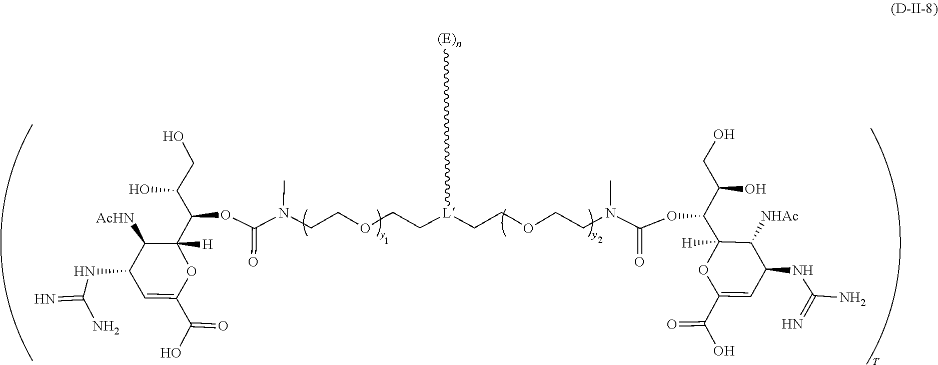
wherein L′ is the remainder of L, and
y₁ and y₂ are each independently an integer from 1-20,
or a pharmaceutically acceptable salt thereof.

11. The conjugate of claim 10, wherein the conjugate has the structure of: 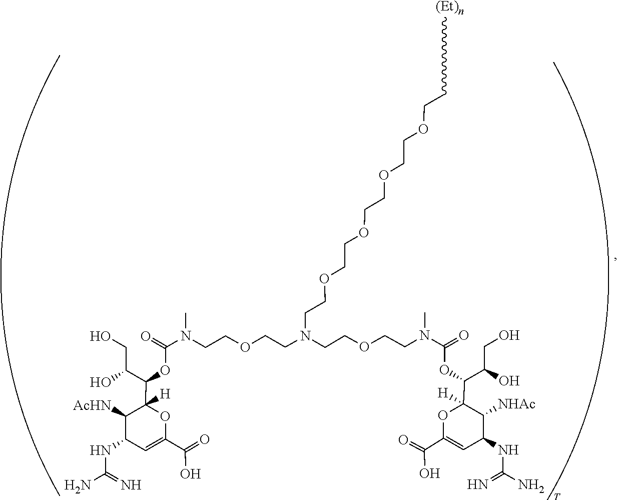
or a pharmaceutically acceptable salt thereof.

12. The conjugate of claim 11, wherein the conjugate is described by 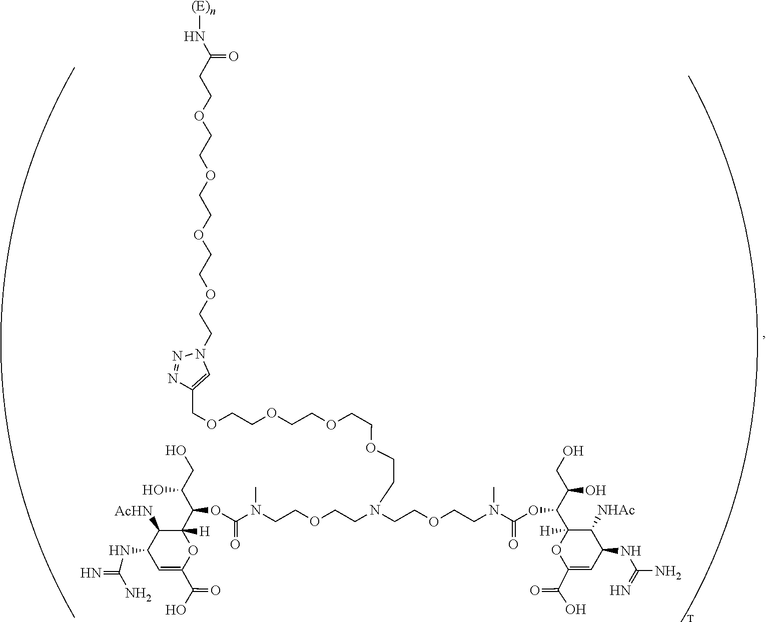
or a pharmaceutically acceptable salt thereof.

13-17. (canceled)

18. The conjugate of claim 1, wherein the Fc domain monomer further comprises amino acid substitutions at positions (i) 252, 254, and 256, (ii) 309, 311, and 434, or (iii) 428 and 434, and wherein the substitution at position 252 is a tyrosine, the substitution at position at position 254 is a threonine, the substitution at position 256 is a glutamic acid, the substitution at position 309 is an aspartic acid, the substitution at position at position 311 is a histidine, the substitution at positions 428 is a leucine, and the substitution at position 434 is a serine.

19. The conjugate of claim 1, wherein the Fc domain monomer comprises:
an amino acid that is not lysine at position 246;
a tyrosine at position 252;
a threonine at position 254; and
a glutamic acid at position 256.

20. The conjugate of claim 1, wherein the Fc domain monomer comprises:
an amino acid that is not lysine at position 246;
an aspartic acid at position 309;
a histidine at position 311; and
a serine at position 434.

21. The conjugate of claim 1, wherein the Fc domain monomer comprises:
an amino acid that is not lysine at position 246;
a leucine at position 428; and
a serine at position 434.

22. The conjugate of claim 1, wherein the amino acid at position 246 is selected from serine, glycine, alanine, threonine, asparagine, glutamine, arginine, histidine, glutamic acid, or aspartic acid.

23. (canceled)

24. The conjugate of claim 1, wherein the Fc domain monomer further comprises a serine substitution at position 220.

25-27. (canceled)

28. The conjugate of claim 1, wherein the Fc domain monomer comprises the amino acid sequence of any one of SEQ ID NOs: 1-29.

29. The conjugate of claim 1, wherein the squiggly line connected to E indicates that the L of each A₁-L-A₂ is covalently attached to a nitrogen atom of a solvent-exposed lysine of E.

30. The conjugate of claim 1, wherein n is 2, and each E dimerizes to form an Fc domain.

31. The conjugate of claim 1, wherein T is 1, 2, 3, 4, 5, 6, 7, 8, 9, or 10.

32. A population of conjugates of claim 1, wherein the average value of T is 1 to 10.

33. A pharmaceutical composition comprising the conjugate or the population of conjugates of claim 1, or a pharmaceutically acceptable salt thereof, and a pharmaceutically acceptable excipient.

34. A method for the treatment of a subject having an influenza viral infection or presumed to have an influenza viral infection, the method comprising administering to the subject an effective amount of the conjugate, the population of conjugates, or the pharmaceutical composition of claim 1.

35. A method for the prophylactic treatment of an influenza viral infection in a subject in need thereof, the method comprising administering to the subject an effective amount of the conjugate, the population of conjugates, or the pharmaceutical composition of claim 1.

36-37. (canceled)


SEQUENCE LISTING (29 sequences)

SEQ ID NO: 1 - protein, 247 aa, Artificial Sequence
  Synthetic Construct
  MISC_FEATURE (1)..(1): Xaa is Asn or absent
  MISC_FEATURE (20)..(20): Xaa is Cys or Ser
  MISC_FEATURE (46)..(46): Xaa is Ser, Gly, Ala, Thr, Asn, Gln, Arg, His, Glu, or Asp
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  MISC_FEATURE (97)..(97): Xaa is Asn or Ala
  MISC_FEATURE (109)..(109): Xaa is Leu or Asp
  MISC_FEATURE (111)..(111): Xaa is Gln or His
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
  MISC_FEATURE (247)..(247): Xaa is Lys or absent
XVNHKPSNTKVDKKVEPKSXDKTHTCPPCPAPELLGGPSVFLFPPXPKDTLXIXRXPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 2 - protein, 247 aa, Artificial Sequence
  Synthetic Construct
  MISC_FEATURE (1)..(1): Xaa is Asn or absent
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
  MISC_FEATURE (247)..(247): Xaa is Lys or absent
XVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 3 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 246 aa, Artificial Sequence
  Synthetic Construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 246 aa, Artificial Sequence
  Synthetic Construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 247 aa, Artificial Sequence
  Synthetic Construct
  MISC_FEATURE (1)..(1): Xaa is Asn or absent
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
  MISC_FEATURE (247)..(247): Xaa is Lys or absent
XVNHKPSNTKVDKKVEPKSSDKTHTCPPCPAPELLGGPSVFLFPPXPKDTLYITREPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 10 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLYITREPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 246 aa, Artificial Sequence
  Synthetic Construct
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 246 aa, Artificial Sequence
  Synthetic Construct
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 247 aa, Artificial Sequence
  Synthetic Construct
  MISC_FEATURE (1)..(1): Xaa is Asn or absent
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
  MISC_FEATURE (247)..(247): Xaa is Lys or absent
XVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGX

SEQ ID NO: 17 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 18 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 19 - protein, 246 aa, Artificial Sequence
  Synthetic Construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 20 - protein, 246 aa, Artificial Sequence
  Synthetic Construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 21 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 22 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 23 - protein, 247 aa, Artificial Sequence
  Synthetic Construct
  MISC_FEATURE (1)..(1): Xaa is Asn or absent
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
  MISC_FEATURE (247)..(247): Xaa is Lys or absent
XVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPGX

SEQ ID NO: 24 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 25 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 26 - protein, 246 aa, Artificial Sequence
  Synthetic Construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 27 - protein, 246 aa, Artificial Sequence
  Synthetic Construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 28 - protein, 246 aa, Artificial Sequence
  Synthetic Construct
  MISC_FEATURE (46)..(46): Xaa is Ser, Gly, Ala, Thr, Asn, Gln, Arg, His, Glu, or Asp
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 29 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG